(19) United States
(12) Patent Application Publication
(10) Pub. No.: US 2022/0356497 A1
(43) Pub. Date: Nov. 10, 2022

(54) COMPOSITIONS AND METHODS FOR SYNTHESIS OF TERPENOIDS

(71) Applicant: Zymergen Inc., Emeryville, CA (US)

(72) Inventors: Stefan DE KOK, Emeryville, CA (US); Warren LAU, Emeryville, CA (US); Fern MCSORLEY, Oakland, CA (US); Hermann-Josef MEYER, El Sobrante, CA (US); Zach SERBER, Sausalito, CA (US); Grayson WAWRZYN, Oakland, CA (US)

(21) Appl. No.: 17/622,619

(22) PCT Filed: Jun. 26, 2020
(86) PCT No.: PCT/US20/39959

§ 371 Date: Dec. 23, 2021

Related U.S. Application Data
(60) Provisional application No. 62/867,199, filed on Jun. 26, 2019.

(51) Int. Cl. C12P 17/06; C12P 5/00; C12N 1/18; C12N 15/81; C12N 15/52

(52) U.S. Cl. CPC C12P 17/06; C12P 5/007; C12N 1/18; C12N 15/81; C12N 15/52; C12Y 101/01324; C12Y 114/13169; C12Y 103/01; C12Y 101/01; C12N 2800/102

(57) ABSTRACT

The disclosure relates to the biosynthesis of terpenoids, such as, for example, geraniol and derivatives thereof, using genetic engineering. In particular, the disclosure relates to the biosynthesis of nepetalactol, nepetalactone, dihydronepetalactone, and derivatives thereof. The disclosure provides recombinant cells genetically engineered to produce high levels of nepetalactol, nepetalactone and/or dihydronepetalactone. The disclosure also provides methods of producing nepetalactol, nepetalactone and dihydronepetalactone using cell-based systems as well as cell-free systems.

### CROSS REFERENCE TO RELATED APPLICATIONS

The present application claims the benefit of priority to U.S. Provisional Application No. 62/867,199, filed on Jun. 26, 2019, the contents of which are hereby incorporated by reference in their entirety.

### TECHNICAL FIELD

The present disclosure is generally related to the biosynthesis of terpenoids, such as, for example, geraniol and derivatives thereof produced in microorganisms, using genetic engineering.

### INCORPORATION BY REFERENCE OF SEQUENCE LISTING

The contents of the text file named “ZYMR_041_01WO_SeqList_ST25.txt”, which was created on Jun. 26, 2020 and is 5.53 megabytes in size, are hereby incorporated by reference in its entirety.

### BACKGROUND

Dihydronepetalactone is an effective active ingredient for insect repellents. Current ingredients used for insect repellents such as N, N-Diethyl-meta-toluamide (DEET) pose health concerns, while other natural alternatives only offer short-term protection. Dihydronepetalactone and its direct precursor nepetalactone are derived primarily from Nepeta spp., but are produced at low levels with the latter being more abundant. Yields are subject to environmental factors, such as climate and pests, creating an unreliable supply for large-scale commercial use. Chemical synthesis is feasible, but not economical.

Thus far, attempts to synthesize nepetalactone and its derivatives using biosynthetic approaches have been met with several hurdles. First, the level of production of nepetalactone and its derivatives using biosynthetic approaches has been low. Second, it has not been possible thus far to produce nepetalactone and its derivatives in vivo using glucose as a precursor at industrial-scales or even lower levels. Third, the toxicity of monoterpenes presents additional challenges for the industrial-scale biosynthesis of nepetalactone and its derivatives in host cells. Finally, fermentation processes that would allow for rapid growth of host cells are needed to enable high-level production of nepetalactone and its derivatives. Therefore, there remains a pressing need to develop biosynthetic approaches that are capable of generating large quantities of nepetalactone and its derivatives in a commercially viable manner.

### SUMMARY

The disclosure provides recombinant microbial cell capable of producing nepetalactol from glucose without additional precursor supplementation.

The disclosure further provides methods for the production of nepetalactol from a glucose substrate, said method comprising: (a) providing any one of the recombinant microbial cells of this disclosure; and (b) cultivating the recombinant microbial cell in a suitable cultivation medium comprising glucose, thereby producing nepetalactol. The disclosure provides methods for the production of nepetalactone from a glucose substrate, said method comprising: (a) providing any one of the recombinant microbial cells of this disclosure; and (b) cultivating the recombinant microbial cell in a suitable cultivation medium comprising glucose, thereby producing nepetalactone. The disclosure also provides methods for the production of dihydronepetalactone from a glucose substrate, said method comprising: (a) providing a recombinant microbial cell according to any one of the recombinant microbial cells of this disclosure; and (b) cultivating the recombinant microbial cell in a suitable cultivation medium comprising glucose, thereby producing dihydronepetalactone.

The disclosure provides recombinant microbial cells capable of producing nepetalactone, wherein said recombinant microbial cell comprises a nucleic acid encoding for a heterologous nepetalactol oxidoreductase (NOR) enzyme that catalyzes the reduction of nepetalactol to nepetalactone. The disclosure provides methods for the production of nepetalactone, said method comprising: (a) providing any one of the recombinant microbial cells disclosed herein; (b) cultivating the recombinant microbial cell in a suitable cultivation medium; and (c) contacting the recombinant microbial cell with nepetalactol substrate to form nepetalactone.

The disclosure provides recombinant microbial cells capable of producing dihydronepetalactone, wherein said recombinant microbial cell comprises a nucleic acid encoding for a heterologous dihydronepetalactone dehydrogenase (DND) enzyme capable of converting nepetalactone to dihydronepetalactone. The disclosure provides method for the production of dihydronepetalactone, said method comprising: (a) providing any one of the recombinant microbial cells disclosed herein; (b) cultivating the recombinant microbial cell in a suitable cultivation medium; and (c) contacting the recombinant microbial cell with nepetalactone substrate to form dihydronepetalactone.

The disclosure provides a fermentation process for producing a desired product selected from the group consisting of nepetalactol, nepetalactone, and dihydronepetalactone, wherein said fermentation process utilizes a composition comprising a first phase and a second phase, wherein the first phase is an aqueous phase comprising a microbial cell capable of synthesizing the product, and wherein the second phase comprises an organic solvent and at least a portion of the desired product synthesized by the microbial cell. The disclosure further provides methods of producing a desired product selected from the group consisting of nepetalactol, nepetalactone, and dihydronepetalactone, said method comprising the steps of: a) growing an aqueous culture of microbial cells configured to produce the desired product in response to a chemical inducer, or absence of a chemical repressor; b) contacting the microbial cells with the chemical inducer or lack thereof a chemical repressor; and c) adding an organic solvent to the induced/derepressed aqueous culture, said organic solvent having low solubility with the aqueous culture, wherein product secreted by the microbial cells accumulates in the organic solvent, thereby reducing contact of the product with the microbial cells.

### BRIEF DESCRIPTION OF THE FIGURES

FIG. 1A shows a schematic of the mevalonate pathway, comprising the conversion of acetyl CoA to IPP/DMAPP through a series of enzymatically catalyzed steps.

FIGS. 1B and 1C show the nepetalactone biosynthetic pathway, comprising the conversion of IPP/DMAPP to 8-hydroxygeraniol (FIG. 1B) and from 8-hydroxygeraniol to nepetalactone through a series of enzymatically catalyzed steps (FIG. 1C). FIG. 1C also shows the conversion of nepetalactone to dihydronepetalactone by dihydronepetalactone dehydrogenase (DND).

FIGS. 2A-B show the conversion of nepetalactol to nepetalactone by candidate nepetalactol oxidoreductases (NORs). See Example 1. FIG. 2A shows nepetalactone produced in the presence of NAD+ (nicotinamide adenine dinucleotide, NAD) and/or NADP+ (nicotinamide adenine dinucleotide phosphate, NADP) in clarified cell lysates from cells expressing various candidate NORs. FIG. 2B shows the concentration of residual nepetalactol after reaction. The results show that three candidate NORs (NcatNOR15, NcatNOR21, and NcatNOR34) can convert nepetalactol to nepetalactone. (In FIGS. 2A-B, “uM” is used to refer to “μM.”)

FIG. 3 shows the in vitro conversion of 8-oxogeranial to nepetalactol in the presence of iridoid synthase (ISY, IS), NADH, and NADPH. The symbols for “IS reaction no cofactors” and “IS reaction no substrate” overlap for N mussinii. See Example 3.

FIG. 4 shows the in vitro conversion of 8-oxogeranial in the presence of iridoid synthase (ISY, IS), nepetalactol synthase (NEPS) and NADPH. Catharanthus roseus IS del22 is truncated at the N-terminus by 22 amino acids. (In FIG. 4, “ug” is used to refer to “μg.”). See Example 4.

FIG. 5 shows the in vitro conversion of 8-hydroxygeraniol to nepetalactol by 8HGOs coupled to Nepeta mussinii iridoid synthase (ISY) and C. roseus nepetalactol synthase (NEPS 1) in the presence of NAD+ and NADPH. The nepetalactol produced is cis,trans-nepetalactol, as determined by liquid chromatography-mass spectrometry (no other stereoisomers were detected by this method). (In FIG. 5, “ug” is used to refer to “μg.”). See Example 5.

FIG. 6 shows the titers of nepetalactol and nepetalactone in engineered strains compared to wild-type and a non-inoculated control. Geraniol or 8-hydroxygeraniol were provided as substrate feeds at a final concentration of 500 mg/L. Only the cis,trans-nepetalactone isomer was produced. Genotypes of tested strains are described in Table 10 of this document.

FIG. 7 shows the production of nepetalactone from nepetalactol in engineered Saccharomyces cerevisiae strains expressing NOR candidates from a 2p plasmid (pESCURA). See Example 6.

FIG. 8 shows an alignment of the amino acid sequences of nepetalactol cyclases (NEPSs) comprising the amino acid sequences of SEQ ID NO. 730-733.

FIG. 9 shows the results of a MUSCLE alignment of NOR enzymes comprising the amino acid sequences of SEQ ID NO 605, 718, 728, 1642-1644 and 520.

FIG. 10 depicts a distribution of three geraniol-derived terpenoids, geranic acid, nepetalactol, and nepetalactone from strains 7000445150 (see Example 9) and strains 7000552966 & 7000553262 (see Example 10). The strains were grown using the biphasic fermentation process disclosed herein. The first strain, 7000445150, accumulates >1.5 g/L of geranic acid, >0.5 g/L nepetalactone, and <0.1 g/L nepetalactol. After a subsequent round of engineering, the two additional strains, 7000552966 & 7000553262, show <0.25 g/L of geranic acid, and >1 g/L of both nepetalactol and nepetalactone. Data shown here are the average of at least four replicates, with error bars indicating a 95% confidence interval.

FIG. 11 shows a schematic of the DXP/MEP pathway, comprising the conversion of pyruvate to IPP/DMAPP through a series of enzymatically catalyzed steps.

FIG. 12A shows the titers of geranic acid, nepetalactol and nepetalactone, and the combined titer of nepetalactol and nepetalactone in engineered strains compared to their parent strain (Parent). Gene deletions in the parent strain are indicated by ‘d’ in front of the gene name. Promoter insertions in the parent strain are indicated by ‘<’. For example, pTDH3<SWT21 indicates an insertion of the TDH3 promoter between the native SWT21 promoter and the coding sequence. FIG. 12B shows the titers of geranic acid, nepetalactol, nepetalactone, and the combined titer of nepetalactol and nepetalactone in engineered strains compared to a parent strain (Parent; parent strain is different from that shown in FIG. 12A). Engineered strains each contain an inserted gene cassette at a neutral locus. For example, ihol1: pGAL7<NCP1, indicates that a gene cassette with the GAL7 promoter driving the expression of the gene NCP1 was inserted at the ihol1 site, an intergenic region between HOL1 and a proximal gene.

### DETAILED DESCRIPTION

The disclosure provides recombinant microbial cells and methods for producing high levels of nepetalactol and/or nepetalactone through (a) extensive genetic manipulations strategically directed at increasing the flux to key metabolic nodes such as, acetoacetyl CoA and geranyl pyrophosphate (GPP); (b) reducing negative feedback and unwanted side products within the biosynthetic pathway; and (c) addition of heterologous enzymes capable of catalyzing multiple steps in the nepetalactol/nepetalactone synthesis pathway. Further, the disclosure also provides methods of converting nepetalactone to dihydronepetalactone based on the discovery of dihydronepetalactone dehydrogenase (DND) disclosed herein.

Additionally, the disclosure provides genetic solutions for dynamically controlling the expression of various heterologous enzymes in the recombinant microbial cells disclosed herein. These genetic switches provide tight control of the nepetalactol/nepetalactone/dihydronepetalactone synthesis pathway, allowing for induction under conditions that mitigate toxicity and are economical. The disclosure also provides a phased-fermentation process that allows for growth of the recombinant microbial cell of this disclosure to high cell density and provides conditions amenable for high-level production of nepetalactol/nepetalactone/dihydronepetalactone, while mitigating the toxicity of product accumulation.

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or processes known to those skilled in the art, and so forth.

As used herein, the term “about” or “approximately” when preceding a numerical value indicates the value plus or minus a range of 10%, unless otherwise stated or otherwise evident by the context (except where such a range would exceed 100% of a possible value, or fall below 0% of a possible value, such as less than 0 expression, or more than 100% of available protein).

As used herein the terms “cellular organism” “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure refers to the “microorganisms” or “cellular organisms” or “microbes” of lists/tables and figures present in the disclosure. This characterization can refer to not only the identified taxonomic genera of the tables and figures, but also the identified taxonomic species, as well as the various novel and newly identified or designed strains of any organism in said tables or figures. The same characterization holds true for the recitation of these terms in other parts of the Specification, including the Examples.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

The terms “genetically modified host cell,” “recombinant host cell,” and “recombinant strain” are used interchangeably herein and refer to host cells that have been genetically modified by the cloning and transformation methods of the present disclosure. Thus, the terms include a host cell (e.g., bacteria, yeast cell, fungal cell, CHO, human cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring organism from which it was derived. It is understood that in some embodiments, the terms refer not only to the particular recombinant host cell in question, but also to the progeny or potential progeny of such a host cell.

The term “wild type”, abbreviated as “WT”, is a term of the art understood by skilled persons and means the typical form of an organism, strain, gene, protein, or characteristic as it occurs in nature as distinguished from mutant or variant forms. For example, a WT protein is the typical form of that protein as it occurs in nature. As another example, the term “wild-type microorganism” or “wild-type host cell” describes a cell that occurs in nature, i.e. a cell that has not been genetically modified.

The term “genetically engineered” may refer to any manipulation of a host cell's genome (e.g. by insertion, deletion, mutation, or replacement of nucleic acids). In some embodiments, the manipulation comprises rearrangement of nucleic acids such that a polynucleotide is moved from its native location to another non-native location.

The term “control” or “control host cell” refers to an appropriate comparator host cell for determining the effect of a genetic modification or experimental treatment. In some embodiments, the control host cell is a wild type cell. In other embodiments, a control host cell is genetically identical to the genetically modified host cell, save for the genetic modification(s) differentiating the treatment host cell.

As used herein, the term “allele(s)” means any of one or more alternative forms of a gene, all of which alleles relate to at least one trait or characteristic. In a diploid cell, the two alleles of a given gene occupy corresponding loci on a pair of homologous chromosomes.

As used herein, the term “locus” (loci plural) means a specific place or places or a site on a chromosome where for example a gene or genetic marker is found.

As used herein, the term “genetically linked” refers to two or more traits that are co-inherited at a high rate during breeding such that they are difficult to separate through crossing.

A “recombination” or “recombination event” as used herein refers to a chromosomal crossing over or independent assortment.

As used herein, the term “phenotype” refers to the observable characteristics of an individual cell, cell culture, organism, or group of organisms which results from the interaction between that individual's genetic makeup (i.e., genotype) and the environment.

As used herein, the term “chimeric” when describing a nucleic acid sequence or a protein sequence refers to a nucleic acid, or a protein sequence, that links at least two heterologous polynucleotides, or two heterologous polypeptides, into a single macromolecule, or that re-arranges one or more elements of at least one natural nucleic acid or protein sequence. For example, the term “chimeric” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “endogenous” or “endogenous gene,” refers to the naturally occurring gene, in the location in which it is naturally found within the host cell genome. In the context of the present disclosure, operably linking a heterologous promoter to an endogenous gene means genetically inserting a heterologous promoter sequence in front of an existing gene, in the location where that gene is naturally present. An endogenous gene as described herein can include alleles of naturally occurring genes that have been mutated according to any of the methods of the present disclosure.

As used herein, the term “exogenous” is used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458.

For PCR amplification of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3^{rd} ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

As used herein, “promoter” refers to a DNA sequence capable of controlling the expression of a coding sequence or functional RNA. In some embodiments, the promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

“Operably linked” means in this context, the sequential arrangement of the promoter polynucleotide according to the disclosure with a further oligo- or polynucleotide, resulting in transcription of said further polynucleotide.

The term “product of interest” or “biomolecule” as used herein refers to any product produced by microbes from feedstock. In some cases, the product of interest may be nepetalactol, nepetalactone, and/or dihydronepetalactone.

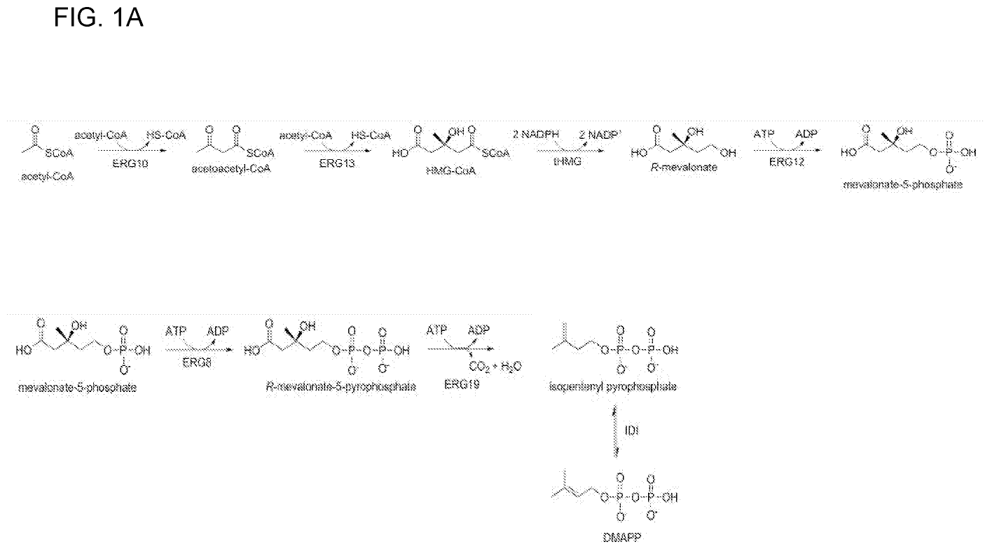

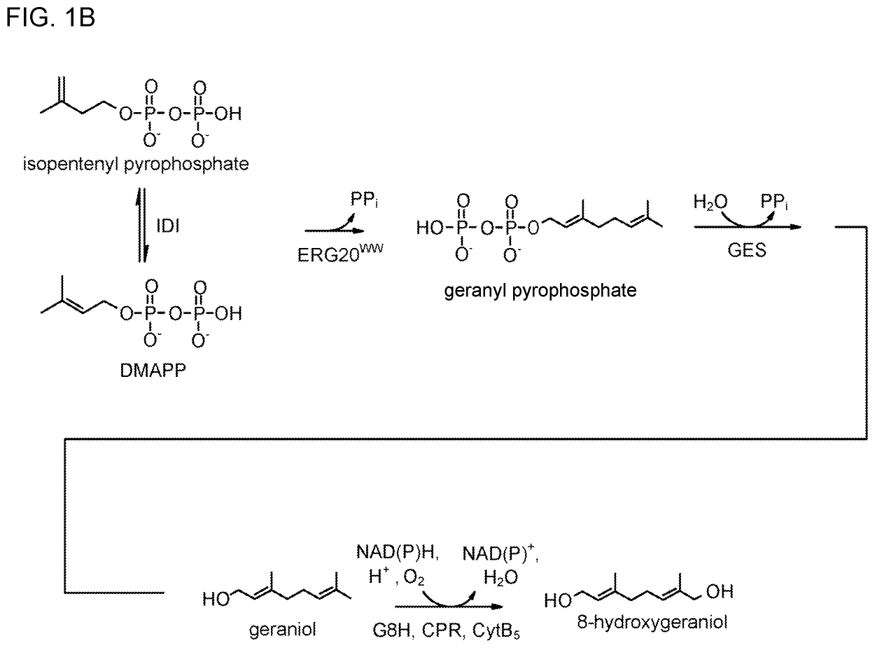

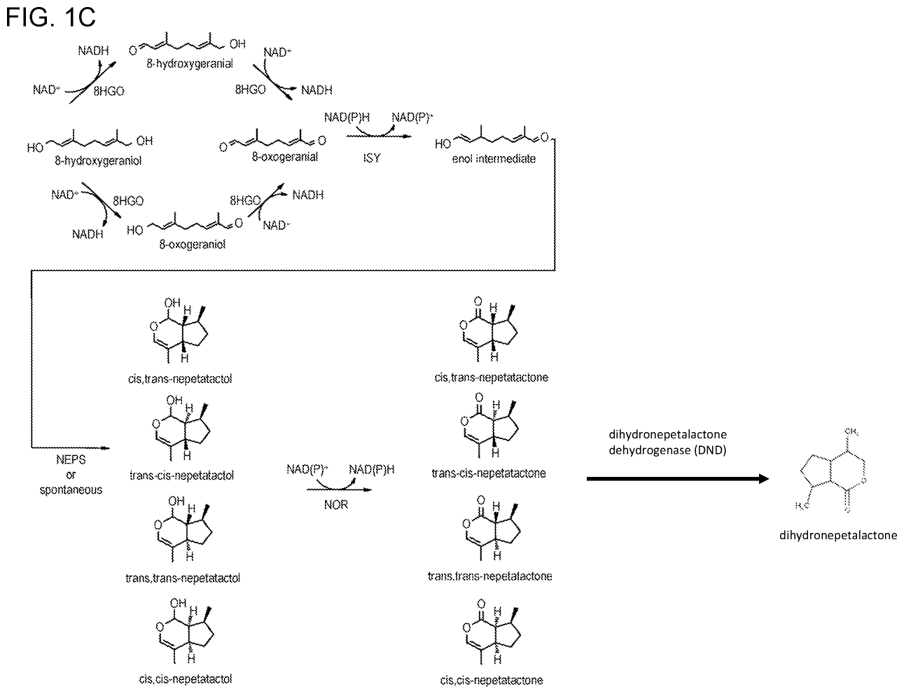

As used herein, the term “precursor” refers to a molecule or a chemical compound that is transformed into another molecule or chemical compound in the biosynthetic pathway that leads to the generation of the “product of interest”. For example, a “nepetalactol precursor” refers to a compound that precedes nepetalactol in the biosynthetic pathway that leads to the generation of nepetalactol, such as those depicted in FIGS. 1A, 1B and 1C; a “nepetalactone precursor” refers to a compound that precedes nepetalactone in the biosynthetic pathway that leads to the generation of nepetalactone, such as those depicted in FIGS. 1A, 1B and IC; and a “dihydronepetalactone precursor” refers to a compound that precedes dihydronepetalactone in the biosynthetic pathway that leads to the generation of dihydronepetalactone, such as those depicted in FIGS. 1A, 1B and 1C.

The term “carbon source” generally refers to a substance suitable to be used as a source of carbon for cell growth. Carbon sources include, but are not limited to, biomass hydrolysates, starch, sucrose, cellulose, hemicellulose, xylose, and lignin, as well as monomeric components of these substrates. Carbon sources can comprise various organic compounds in various forms, including, but not limited to polymers, carbohydrates, acids, alcohols, aldehydes, ketones, amino acids, peptides, etc. These include, for example, various monosaccharides such as glucose, dextrose (D-glucose), maltose, oligosaccharides, polysaccharides, saturated or unsaturated fatty acids, succinate, lactate, acetate, ethanol, etc., or mixtures thereof. Photosynthetic organisms can additionally produce a carbon source as a product of photosynthesis. In some embodiments, carbon sources may be selected from biomass hydrolysates and glucose. In some embodiments, carbon sources include glucose, sucrose, maltose, lactose, glycerol, and ethanol.

The term “feedstock” or “microbial feedstock” refers to the minimum amount of nutrients required to sustain the growth of a microorganism. In some embodiments, feedstock comprises a carbon source, such as biomass or carbon compounds derived from biomass. In some embodiments, a feedstock comprises nutrients other than a carbon source. In some embodiments, feedstock is a raw material, or mixture of raw materials, supplied to a microorganism or fermentation process from which other products can be made. In some embodiments, feedstock is used by a microorganism that produces a product of interest (e.g. small molecule, peptide, synthetic compound, fuel, alcohol, etc.) in a fermentation process. In some embodiments, a microbial feedstock does not comprise greater than 0.5% precursor molecules, as defined above.

The term “volumetric productivity” or “production rate” is defined as the amount of product formed per volume of broth per unit of time. Volumetric productivity can be reported in gram per liter per hour (g/L/h), where grams refer to the grams of product of interest, and liter is liters of culture medium.

The term “specific productivity” is defined as the rate of formation of the product. Specific productivity is herein further defined as the specific productivity in gram product per gram of cell dry weight (CDW) per hour (g/g CDW/h). Using the relation of CDW to OD₆₀₀ for the given microorganism specific productivity can also be expressed as gram product per liter culture medium per optical density of the culture broth at 600 nm (OD) per hour (g/L/h/OD).

The term “yield” is defined as the amount of product obtained per unit weight of raw material and may be expressed as g product per g substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product.

The term “titre” or “titer” is defined as the strength of a solution or the concentration of a substance in solution. For example, the titre of a product of interest (e.g. small molecule, peptide, synthetic compound, fuel, alcohol, etc.) in a fermentation broth is described as g of product of interest in solution per liter of culture broth (g/L).

The term “total titer” is defined as the sum of all product of interest produced in a process, including but not limited to the product of interest in solution, the product of interest in gas phase if applicable, and any product of interest removed from the process and recovered relative to the initial volume in the process or the operating volume in the process.

The term “mutant protein” or “recombinant protein” is a term of the art understood by skilled persons and refers to a protein that is distinguished from the WT form of the protein on the basis of the presence of amino acid modifications, such as, for example, amino acid substitutions, insertions and/or deletions.

Amino acid modifications may be amino acid substitutions, amino acid deletions and/or amino acid insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g. charge, hydrophobicity and size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine, and the like. The mutant peptides can be chemically synthesized, or the isolated gene can be site-directed mutagenized, or a synthetic gene can be synthesized and expressed in bacteria, yeast, baculovirus, tissue culture, and the like.

A “vector” is used to transfer genetic material into a target cell. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, adenoviruses, lentiviruses, and adeno-associated viruses). In embodiments, a viral vector may be replication incompetent. Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

As used herein “sequence identity” refers to the extent to which two optimally aligned polynucleotides or polypeptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in the reference sequence segment, i.e. the entire reference sequence or a smaller defined part of the reference sequence. “Percent identity” is the identity fraction times 100. A comparison of sequences to determine the percent identity can be accomplished by a number of well-known methods, including for example by using mathematical algorithms, such as, for example, those in the BLAST suite of sequence analysis programs.

### Mevalonate and Nepetalatone Synthesis Pathways

The mevalonate pathway catalyzes the conversion of acetyl CoA to isopentenyl pyrophosphate (IPP) or DMAPP through a series of enzyme catalyzed reactions, as shown in the schematic in FIG. 1A. The enzymes involved in the mevalonate pathway are listed below in Table 1.

TABLE 1
Enzymes of the mevalonate pathway

| Enzyme | | | |
|---|---|---|---|
| abbreviation | Enzyme name | Substrate | Product |
| ERG10 | acetoacetyl-CoA thiolase | Acetyl CoA | Acetoacetyl-CoA |
| ERG13 | HMG-CoA synthase | Acetoacetyl-CoA | HMG-CoA |
| tHMG or HMG | HMG-CoA reductase | HMG-CoA | R-mevalonate |
| ERG12 | mevalonate kinase | R-mevalonate | Mevalonate-5-phosphate |
| ERG8 | phosphomevalonate kinase | Mevalonate-5- | R-mevalonate-5-pyrophosphate |
| | | phosphate | |
| ERG19 or MVD1 | diphosphomevalonate | R-mevalonate-5- | isopentenyl pyrophosphate (IPP) or |
| | decarboxylase | pyrophosphate | dimethylallyl pyrophosphate (DMAPP) |
| IDI | isopentenyl diphosphate isomerase | IPP/DMAPP | DMAPP/IPP |

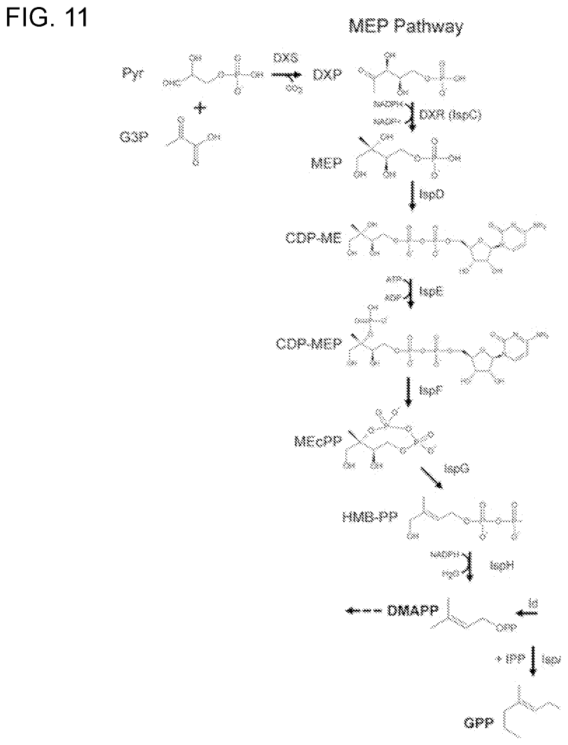

The nepetalactone synthesis pathway catalyzes the conversion of precursor metabolites, dimethylallyl pyrophosphate (DMAPP) and isopentenyl pyrophosphate (IPP) into geranyl pyrophosphate and geraniol; the conversion of geraniol to 8-hydroxygeraniol; the conversion of 8-hydroxygeraniol to 8-oxogeranial (see FIG. 11B); the formation of an enol intermediate (8-oxocitronellyl enol) by iridoid synthase (ISY) and the cyclization of the enol intermediate into nepetalactol by nepetalactol synthase (NEPS) (see FIG. 1C). The cyclization of the enol intermediate has also been shown to occur spontaneously at trace levels. Nepetalactol is converted to nepetalactone by a previously uncharacterized oxidoreductase (nepetalactol oxidoreductase, NOR). The enzymes involved in the nepetalactone synthesis pathway are listed below in Table 2.

TABLE 2
Enzymes of the nepetalactone synthesis pathway

| Enzyme | | | |
|---|---|---|---|
| abbreviation | Enzyme name | Substrate | Product |
| GPPS or | geranyl diphosphate | IPP/DMAPP | Geranyl |
| ERG20^{ww} | synthase | | pyrophosphate |
| GES | geraniol synthase | Geranyl | Geraniol |
| | | pyrophosphate | |
| G8H; CPR; | geraniol-8- | Geraniol | 8- |
| CYB5 | hydroxylase; | | hydroxygeraniol |
| | cytochrome P450 | | |
| | reductase; | | |
| | cytochrome B5 | | |
| 8HGO | 8-hydroxygeraniol | 8- | 8-oxogeranial |
| | oxidoreductase | hydroxygeraniol | |
| ISY | iridoid synthase | 8-oxogeramal | Enol intermediate |
| NEPS | nepetalactol | Enol intermediate | Nepetalactol |
| | synthase | | |
| NOR | nepetalactol | Nepetalactol | Nepetalactone |
| | oxidoreductase | | |

Finally, the conversion of nepetalactone to dihydronepetalactone is catalyzed by dihydronepetalactone dehydrogenase (DND), as shown in FIG. 1C.

### Biosynthesis of Nepetalactol Using a Recombinant NEPS Enzyme

The disclosure provides recombinant microbial cells capable of producing nepetalactol. In some embodiments, the recombinant microbial cells produce nepetalactol from glucose or other comparable carbon sources, such as galactose, glycerol and ethanol. In some embodiments, the recombinant microbial cells produce nepetalactol from glucose without additional precursor supplementation. In some embodiments, the recombinant microbial cells produce nepetalactol from any one of the intermediate substrates of the mevalonate pathway and/or the nepetalactone synthesis pathway. For example, in some embodiments, the recombinant microbial cells produce nepetalactol when supplemented with any one or more of the substrates listed in Table 1 or Table 2. In some embodiments, the recombinant microbial cells of this disclosure comprise one or more polynucleotides encoding a heterologous nepetalactol synthase (NEPS).

Prior to this disclosure, the reconstitution of the enzymatic pathways required for the conversion of nepetalactol from glucose (without additional precursor supplementation) has not been shown in any microbial cell. Moreover, while the spontaneous conversion of an enol intermediate to small amounts of nepetalactol in vitro has been observed (Campbell, Alex, Thesis, 2016, the contents of which are incorporated herein by reference in its entirety), there have been no reports of enzymatically catalyzing the synthesis of nepetalactol in vivo using an NEPS enzyme. Finally, the function of NEPS in controlling the stereochemistry of cyclization in vivo has not been described prior to this disclosure. Identification of this function enables the development of methods of specifically producing one or more nepetalactol stereoisomers, such as, cis, trans-nepetalactol, trans, cis-nepetalactol, trans, trans-nepetalactol, and/or cis, cis-nepetalactol, as described in this disclosure.

In some embodiments, the recombinant microbial cells of this disclosure express a heterologous NEPS enzyme. In some embodiments, the NEPS enzyme comprises a Pfam domain pfam12697, which may be identified by any in silico analysis program known in the art for the identification of protein domains. In some embodiments, the NEPS enzyme belongs to a large superfamily of alpha/beta hydrolases. The presence of the Pfam domain pfam12697 distinguishes the NEPS enzymes disclosed herein from the NEPS enzymes described thus far (see, for e.g., Lichman et al., Nature Chemical Biology, Vol. 15 Jan. 2019, 71-79, the contents of which are incorporated herein by reference in its entirety), which do not contain this protein domain.

In some embodiments, the polynucleotide encoding a heterologous NEPS comprises a nucleic acid sequence of at least about 80% identity to a nucleic acid sequence selected from SEQ ID Nos 1506-1562. In some embodiments, the polynucleotide comprises a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid sequence selected from SEQ ID Nos 1506-1562, including any ranges and subranges therebetween. In some embodiments, the polynucleotide consists of a nucleic acid sequence selected from SEQ ID Nos. 1506-1562.

In some embodiments, the NEPS enzymes of this disclosure exhibit cyclase activity, and thereby catalyze and enhance nepetalactol formation. In some embodiments, the NEPS enzyme comprises an amino acid sequence of at least about 80% identity to an amino acid sequence selected from SEQ ID Nos. 718-774. In some embodiments, the NEPS enzyme comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 718-774, including any ranges and subranges therebetween. In some embodiments, the NEPS enzyme consists of an amino acid sequence selected from SEQ ID Nos. 718-774.

In some embodiments, the polynucleotide encoding a heterologous NEPS comprises a nucleic acid sequence of at least about 80% identity to a nucleic acid sequence selected from SEQ ID Nos 1518-1521. In some embodiments, the polynucleotide comprises a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid sequence selected from SEQ ID Nos 1518-1521, including any ranges and subranges therebetween. In some embodiments, the polynucleotide consists of a nucleic acid sequence selected from SEQ ID Nos. 1518-1521.

In some embodiments, the NEPS enzyme comprises an amino acid sequence of at least about 80% identity to an amino acid sequence selected from SEQ ID Nos. 730-733. In some embodiments, the NEPS enzyme comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 730-733, including any ranges and subranges therebetween. In some embodiments, the NEPS enzyme consists of an amino acid sequence selected from SEQ ID Nos. 730-733.

In some embodiments, the polynucleotide encoding a heterologous NEPS comprises a nucleic acid sequence of at least about 80% identity to a nucleic acid sequence selected from SEQ ID Nos 1508-1515. In some embodiments, the polynucleotide comprises a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid sequence selected from SEQ ID Nos 1508-1515, including any ranges and subranges therebetween. In some embodiments, the polynucleotide consists of a nucleic acid sequence selected from SEQ ID Nos. 1508-1515.

In some embodiments, the NEPS enzyme comprises an amino acid sequence of at least about 80% identity to an amino acid sequence selected from SEQ ID Nos. 720-727. In some embodiments, the NEPS enzyme comprises an amino acid sequence having about 80%, about 81%, about 82%, about 83%, about 84%, about 85%, about 86%, about 87%, about 88%, about 89%, about 90%0, about 91%, about 92%, about 93%, about 94%, about 95%, about 96%, about 97%, about 98%, about 99%, or about 100% identity to an amino acid sequence selected from SEQ ID Nos. 720-727, including any ranges and subranges therebetween. In some embodiments, the NEPS enzyme consists of an amino acid sequence selected from SEQ ID Nos. 720-727.

In some embodiments, the polynucleotide encoding a heterologous NEPS comprises a nucleic acid sequence of at least about 80% identity to a nucleic acid sequence selected from SEQ ID Nos 1522-1562. In some embodiments, the polynucleotide comprises a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid sequence selected from SEQ ID Nos 1522-1562, including any ranges and subranges therebetween. In some embodiments, the polynucleotide consists of a nucleic acid sequence selected from SEQ ID Nos. 1522-1562.

In some embodiments, the NEPS enzyme comprises an amino acid sequence of at least about 80% identity to an amino acid sequence selected from SEQ ID Nos. 734-774. In some embodiments, the NEPS enzyme comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 734-774, including any ranges and subranges therebetween. In some embodiments, the NEPS enzyme consists of an amino acid sequence selected from SEQ ID Nos. 734-774.

In some embodiments, the heterologous NEPS enzyme is selected from the NEPS enzymes listed in Table 3.

TABLE 3
Exemplary NEPS enzymes for use in the methods disclosed herein

| SEQ ID | | |
|---|---|---|
| NO. | Enzyme Name | Source Organism |
| 718 | NEPS | Nepeta mussinii |
| 719 | NEPS | Nepeta mussinii |
| 720 | NEPS | Catharanthus roseus |
| 721 | NEPS | Camptotheca acuminata |
| 722 | NEPS | Vinca minor |
| 723 | NEPS | Rauvolfia serpentina |
| 724 | NEPS | Catharanthus roseus |
| 725 | NEPS | Camptotheca acuminata |
| 726 | NEPS | Vinca minor |
| 727 | NEPS | Rauvolfia serpentina |
| 728 | NEPS | Nepeta mussinii |
| 729 | NEPS | Nepeta mussinii |
| 730 | NEPS | Catharanthus roseus |
| 731 | NEPS | Camptotheca acuminata |
| 732 | NEPS | Vinca minor |
| 733 | NEPS | Rauvolfia serpentina |
| 734 | NEPS | Andrographis_{—}paniculata |
| 735 | NEPS | Gentiana triflora |
| 736 | NEPS | Coffea canephora |
| 737 | NEPS | Ophiorrhiza_{—}pumila |
| 738 | NEPS | Phelline_{—}lucida |
| 739 | NEPS | Vitex_{—}agnus_{—}castus |
| 740 | NEPS | Valeriana_{—}officianalis |
| 741 | NEPS | Stylidium_{—}adnatum |
| 742 | NEPS | Verbena_{—}hastata |
| 743 | NEPS | Byblis_{—}gigantea |
| 744 | NEPS | Pogostemon sp. |
| 745 | NEPS | Strychnos_{—}spinosa |
| 746 | NEPS | Corokia_{—}cotoneaster |
| 747 | NEPS | Oxera_{—}neriifolia |
| 748 | NEPS | Buddleja_sp. |
| 749 | NEPS | Gelsemium_{—}sempervirens |
| 750 | NEPS | Utricularia_sp. |
| 751 | NEPS | Scaevola_sp. |
| 752 | NEPS | Menyanthes_{—}trifoliata |
| 753 | NEPS | Pinguicula_{—}caudata |
| 754 | NEPS | Psychotria_{—}ipecacuanha |
| 755 | NEPS | Dipsacus_{—}sativum |
| 756 | NEPS | Exacum_{—}affine |
| 757 | NEPS | Chionanthus_{—}retusus |
| 758 | NEPS | Allamanda_{—}cathartica |
| 759 | NEPS | Phyla_{—}dulcis |
| 760 | NEPS | Ligustrum_{—}sinense |
| 761 | NEPS | Pyrenacantha_{—}malvifolia |
| 762 | NEPS | Sambucus_{—}canadensis |
| 763 | NEPS | Leonurus_{—}japonicus |
| 764 | NEPS | Ajuga_{—}reptans |
| 765 | NEPS | Paulownia_{—}fargesii |
| 766 | NEPS | Caiophora_{—}chuquitensis |
| 767 | NEPS | Plantago_{—}maritima |
| 768 | NEPS | Antirrhinum_{—}braun |
| 769 | NEPS | Cyrilla_{—}racemiflora |
| 770 | NEPS | Hydrangea_{—}quercifolia |
| 771 | NEPS | Cinchona pubescens |
| 772 | NEPS | Actinidia chinensis var. chinensis |
| 773 | NEPS | Swertia japonica |
| 774 | NEPS | Sesamum indicum |

In some embodiments, the recombinant microbial cells of this disclosure are capable of producing detectable quantities of nepetalactol. In some embodiments, the recombinant microbial cells of this disclosure are capable of producing detectable quantities of nepetalactol and its derivatives. In yet other embodiments, the recombinant microbial cells of this disclosure are capable of producing detectable quantities of nepetalactol and/or nepetalactone as an intermediate to other downstream products. In some embodiments, the methods and/or engineered microbes described herein are capable of producing nepetalactone and/or nepetalactol at a level of at least about: 0.01 g/L, 0.02 g/L, 0.03 g/L, 0.04 g/L, 0.05 g/L, 0.06 g/L, 0.07 g/L, 0.08 g/L, 0.09 g/L, 0.10 g/L, 0.20 g/L, 0.30 g/L, 0.40 g/L, 0.50 g/L, 0.60 g/L, 0.70 g/L, 0.80 g/L, 0.90 g/L, 1.00 g/L, 2.00 g/L, 3.00 g/L, 4.00 g/L, 5.00 g/L, 6.00 g/L, 7.00 g/L, 8.00 g/L, 9.00 g/L, 10.00 g/L, 20.00 g/L, 30.00 g/L, 40.00 g/L, 50.00 g/L, or more of cell lysate or culture medium. In some embodiments, the methods and/or engineered microbes described herein are capable of producing nepetalactone and/or nepetalactol at a level of at most about: 0.01 g/L, 0.02 g/L, 0.03 g/L, 0.04 g/L, 0.05 g/L, 0.06 g/L, 0.07 g/L, 0.08 g/L, 0.09 g/L, 0.10 g/L, 0.20 g/L, 0.30 g/L, 0.40 g/L, 0.50 g/L, 0.60 g/L, 0.70 g/L, 0.80 g/L, 0.90 g/L, 1.00 g/L, 2.00 g/L, 3.00 g/L, 4.00 g/L, 5.00 g/L, 6.00 g/L, 7.00 g/L, 8.00 g/L, 9.00 g/L, 10.00 g/L, 20.00 g/L, 30.00 g/L, 40.00 g/L, or 50.00 g/L of cell lysate or culture medium. In some embodiments, the methods and/or engineered microbes described herein are capable of producing nepetalactone and/or nepetalactol at a level between about: 0.01-50.00 g/L, 0.05-50.00 g/L, 0.10-50.00 g/L, 0.20-50.00 g/L, 0.30-50.00 g/L, 0.40-50.00 g/L, 0.50-50.00 g/L, 0.60-50.00 g/L, 0.70-50.00 g/L, 0.80-50.00 g/L, 0.90-50.00 g/L, 1.00-50.00 g/L, 5.00-50.00 g/L, 10.00-50.00 g/L, 15.00-50.00 g/L, 20.00-50.00 g/L, 25.00-50.00 g/L, 30.00-50.00 g/L, 35.00-50.00 g/L, 40.00-50.00 g/L, 0.01-40.00 g/L, 0.05-40.00 g/L, 0.10-40.00 g/L, 0.20-40.00 g/L, 0.30-40.00 g/L, 0.40-40.00 g/L, 0.50-40.00 g/L, 0.60-40.00 g/L, 0.70-40.00 g/L, 0.80-40.00 g/L, 0.90-40.00 g/L, 1.00-40.00 g/L, 5.00-40.00 g/L, 10.00-40.00 g/L, 15.00-40.00 g/L, 20.00-40.00 g/L, 25.00-40.00 g/L, 30.00-40.00 g/L, 0.01-30.00 g/L, 0.05-30.00 g/L, 0.10-30.00 g/L, 0.20-30.00 g/L, 0.30-30.00 g/L, 0.40-30.00 g/L, 0.50-30.00 g/L, 0.60-30.00 g/L, 0.70-30.00 g/L, 0.80-30.00 g/L, 0.90-30.00 g/L, 1.00-30.00 g/L, 5.00-30.00 g/L, 10.00-30.00 g/L, 15.00-30.00 g/L, 20.00-30.00 g/L, 0.01-20.00 g/L, 0.05-20.00 g/L, 0.10-20.00 g/L, 0.20-20.00 g/L, 0.30-20.00 g/L, 0.40-20.00 g/L, 0.50-20.00 g/L, 0.60-20.00 g/L, 0.70-20.00 g/L, 0.80-20.00 g/L, 0.90-20.00 g/L, 1.00-20.00 g/L, 5.00-20.00 g/L, 10.00-20.00 g/L, 0.01-10.00 g/L, 0.05-10.00 g/L, 0.10-10.00 g/L, 0.20-10.00 g/L, 0.30-10.00 g/L, 0.40-10.00 g/L, 0.50-10.00 g/L, 0.60-10.00 g/L, 0.70-10.00 g/L, 0.80-10.00 g/L, 0.90-10.00 g/L, 1.00-10.00 g/L, 5.00-10.00 g/L, 0.10-5.00 g/L, 0.20-5.00 g/L, 0.30-5.00 g/L, 0.40-5.00 g/L, 0.50-5.00 g/L, 0.60-5.00 g/L, 0.70-5.00 g/L, 0.80-5.00 g/L, 0.90-5.00 g/L, 1.00-5.00 g/L, 2.00-5.00 g/L, 3.00-5.00 g/L, 0.20-3.00 g/L, 0.30-3.00 g/L, 0.40-3.00 g/L, 0.50-3.00 g/L, 0.60-3.00 g/L, 0.70-3.00 g/L, 0.80-3.00 g/L, 0.90-3.00 g/L, 1.00-3.00 g/L, 2.00-3.00 g/L, 0.20-2.00 g/L, 0.30-2.00 g/L, 0.40-2.00 g/L, 0.50-2.00 g/L, 0.60-2.00 g/L, 0.70-2.00 g/L, 0.80-2.00 g/L, 0.90-2.00 g/L, or 1.00-2.00 g/L of cell lysate or culture medium.

In some embodiments, the recombinant microbial cells of this disclosure are capable of producing industrially relevant quantities of nepetalactol. In some embodiments, the recombinant microbial cells of this disclosure are capable of producing industrially relevant quantities of nepetalactol and its derivatives. In yet other embodiments, the recombinant microbial cells of this disclosure are capable of producing industrially relevant quantities of nepetalactol and/or nepetalactone as an intermediate to other downstream products. As used herein, “industrially relevant quantities” refer to amounts greater than about 0.25 gram per liter of fermentation or culture broth. In some embodiments, the recombinant microbial cells of this disclosure are capable of producing nepetalactol in an amount greater than about 0.25 gram per liter of fermentation or culture broth, for example, greater than about 0.5 gram per liter, greater than about 1 gram per liter, greater than about 5 gram per liter, greater than about 10 gram per liter, greater than about 15 gram per liter, greater than about 20 gram per liter, greater than about 25 gram per liter, greater than about 30 gram per liter, greater than about 35 gram per liter, greater than about 40 gram per liter, greater than about 45 gram per liter, greater than about 50 gram per liter, greater than about 60 gram per liter, greater than about 70 gram per liter, greater than about 80 gram per liter, greater than about 90 gram per liter, or greater than about 100 gram per liter of fermentation or culture broth, including all subranges and values that lie therebetween.

### Biosynthesis of Nepetalactone Using a Recombinant NOR Enzyme

The disclosure provides recombinant microbial cells capable of producing nepetalactone. In some embodiments, the recombinant microbial cells produce nepetalactone from glucose or other comparable carbon sources, such as galactose, glycerol and ethanol. In some embodiments, the recombinant microbial cells produce nepetalactone from glucose without additional precursor supplementation. In some embodiments, the recombinant microbial cells produce nepetalactone from any one of the intermediate substrates of the mevalonate pathway and/or the nepetalactone synthesis pathway. For example in some embodiments, the recombinant microbial cells produce nepetalactone when supplemented with any one or more of the substrates listed in Table 1 or Table 2. In some embodiments, the recombinant microbial cell of this disclosure comprise one or more polynucleotides encoding a heterologous nepetalactol oxidoreductase (NOR).

NOR is a previously uncharacterized enzyme; and the production of nepetalactone from its immediate precursor, nepetalactol, has not been demonstrated in vivo thus far, which underscores the novelty of the recombinant microbial cells of this disclosure capable of producing nepetalactone. Although Lichman et al., Nature Chemical Biology, Vol. 15 Jan. 2019, 71-79 describes NEPS1, an enzyme that can catalyze the oxidation of nepetalactol to nepetalactone, NEPS1 is, in fact, a multifunctional cyclase-dehydrogenase, which is also capable of converting an enol intermediate to nepetalactol through its cyclase activity. Importantly, there is less than 20% sequence identity between the NOR amino acid sequences disclosed herein and the NEPS1 of Lichman et al., demonstrating that the genus of NOR enzymes of this disclosure are novel over those described in the art (See Example 7).

In some embodiments, the polynucleotide encoding NOR comprises a nucleic acid sequence having at least about 80% identity to a nucleic acid sequence selected from SEQ ID Nos. 1308-1395, 1563-1570 and 1725-1727. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1308-1395, 1563-1570 and 1725-1727, including any ranges and subranges therebetween. In some embodiments, the polynucleotide consists of a nucleic acid sequence selected from SEQ ID Nos. 1308-1395, 1563-1570 and 1725-1727. In some embodiments, the NOR polynucleotide consists of the nucleic acid sequence of SEQ ID NO. 1393.

In some embodiments, the NOR comprises an amino acid sequence with at least about 80% identity to an amino acid sequence selected from SEQ ID Nos. 520-607, 775-782 and 1642-1644. For example, in some embodiments, the NOR comprises about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 520-607, 775-782 or 1642-1644, including any ranges and subranges therebetween. In some embodiments, the NOR consists of an amino acid sequence selected from SEQ ID Nos. 520-607, 775-782 or 1642-1644. In some embodiments, the NOR consists of the amino acid sequence of SEQ ID NO. 605.

In some embodiments, the NOR is a mutant NOR, which comprises at least one amino acid modification compared to the wild type NOR sequence. In some embodiments, the mutant NOR enzyme is more catalytically active than the corresponding wild type NOR enzyme. In some embodiments, the NOR enzyme has a higher k_{Cat}, as compared to the wild type enzyme. As used herein, k_{Cat} refers to the turnover number or the number of substrate molecules each enzyme site converts to product per unit time. In some embodiments, the mutant NOR enzyme that is more catalytically active than the wild type enzyme, and/or is insensitive to negative regulation, such as, for example, allosteric inhibition.

In some embodiments, the recombinant microbial cell comprises a polynucleotide encoding a mutant NOR. In some embodiments, the polynucleotide comprises a nucleic acid sequence having at least about 80% identity to a nucleic acid sequence selected from SEQ ID Nos. 1312-1317 and 1319-1321. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1312-1317 and 1319-1321, including any ranges and subranges therebetween.

In some embodiments, the mutant NOR comprises an amino acid sequence with at least 80% identity to an amino acid sequence selected from SEQ ID Nos: 524-529, or 531-533. For example, in some embodiments, the mutant NOR comprises about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 524-529, or 531-533, including any ranges and subranges therebetween. In some embodiments, the NOR consists of an amino acid sequence selected from SEQ ID Nos. 524-529, or 531-533.

In some embodiments, the heterologous NOR enzyme is selected from the enzymes listed in Table 4.

TABLE 4
Exemplary NOR enzymes

| Protein | | |
|---|---|---|
| SEQ ID | | |
| NO: | Enzyme | Source organism |
| 520 | NOR | Nepeta mussinii |
| 521 | NOR | Nepeta mussinii |
| 522 | NOR | Nepeta cataria |
| 523 | NOR | Nepeta cataria |
| 524 | NOR | Nepeta cataria |
| 525 | NOR | Nepeta cataria |
| 526 | NOR | Nepeta cataria |
| 527 | NOR | Nepeta cataria |
| 528 | NOR | Nepeta cataria |
| 529 | NOR | Nepeta cataria |
| 530 | NOR | Nepeta cataria |
| 531 | NOR | Nepeta cataria |
| 532 | NOR | Nepeta cataria |
| 533 | NOR | Nepeta cataria |
| 534 | NOR | Nepeta cataria |
| 535 | NOR | Nepeta cataria or Nepeta mussinii |
| 536 | NOR | Nepeta cataria or Nepeta mussinii |
| 537 | NOR | Nepeta cataria or Nepeta mussinii |
| 538 | NOR | Nepeta cataria or Nepeta mussinii |
| 539 | NOR | Nepeta cataria or Nepeta mussinii |
| 540 | NOR | Nepeta cataria or Nepeta mussinii |
| 541 | NOR | Nepeta cataria or Nepeta mussinii |
| 542 | NOR | Nepeta cataria or Nepeta mussinii |
| 543 | NOR | Nepeta cataria or Nepeta mussinii |
| 544 | NOR | Nepeta cataria or Nepeta mussinii |
| 545 | NOR | Nepeta cataria or Nepeta mussinii |
| 546 | NOR | Nepeta cataria or Nepeta mussinii |
| 547 | NOR | Nepeta cataria or Nepeta mussinii |
| 548 | NOR | Nepeta cataria or Nepeta mussinii |
| 549 | NOR | Nepeta cataria or Nepeta mussinii |
| 550 | NOR | Nepeta cataria or Nepeta mussinii |
| 551 | NOR | Nepeta cataria or Nepeta mussinii |
| 552 | NOR | Nepeta cataria |
| 553 | NOR | Nepeta cataria |
| 554 | NOR | Nepeta cataria |
| 555 | NOR | Nepeta cataria |
| 556 | NOR | Nepeta cataria |
| 557 | NOR | Nepeta cataria |
| 558 | NOR | Nepeta cataria |
| 559 | NOR | Nepeta cataria |
| 560 | NOR | Nepeta cataria |
| 561 | NOR | Nepeta cataria |
| 562 | NOR | Nepeta cataria |
| 563 | NOR | Nepeta cataria |
| 564 | NOR | Nepeta cataria |
| 565 | NOR | Nepeta cataria |
| 566 | NOR | Nepeta cataria |
| 567 | NOR | Nepeta cataria |
| 568 | NOR | Nepeta cataria |
| 569 | NOR | Nepeta cataria |
| 570 | NOR | Nepeta cataria |
| 571 | NOR | Nepeta cataria |
| 572 | NOR | Nepeta cataria |
| 573 | NOR | Nepeta cataria |
| 574 | NOR | Nepeta cataria |
| 575 | NOR | Nepeta cataria |
| 576 | NOR | Nepeta cataria |
| 577 | NOR | Nepeta cataria |
| 578 | NOR | Nepeta cataria |
| 579 | NOR | Nepeta cataria |
| 580 | NOR | Nepeta cataria |
| 581 | NOR | Nepeta cataria |
| 582 | NOR | Nepeta cataria |
| 583 | NOR | Nepeta cataria |
| 584 | NOR | Nepeta cataria |
| 585 | NOR | Nepeta cataria |
| 586 | NOR | Nepeta cataria |
| 587 | NOR | Nepeta cataria |
| 588 | NOR | Nepeta cataria |
| 589 | NOR | Nepeta cataria |
| 590 | NOR | Nepeta cataria |
| 591 | NOR | Nepeta cataria/mussinii |
| 592 | NOR | Nepeta cataria/mussinii |
| 593 | NOR | Nepeta cataria/mussinii |
| 594 | NOR | Nepeta cataria/mussinii |
| 595 | NOR | Nepeta cataria/mussinii |
| 596 | NOR | Nepeta cataria/mussinii |
| 597 | NOR | Nepeta cataria/mussinii |
| 598 | NOR | Nepeta cataria/mussinii |
| 599 | NOR | Nepeta cataria/mussinii |
| 600 | NOR | Nepeta cataria/mussinii |
| 601 | NOR | Nepeta cataria/mussinii |
| 602 | NOR | Nepeta cataria/mussinii |
| 603 | NOR | Nepeta cataria/mussinii |
| 604 | NOR | Nepeta cataria/mussinii |
| 605 | NOR | Nepeta cataria/mussinii |
| 606 | NOR | Nepeta cataria/mussinii |
| 607 | NOR | Nepeta cataria/mussinii |
| 775 | NOR | Isodon_{—}rubescens |
| 776 | NOR | Prunella_{—}vulgaris |
| 777 | NOR | Agastache_{—}rugosa |
| 778 | NOR | Melissa_{—}officinalis |
| 779 | NOR | Micromeria_{—}fruticosa |
| 780 | NOR | Plectranthus_{—}caninus |
| 781 | NOR | Rosmarinus officinalis |
| 782 | NOR | Nepeta mussinii |
| 1642 | NOR | Nepeta cataria |
| 1643 | NOR | Nepeta cataria |
| 1644 | NOR | Nepeta cataria |

In some embodiments, the recombinant microbial cells of this disclosure are capable of producing industrially relevant quantities of nepetalactone. As used herein, “industrially relevant quantities” refer to amounts greater than about 0.25 gram per liter of fermentation broth. In some embodiments, the recombinant microbial cells of this disclosure are capable of producing nepetalactone in an amount greater than about 0.25 gram per liter of fermentation broth, for example, greater than about 0.5 gram per liter, greater than about 1 gram per liter, greater than about 5 gram per liter, greater than about 10 gram per liter, greater than about 15 gram per liter, greater than about 20 gram per liter, greater than about 25 gram per liter, greater than about 30 gram per liter, greater than about 35 gram per liter, greater than about 40 gram per liter, greater than about 45 gram per liter, or greater than about 50 gram per liter of fermentation broth, including all subranges and values that lie therebetween.

### Biosynthesis of Dihydronepetalactone Using a Recombinant DND Enzyme

The disclosure provides recombinant microbial cells capable of producing dihydronepetalactone from nepetalactone. Prior to this disclosure, the production of dihydronepetalactone from nepetalactone had not been demonstrated either in vitro or in vivo, further underscoring the novelty of the recombinant microbial cells of this disclosure capable of producing dihydronepetalactone, over the existing knowledge in the art.

In some embodiments, the recombinant microbial cells produce dihydronepetalactone from glucose or other comparable carbon sources, such as galactose, glycerol and ethanol. In some embodiments, the recombinant microbial cells produce dihydronepetalactone from glucose without additional precursor supplementation. In some embodiments, the recombinant microbial cells produce dihydronepetalactone from any one of the intermediate substrates of the mevalonate pathway and/or the nepetalactone/dihydronepetalactone synthesis pathway. For example, in some embodiments, the recombinant microbial cells produce dihydronepetalactone when supplemented with any one or more of the substrates listed in Table 1 or Table 2.

In some embodiments, the recombinant microbial cell of this disclosure comprises one or more polynucleotides encoding a heterologous dihydronepetalactone dehydrogenase (DND).

In some embodiments, the recombinant microbial cells of this disclosure are capable of producing industrially relevant quantities of dihydronepetalactone. As used herein, “industrially relevant quantities” refer to amounts greater than about 0.25 gram per liter of fermentation broth. In some embodiments, the recombinant microbial cells of this disclosure are capable of producing dihydronepetalactone in an amount greater than about 0.25 gram per liter of fermentation broth, for example, greater than about 0.5 gram per liter, greater than about 1 gram per liter, greater than about 5 gram per liter, greater than about 10 gram per liter, greater than about 15 gram per liter, greater than about 20 gram per liter, greater than about 25 gram per liter, greater than about 30 gram per liter, greater than about 35 gram per liter, greater than about 40 gram per liter, greater than about 45 gram per liter, or greater than about 50 gram per liter of fermentation broth, including all subranges and values that lie therebetween.

### Genetic Engineering of the Mevalonate Pathway

In some embodiments, the recombinant microbial cells of this disclosure may comprise one or more polynucleotide(s) encoding one or more of the enzymes of mevalonate (MVA) pathway listed in Table 1. For instance, in some embodiments, the recombinant microbial cells of this disclosure may comprise one or more polynucleotide(s) encoding one or more of the following enzymes of the mevalonate pathway: acetyl-CoA C-acetyltransferase (acetoacetyl-CoA thiolase, ERG10), 3-hydroxy-3-methylglutaryl-CoA (HMG-CoA) synthase (ERG13), HMG-CoA reductase (tHMG), Mevalonate kinase (ERG12), Phosphomevalonate kinase (ERG8), Mevalonate pyrophosphate decarboxylase (MVD1, ERG19), and Isopentenyl diphosphate:dimethylallyl diphosphate isomerase (IDI). In some embodiments, the recombinant microbial cell comprises one or more polynucleotide(s) encoding each of the enzymes of mevalonate pathway listed in Table 1.

Without being bound by theory, it is thought that the overexpression of one or more enzymes of the mevalonate synthesis pathway may increase the flux through the mevalonate pathway to increase the amounts of IPP or DMAPP produced in the recombinant microbial cells of this disclosure, and thereby contribute to the increase in flux through the nepetalactol synthesis pathway, resulting in an increased amount of nepetalactol/nepetalactone/dihydronepetalactone in the recombinant microbial cells of this disclosure.

In some embodiments, the recombinant microbial cell is engineered to overexpress one or more of the enzymes of the mevalonate pathway listed in Table 1. In some embodiments, the recombinant microbial cell is engineered to overexpress all of the enzymes of the mevalonate pathway listed in Table 1. The amount of the enzyme expressed by the recombinant microbial cell may be higher than the amount of that corresponding enzyme in a wild type microbial cell by about 1.25 fold to about 20 fold, for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 75 fold, about 80 fold, about 85 fold, about 90 fold, about 95 fold, or about 100 fold, including all the subranges and values that lie therebetween.

In some embodiments the recombinant microbial cell has been modified to contain a heterologous promoter operably linked to one or more endogenous MVA gene (i.e., operably linked to one or more gene from Table 1). In some embodiments, the heterologous promoter is a stronger promoter, as compared to the native promoter. In some embodiments, the recombinant microbial cell is engineered to express an enzyme of the MVA synthesis pathway constitutively. For instance, in some embodiments, the recombinant microbial cell may express an enzyme of the MVA synthesis pathway at a time when the enzyme is not expressed by the wild type microbial cell.

In other embodiments, the present disclosure envisions overexpressing one or more MVA genes by increasing the copy number of said MVA gene. Thus, in some embodiments, the recombinant microbial cell comprises at least one additional copy of a DNA sequence encoding an enzyme of the mevalonate synthesis pathway, as compared to a wild type microbial cell. In some embodiments, the recombinant microbial cell comprises 1 to 40 additional copies of a DNA sequence encoding an enzyme of the mevalonate synthesis pathway, as compared to a wild type microbial cell. For instance, the recombinant microbial cell may comprise 1, 2, 3, 4, 5, 6, 7, 8, 9, 10, 15, 20, 25, 30, 35, or 40 additional copies of the DNA sequence, compared to a wild type microbial cell, including any ranges and subranges therebetween. For example, in some embodiments, the recombinant microbial cell comprises one or two additional copies of a DNA sequence encoding an enzyme of the mevalonate synthesis pathway listed in Table 1. In some embodiments, the recombinant microbial cell comprises 1-5 additional copies of a DNA sequence encoding HMG.

In some embodiments, the present disclosure teaches methods of increasing nepetalactol biosynthesis by expressing one or more mutant MVA genes. Thus, in some embodiments, the recombinant microbial cell comprises a DNA sequence encoding for one or more mutant MVA synthesis enzymes. In some embodiments, the one or more mutant MVA synthesis enzymes are more catalytically active than the corresponding wild type enzyme. In some embodiments, the one or more mutant MVA enzymes have a higher k_{Cat}, as compared to the wild type enzyme. In some embodiments, the one or more mutant MVA enzymes that are more catalytically active than the wild type enzyme, are insensitive to negative regulation, such as, for example, allosteric inhibition.

In some embodiments, the recombinant microbial cell comprises a polynucleotide encoding an enzyme of the mevalonate synthesis pathway, wherein the polynucleotide comprises a nucleic acid sequence having at least about 80% identity to the nucleic acid sequence of the corresponding wild type form of the polynucleotide present in the wild type microbial cell. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to the corresponding wild type form of the polynucleotide present in the wild type microbial cell, including any ranges and subranges therebetween.

Thus, in some embodiments, the recombinant microbial cell comprises a polynucleotide encoding an enzyme of the mevalonate synthesis pathway, wherein the polynucleotide comprises a nucleic acid sequence having at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a polynucleotide encoding an MVA enzyme selected from those listed in Table 5, including any ranges and subranges therebetween.

In some embodiments, the recombinant microbial cell expresses an enzyme of the mevalonate synthesis pathway, wherein the enzyme comprises an amino acid sequence comprising at least 80% identity to the sequence of the corresponding enzyme expressed by the wild type microbial cell. In some embodiments, the enzyme expressed by the recombinant microbial cell comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to the corresponding wild type enzyme expressed by the wild type microbial cell, including any ranges and subranges therebetween.

Thus, in some embodiments, the recombinant microbial cell comprises an enzyme of the mevalonate synthesis pathway, wherein the enzyme comprises an amino acid sequence having at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an MVA enzyme listed in Table 5, including any ranges and subranges therebetween.

Without being bound by theory, it is thought that HMG is a rate-limiting enzyme in the mevalonate pathway, and therefore, that a truncated version of HMG lacking its regulatory domain may increase the flux through this pathway. Therefore, in some embodiments, the recombinant microbial cell is engineered to express a truncated version of HMG. In some embodiments, the truncated version of HMG lacks the regulatory function of wild type HMG.

In some embodiments, HMG comprises a membrane-binding region in its N-terminal region and a catalytically active region in its C-terminal region. In some embodiments, the truncated HMG lacks the N-terminal membrane-binding region. As used herein, the membrane binding region enables the binding and/or association of HMG to a membrane, such as, for example, the endoplasmic reticulum membrane. Therefore, in some embodiments, the truncated HMG that lacks its membrane binding region is not associated with and/or bound to a membrane. In some embodiments, the membrane-binding region comprises an amino acid sequence spanning amino acid residue 1 to amino acid residue 552 of SEQ ID NO: 1810. Therefore, in some embodiments, when HMG comprises the amino acid sequence of SEQ ID NO: 1810, the truncated HMG does not comprise the amino acid sequence spanning amino acid residue 1 to amino acid residue 552 of SEQ ID NO: 1810. Further details of truncations of HMG are provided in Polakowski et al., C. Appl Microbiol Biotechnol (1998) 49: 66, which is incorporated herein by reference in its entirety for all purposes.

Thus, in some embodiments, the HMG enzyme expressed by the recombinant microbial cell may comprise an amino acid sequence that is truncated as compared to the wild type enzyme expressed by the wild type microbial cell. For example, in some embodiments, the recombinant microbial cell is engineered to express 1-5 additional copies of a truncated version of HMG.

In some embodiments, the recombinant microbial cells of this disclosure are engineered to reduce the expression of one or more of the followings enzymes: Farnesyl pyrophosphate synthetase (ERG20) and Farnesyl-diphosphate farnesyl transferase (squalene synthase; ERG9).

Without being bound by theory, it is thought that the downregulation of one or both of the ERG20 and ERG9 enzymes may increase flux towards the production of GPP, thereby increasing the flux through the nepetalactol synthesis pathway and increasing the production of nepetalactol/nepetalactone/dihydronepetalactone. In some embodiments, the recombinant microbial cells are engineered to reduce the expression of one or more of the ERG20 and ERG9 enzymes by replacing their native promoters with a heterologous promoter that is weaker than the native promoter. In some embodiments, the recombinant microbial cells are engineered to reduce the expression of one or more of the ERG20 and ERG9 enzymes by introducing one or more mutations into the coding and/or the non-coding regions of the polynucleotide encoding the enzyme. In some embodiments, the recombinant microbial cells are engineered to reduce the expression of one or more of the ERG20 and ERG9 enzymes by deleting at least a portion of their respective coding genes or their promoters.

In some embodiments, the recombinant microbial cell expresses a recombinant enzyme of the mevalonate synthesis pathway. In some embodiments, the recombinant enzyme is a homolog derived from another microbial species, a plant cell or a mammalian cell. In some embodiments, the homolog is more catalytically active as compared to the wild type enzyme expressed by the wild type microbial cell. In some embodiments, the homolog is selected from the MVA pathway enzyme homologs listed in Table 5.

TABLE 5
An exemplary list of homologs of MVA pathway enzymes identified using BLAST searches

| | % | | | |
|---|---|---|---|---|
| | Pairwise | | | Query |
| | Identity | | | protein |
| | with | | Organism of the | used in |
| Homolog | query | | homolog protein | BLAST |
| Name | protein | Description of the homolog | identified by BLAST | search |
| CDF91480 | 63.70% | ZYBA0S11-03796g1_1 [Zygosaccharomyces bailii CLIB 213] | Zygosaccharomyces bailii | HMG1 |
| CDF91138 | 75.00% | ZYBA0S10-00562g1_1 [Zygosaccharomyces bailii CLIB 213] | Zygosaccharomyces bailii | ERG13 |
| EDZ69577 | 99.50% | YNR043Wp-like protein [Saccharomyces cerevisiae AWRI1631] | Saccharomyces cerevisiae | MVD1 |
| AAT93171 | 99.70% | YNR043W [Saccharomyces cerevisiae] | Saccharomyces cerevisiae | MVD1 |
| EDZ70002 | 99.20% | YMR220Wp-like protein [Saccharomyces cerevisiae AWRI1631] | Saccharomyces cerevisiae | ERG8 |
| EDZ70019 | 99.70% | YMR208Wp-like protein, partial [Saccharomyces cerevisiae AWRI1631] | Saccharomyces cerevisiae | ERG12 |
| EDZ70357 | 99.50% | YLR450Wp-like protein, partial [Saccharomyces cerevisiae AWRI1631] | Saccharomyces cerevisiae | HMG2 |
| AAT92819 | 99.90% | YLR450W [Saccharomyces cerevisiae] | Saccharomyces cerevisiae | HMG2 |
| CDO95793 | 70.90% | unnamed protein product [Kluyveromyces dobzhanskii CBS 2104] | Kluyveromyces dobzhanskii | MVD1 |
| CDO95247 | 68.50% | unnamed protein product [Kluyveromyces dobzhanskii CBS 2104] | Kluyveromyces dobzhanskii | IDI1 |
| CDO93808 | 76.40% | unnamed protein product [Kluyveromyces dobzhanskii CBS 2104] | Kluyveromyces dobzhanskii | ERG10 |
| CDO93737 | 79.90% | unnamed protein product [Kluyveromyces dobzhanskii CBS 2104] | Kluyveromyces dobzhanskii | ERG13 |
| CDO93041 | 51.10% | unnamed protein product [Kluyveromyces dobzhanskii CBS 2104] | Kluyveromyces dobzhanskii | ERG8 |
| XP_002497669 | 73.20% | uncharacterized protein ZYRO0F10846g [Zygosaccharomyces rouxii] | Zygosaccharomyces rouxii | IDI1 |
| XP_002497603 | 57.20% | uncharacterized protein ZYRO0F09328g [Zygosaccharomyces rouxii] | Zygosaccharomyces rouxii | ERG12 |
| XP_002497180 | 70.50% | uncharacterized protein ZYRO0D17270g [Zygosaccharomyces rouxii] | Zygosaccharomyces rouxii | MVD1 |
| XP_002495578 | 61.50% | uncharacterized protein ZYRO0B14696g [Zygosaccharomyces rouxii] | Zygosaccharomyces rouxii | HMG1 |
| XP_002494634 | 51.50% | uncharacterized protein ZYRO0A06072g [Zygosaccharomyces rouxii] | Zygosaccharomyces rouxii | ERG8 |
| XP_002494490 | 80.70% | uncharacterized protein ZYRO0A02728g [Zygosaccharomyces rouxii] | Zygosaccharomyces rouxii | ERG10 |
| XP_002494408 | 75.70% | uncharacterized protein ZYRO0A00770g [Zygosaccharomyces rouxii] | Zygosaccharomyces rouxii | ERG13 |
| XP_022630313 | 70.30% | uncharacterized protein LALA0_S10e02344g [Lachancea lanzarotensis] | Lachancea lanzarotensis | IDI1 |
| XP_022628206 | 75.90% | uncharacterized protein LALA0_S04e04918g [Lachancea lanzarotensis] | Lachancea lanzarotensis | ERG10 |
| XP_022626422 | 50.20% | uncharacterized protein LALA0_S01e04742g [Lachancea lanzarotensis] | Lachancea lanzarotensis | ERG12 |
| XP_022626264 | 77.60% | uncharacterized protein LALA0_S01e01156g [Lachancea lanzarotensis] | Lachancea lanzarotensis | ERG13 |
| XP_022461986 | 72.80% | uncharacterized protein KUCA_T00006002001 [Kuraishia capsulata CBS | Kuraishia capsulata | ERG13 |
| | | 1993] | | |
| XP_455548 | 71.90% | uncharacterized protein KLLA0_F10285g [Kluyveromyces lactis] | Kluyveromyces lactis | MVD1 |
| XP_455121 | 69.10% | uncharacterized protein KLLA0_F00924g [Kluyveromyces lactis] | Kluyveromyces lactis | IDI1 |
| XP_453599 | 77.40% | uncharacterized protein KLLA0_D12056g [Kluyveromyces lactis] | Kluyveromyces lactis | ERG10 |
| XP_453529 | 79.70% | uncharacterized protein KLLA0_D10505g [Kluyveromyces lactis] | Kluyveromyces lactis | ERG13 |
| XP_449306 | 81.20% | uncharacterized protein CAGL0L12364g [[Candida] glabrata] | | ERG10 |
| XP_449268 | 66.10% | uncharacterized protein CAGL0L11506g [[Candida] glabrata] | | HMG1 |
| XP_448008 | 76.10% | uncharacterized protein CAGL0J06952g [[Candida] glabrata] | | IDI1 |
| XP_446972 | 76.60% | uncharacterized protein CAGL0H04081g [[Candida] glabrata] | | ERG13 |
| XP_446138 | 55.10% | uncharacterized protein CAGL0F03861g [[Candida] glabrata] | | ERG12 |
| XP_445335 | 72.10% | uncharacterized protein CAGL0C03630g [[Candida] glabrata] | | MVD1 |
| SMN22164 | 65.40% | similar to Saccharomyces cerevisiae YPL117C IDI1 Isopentenyl | Kazachstania saulgeensis | IDI1 |
| | | diphosphate: dimethylallyl diphosphate isomerase (IPP isomerase) | | |
| | | [Kazachstania saulgeensis] | | |
| SMN22812 | 82.10% | similar to Saccharomyces cerevisiae YPL028W ERG10 Acetyl-CoA C- | Kazachstania saulgeensis | ERG10 |
| | | acetyltransferase (acetoacetyl-CoA thiolase) [Kazachstania saulgeensis] | | |
| SMN21601 | 71.30% | similar to Saccharomyces cerevisiae YNR043W MVD1 Mevalonate | Kazachstania saulgeensis | MVD1 |
| | | pyrophosphate decarboxylase, essential enzyme involved in the biosynthesis | | |
| | | of isoprenoids and sterols, including ergosterol [Kazachstania saulgeensis] | | |
| SMN22092 | 50.10% | similar to Saccharomyces cerevisiae YMR220W ERG8 Phosphomevalonate | Kazachstania saulgeensis | ERG8 |
| | | kinase [Kazachstania saulgeensis] | | |
| SMN22016 | 79.80% | similar to Saccharomyces cerevisiae YML126C ERG13 3-hydroxy-3- | Kazachstania saulgeensis | ERG13 |
| | | methylglutatyl-CoA (HMG-CoA) synthase, catalyzes the formation of HMG- | | |
| | | CoA from acetyl-CoA and acetoacetyl-CoA [Kazachstania saulgeensis] | | |
| CDH15668 | 51.70% | related to Phosphomevalonate kinase [Zygosaccharomyces bailii ISA1307] | Zygosaccharomyces bailii | ERG8 |
| SJM84816 | 51.70% | related to Phosphomevalonate kinase [Zygosaccharomyces ballii] | Zygosaccharomyces bailii | ERG8 |
| SSD62030 | 49.30% | related to Phosphomevalonate kinase [Saccharomycodes ludwigii] | Saccharomycodes ludwigii | ERG8 |
| CDH08870 | 55.30% | related to Mevalonate kinase [Zygosaccharomyces bailii ISA1307] | Zygosaccharomyces bailii | ERG12 |
| SJM85219 | 55.30% | related to Mevalonate kinase [Zygosaccharomyces bailii] | Zygosaccharomyces bailii | ERG12 |
| SJM88302 | 72.90% | probable Isopentenyl-diphosphate Delta-isomerase [Zygosaccharomyces | Zygosaccharomyces bailii | IDI1 |
| | | bailii] | | |
| SSD61603 | 68.00% | probable Isopentenyl-diphosphate Delta-isomerase [Saccharomycodes | Saccharomycodes ludwigii | IDI1 |
| | | ludwigii] | | |
| CDH11232 | 74.80% | probable Hydroxymethylglutaryl-CoA synthase [Zygosaccharomyces bailii | Zygosaccharomyces bailii | ERG13 |
| | | ISA1307] | | |
| SSD60462 | 78.70% | probable Hydroxymethylglutaryl-CoA synthase [Saccharomycodes ludwigii] | Saccharomycodes ludwigii | ERG13 |
| CDH11390 | 63.50% | probable 3-hydroxy-3-methylglutaryl-coenzyme A reductase 1 | Zygosaccharomyces bailii | HMG1 |
| | | [Zygosaccharomyces bailii ISA1307] | | |
| SJM86712 | 63.70% | probable 3-hydroxy-3-methylglutaryl-coenzyme A reductase 1 | Zygosaccharomyces bailii | HMG1 |
| | | [Zygosaccharomyces bailii] | | |
| SCV13952 | 65.00% | probable 3-hydroxy-3-methylglutaryl-coenzyme A reductase 1 [[Candida] glabrata] | | HMG1 |
| GCE98125 | 51.00% | phosphomevalonate kinase [Zygosaccharomyces mellis] | Zygosaccharomyces mellis | ERG8 |
| NP_013947 | 100.00% | phosphomevalonate kinase [Saccharomyces cerevisiae S288C] | Saccharomyces cerevisiae | ERG8 |
| ONH80977 | 99.30% | Phosphomevalonate kinase [Saccharomyces cerevisiae] | Saccharomyces cerevisiae | ERG8 |
| AAA34596 | 98.60% | phosphomevalonate kinase [Saccharomyces cerevisiae] | Saccharomyces cerevisiae | ERG8 |
| AJT30847 | 99.00% | Mvd1p [Saccharomyces cerevisiae YJM1460] | Saccharomyces cerevisiae | MVD1 |
| AJT26802 | 99.00% | Mvd1p [Saccharomyces cerevisiae YJM1402] | Saccharomyces cerevisiae | MVD1 |
| AJT25337 | 98.50% | Mvd1p [Saccharomyces cerevisiae YJM1389] | Saccharomyces cerevisiae | MVD1 |
| AJT22350 | 99.50% | Mvd1p [Saccharomyces cerevisiae YJM1355] | Saccharomyces cerevisiae | MVD1 |
| AJT18309 | 99.50% | Mvd1p [Saccharomyces cerevisiae YJM1252] | Saccharomyces cerevisiae | MVD1 |
| AJT16805 | 99.50% | Mvd1p [Saccharomyces cerevisiae YJM1242] | Saccharomyces cerevisiae | MVD1 |
| AHY77130 | 99.70% | Mvd1p [Saccharomyces cerevisiae YJM993] | Saccharomyces cerevisiae | MVD1 |
| AJT08512 | 99.20% | Mvd1p [Saccharomyces cerevisiae YJM627] | Saccharomyces cerevisiae | MVD1 |
| AJT07024 | 99.00% | Mvd1p [Saccharomyces cerevisiae YJM470] | Saccharomyces cerevisiae | MVD1 |
| AJT04786 | 99.00% | Mvd1p [Saccharomyces cerevisiae YJM326] | Saccharomyces cerevisiae | MVD1 |
| AJT04410 | 99.00% | Mvd1p [Saccharomyces cerevisiae YJM320] | Saccharomyces cerevisiae | MVD1 |
| AJT04035 | 99.00% | Mvd1p [Saccharomyces cerevisiae YJM271] | Saccharomyces cerevisiae | MVD1 |
| AJT02547 | 99.00% | Mvd1p [Saccharomyces cerevisiae YJM195] | Saccharomyces cerevisiae | MVD1 |
| EHN00406 | 96.20% | Mvd1p [Saccharomyces cerevisiae × Saccharomyces kudriavzevii VIN7] | Saccharomyces cerevisiae | MVD1 |
| EEU08298 | 99.50% | Mvd1p [Saccharomyces cerevisiae JAY291] | Saccharomyces cerevisiae | MVD1 |
| EJS41872 | 95.20% | mvd1p [Saccharomyces arboricola H-6] | Saccharomyces arboricola | MVD1 |
| XP_018219912 | 93.20% | MVD1-like protein [Saccharomyces eubayanus] | Saccharomyces eubayanus | MVD1 |
| GCE98861 | 59.40% | mevalonate kinase [Zygosaccharomyces mellis] | Zygosaccharomyces mellis | ERG12 |
| NP_013935 | 100.00% | mevalonate kinase [Saccharomyces cerevisiae S288C] | Saccharomyces cerevisiae | ERG12 |
| EDV11699 | 99.50% | mevalonate kinase [Saccharomyces cerevisiae RM11-1a] | Saccharomyces cerevisiae | ERG12 |
| XP_022676263 | 50.80% | mevalonate kinase [Kluyveromyces marxianus DMKU3-1042] | Kluyveromyces marxianus | ERG12 |
| KTA97153 | 55.10% | Mevalonate kinase [[Candida] glabrata] | | ERG12 |
| BAA24409 | 100.00% | mevalonate kinase, partial [Saccharomyces cerevisiae] | Saccharomyces cerevisiae | ERG12 |
| CUS24402 | 76.60% | LAQU0S16e00892g1_1 [Lachancea quebecensis] | Lachancea quebecensis | ERG10 |
| CUS23819 | 78.40% | LAQU0S12e00738g1_1 [Lachancea quebecensis] | Lachancea quebecensis | ERG13 |
| CUS23399 | 69.20% | LAQU0S09e03884g1_1 [Lachancea quebecensis] | Lachancea quebecensis | MVD1 |
| CUS20468 | 70.30% | LAQU0S01e07272g1_1 [Lachancea quebecensis] | Lachancea quebecensis | IDI1 |
| CUS20353 | 51.20% | LAQU0S01e04720g1_1 [Lachancea quebecensis] | Lachancea quebecensis | ERG12 |
| SCV05860 | 51.50% | LANO_0H16776g1_1 [Lachancea nothofagi CBS 11611] | Lachancea nothofagi | ERG12 |
| SCV05741 | 72.50% | LANO_0H14158g1_1 [Lachancea nothofagi CBS 11611] | Lachancea nothofagi | IDI1 |
| SCO95413 | 78.60% | LANO_0E10286g1_1 [Lachancea nothofagi CBS 11611] | Lachancea nothofagi | ERG10 |
| SCU83042 | 78.50% | LANO_0B08174g1_1 [Lachancea nothofagi CBS 11611] | Lachancea nothofagi | ERG13 |
| SCU77684 | 68.70% | LANO_0A01002g1_1 [Lachancea nothofagi CBS 11611] | Lachancea nothofagi | MVD1 |
| SCV02723 | 77.10% | LAMI_0H02344g1_1 [Lachancea mirantina] | Lachanceamirantina | ERG10 |
| SCU93876 | 73.60% | LAMI_0E15896g1_1 [Lachancea mirantina] | Lachancea mirantina | ERG13 |
| SCU85068 | 71.00% | LAMI_0C10022g1_1 [Lachancea mirantina] | Lachancea mirantina | IDI1 |
| SCU78406 | 53.50% | LAMI_0A04522g1_1 [Lachancea mirantina] | Lachancea mirantina | ERG12 |
| SCC77416 | 68.80% | LAMI_0A01068g1_1 [Lachancea mirantina] | Lachancea mirantina | MVD1 |
| SCV03806 | 69.90% | LAME_0H13366g1_1 [Lachancea meyersii CBS 8951] | Lachancea meyersii | IDI1 |
| SCV03282 | 76.60% | LAME_0H09164g1_1 [Lachancea meyersii CBS 8951] | Lachancea meyersii | ERG10 |
| SCV02561 | 52.30% | LAME_0H02784g1_1 [Lachancea meyersii CBS 8951] | Lachancea meyersii | ERG12 |
| SCV01971 | 77.60% | LAME_0G19746g1_1 [Lachancea meyersii CBS 8951] | Lachancea meyersii | ERG13 |
| SCW04032 | 79.30% | LAFE_0H04412g1_1 [Lachancea fermentati] | Lachancea fermentati | ERG10 |
| SCW03437 | 74.30% | LAFE_0G10396g1_1 [Lachancea fermentati] | Lachancea fermentati | IDI1 |
| SCW01722 | 55.60% | LAFE_0E05820g1_1 [Lachancea fermentati] | Lachancea fermentati | ERG12 |
| SCW00288 | 71.90% | LAFE_0C00848g1_1 [Lachancea fermentati] | Lachancea fermentati | MVD1 |
| SCW00227 | 77.10% | LAFE_0B12244g1_1 [Lachancea fermentati] | Lachancea fermentati | ERG13 |
| SCV99364 | 64.20% | LAFE_0A01552g1_1 [Lachancea fermentati] | Lachancea fermentati | HMG1 |
| SCU90991 | 76.50% | LAFA_0F01244g1_1 [Lachancea sp. CBS 6924] | Lachancea sp. | ERG13 |
| SCU89429 | 71.70% | LAFA_0E17964g1_1 [Lachancea sp. CBS 6924] | Lachancea sp. | IDI1 |
| SCU88301 | 77.90% | LAFA_0E11870g1_1 [Lachancea sp. CBS 6924] | Lachancea sp. | ERG10 |
| SCU79660 | 50.50% | LAFA_0B04720g1_1 [Lachancea sp. CBS 6924] | Lachancea sp. | ERG12 |
| SCU92187 | 68.80% | LADA_0F14950g1_1 [Lachancea dasiensis CBS 10888] | Lachancea dasiensis | MVD1 |
| SCU86145 | 76.10% | LADA_0D12596g1_1 [Lachancea dasiensis CBS 10888] | Lachancea dasiensis | ERG13 |
| SCU85163 | 75.90% | LADA_0D06018g1_1 [Lachancea dasiensis CBS 10888] | Lachancea dasiensis | ERG10 |
| SCU82873 | 72.50% | LADA_0C08416g1_1 [Lachancea dasiensis CBS 10888] | Lachancea dasiensis | IDI1 |
| SCU82514 | 49.70% | LADA_0C05908g1_1 [Lachancea dasiensis CBS 10888] | Lachancea dasiensis | ERG12 |
| XP_002554184 | 77.90% | KLTH0E16192p [Lachancea thermotolerans CBS 6340] | Lachancea thermotolerans | ERG13 |
| XP_002553961 | 75.60% | KLTH0E11154p [Lachancea thermotolerans CBS 6340] | Lachancea thermotolerans | ERG10 |
| XP_002553243 | 50.10% | KLTH0D12232p [Lachancea thermotolerans CBS 6340] | Lachancea thermotolerans | ERG12 |
| XP_002553130 | 70.70% | KLTH0D09658p [Lachancea thermotolerans CBS 6340] | Lachancea thermotolerans | IDI1 |
| XP_002551773 | 69.90% | KLTH0A07238p [Lachancea thermotolerans CBS 6340] | Lachancea thermotolerans | MVD1 |
| GAA25304 | 99.60% | K7_Hmg2p [Saccharomyces cerevisiae Kyokai no. 7] | Saccharomyces cerevisiae | HMG2 |
| GAA25373 | 62.00% | K7_Hmg1p [Saccharomyces cerevisiae Kyokai no. 7] | Saccharomyces cerevisiae | HMG2 |
| GAA25373 | 99.90% | K7_Hmg1p [Saccharomyces cerevisiae Kyokai no. 7] | Saccharomyces cerevisiae | HMG1 |
| GAA25670 | 98.70% | K7_Erg8p [Saccharomyces cerevisiae Kyokai no. 7] | Saccharomyces cerevisiae | ERG8 |
| GCF00844 | 69.20% | isopentenyl-diphosphate delta-isomerase idi1 [Zygosaccharomyces mellis] | Zygosaccharomyces mellis | IDI1 |
| NP_015208 | 100.00% | isopentenyl-diphosphate delta-isomerase IDI1 [Saccharomyces cerevisiae | Saccharomyces cerevisiae | IDI1 |
| | | S288C] | | |
| PTN17316 | 99.70% | isopentenyl-diphosphate delta-isomerase IDI1 [Saccharomyces cerevisiae] | Saccharomyces cerevisiae | IDI1 |
| XP_022676509 | 69.60% | isopentenyl-diphosphate Delta-isomerase [Kluyveromyces marxianus | Kluyveromyces marxianus | IDI1 |
| | | DMKU3-1042] | | |
| OEJ82916 | 69.70% | Isopentenyl-diphosphate Delta-isomerase [Hanseniaspora osmophila] | Hanseniaspora osmophila | IDI1 |
| OEJ89771 | 54.90% | Isopentenyl-diphosphate Delta-isomerase [Hanseniaspora opuntiae] | Hanseniaspora opuntiae | IDI1 |
| KTA98145 | 75.70% | Isopentenyl-diphosphate Delta-isomerase [[Candida] glabrata] | | IDI1 |
| KQC45842 | 100.00% | Isopentenyl diphosphate: dimethylallyl diphosphate isomerase | Saccharomyces sp. | IDI1 |
| | | [Saccharomyces sp. ‘boulardii’] | | |
| AJV93575 | 99.70% | Idi1p [Saccharomyces cerevisiae YJM1527] | Saccharomyces cerevisiae | IDI1 |
| AJW10036 | 99.70% | Idi1p [Saccharomyces cerevisiae YJM1450] | Saccharomyces cerevisiae | IDI1 |
| AJW03938 | 99.70% | Idi1p [Saccharomyces cerevisiae YJM1399] | Saccharomyces cerevisiae | IDI1 |
| AJW14676 | 99.70% | Idi1p [Saccharomyces cerevisiae YJM1250] | Saccharomyces cerevisiae | IDI1 |
| AJV96549 | 99.30% | Idi1p [Saccharomyces cerevisiae YJM195] | Saccharomyces cerevisiae | IDI1 |
| EHM99886 | 92.00% | Idi1p [Saccharomyces cerevisiae × Saccharomyces kudriavzevii VIN7] | Saccharomyces cerevisiae | IDI1 |
| EGA72621 | 100.00% | Idi1p [Saccharomyces cerevisiae AWRI796] | Saccharomyces cerevisiae | IDI1 |
| EJS41430 | 89.90% | idi1p [Saccharomyces arboricola H-6] | Saccharomyces arboricola | IDI1 |
| EJT41267 | 91.70% | IDI1-like protein [Saccharomyces kudriavzevii IFO 1802] | Saccharomyces kudriavzevii | IDI1 |
| XP_018218918 | 94.40% | IDI1-like protein [Saccharomyces eubayanus] | Saccharomyces eubayanus | IDI1 |
| AQZ18416 | 72.90% | IDI1 (YPL117C) [Zygosaccharomyces parabailii] | Zygosaccharomyces parabailii | IDI1 |
| AQZ12067 | 72.50% | IDI1 (YPL117C) [Zygosaccharomyces parabailii] | Zygosaccharomyces parabailii | IDI1 |
| GAV50238 | 72.50% | hypothetical protein ZYGR_0U00940 [Zygosaccharomyces rouxii] | Zygosaccharomyces rouxii | IDI1 |
| GAV49333 | 70.50% | hypothetical protein ZYGR_0N07400 [Zygosaccharomyces rouxii] | Zygosaccharomyces rouxii | MVD1 |
| GAV56087 | 74.60% | hypothetical protein ZYGR_0AZ02590 [Zygosaccharomyces rouxii] | Zygosaccharomyces rouxii | ERG13 |
| GAV55144 | 72.10% | hypothetical protein ZYGR_0AS04680 [Zygosaccharomyces rouxii] | Zygosaccharomyces rouxii | IDI1 |
| GAV55077 | 56.00% | hypothetical protein ZYGR_0AS04000 [Zygosaccharomyces rouxii] | Zygosaccharomyces rouxii | ERG12 |
| GAV54242 | 70.80% | hypothetical protein ZYGR_0AK07440 [Zygosaccharomyces rouxii] | Zygosaccharomyces rouxii | MVD1 |
| GAV52631 | 61.20% | hypothetical protein ZYGR_0AG06220 [Zygosaccharomyces rouxii] | Zygosaccharomyces rouxii | HMG1 |
| GAV51699 | 50.30% | hypothetical protein ZYGR_0AF01700 [Zygosaccharomyces rouxii] | Zygosaccharomyces rouxii | ERG8 |
| GAV51555 | 81.40% | hypothetical protein ZYGR_0AF00260 [Zygosaccharomyces rouxii] | Zygosaccharomyces rouxii | ERG10 |
| GAV46674 | 51.50% | hypothetical protein ZYGR_0A02670 [Zygosaccharomyces rouxii] | Zygosaccharomyces rouxii | ERG8 |
| XP_003688208 | 70.40% | hypothetical protein TPHA_0M01990 [Tetrapisispora phaffii CBS 4417] | Tetrapisispora phaffii | MVD1 |
| XP_003686340 | 55.20% | hypothetical protein TPHA_0G00700 [Tetrapisispora phaffii CBS 4417] | Tetrapisispora phaffii | ERG12 |
| XP_003686328 | 50.90% | hypothetical protein TPHA_0G00580 [Tetrapisispora phaffii CBS 4417] | Tetrapisispora phaffii | ERG8 |
| XR_003684770 | 78.40% | hypothetical protein TPHA_0C01800 [Tetrapisispora phaffii CBS 4417] | Tetrapisispora phaffii | ERG10 |
| XP_003683627 | 76.10% | hypothetical protein TPHA_0A01080 [Tetrapisispora phaffii CBS 4417] | Tetrapisispora phaffii | IDI1 |
| XP_003680869 | 65.80% | hypothetical protein TDEL_0D00740 [Torulaspora delbrueckii] | Torulaspora delbrueckii | HMG1 |
| XP_003679712 | 50.20% | hypothetical protein TDEL_0B03720 [Torulaspora delbrueckii] | Torulaspora delbrueckii | ERG8 |
| XP_003679497 | 85.70% | hypothetical protein TDEL_0B01570 [Torulaspora delbrueckii] | Torulaspora delbrueckii | ERG10 |
| XP_003679373 | 76.70% | hypothetical protein TDEL_0B00330 [Torulaspora delbrueckii] | Torulaspora delbrueckii | ERG13 |
| XP_003679320 | 70.20% | hypothetical protein TDEL_0A07770 [Torulaspora delbrueckii] | Torulaspora delbrueckii | MVD1 |
| XP_003679206 | 54.10% | hypothetical protein TDEL_0A06630 [Torulaspora delbrueckii] | Torulaspora delbrueckii | ERG12 |
| XP_003679098 | 76.60% | hypothetical protein TDEL_0A05550 [Torulaspora delbrueckii] | Torulaspora delbrueckii | IDI1 |
| XP_004178780 | 67.00% | hypothetical protein TBLA_0B04230 [Tetrapisispora blattae CBS 6284] | Tetrapisispora blattae | IDI1 |
| XP_003672455 | 76.50% | hypothetical protein NDAI_0K00230 [Naumovozyma dairenensis CBS 421] | Naumovozyma dairenensis | ERG13 |
| XP_003670380 | 81.40% | hypothetical protein NDAI_0E03200 [Naumovozyma dairenensis CBS 421] | Naumovozyma dairenensis | ERG10 |
| XP_003670305 | 71.10% | hypothetical protein NDAI_0E02450 [Naumovozyma dairenensis CBS 421] | Naumovozyma dairenensis | IDI1 |
| XP_003669874 | 64.90% | hypothetical protein NDAI_0D03170 [Naumovozyma dairenensis CBS 421] | Naumovozyma dairenensis | HMG1 |
| XP_003675606 | 80.90% | hypothetical protein NCAS_0C02500 [Naumovozyma castellii CBS 4309] | Naumovozyma castellii | ERG10 |
| XP_003675530 | 75.40% | hypothetical protein NCAS_0C01740 [Naumovozyma castellii CBS 4309] | Naumovozyma castellii | IDI1 |
| XP_003675374 | 80.10% | hypothetical protein NCAS_0C00150 [Naumovozyma castellii CBS 4309] | Naumovozyma castellii | ERG13 |
| XP_003673559 | 65.90% | hypothetical protein NCAS_0A06180 [Naumovozyma castellii CBS 4309] | Naumovozyma castellii | HMG1 |
| XP_003673492 | 70.10% | hypothetical protein NCAS_0A05510 [Naumovozyma castellii CBS 4309] | Naumovozyma castellii | MVD1 |
| XP_001644409 | 55.90% | hypothetical protein Kpol_1064p33 [Vanderwaltozyma polyspora DSM 70294] | Vanderwaltozyma polyspora | ERG12 |
| XP_001646609 | 70.40% | hypothetical protein Kpol_1028p24 [Vanderwaltozyma polyspora DSM 70294] | Vanderwaltozyma polyspora | MVD1 |
| XP_001642889 | 78.10% | hypothetical protein Kpol_1007p15 [Vanderwaltozyma polyspora DSM 70294] | Vanderwaltozyma polyspora | ERG10 |
| XP_001643950 | 63.20% | hypothetical protein Kpol_1001p4 [Vanderwaltozyma polyspora DSM 70294] | Vanderwaltozyma polyspora | HMG1 |
| XP_001645637 | 70.00% | hypothetical protein Kpol_541p22 [Vanderwaltozyma polyspora DSM 70294] | Vanderwaltozyma polyspora | ERG13 |
| XP_001643379 | 75.40% | hypothetical protein Kpol_479p9 [Vanderwaltozyma polyspora DSM 70294] | Vanderwaltozyma polyspora | IDI1 |
| XP_022466532 | 49.90% | hypothetical protein KNAG_0J02060 [Kazachstania naganishii CBS 8797] | Kazachstania naganishii | ERG8 |
| XP_022466344 | 74.90% | hypothetical protein KNAG_0J00160 [Kazachstania naganishii CBS 8797] | Kazachstania naganishii | ERG13 |
| XP_022465813 | 60.30% | hypothetical protein KNAG_0H01540 [Kazachstania naganishii CBS 8797] | Kazachstania naganishii | IDI1 |
| XP_022464025 | 67.80% | hypothetical protein KNAG_0D00260 [Kazachstania naganishii CBS 8797] | Kazachstania naganishii | MVD1 |
| XP_022462169 | 77.40% | hypothetical protein KNAG_0A02340 [Kazachstania naganishii CBS 8797] | Kazachstania naganishii | ERG10 |
| XP_003959952 | 77.20% | hypothetical protein KAFR_0L02060 [Kazachstania africana CBS 2517] | Kazachstania africana | ERG13 |
| XP_003958824 | 63.80% | hypothetical protein KAFR_0H02800 [Kazachstania africana CBS 2517] | Kazachstania africana | IDI1 |
| XP_003958701 | 82.20% | hypothetical protein KAFR_0H01560 [Kazachstania africana CBS 2517] | Kazachstania africana | ERG10 |
| XP_003956599 | 70.20% | hypothetical protein KAFR_0C04730 [Kazachstania africana CBS 2517] | Kazachstania africana | MVD1 |
| XP_003955761 | 51.00% | hypothetical protein KAFR_0B03290 [Kazachstania africana CBS 2517] | Kazachstania africana | ERG8 |
| XP_003955749 | 50.90% | hypothetical protein KAFR_0B03180 [Kazachstania africana CBS 2517] | Kazachstania africana | ERG12 |
| XP_003648389 | 71.40% | Hypothetical protein Ecym_8293 [Eremothecium cymbaiariae DBVPG#7215] | Eremothecium cymbaiariae | IDI1 |
| XP_003647444 | 49.80% | hypothetical protein Ecym_6245 [Eremothecium cymbaiariae DBVPG#7215] | Eremothecium cymbaiariae | ERG8 |
| XP_003647425 | 53.80% | hypothetical protein Ecym_6226 [Eremothecium cymbaiariae DBVPG#7215] | Eremothecium cymbaiariae | ERG12 |
| XP_003647263 | 74.90% | hypothetical protein Ecym_6042 [Eremothecium cymbaiariae DBVPG#7215] | Eremothecium cymbaiariae | ERG10 |
| XP_003646450 | 75.00% | hypothetical protein Ecym_4602 [Eremothecium cymbaiariae DBVPG#7215] | Eremothecium cymbaiariae | ERG13 |
| ODV84891 | 72.80% | hypothetical protein CANARDRAFT_28632 [[Candida] arabinofermentans NRRL YB-2248] | | ERG13 |
| XP_018983430 | 72.00% | hypothetical protein BABINDRAFT_40366 [Babjeviella inositovora NRRL V- | Babjeviella inositovora | ERG13 |
| | | 12698] | | |
| OXB41221 | 66.20% | hypothetical protein B1J91_L11506g [[Candida] glabrata] | | HMG1 |
| OXB44968 | 72.10% | hypothetical protein B1J91_C03630g [[Candida] glabrata] | | MVD1 |
| NP_013580 | 100.00% | hydroxymethylglutaryl-CoA synthase [Saccharomyces cerevisiae S288C] | Saccharomyces cerevisiae | ERG13 |
| PTN15827 | 99.80% | hydroxymethylglutaryl-CoA synthase [Saccharomyces cerevisiae] | Saccharomyces cerevisiae | ERG13 |
| XP_022677516 | 79.40% | hydroxymethylglutaryl-CoA synthase [Kluyveromyces marxianus DMKU3- | Kluyveromyces marxianus | ERG13 |
| | | 1042] | | |
| BAP73180 | 80.00% | hydroxymethylglutaryl-CoA synthase [Kluyveromyces marxianus] | Kluyveromyces marxianus | ERG13 |
| XP_020069485 | 73.70% | hydroxymethylglutaryl-CoA synthase [Cyberlindnera jadinii NRRL Y-1542] | Cyberlindnera jadinii | ERG13 |
| NP_013555 | 100.00% | hydroxymethylglutaryl-CoA reductase (NADPH) HMG2 [Saccharomyces | Saccharomyces cerevisiae | HMG2 |
| | | cerevisiae S288C] | | |
| PTN30829 | 99.50% | hydroxymethylglutaryl-CoA reductase (NADPH) HMG2 [Saccharomyces | Saccharomyces cerevisiae | HMG2 |
| | | cerevisiae] | | |
| PTN23346 | 99.40% | hydroxymethylglutaryl-CoA reductase (NADPH) HMG2 [Saccharomyces | Saccharomyces cerevisiae | HMG2 |
| | | cerevisiae] | | |
| NP_013636 | 100.00% | hydroxymethylglutaryl-CoA reductase (NADPH) HMG1 [Saccharomyces | Saccharomyces cerevisiae | HMG1 |
| | | cerevisiae S288C] | | |
| PTN24696 | 62.80% | hydroxymethylglutaryl-CoA reductase (NADPH) HMG1 [Saccharomyces | Saccharomyces cerevisiae | HMG2 |
| | | cerevisiae] | | |
| PTN24696 | 99.70% | hydroxymethylglutaryl-CoA reductase (NADPH) HMG1 [Saccharomyces | Saccharomyces cerevisiae | HMG1 |
| | | cerevisiae] | | |
| KOH49325 | 99.60% | HMG2p HMG-CoA reductase [Saccharomyces sp. ‘boulardii’] | Saccharomyces sp. | HMG2 |
| AJV68413 | 99.60% | Hmg2p [Saccharomyces cerevisiae YJM1478] | Saccharomyces cerevisiae | HMG2 |
| AJV67508 | 99.40% | Hmg2p [Saccharomyces cerevisiae YJM1463] | Saccharomyces cerevisiae | HMG2 |
| AJV66156 | 99.50% | Hmg2p [Saccharomyces cerevisiae YJM1447] | Saccharomyces cerevisiae | HMG2 |
| AJV63093 | 99.90% | Hmg2p [Saccharomyces cerevisiae YJM1418] | Saccharomyces cerevisiae | HMG2 |
| AJV60837 | 99.80% | Hmg2p [Saccharomyces cerevisiae YJM1400] | Saccharomyces cerevisiae | HMG2 |
| AJV60387 | 99.20% | Hmg2p [Saccharomyces cerevisiae YJM1399] | Saccharomyces cerevisiae | HMG2 |
| AJV57705 | 99.80% | Hmg2p [Saccharomyces cerevisiae YJM1383] | Saccharomyces cerevisiae | HMG2 |
| AJV56799 | 99.60% | Hmg2p [Saccharomyces cerevisiae YJM1356] | Saccharomyces cerevisiae | HMG2 |
| AJV56344 | 99.70% | Hmg2p [Saccharomyces cerevisiae YJM1355] | Saccharomyces cerevisiae | HMG2 |
| AJV55892 | 99.90% | Hmg2p [Saccharomyces cerevisiae YJM1342] | Saccharomyces cerevisiae | HMG2 |
| AJV55003 | 99.90% | Hmg2p [Saccharomyces cerevisiae YJM1338] | Saccharomyces cerevisiae | HMG2 |
| AJV54558 | 99.60% | Hmg2p [Saccharomyces cerevisiae YJM1336] | Saccharomyces cerevisiae | HMG2 |
| AJV52757 | 99.50% | Hmg2p [Saccharomyces cerevisiae YJM1307] | Saccharomyces cerevisiae | HMG2 |
| AJV52306 | 99.70% | Hmg2p [Saccharomyces cerevisiae YJM1304] | Saccharomyces cerevisiae | HMG2 |
| AJV5J863 | 99.70% | Hmg2p [Saccharomyces cerevisiae YJM1273] | Saccharomyces cerevisiae | HMG2 |
| AJV50514 | 99.70% | Hmg2p [Saccharomyces cerevisiae YJM1248] | Saccharomyces cerevisiae | HMG2 |
| AJV49196 | 99.60% | Hmg2p [Saccharomyces cerevisiae YJM1208] | Saccharomyces cerevisiae | HMG2 |
| AJV47381 | 99.70% | Hmg2p [Saccharomyces cerevisiae YJM1133] | Saccharomyces cerevisiae | HMG2 |
| AJV46930 | 99.70% | Hmg2p [Saccharomyces cerevisiae YJM1129] | Saccharomyces cerevisiae | HMG2 |
| AJV46478 | 99.60% | Hmg2p [Saccharomyces cerevisiae YJM1083] | Saccharomyces cerevisiae | HMG2 |
| AHY78797 | 99.60% | Hmg2p [Saccharomyces cerevisiae YJM993] | Saccharomyces cerevisiae | HMG2 |
| AJV78151 | 99.70% | Hmg2p [Saccharomyces cerevisiae YJM456] | Saccharomyces cerevisiae | HMG2 |
| AJV75447 | 99.50% | Hmg2p [Saccharomyces cerevisiae YJM320] | Saccharomyces cerevisiae | HMG2 |
| AJV74606 | 99.50% | Hmg2p [Saccharomyces cerevisiae YJM270] | Saccharomyces cerevisiae | HMG2 |
| AJV73338 | 99.70% | Hmg2p [Saccharomyces cerevisiae YJM195] | Saccharomyces cerevisiae | HMG2 |
| EHN05753 | 99.60% | Hmg2p [Saccharomyces cerevisiae × Saccharomyces kudriavzevii VIN7] | Saccharomyces cerevisiae | HMG2 |
| EHN01037 | 92.50% | Hmg2p [Saccharomyces cerevisiae × Saccharomyces kudriavzevii VIN7] | Saccharomyces cerevisiae | HMG2 |
| EGA77584 | 99.70% | Hmg2p [Saccharomyces cerevisiae Vin13] | Saccharomyces cerevisiae | HMG2 |
| EWG89789 | 99.60% | Hmg2p [Saccharomyces cerevisiae P301] | Saccharomyces cerevisiae | HMG2 |
| EGA81622 | 99.60% | Hmg2p [Saccharomyces cerevisiae Lalvin QA23] | Saccharomyces cerevisiae | HMG2 |
| EJT44740 | 91.80% | HMG2-like protein [Saccharomyces kudriavzevii IFO 1802] | Saccharomyces kudriavzevii | HMG2 |
| XP_018220830 | 91.00% | HMG2-like protein [Saccharomyces eubayanus] | Saccharomyces eubayanus | HMG2 |
| AQZ18362 | 63.60% | HMG2 (YLR450W) and HMG1 (YML075C) [Zygosaccharomyces parabailii] | Zygosaccharomyces parabailii | HMG1 |
| AQZ15653 | 63.60% | HMG2 (YLR450W) and HMG1 (YML075C) [Zygosaccharomyces parabailii] | Zygosaccharomyces parabailii | HMG1 |
| AJT00194 | 61.90% | Hmg1p [Saccharomyces cerevisiae YJM1574] | Saccharomyces cerevisiae | HMG2 |
| AJT00194 | 99.80% | Hmg1p [Saccharomyces cerevisiae YJM1574] | Saccharomyces cerevisiae | HMG1 |
| AJS96703 | 99.50% | Hmg1p [Saccharomyces cerevisiae YJM1463] | Saccharomyces cerevisiae | HMG1 |
| AJS96264 | 99.90% | Hmg1p [Saccharomyces cerevisiae YJM1460] | Saccharomyces cerevisiae | HMG1 |
| AJS90608 | 99.90% | Hmg1p [Saccharomyces cerevisiae YJM1401] | Saccharomyces cerevisiae | HMG1 |
| AJS90173 | 99.60% | Hmg1p [Saccharomyces cerevisiae YJM1400] | Saccharomyces cerevisiae | HMG1 |
| AJS88421 | 61.90% | Hmg1p [Saccharomyces cerevisiae YJM1387] | Saccharomyces cerevisiae | HMG2 |
| AJS88421 | 99.70% | Hmg1p [Saccharomyces cerevisiae YJM1387] | Saccharomyces cerevisiae | HMG1 |
| AJS85371 | 62.50% | Hmg1p [Saccharomyces cerevisiae YJM1342] | Saccharomyces cerevisiae | HMG2 |
| AJS85371 | 99.60% | Hmg1p [Saccharomyces cerevisiae YJM1342] | Saccharomyces cerevisiae | HMG1 |
| AJS81024 | 99.80% | Hmg1p [Saccharomyces cerevisiae YJM1252] | Saccharomyces cerevisiae | HMG1 |
| AJS80590 | 99.80% | Hmg1p [Saccharomyces cerevisiae YJM1250] | Saccharomyces cerevisiae | HMG1 |
| AJS79281 | 61.90% | Hmg1p [Saccharomyces cerevisiae YJM1242] | Saccharomyces cerevisiae | HMG2 |
| AJS79281 | 99.80% | Hmg1p [Saccharomyces cerevisiae YJM1242] | Saccharomyces cerevisiae | HMG1 |
| AJS76667 | 99.80% | Hmg1p [Saccharomyces cerevisiae YJM1129] | Saccharomyces cerevisiae | HMG1 |
| AHY76391 | 99.90% | Hmg1p [Saccharomyces cerevisiae YJM993] | Saccharomyces cerevisiae | HMG1 |
| AHY76391 | 61.90% | Hmg1p [Saccharomyces cerevisiae YJM993] | Saccharomyces cerevisiae | HMG2 |
| AJS72296 | 99.80% | Hmg1p [Saccharomyces cerevisiae YJM969] | Saccharomyces cerevisiae | HMG1 |
| AJS71856 | 99.90% | Hmg1p [Saccharomyces cerevisiae YJM693] | Saccharomyces cerevisiae | HMG1 |
| AJS70550 | 99.70% | Hmg1p [Saccharomyces cerevisiae YJM682] | Saccharomyces cerevisiae | HMG1 |
| AJS69670 | 99.60% | Hmg1p [Saccharomyces cerevisiae YJM627] | Saccharomyces cerevisiae | HMG1 |
| AJS64422 | 99.80% | Hmg1p [Saccharomyces cerevisiae YJM271] | Saccharomyces cerevisiae | HMG1 |
| AJS63986 | 62.30% | Hmg1p [Saccharomyces cerevisiae YJM270] | Saccharomyces cerevisiae | HMG2 |
| AJS63986 | 99.70% | Hmg1p [Saccharomyces cerevisiae YJM270] | Saccharomyces cerevisiae | HMG1 |
| AJS62677 | 99.80% | Hmg1p [Saccharomyces cerevisiae YJM195] | Saccharomyces cerevisiae | HMG1 |
| AJS62242 | 99.80% | Hmg1p [Saccharomyces cerevisiae YJM193] | Saccharomyces cerevisiae | HMG1 |
| EGA77439 | 100.00% | Hmg1p [Saccharomyces cerevisiae Vin13] | Saccharomyces cerevisiae | HMG1 |
| EWG94281 | 99.80% | Hmg1p [Saccharomyces cerevisiae R103] | Saccharomyces cerevisiae | HMG1 |
| EWG83860 | 99.80% | Hmg1p [Saccharomyces cerevisiae R008] | Saccharomyces cerevisiae | HMG1 |
| EEU05004 | 99.70% | Hmg1p [Saccharomyces cerevisiae JAY291] | Saccharomyces cerevisiae | HMG1 |
| EGA57422 | 99.50% | Hmg1p [Saccharomyces cerevisiae FostersB] | Saccharomyces cerevisiae | HMG1 |
| CAY81746 | 62.60% | Hmg1p [Saccharomyces cerevisiae EC1118] | Saccharomyces cerevisiae | HMG2 |
| CAY81746 | 99.60% | Hmg1p [Saccharomyces cerevisiae EC1118] | Saccharomyces cerevisiae | HMG1 |
| EJS42513 | 91.90% | hmg1p [Saccharomyces arboricola H-6] | Saccharomyces arboricola | HMG1 |
| XP_018219995 | 91.00% | HMG1-like protein [Saccharomyces eubayanus] | Saccharomyces eubayanus | HMG1 |
| KZV08767 | 61.90% | HMG1 [Saccharomyces cerevisiae] | Saccharomyces cerevisiae | HMG2 |
| KZV08767 | 99.70% | HMG1 [Saccharomyces cerevisiae] | Saccharomyces cerevisiae | HMG1 |
| XP_017988495 | 74.10% | HER221Cp [Eremothecium sinecaudum] | Eremothecium sinecaudum | ERG13 |
| XP_017986617 | 72.20% | HCL530Cp [Eremothecium sinecaudum] | Eremothecium sinecaudum | IDI1 |
| AEY98585 | 68.50% | FAGL232Cp [Eremothecium gossypii FDAG1] | Eremothecium gossypii | MVD1 |
| AJS92313 | 99.80% | Erg13p [Saccharomyces cerevisiae YJM1418] | Saccharomyces cerevisiae | ERG13 |
| AJS89693 | 99.80% | Erg13p [Saccharomyces cerevisiae YJM1399] | Saccharomyces cerevisiae | ERG13 |
| AJS82290 | 99.60% | Erg13p [Saccharomyces cerevisiae YJM1307] | Saccharomyces cerevisiae | ERG13 |
| AJS67872 | 99.80% | Erg13p [Saccharomyces cerevisiae YJM470] | Saccharomyces cerevisiae | ERG13 |
| AJS66556 | 99.60% | Erg13p [Saccharomyces cerevisiae YJM451] | Saccharomyces cerevisiae | ERG13 |
| AJS65680 | 99.80% | Erg13p [Saccharomyces cerevisiae YJM428] | Saccharomyces cerevisiae | ERG13 |
| AJS63065 | 99.80% | Erg13p [Saccharomyces cerevisiae YJM244] | Saccharomyces cerevisiae | ERG13 |
| EWG94231 | 99.80% | Erg13p [Saccharomyces cerevisiae R103] | Saccharomyces cerevisiae | ERG13 |
| EWG89196 | 99.80% | Erg13p [Saccharomyces cerevisiae P301] | Saccharomyces cerevisiae | ERG13 |
| EGA57459 | 99.80% | Erg13p [Saccharomyces cerevisiae FostersB] | Saccharomyces cerevisiae | ERG13 |
| EGA81523 | 100.00% | Erg13p, partial [Saccharomyces cerevisiae Lalvin QA23] | Saccharomyces cerevisiae | ERG13 |
| EJT44320 | 97.40% | ERG13-like protein [Saccharomyces kudriavzevii IFO 1802] | Saccharomyces kudriavzevii | ERG13 |
| XP_018219948 | 95.90% | ERG13-like protein [Saccharomyces eubayanus] | Saccharomyces eubayanus | ERG13 |
| AQZ15814 | 75.10% | ERG13 (YML126C) [Zygosaccharomyces parabailii] | Zygosaccharomyces parabailii | ERG13 |
| AJS98710 | 99.50% | Erg12p [Saccharomyces cerevisiae YJM1526] | Saccharomyces cerevisiae | ERG12 |
| AJS96096 | 99.50% | Erg12p [Saccharomyces cerevisiae YJM1450] | Saccharomyces cerevisiae | ERG12 |
| AJS95662 | 99.50% | Erg12p [Saccharomyces cerevisiae YJM1447] | Saccharomyces cerevisiae | ERG12 |
| AJS90876 | 99.50% | Erg12p [Saccharomyces cerevisiae YJM1401] | Saccharomyces cerevisiae | ERG12 |
| AJS90009 | 99.50% | Erg12p [Saccharomyces cerevisiae YJM1399] | Saccharomyces cerevisiae | ERG12 |
| AJS81726 | 99.50% | Erg12p [Saccharomyces cerevisiae YJM1273] | Saccharomyces cerevisiae | ERG12 |
| AJS80425 | 99.50% | Erg12p [Saccharomyces cerevisiae YJM1248] | Saccharomyces cerevisiae | ERG12 |
| AJS77376 | 99.50% | Erg12p [Saccharomyces cerevisiae YJM1133] | Saccharomyces cerevisiae | ERG12 |
| AJP40902 | 99.50% | Erg12p [Saccharomyces cerevisiae YJM1078] | Saccharomyces cerevisiae | ERG12 |
| AHY76662 | 99.80% | Erg12p [Saccharomyces cerevisiae YJM993] | Saccharomyces cerevisiae | ERG12 |
| AJS68191 | 99.80% | Erg12p [Saccharomyces cerevisiae YJM470] | Saccharomyces cerevisiae | ERG12 |
| AJS65126 | 99.30% | Erg12p [Saccharomyces cerevisiae YJM320] | Saccharomyces cerevisiae | ERG12 |
| AJS64256 | 99.50% | Erg12p [Saccharomyces cerevisiae YJM270] | Saccharomyces cerevisiae | ERG12 |
| AJS63818 | 99.30% | Erg12p [Saccharomyces cerevisiae YJM248] | Saccharomyces cerevisiae | ERG12 |
| AJS62946 | 99.50% | Erg12p [Saccharomyces cerevisiae YJM195] | Saccharomyces cerevisiae | ERG12 |
| EHN05445 | 99.60% | Erg12p [Saccharomyces cerevisiae × Saccharomyces kudriavzevii VIN7] | Saccharomyces cerevisiae | ERG12 |
| EHN00772 | 89.40% | Erg12p [Saccharomyces cerevisiae × Saccharomyces kudriavzevii VIN7] | Saccharomyces cerevisiae | ERG12 |
| EGA77322 | 99.30% | Erg12p [Saccharomyces cerevisiae Vin13] | Saccharomyces cerevisiae | ERG12 |
| EGA73546 | 99.30% | Erg12p [Saccharomyces cerevisiae AWRI796] | Saccharomyces cerevisiae | ERG12 |
| EJS44170 | 88.70% | erg12p [Saccharomyces arboricola H-6] | Saccharomyces arboricola | ERG12 |
| EJT42123 | 89.80% | ERG12-like protein [Saccharomyces kudriavzevii IFO 1802] | Saccharomyces kudriavzevii | ERG12 |
| XP_018220256 | 87.40% | ERG12-like protein [Saccharomyces eubayanus] | Saccharomyces eubayanus | ERG12 |
| AQZ14941 | 55.10% | ERG12 (YMR208W) [Zygosaccharomyces parabailii] | Zygosaccharomyces parabailii | ERG12 |
| AQZ10756 | 55.30% | ERG12 (YMR208W) [Zygosaccharomyces parabailii] | Zygosaccharomyces parabailii | ERG12 |
| AJV94633 | 99.70% | Erg10p [Saccharomyces cerevisiae YJM1574] | Saccharomyces cerevisiae | ERG10 |
| AJV91203 | 99.70% | Erg10p [Saccharomyces cerevisiae YJM1460] | Saccharomyces cerevisiae | ERG10 |
| AJW10118 | 99.70% | Erg10p [Saccharomyces cerevisiae YJM1450] | Saccharomyces cerevisiae | ERG10 |
| AJW07512 | 99.50% | Erg10p [Saccharomyces cerevisiae YJM1433] | Saccharomyces cerevisiae | ERG10 |
| AJW04020 | 99.70% | Erg10p [Saccharomyces cerevisiae YJM1399] | Saccharomyces cerevisiae | ERG10 |
| AJW19535 | 99.70% | Erg10p [Saccharomyces cerevisiae YJM1342] | Saccharomyces cerevisiae | ERG10 |
| AJW25866 | 99.70% | Erg10p [Saccharomyces cerevisiae YJM969] | Saccharomyces cerevisiae | ERG10 |
| AJW25209 | 99.70% | Erg10p [Saccharomyces cerevisiae YJM689] | Saccharomyces cerevisiae | ERG10 |
| AJV98817 | 99.70% | Erg10p [Saccharomyces cerevisiae YJM320] | Saccharomyces cerevisiae | ERG10 |
| EHN04392 | 99.80% | Erg10p [Saccharomyces cerevisiae × Saccharomyces kudriavzevii VIN7] | Saccharomyces cerevisiae | ERG10 |
| EGA76382 | 100.00% | Erg10p [Saccharomyces cerevisiae Vin13] | Saccharomyces cerevisiae | ERG10 |
| EJS41294 | 96.00% | erg10p [Saccharomyces arboricola H-6] | Saccharomyces arboricola | ERG10 |
| XP_018218998 | 95.50% | ERG10-like protein [Saccharomyces eubayanus] | Saccharomyces eubayanus | ERG10 |
| AQZ14383 | 82.20% | ERG10 (YPL028W) [Zygosaccharomyces parabailii] | Zygosaccharomyces parabailii | ERG10 |
| AQZ10340 | 82.70% | ERG10 (YPL028W) [Zygosaccharomyces parabailii] | Zygosaccharomyces parabailii | ERG10 |
| GCE99731 | 81.20% | erg10, acetyl-CoA C-acetyltransferase [Zygosaccharomyces mellis] | Zygosaccharomyces mellis | ERG10 |
| AJT01353 | 98.20% | Erg8p [Saccharomyces cerevisiae YJM1615] | Saccharomyces cerevisiae | ERG8 |
| AJS97853 | 99.30% | Erg8p [Saccharomyces cerevisiae YJM1478] | Saccharomyces cerevisiae | ERG8 |
| AJS96980 | 98.20% | Erg8p [Saccharomyces cerevisiae YJM1463] | Saccharomyces cerevisiae | ERG8 |
| AJS95674 | 98.20% | Erg8p [Saccharomyces cerevisiae YJM1447] | Saccharomyces cerevisiae | ERG8 |
| AJS92643 | 98.90% | Erg8p [Saccharomyces cerevisiae YJM1418] | Saccharomyces cerevisiae | ERG8 |
| AJS91766 | 99.30% | Erg8p [Saccharomyces cerevisiae YJM1415] | Saccharomyces cerevisiae | ERG8 |
| AJS90021 | 98.40% | Erg8p [Saccharomyces cerevisiae YJM1399] | Saccharomyces cerevisiae | ERG8 |
| AJS89145 | 98.20% | Erg8p [Saccharomyces cerevisiae YJM1388] | Saccharomyces cerevisiae | ERG8 |
| AJS87837 | 99.10% | Erg8p [Saccharomyces cerevisiae YJM1385] | Saccharomyces cerevisiae | ERG8 |
| AJS85654 | 98.40% | Erg8p [Saccharomyces cerevisiae YJM1342] | Saccharomyces cerevisiae | ERG8 |
| AJS84771 | 98.20% | Erg8p [Saccharomyces cerevisiae YJM1338] | Saccharomyces cerevisiae | ERG8 |
| AJS81738 | 98.20% | Erg8p [Saccharomyces cerevisiae YJM1273] | Saccharomyces cerevisiae | ERG8 |
| AJS80865 | 98.40% | Erg8p [Saccharomyces cerevisiae YJM1250] | Saccharomyces cerevisiae | ERG8 |
| AJS80437 | 98.40% | Erg8p [Saccharomyces cerevisiae YJM1248] | Saccharomyces cerevisiae | ERG8 |
| AJS78262 | 98.20% | Erg8p [Saccharomyces cerevisiae YJM1199] | Saccharomyces cerevisiae | ERG8 |
| AJS77388 | 98.40% | Erg8p [Saccharomyces cerevisiae YJM1133] | Saccharomyces cerevisiae | ERG8 |
| AHY76674 | 99.60% | Erg8p [Saccharomyces cerevisiae YJM993] | Saccharomyces cerevisiae | ERG8 |
| AJS72138 | 98.90% | Erg8p [Saccharomyces cerevisiae YJM693] | Saccharomyces cerevisiae | ERG8 |
| AJS70390 | 98.20% | Erg8p [Saccharomyces cerevisiae YJM681] | Saccharomyces cerevisiae | ERG8 |
| AJS68638 | 98.40% | Erg8p [Saccharomyces cerevisiae YJM541] | Saccharomyces cerevisiae | ERG8 |
| AJS68203 | 99.30% | Erg8p [Saccharomyces cerevisiae YJM470] | Saccharomyces cerevisiae | ERG8 |
| AJS66886 | 99.30% | Erg8p [Saccharomyces cerevisiae YJM451] | Saccharomyces cerevisiae | ERG8 |
| AJS65138 | 98.40% | Erg8p [Saccharomyces cerevisiae YJM320] | Saccharomyces cerevisiae | ERG8 |
| AJS62958 | 98.20% | Erg8p [Saccharomyces cerevisiae YJM195] | Saccharomyces cerevisiae | ERG8 |
| EHN00784 | 82.70% | Erg8p [Saccharomyces cerevisiae × Saccharomyces kudriavzevii VIN7] | Saccharomyces cerevisiae | ERG8 |
| EWG84132 | 99.30% | Erg8p [Saccharomyces cerevisiae R008] | Saccharomyces cerevisiae | ERG8 |
| EEU06624 | 98.20% | Erg8p [Saccharomyces cerevisiae JAY291] | Saccharomyces cerevisiae | ERG8 |
| EGA57236 | 99.30% | Erg8p [Saccharomyces cerevisiae FostersB] | Saccharomyces cerevisiae | ERG8 |
| EJS44177 | 80.50% | erg8p [Saccharomyces arboricola H-6] | Saccharomyces arboricola | ERG8 |
| AQZ17926 | 51.20% | ERG8 (YMR220W) [Zygosaccharomyces parabailii] | Zygosaccharomyces parabailii | ERG8 |
| AQZ11848 | 51.70% | ERG8 (YMR220W) [Zygosaccharomyces parabailii] | Zygosaccharomyces parabailii | ERG8 |
| NP_014441 | 100.00% | diphosphomevalonate decarboxylase MVD1 [Saccharomyces cerevisiae | Saccharomyces cerevisiae | MVD1 |
| | | S288C] | | |
| GCE98064 | 69.80% | diphosphomevalonate decarboxylase [Zygosaccharomyces mellis] | Zygosaccharomyces mellis | MVD1 |
| XP_011275729 | 69.80% | Diphosphomevalonate decarboxylase [Wickerhamomyces ciferrii] | Wickerhamomyces ciferrii | MVD1 |
| XP_022674578 | 72.20% | diphosphomevalonate decarboxylase [Kluyveromyces marxianus DMKU3- | Kluyveromyces marxianus | MVD1 |
| | | 1042] | | |
| ONH68647 | 68.20% | Diphosphomevalonate decarboxylase [Cyberlindnera fabianii] | Cyberlindnera fabianii | MVD1 |
| KTB12572 | 72.10% | Diphosphomevalonate decarboxylase [[Candida] glabrata] | | MVD1 |
| KTA97751 | 72.10% | Diphosphomevalonate decarboxylase [[Candida] glabrata] | | MVD1 |
| CDR37714 | 68.40% | CYFA0S01e15566g1_1 [Cyberlindnera fabianii] | Cyberlindnera fabianii | MVD1 |
| IFI4_A | 97.80% | Chain A, MEVALONATE 5-DIPHOSPHATE DECARBOXYLASE | Saccharomyces cerevisiae | MVD1 |
| | | [Saccharomyces cerevisiae] | | |
| 5XZ5_A | 100.00% | Chain A, Acetyl-CoA acetyltransferase [Saccharomyces cerevisiae S288C] | Saccharomyces cerevisiae | ERG10 |
| 5XYJ_A | 99.70% | Chain A, Acetyl-CoA acetyltransferase [Saccharomyces cerevisiae S288C] | Saccharomyces cerevisiae | ERG10 |
| NP_986435 | 68.50% | AGL232Cp [Eremothecium gossypii ATCC 10895] | Eremothecium gossypii | MVD1 |
| NP_984262 | 76.60% | ADR165Cp [Eremothecium gossypii ATCC 10895] | Eremothecium gossypii | ERG10 |
| NP_983739 | 75.60% | ADL356Cp [Eremothecium gossypii ATCC 10895] | Eremothecium gossypii | ERG13 |
| NP_983828 | 71.40% | ADL268Cp [Eremothecium gossypii ATCC 10895] | Eremothecium gossypii | IDI1 |
| NP_015297 | 100.00% | acetyl-CoA C-acetyltransferase [Saccharomyces cerevisiae S288C] | Saccharomyces cerevisiae | ERG10 |
| GAX68822 | 99.50% | acetyl-CoA C-acetyltransferase [Saccharomyces cerevisiae] | Saccharomyces cerevisiae | ERG10 |
| CDH13613 | 82.20% | Acetyl-CoA acetyltransferase [Zygosaccharomyces bailii ISA1307] | Zygosaccharomyces bailii | ERG10 |
| XP_022677456 | 76.70% | acetyl-CoA acetyltransferase [Kluyveromyces marxianus DMKU3-1042] | Kluyveromyces marxianus | ERG10 |
| BAP73114 | 76.90% | acetyl-CoA acetyltransferase [Kluyveromyces marxianus] | Kluyveromyces marxianus | ERG10 |
| KTA99270 | 81.40% | Acetyl-CoA acetyltransferase [[Candida] glabrata] | | ERG10 |
| CCA60775 | 96.00% | acetoacetyl CoA thiolase [Saccharomyces uvarum] | Saccharomyces uvarum | ERG10 |
| AGO14103 | 77.40% | AaceriADR165Cp [Saccharomycetaceae sp. ‘Ashbya aceri’] | Saccharomycetaceae sp. | ERG10 |
| AGO12980 | 71.00% | AaceriADL268Cp [Saccharomycetaceae sp. ‘Ashbya aceri’] | Saccharomycetaceae sp. | IDI1 |
| GCE98385 | 73.80% | 3-hydroxy-3-methylglutaryl coenzyme A synthase [Zygosaccharomyces | Zygosaccharomyces mellis | ERG13 |
| | | mellis] | | |
| ONH78258 | 99.90% | 3-hydroxy-3-methylglutaryl-coenzyme A reductase [Saccharomyces | Saccharomyces cerevisiae | HMG1 |
| | | cerevisiae] | | |
| ONH76081 | 99.50% | 3-hydroxy-3-methylglutaryl-coenzyme A reductase [Saccharomyces | Saccharomyces cerevisiae | HMG2 |
| | | cerevisiae] | | |

| | | | |
|---|---|---|---|
| KTB22480 | 66.20% | 3-hydroxy-3-methylglutaryl-coenzyme A reductase 1 [[Candida] glabrata] | HMG1 |
| KTA97912 | 66.10% | 3-hydroxy-3-methylglutaryl-coenzyme A reductase 1 [[Candida] glabrata] | HMG1 |

### Genetic Engineering of the Acetyl-CoA (PDH Bypass) Pathway

In some embodiments, the recombinant microbial cell is engineered to possess one or more enzyme activities that results in an increased flux through the PDH bypass pathway, to thereby increase the amount of cytosolic acetyl-CoA. In some embodiments, the one or more enzymatic activities is selected from pyruvate decarboxylase activity, acetyl-CoA synthetase activity, acetyl-CoA synthetase isoform 2 activity, and acetaldehyde dehydrogenase activity. In some embodiments, the recombinant microbial cell comprises one or more polynucleotide(s) encoding one or more of the following enzymes of the acetyl-CoA (PDH bypass) pathway: pyruvate decarboxylase (PDC), acetyl-CoA synthetase isoform 1 (ACS1), acetyl-CoA synthetase isoform 2 (ACS2), and acetaldehyde dehydrogenase (ALD6). In some embodiments, the one or more polynucleotide(s) encoding one or more enzymes of the acetyl-CoA (PDH bypass) pathway is derived from Saccharomyces cerevisiae.

Without being bound by theory, it is thought that the overexpression of one or more enzymes of the acetyl-CoA (PDH bypass) pathway may increase the flux through PDH bypass pathway to increase the amount of cytosolic acetyl-CoA in the recombinant microbial cells of this disclosure, which may in turn increase the flux through the mevalonate and nepetalactol synthesis pathways, ultimately resulting in an increased production of nepetalactol/nepetalactone/dihydronepetalactone.

In some embodiments, the recombinant microbial cell is engineered to overexpress one or more of the enzymes of the PDH bypass pathway. In some embodiments, the recombinant microbial cell is engineered to overexpress all of the enzymes of the PDH bypass pathway. The amount of the enzyme expressed by the recombinant microbial cell may be higher than the amount of that corresponding enzyme in a wild type microbial cell by about 1.25 fold to about 20 fold, for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 8 fold, about 9 fold, about 10 fold, about 15 fold, about 20 fold, including all the subranges and values that lie therebetween.

In some embodiments the recombinant microbial cell has been modified to contain a heterologous promoter operably linked to one or more endogenous PDH bypass pathway genes. In some embodiments, the heterologous promoter is a stronger promoter, as compared to the native promoter of the PDH bypass pathway gene. In some embodiments, the recombinant microbial cell is engineered to express an enzyme of the PDH bypass pathway constitutively. For instance, in some embodiments, the recombinant microbial cell may express an enzyme of the PDH bypass pathway at a time when the enzyme is not expressed by the wild type microbial cell.

In other embodiments, the present disclosure envisions overexpressing one or more PDH bypass genes by increasing the copy number of said PDH bypass gene. Thus, in some embodiments, the recombinant microbial cell comprises at least one additional copy of a DNA sequence encoding an enzyme of the PDH bypass pathway, as compared to a wild type microbial cell. In some embodiments, the recombinant microbial cell comprises at least one additional copy of a DNA sequence encoding an enzyme of PDH bypass pathway, as compared to a wild type microbial cell. In some embodiments, the recombinant microbial cell comprises 1 to 40 additional copies of a DNA sequence encoding an enzyme of the PDH bypass pathway, as compared to a wild type microbial cell. For instance, the recombinant microbial cell may comprise 1, 2, 3, 4, 5, 6, 7, 8, 9, 10, 15, 20, 25, 30, 35, or 40 additional copies of the DNA sequence, compared to a wild type microbial cell, including any ranges and subranges therebetween. In some embodiments, the recombinant microbial cell comprises 1 to 2 additional copies of a DNA sequence encoding an enzyme of the PDH bypass pathway, as compared to a wild type microbial cell. In some embodiments, the recombinant microbial cell comprises 1 to 2 additional copies of a DNA sequence encoding each of the enzymes of the PDH bypass pathway, as compared to a wild type microbial cell.

In some embodiments, the present disclosure teaches methods of increasing nepetalactol biosynthesis by expressing one or more mutant PDH bypass pathway genes. Thus, in some embodiments, the recombinant microbial cell comprises a DNA sequence encoding for one or more mutant PDH bypass pathway enzymes. In some embodiments, the one or more mutant PDH bypass pathway enzymes are more catalytically active that the corresponding wild type enzyme. In some embodiments, the one or more mutant PDH bypass pathway enzymes have a higher k_{Cat}, as compared to the wild type enzyme. In some embodiments, the one or more mutant PDH bypass pathway enzymes that are more catalytically active than the wild type enzyme, are insensitive to negative regulation, such as, for example, allosteric inhibition.

In some embodiments, the recombinant microbial cell comprises a polynucleotide encoding an enzyme of the PDH bypass pathway, wherein the polynucleotide comprises a nucleic acid sequence having at least about 80% identity to the nucleic acid sequence of the corresponding wild type form of the polynucleotide present in the wild type microbial cell. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%/o, or about 100% identity to the corresponding wild type form of the polynucleotide present in the wild type microbial cell, including any ranges and subranges therebetween.

In some embodiments, the recombinant microbial cell expresses an enzyme of the PDH bypass pathway, wherein the enzyme comprises an amino acid sequence comprising at least 80% identity to the sequence of the corresponding enzyme expressed by the wild type microbial cell. In some embodiments, the enzyme expressed by the recombinant microbial cell comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to the corresponding wild type enzyme expressed by the wild type microbial cell. In some embodiments, the enzyme expressed by the recombinant microbial cell may comprise an amino acid sequence that is truncated as compared to the wild type enzyme expressed by the wild type microbial cell, including any ranges and subranges therebetween.

In some embodiments, the recombinant microbial cell expresses a recombinant enzyme of the PDH bypass pathway. In some embodiments, the recombinant enzyme is a homolog derived from another microbial species, a plant cell or a mammalian cell. In some embodiments, the homolog is more catalytically active as compared to the wild type enzyme expressed by the wild type microbial cell.

### Genetic Engineering of the Nepetalactol Pathway

In some embodiments, the recombinant microbial cell comprises one or more polynucleotide(s) encoding one or more of the enzymes of the nepetalactol synthesis pathway listed in Table 2. For instance, in some embodiments, the recombinant microbial cell comprises one or more polynucleotide(s) encoding one or more of the following enzymes of the nepetalactol synthesis pathway: geraniol diphosphate synthase (GPPS), a geranyl diphosphate diphosphatase (geraniol synthase, GES), a geraniol 8-hydroxylase (G8H), a cytochrome P450 reductase (CPR) capable of promoting regeneration of the redox state of the G8H, cytochrome B5 reductase (CYBR or CYB5R), an 8-hydroxygeraniol dehydrogenase (8HGO), an iridoid synthase (ISY) and NEPS. In some embodiments, the recombinant microbial cell comprises one or more polynucleotide(s) encoding each of the enzymes of the nepetalactol synthesis pathway listed in Table 2.

Without wishing to be bound by one theory, it is thought that the expression of one or more enzymes of the nepetalactone pathway may result in increased amounts of nepetalactol/nepetalactone/dihydronepetalactone in the recombinant microbial cells of this disclosure.

In some embodiments, the recombinant microbial cell comprises one or more polynucleotide(s) encoding cytochrome B5 (CytB5 or CYB5), which is capable of promoting the regeneration of redox state of G8H. The expression of CytB5 in a recombinant microbial cell for the production of nepetalactol/nepetalactone/dihydronepetalactone has not been described previously in the art (for example, see Campbell, Alex, Thesis, 2016), thus further distinguishing the recombinant microbial cells and the methods of this disclosure from the existing art.

In some embodiments, the recombinant microbial cell comprises 1 to 40 copies of a DNA sequence encoding an enzyme of the nepetalactol synthesis pathway. For instance, the recombinant microbial cell may comprise 1, 2, 3, 4, 5, 6, 7, 8, 9, 10, 15, 20, 25, 30, 35, or 40 copies of the DNA sequence, including all ranges and subranges therebetween. For example, in some embodiments, the recombinant microbial cell comprises at least one copy of a DNA sequence encoding one or more of the following: GPPS, GES, G8H, CPR, CytB5, CYBR, 8HGO, ISY, and NEPS. In some embodiments, the recombinant microbial cell comprises 3-5 copies of a DNA sequence encoding one or more of the following enzymes: GPPS, G8H, CPR, and CYBR In some embodiments, the recombinant microbial cell comprises 3-5 copies of a DNA sequence encoding CytB5. In some embodiments, the recombinant microbial cell comprises 6-20 copies of a DNA sequence encoding GPPS and/or G8H.

In some embodiments, the recombinant microbial cell is engineered to express one or more of the enzymes of the nepetalactol synthesis pathway listed in Table 2. In some embodiments, the recombinant microbial cell is engineered to express each of the enzymes of the nepetalactol synthesis pathway listed in Table 2.

In some embodiments, the recombinant microbial cell comprises a polynucleotide encoding GPPS, wherein the polynucleotide comprises a nucleic acid sequence having at least about 80% identity to a nucleic acid sequence selected from SEQ ID Nos. 789-927. In some embodiments, the recombinant microbial cell comprises a polynucleotide comprising a nucleic acid sequence having about 80%, about 81%, about 82%, about 83%, about 84%, about 85%, about 86%, about 87%0, about 88%, about 89%, about 90%, about 91%, about 92%, about 93%, about 94%, about 95%, about 96%, about 970, about 98%, about 990, or about 100% identity to a nucleic acid selected from SEQ ID Nos. 789-927, including all ranges and subranges therebetween.

In some embodiments, the recombinant microbial cell expresses an enzyme of the nepetalactol synthesis pathway, wherein the enzyme is GPPS, and GPPS comprises an amino acid sequence comprising at least 80% identity to an amino acid sequence selected from SEQ ID Nos. 1-139. In some embodiments, the enzyme expressed by the recombinant microbial cell comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 1-139, including all ranges and subranges therebetween.

In some embodiments, the recombinant microbial cell comprises a polynucleotide encoding GES, wherein the polynucleotide comprises a nucleic acid sequence having at least about 80% identity to a nucleic acid sequence selected from SEQ ID Nos. 928-1037. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 928-1037, including all ranges and subranges therebetween.

In some embodiments, the recombinant microbial cell expresses an enzyme of the nepetalactol synthesis pathway, wherein the enzyme is GES, and GES comprises an amino acid sequence comprising at least 80% identity to an amino acid sequence selected from SEQ ID Nos. 140-249. In some embodiments, the enzyme expressed by the recombinant microbial cell comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 140-249, including all ranges and subranges therebetween.

In some embodiments, the recombinant microbial cell comprises a polynucleotide encoding G8H, wherein the polynucleotide comprises a nucleic acid sequence having at least about 80% identity to a nucleic acid sequence selected from SEQ ID Nos. 1038-1072 and 1088-1110. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1038-1072 and 1088-1110, including all ranges and subranges therebetween.

In some embodiments, the recombinant microbial cell expresses an enzyme of the nepetalactol synthesis pathway, wherein the enzyme is G8H, and G8H comprises an amino acid sequence comprising at least 80% identity to an amino acid sequence selected from SEQ ID Nos. 250-284 and 300-322. In some embodiments, the enzyme expressed by the recombinant microbial cell comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 250-284 and 300-322, including all ranges and subranges therebetween.

In some embodiments, the recombinant microbial cell comprises a polynucleotide encoding CPR, wherein the polynucleotide comprises a nucleic acid sequence having at least about 80% identity to a nucleic acid sequence selected from SEQ ID Nos. 1073-1087. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1073-1087, including all ranges and subranges therebetween.

In some embodiments, the recombinant microbial cell expresses an enzyme of the nepetalactol synthesis pathway, wherein the enzyme is CPR, and CPR comprises an amino acid sequence comprising at least 80% identity to an amino acid sequence selected from SEQ ID Nos. 285-299. In some embodiments, the enzyme expressed by the recombinant microbial cell comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 285-299, including all ranges and subranges therebetween.

In some embodiments, the recombinant microbial cell comprises a polynucleotide encoding CYB5, wherein the polynucleotide comprises a nucleic acid sequence having at least about 80% identity to a nucleic acid sequence selected from SEQ ID Nos. 1111-1117. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1111-1117, including all ranges and subranges therebetween.

In some embodiments, the recombinant microbial cell expresses an enzyme of the nepetalactol synthesis pathway, wherein the enzyme is CYB5, and CYB5 comprises an amino acid sequence comprising at least 80% identity to an amino acid sequence selected from SEQ ID Nos. 323-329. In some embodiments, the enzyme expressed by the recombinant microbial cell comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 323-329.

In some embodiments, the recombinant microbial cell comprises a polynucleotide encoding 8HGO, wherein the polynucleotide comprises a nucleic acid sequence having at least about 80% identity to a nucleic acid sequence selected from SEQ ID Nos. 1118-1156. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1118-1156, including all ranges and subranges therebetween.

In some embodiments, the recombinant microbial cell expresses an enzyme of the nepetalactol synthesis pathway, wherein the enzyme is 8HGO, and 8HGO comprises an amino acid sequence comprising at least 80% identity to an amino acid sequence selected from SEQ ID Nos. 330-368. In some embodiments, the enzyme expressed by the recombinant microbial cell comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 330-368, including all ranges and subranges therebetween.

In some embodiments, the recombinant microbial cell comprises a polynucleotide encoding ISY, wherein the polynucleotide comprises a nucleic acid sequence having at least about 80% identity to a nucleic acid sequence selected from SEQ ID Nos. 1157-1307 and 1778-1807. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1157-1307 and 1778-1807, including all ranges and subranges therebetween.

In some embodiments, the recombinant microbial cell expresses an enzyme of the nepetalactol synthesis pathway, wherein the enzyme is ISY, and ISY comprises an amino acid sequence comprising at least 80% identity to an amino acid sequence selected from SEQ ID Nos. 369-519 and 1695-1724. In some embodiments, the enzyme expressed by the recombinant microbial cell comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 369-519 and 1695-1724, including all ranges and subranges therebetween.

In some embodiments, the recombinant microbial cell comprises a polynucleotide encoding CYB5R, wherein the polynucleotide comprises a nucleic acid sequence having at least about 80% identity to a nucleic acid sequence selected from SEQ ID Nos. 1571-1576. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1571-1576, including all ranges and subranges therebetween.

In some embodiments, the recombinant microbial cell expresses an enzyme of the nepetalactol synthesis pathway, wherein the enzyme is CYB5R, and CYB5R comprises an amino acid sequence comprising at least 80% identity to an amino acid sequence selected from SEQ ID Nos. 783-788. In some embodiments, the enzyme expressed by the recombinant microbial cell comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 783-788, including all ranges and subranges therebetween.

In some embodiments, the recombinant microbial cell expresses homolog of an enzyme of the nepetalactol synthesis pathway derived from another microbial species, a plant cell or a mammalian cell. In some embodiments, the homolog is selected from the nepetalactol synthesis pathway enzyme homologs listed in Table 6.

TABLE 6
An exemplary list of homologs of nepetalactol synthesis pathway enzymes

| Protein | | |
|---|---|---|
| SEQ ID | Gene | |
| NO. | name | Source organism |
| 1 | GPPS | Saccharomyces cerevisiae |
| 2 | GPPS | Saccharomyces cerevisiae |
| 3 | GPPS | Abies grandis |
| 4 | GPPS | Catharanthus roseus |
| 5 | GPPS | Picea abies |
| 6 | GPPS | Geobacillussp.WSUCF1 |
| 7 | GPPS | Saccharomyces cerevisiae(strainATCC204508/S288c)(Baker'syeast) |
| 8 | GPPS | Saccharomyces cerevisiae(strainATCC204508/S288c)(Baker'syeast) |
| 9 | GPPS | Saccharomyces cerevisiae(strainATCC204508/S288c)(Baker'syeast) |
| 10 | GPPS | Neosartorya fumigata (strain ATCC MYA-4609/Af293/CBS 101355/FGSC A1100) |
| | | (Aspergillus fumigatus) |
| 11 | GPPS | Catharanthus roseus (Madagascar periwinkle) (Vinca rosea) |
| 12 | GPPS | Rhizobium acidisoli |
| 13 | GPPS | Escherichiacoli(strainK12) |
| 14 | GPPS | Escherichiacoli(strainK12) |
| 15 | GPPS | Brucella suis (strain ATCC 23445/NCTC 10510) |
| 16 | GPPS | Arabidopsisthaliana(Mouse-earcress) |
| 17 | GPPS | Buchneraaphidicolasubsp.Acyrthosiphonpisum(strainAPS)(Acyrthosiphonpisumsymbioticbacterium) |
| 18 | GPPS | Dendroctonus ponderosae (Mountain pine beetle) |
| 19 | GPPS | Picea abies (Norway spruce) (Picea excelsa) |
| 20 | GPPS | Abies grandis (Grand fir) (Pinus grandis) |
| 21 | GPPS | Corynebacterium glutamicum (strain ATCC 13032/DSM 20300/JCM 1318/LMG 3730/NCIMB 10025) |
| 22 | GPPS | Vitisvinifera(Grape) |
| 23 | GPPS | Picea abies (Norway spruce) (Picea excelsa) |
| 24 | GPPS | Picea abies (Norway spruce) (Picea excelsa) |
| 25 | GPPS | Sus scrofa (Pig) |
| 26 | GPPS | Acyrthosiphon pisum (Pea aphid) |
| 27 | GPPS | Mycobacteriumtuberculosis |
| 28 | GPPS | Staphylococcus aureus (strain NCTC 8325) |
| 29 | GPPS | Geobacillussp.WSUCF1 |
| 30 | GPPS | Saccharomycescerevisiae(strainATCC204508/S288c)(Baker'syeast) |
| 31 | GPPS | Neosartorya fumigata (strain ATCC MYA-4609/Af293/CBS 101355/FGSC A1100) |
| | | (Aspergillus fumigatus) |
| 32 | GPPS | Neosartorya fumigata (strain ATCC MYA-4609/Af293/CBS 101355/FGSC A1100) |
| | | (Aspergillus fumigatus) |
| 33 | GPPS | Catharanthus roseus (Madagascar periwinkle) (Vinca rosea) |
| 34 | GPPS | Rhizobium acidisoli |
| 35 | GPPS | Escherichiacoli(strainK12) |
| 36 | GPPS | Brucella suis (strain ATCC 23445/NCTC 10510) |
| 37 | GPPS | Arabidopsisthaliana(Mouse-earcress) |
| 38 | GPPS | Buchneraaphidicolasubsp.Acyrthosipbonpisum(strainAPS)(Acyrthosiphonpisumsymbioticbacterium) |
| 39 | GPPS | Dendroctonus ponderosae (Mountain pine beetle) |
| 40 | GPPS | Picea abies (Norway spruce) (Picea excelsa) |
| 41 | GPPS | Abies grandis (Grand fir) (Pinus grandis) |
| 42 | GPPS | Corynebacterium glutamicum (strain ATCC 13032/DSM 20300/JCM 1318/LMG 3730/NCIMB 10025) |
| 43 | GPPS | Vitisvinifera(Grape) |
| 44 | GPPS | Picea abies (Norway spruce) (Picea excelsa) |
| 45 | GPPS | Picea abies (Norway spruce) (Picea excelsa) |
| 46 | GPPS | Picea abies (Norway spruce) (Picea excelsa) |
| 47 | GPPS | Picea abies (Norway spruce) (Picea excelsa) |
| 48 | GPPS | Picea abies (Norway spruce) (Picea excelsa) |
| 49 | GPPS | Sus scrofa (Pig) |
| 50 | GPPS | Acyrthosiphon pisum (Pea aphid) |
| 51 | GPPS | Mycobacteriumtuberculosis |
| 52 | GPPS | Staphylococcus aureus (strain NCTC 8325) |
| 53 | GPPS | Geobacillussp.WSUCF1 |
| 54 | GPPS | Geobacillussp.WSUCF1 |
| 55 | GPPS | Geobaciliussp.WSUCF1 |
| 56 | GPPS | Geobacillussp.WSUCF1 |
| 57 | GPPS | Rhizobium acidisoli |
| 58 | GPPS | Rhizobium acidisoli |
| 59 | GPPS | Rhizobium acidisoli |
| 60 | GPPS | Escherichiacoli(strainK12) |
| 61 | GPPS | Escherichiacoli(strainK12) |
| 62 | GPPS | Escherichiacoli(strainK12) |
| 63 | GPPS | Brucella suis (strain ATCC 23445/NCTC 10510) |
| 64 | GPPS | Brucella suis (strain ATCC 23445/NCTC 10510) |
| 65 | GPPS | Buchneraaphidicolasubsp.Acyrthosiphonpisum(strainAPS)(Acyrthosiphonpisumsymbioticbacterium) |
| 66 | GPPS | Buchneraaphidicolasubsp.Acyrthosiphonpisum(strainAPS)(Acyrthosiphonpisumsymbioticbacterium) |
| 67 | GPPS | Buchneraaphidicolasubsp.Acyrthosiphonpisum(strainAPS)(Acyrthosiphonpisumsymbioticbacterium) |
| 68 | GPPS | Dendroctonus ponderosae (Mountain pine beetle) |
| 69 | GPPS | Picea abies (Norway spruce) (Picea excelsa) |
| 70 | GPPS | Picea abies (Norway spruce) (Picea excelsa) |
| 71 | GPPS | Picea abies (Norway spruce) (Picea excelsa) |
| 72 | GPPS | Abies grandis (Grand fir) (Pinus grandis) |
| 73 | GPPS | Abies grandis (Grand fir) (Finns grandis) |
| 74 | GPPS | Abies grandis (Grand fir) (Pinus grandis) |
| 75 | GPPS | Picea abies (Norway spruce) (Picea excelsa) |
| 76 | GPPS | Picea abies (Norway spruce) (Picea excelsa) |
| 77 | GPPS | Picea abies (Norway spruce) (Picea excelsa) |
| 78 | GPPS | Sus scrofa (Pig) |
| 79 | GPPS | Staphylococcus aureus (strain NCTC 8325) |
| 80 | GPPS | Staphylococcus aureus (strain NCTC 8325) |
| 81 | GPPS | Staphylococcus aureus (strain NCTC 8325) |
| 82 | GPPS | Geobacillussp.WSUCF1 |
| 83 | GPPS | Saccharomycescerevisiae(strainATCC204508/S288c)(Baker'syeast) |
| 84 | GPPS | Neosartorya fumigata (strain ATCC MYA-4609/A1293/CBS 101355/FGSC A1100) |
| | | (Aspergillus fumigatus) |
| 85 | GPPS | Catharanthus roseus (Madagascar periwinkle) (Vinca rosea) |
| 86 | GPPS | Catharanthus roseus (Madagascar periwinkle) (Vinca rosea) |
| 87 | GPPS | Catharanthus roseus (Madagascar periwinkle) (Vinca rosea) |
| 88 | GPPS | Rhizobium acidisoli |
| 89 | GPPS | Escherichiacoli(strainK12) |
| 90 | GPPS | Brucella suis (strain ATCC 23445/NCTC 10510) |
| 91 | GPPS | Arabidopsisthaliana(Mouse-earcress) |
| 92 | GPPS | Arabidopsisthaliana(Mouse-earcress) |
| 93 | GPPS | Arabidopsisthaliana(Mouse-earcress) |
| 94 | GPPS | Buchneraaphidicolasubsp.Acyrthosiphonpisum(strainAPS)(Acyrthosiphonpisumsymbioticbacterium) |
| 95 | GPPS | Dendroctonus ponderosae (Mountain pine beetle) |
| 96 | GPPS | Picea abies (Norway spruce) (Picea excelsa) |
| 97 | GPPS | Abies grandis (Grand fir) (Pinus grandis) |
| 98 | GPPS | Corynebacterium glutamicum (strain ATCC 13032/DSM 20300/JCM 1318/LMG 3730/NC1MB 10025) |
| 99 | GPPS | Vitisvinifera(Grape) |
| 100 | GPPS | Vitisvinifera(Grape) |
| 101 | GPPS | Vitisvinifera(Grape) |
| 102 | GPPS | Picea abies (Norway spruce) (Picea excelsa) |
| 103 | GPPS | Sus scrofa (Pig) |
| 104 | GPPS | Acyrthosiphon pisum (Pea aphid) |
| 105 | GPPS | Mycobacteriumtuberculosis |
| 106 | GPPS | Mycobacteriumtuberculosis |
| 107 | GPPS | Mycobacteriumtuberculosis |
| 108 | GPPS | Staphylococcus aureus (strain NCTC 8325) |
| 109 | GPPS | Picea abies |
| 110 | GPPS | Abies grandis |
| 111 | GPPS | Catharanthus roseus |
| 112 | GPPS | Picea abies |
| 113 | GPPS | Abies grandis |
| 114 | GPPS | Catharanthus roseus |
| 115 | GPPS | Abies grandis |
| 116 | GPPS | Catharanthus roseus and S. cerevisiae |
| 117 | GPPS | Picea abies |
| 118 | GPPS | Humulus lupulus |
| 119 | GPPS | Humulus lupulus |
| 120 | GPPS | Mentha × piperita |
| 121 | GPPS | Mentha × piperita |
| 122 | GPPS | Catharanthus roseus |
| 123 | GPPS | Catharanthus roseus |
| 124 | GPPS | Nepeta cataria |
| 125 | GPPS | Nepeta cataria |
| 126 | GPPS | Streptomyces aculeolatus |
| 127 | GPPS | Streptomyces sp. KO-3988 |
| 128 | GPPS | Streptomyces cinnamonensis |
| 129 | GPPS | Streptomyces longwoodensis |
| 130 | GPPS | Streptomyces sp. GKU 895 |
| 131 | GPPS | Streptomyces sp. NRRL S-37 |
| 132 | GPPS | Streptomyces aculeolatus |
| 133 | GPPS | Streptomyces sp. KO-3988 |
| 134 | GPPS | Streptomyces cinnamonensis |
| 135 | GPPS | Streptomyces longwoodensis |
| 136 | GPPS | Streptomyces sp. GKU 895 |
| 137 | GPPS | Streptomyces sp. NRRL S-37 |
| 138 | GPPS | Penicillium aethiopicum |
| 139 | GPPS | Penicillium aethiopicum |
| 140 | GES | Ocimum basilicum (Sweet basil) |
| 141 | GES | Catharanthus roseus |
| 142 | GES | Ocimum basilicum |
| 143 | GES | Valeriana officinalis |
| 144 | GES | Catharanthus roseus |
| 145 | GES | Ocimum basilicum |
| 146 | GES | Valeriana officinalis |
| 147 | GES | Catharanthus roseus |
| 148 | GES | Ocimum basilicum |
| 149 | GES | Perilla citriodora |
| 150 | GES | Valeriana officinalis |
| 151 | GES | Rosa hybrid cultivar |
| 152 | GES | Arabidopsis thaliana |
| 153 | GES | Catharanthus roseus |
| 154 | GES | Ocimum basilicum |
| 155 | GES | Perilla citriodora |
| 156 | GES | Valeriana officinalis |
| 157 | GES | Vinca minor |
| 158 | GES | Cinchona pubescens |
| 159 | GES | Rauvolfia serpentina |
| 160 | GES | Swertia japonica |
| 161 | GES | Coffea canephora |
| 162 | GES | Citrus unshiu |
| 163 | GES | Citrus unshiu |
| 164 | GES | Glycine soja |
| 165 | GES | Cynara cardunculus var. scolymus |
| 166 | GES | Dorcoceras hygrometricum |
| 167 | GES | Dorcoceras hygrometricum |
| 168 | GES | Helianthus annuus |
| 169 | GES | Actinidia chinensis var. chinensis |
| 170 | GES | Cinchona ledgeriana |
| 171 | GES | Lonicera japonica |
| 172 | GES | Cinchona pubescens |
| 173 | GES | Nepeta mussinii |
| 174 | GES | Nepeta cataria |
| 175 | GES | Nepeta cataria |
| 176 | GES | Phyla dulcis |
| 177 | GES | Vitis vinifera |
| 178 | GES | Catharanthus roseus |
| 179 | GES | Olea europaea |
| 180 | GES | Valeriana officinalis |
| 181 | GES | Valeriana officinalis |
| 182 | GES | Valeriana officinalis |
| 183 | GES | Pogostemon cablin |
| 184 | GES | Picrorhiza kurrooa |
| 185 | GES | Gentiana rigescens |
| 186 | GES | Camptotheca acuminata |
| 187 | GES | Osmanthus fragrans |
| 188 | GES | synthetic construct |
| 189 | GES | Phaseolus lunatus |
| 190 | GES | unknown |
| 191 | GES | Vigna angularis var. angularis |
| 192 | GES | Vitis vinifera |
| 193 | GES | Coffea arabica |
| 194 | GES | Coffea canephora |
| 195 | GES | Glycine soja |
| 196 | GES | Glycine soja |
| 197 | GES | Vigna angularis |
| 198 | GES | Glycine max |
| 199 | GES | Cajanus cajan |
| 200 | GES | Cajanus cajan |
| 201 | GES | Vitis vinifera |
| 202 | GES | Vitis vinifera |
| 203 | GES | Glycine max |
| 204 | GES | Lupinus angustifolius |
| 205 | GES | Handroanthus impetiginosus |
| 206 | GES | Handroanthus impetiginosus |
| 207 | GES | Lactuca sativa |
| 208 | GES | Parasponia andersonii |
| 209 | GES | Trema orientalis |
| 210 | GES | unknown |
| 211 | GES | unknown |
| 212 | GES | Ricinus communis |
| 213 | GES | Medicago truncatula |
| 214 | GES | Cicer arietinum |
| 215 | GES | Glycine max |
| 216 | GES | Glycine max |
| 217 | GES | Phaseolus vulgaris |
| 218 | GES | Phaseolus vulgaris |
| 219 | GES | Phaseolus vulgaris |
| 220 | GES | Morus notabilis |
| 221 | GES | Vitis vinifera |
| 222 | GES | Sesamum indicum |
| 223 | GES | Jatropha curcas |
| 224 | GES | Erythranthe guttata |
| 225 | GES | Vigna radiata var. radiata |
| 226 | GES | Vigna radiata var. radiata |
| 227 | GES | Arachis duranensis |
| 228 | GES | Vigna angularis |
| 229 | GES | Vigna angularis |
| 230 | GES | Lupinus angustifolius |
| 231 | GES | Cajanus cajan |
| 232 | GES | Cajanus cajan |
| 233 | GES | Manihot esculenta |
| 234 | GES | Hevea brasiliensis |
| 235 | GES | Helianthus annuus |
| 236 | GES | Olea europaea var. sylvestris |
| 237 | GES | Lactuca sativa |
| 238 | GES | Citrus clementina |
| 239 | GES | Medicago truncatula |
| 240 | GES | Cicer arietinum |
| 241 | GES | Citrus sinensis |
| 242 | GES | Vigna angularis |
| 243 | GES | Helianthus annuus |
| 244 | GES | Helianthus annuus |
| 245 | GES | Helianthus annuus |
| 246 | GES | Olea europaea var. sylvestris |
| 247 | GES | Olea europaea var. sylvestris |
| 248 | GES | Olea europaea var. sylvestris |
| 249 | GES | Olea europaea var. sylvestris |
| 250 | G8H | Catharanthus roseus |
| 251 | G8H | Catharanthus roseus |
| 252 | G8H | Catharanthus roseus |
| 253 | G8H | Catharanthus roseus |
| 254 | G8H | Catharanthus roseus |
| 255 | G8H | Catharanthus roseus |
| 256 | G8H | Catharanthus roseus |
| 257 | G8H | Catharanthus roseus |
| 258 | G8H | Catharanthus roseus |
| 259 | G8H | Catharanthus roseus |
| 260 | G8H | Catharanthus roseus |
| 261 | G8H | Catharanthus roseus |
| 262 | G8H | Catharanthus roseus |
| 263 | G8H | Catharanthus roseus |
| 264 | G8H | Nepeta cataria |
| 265 | G8H | Nepeta mussinii |
| 266 | G8H | Nepeta cataria |
| 267 | G8H | Nepeta mussinii |
| 268 | G8H | Nepeta cataria |
| 269 | G8H | Nepeta mussinii |
| 270 | G8H | Nepeta cataria |
| 271 | G8H | Nepeta mussinii |
| 272 | G8H | Vigna angularis |
| 273 | G8H | Bacillus megaterium NBRC 15308 |
| 274 | G8H | Bacillus megaterium NBRC 15308 |
| 275 | G8H | Camptotheca acuminata |
| 276 | G8H | Vinca minor |
| 277 | G8H | Ophiorrhiza pumila |
| 278 | G8H | Rauvolfia serpentina |
| 279 | G8H | Lonicera japonica |
| 280 | G8H | Erythranthe guttata |
| 281 | G8H | Picrorhiza kurrooa |
| 282 | G8H | Olea europaea |
| 283 | G8H | Gentiana rigescens |
| 284 | G8H | Nepeta cataria |
| 285 | CPR | Arabidopsis thaliana |
| 286 | CPR | Catharanthus roseus |
| 287 | CPR | Catharanthus roseus |
| 288 | CPR | Arabidopsis thaliana |
| 289 | CPR | Catharanthus roseus |
| 290 | CPR | Arabidopsis thaliana |
| 291 | CPR | Catharanthus roseus |
| 292 | CPR | Nepeta mussinii |
| 293 | CPR | Camptotheca acuminata |
| 294 | CPR | Arabidopsis thaliana |
| 295 | CPR | Arabidopsis thaliana |
| 296 | CPR | Nepeta mussinii |
| 297 | CPR | Camptotheca acuminata |
| 298 | CPR | Nepeta mussinii |
| 299 | CPR | Camptotheca acuminata |
| 300 | G8H | Swertia mussotii |
| 301 | G8H | Camptotheca acuminata |
| 302 | G8H | Lonicera japonica |
| 303 | G8H | Erythranthe guttata |
| 304 | G8H | Erythranthe guttata |
| 305 | G8H | Nepeta cataria |
| 306 | G8H | Picrorhiza kurrooa |
| 307 | G8H | Picrorhiza kurrooa |
| 308 | G8H | Nepeta mussinii |
| 309 | G8H | Olea europaea |
| 310 | G8H | Sesamum indicum |
| 311 | G8H | Coffea canephora |
| 312 | G8H | Dorcoceras hygrometricum |
| 313 | G8H | Gentiana rigescens |
| 314 | G8H | Vinca minor |
| 315 | G8H | Ophiorrhiza pumila |
| 316 | G8H | Rauvolfia serpentina |
| 317 | G8H | Cinchona calisaya |
| 318 | G8H | Tabernaemontana elegans |
| 319 | G8H | Catharanthus roseus |
| 320 | G8H | Catharanthus roseus |
| 321 | G8H | Catharanthus roseus |
| 322 | G8H | Catharanthus roseus |
| 323 | CYB5 | Catharanthus roseus |
| 324 | CYB5 | Yarrowia lipolytica CLIB122 |
| 325 | CYB5 | Nepeta cataria |
| 326 | CYB5 | Catharanthus roseus |
| 327 | CYB5 | Nepeta cataria |
| 328 | CYB5 | Artemesia annua |
| 329 | CYB5 | Arabidopsis thaliana |
| 330 | 8HGO | Catharanthus roseus (Madagascar periwinkle) (Vinca rosea) |
| 331 | 8HGO | Catharanthus roseus |
| 332 | 8HGO | Nepeta cataria |
| 333 | 8HGO | Sesamum indicum |
| 334 | 8HGO | Camptotheca acuminata |
| 335 | 8HGO | Sesamum indicum |
| 336 | 8HGO | Swertia japonica |
| 337 | 8HGO | Ophiorrhiza pumila |
| 338 | 8HGO | Cinchona ledgeriana |
| 339 | 8HGO | Lonicera japonica |
| 340 | 8HGO | Coffea canephora |
| 341 | 8HGO | Rauvolfia serpentina |
| 342 | 8HGO | Gentiana rigescens |
| 343 | 8HGO | Catharanthus roseus |
| 344 | 8HGO | Nepeta cataria |
| 345 | 8HGO | Ocimum basilicum |
| 346 | 8HGO | Sesamum indicum |
| 347 | 8HGO | Capsicum annuum |
| 348 | 8HGO | Camptotheca acuminata |
| 349 | 8HGO | Solanum tuberosum |
| 350 | 8HGO | Sesamum indicum |
| 351 | 8HGO | Swertia japonica |
| 352 | 8HGO | Ophiorrhiza pumila |
| 353 | 8HGO | Cinchona ledgeriana |
| 354 | 8HGO | Lonicera japonica |
| 355 | 8HGO | Coffea canephora |
| 356 | 8HGO | Rauvolfia serpentina |
| 357 | 8HGO | Gentiana rigescens |
| 358 | 8HGO | Catharanthus roseus |
| 359 | 8HGO | Olea europaea subsp. europaea |
| 360 | 8HGO | Sesamum indicum |
| 361 | 8HGO | Olea europaea |
| 362 | 8HGO | Erythranthe guttata |
| 363 | 8HGO | Catharanthus roseus |
| 364 | 8HGO | Ocimum basilicum |
| 365 | 8HGO | Camptotheca acuminata |
| 366 | 8HGO | Swertia japonica |
| 367 | 8HGO | Cinchona ledgeriana |
| 368 | 8HGO | Rauvolfia serpentina |
| 369 | ISY | Arabidopsis thaliana (Mouse-earcress) |
| 370 | ISY | Digitalis lanata (Grecian foxglove) |
| 371 | ISY | Nepeta mussinii |
| 372 | ISY | Nepeta cataria |
| 373 | ISY | Catharanthus roseus (Madagascar periwinkle) (Vinca rosea) |
| 374 | ISY | Catharanthus roseus |
| 375 | ISY | Nepeta mussinii |
| 376 | ISY | Nepeta cataria |
| 377 | ISY | Olea europaea |
| 378 | ISY | Catharanthus roseus |
| 379 | ISY | Nepeta mussinii |
| 380 | ISY | Nepeta cataria |
| 381 | ISY | Nicotiana tabacum |
| 382 | ISY | Elaeis guineensis |
| 383 | ISY | Citrus clementina |
| 384 | ISY | Sesamum indicum |
| 385 | ISY | Camptotheca acuminata |
| 386 | ISY | Cinchona pubescens |
| 387 | ISY | Ophiorrhiza pumila |
| 388 | ISY | Lonicera japonica |
| 389 | ISY | Digitalis purpurea |
| 390 | ISY | Antirrhinum majus |
| 391 | ISY | Trifolium subterraneum |
| 392 | ISY | Corchorus capsularis |
| 393 | ISY | Nicotiana tabacum |
| 394 | ISY | Panicum hallii |
| 395 | ISY | Medicago truncatula |
| 396 | ISY | Juglans regia |
| 397 | ISY | Triticum urartu |
| 398 | ISY | Citrus clementina |
| 399 | ISY | Panicum hallii |
| 400 | ISY | Prunus persica |
| 401 | ISY | Tarenaya hassleriana |
| 402 | ISY | Capsicum baccatum |
| 403 | ISY | Medicago truncatula |
| 404 | ISY | Nicotiana sylvestris |
| 405 | ISY | Oryza sativa Japonica Group |
| 406 | ISY | Oryza sativa Japonica Group |
| 407 | ISY | Cynara cardunculus var. scolymus |
| 408 | ISY | Ornithogalum longebracteatum |
| 409 | ISY | Allium ursinum |
| 410 | ISY | Convallaria majalis |
| 411 | ISY | Populus trichocarpa |
| 412 | ISY | Sorghum bicolor |
| 413 | ISY | Zea mays |
| 414 | ISY | Daucus carota subsp. sativus |
| 415 | ISY | Nepeta cataria |
| 416 | ISY | Catharanthus roseus |
| 417 | ISY | Dichanthelium oligosanthes |
| 418 | ISY | Sorghum bicolor |
| 419 | ISY | Tarenaya hassleriana |
| 420 | ISY | Citrus sinensis |
| 421 | ISY | Picea sitchensis |
| 422 | ISY | Cajanus cajan |
| 423 | ISY | Citrus clementina |
| 424 | ISY | Aquilegia coerulea |
| 425 | ISY | Lonicera japonica |
| 426 | ISY | Olea europaea subsp. europaea |
| 427 | ISY | Thlaspi densiflorum |
| 428 | ISY | Stellaria media |
| 429 | ISY | Erysimum crepidifolium |
| 430 | ISY | Morus notabilis |
| 431 | ISY | Helianthus annuus |
| 432 | ISY | Capsicum annuum |
| 433 | ISY | Macleaya cordata |
| 434 | ISY | Citrus clementina |
| 435 | ISY | Arachis ipaensis |
| 436 | ISY | Vitis vinifera |
| 437 | ISY | Hevea brasiliensis |
| 438 | ISY | Dorcoceras hygrometricum |
| 439 | ISY | Brassica napus |
| 440 | ISY | Ziziphus jujuba |
| 441 | ISY | Punica granatum |
| 442 | ISY | Capsicum baccatum |
| 443 | ISY | Carica papaya |
| 444 | ISY | Gossypium hirsutum |
| 445 | ISY | Cucumis sativus |
| 446 | ISY | Citrus clementina |
| 447 | ISY | Catharanthus roseus |
| 448 | ISY | Fragaria vesca subsp. vesca |
| 449 | ISY | Prunus avium |
| 450 | ISY | Salvia rosmarinus |
| 451 | ISY | Elaeis guineensis |
| 452 | ISY | Erythranthe guttata |
| 453 | ISY | Helianthus annuus |
| 454 | ISY | Genlisea aurea |
| 455 | ISY | Arabidopsis thaliana |
| 456 | ISY | Lupinus angustifolius |
| 457 | ISY | Ananas comosus |
| 458 | ISY | Beta vulgaris subsp. vulgaris |
| 459 | ISY | Gossypium raimondii |
| 460 | ISY | Citrus sinensis |
| 461 | ISY | Amborella trichopoda |
| 462 | ISY | Musa acuminata subsp. malaccensis |
| 463 | ISY | Zostera marina |
| 464 | ISY | Cephalotus follicularis |
| 465 | ISY | Ipomoea nil |
| 466 | ISY | Ricinus communis |
| 467 | ISY | Elaeis guineensis |
| 468 | ISY | Citrus clementina |
| 469 | ISY | Musa acuminata subsp. malaccensis |
| 470 | ISY | Theobroma cacao |
| 471 | ISY | Gomphocarpus fruticosus |
| 472 | ISY | Lupinus angustifoiius |
| 473 | ISY | Brachypodium distachyon |
| 474 | ISY | Oryza brachyantha |
| 475 | ISY | Catharanthus roseus |
| 476 | ISY | Populus euphratica |
| 477 | ISY | Catharanthus roseus |
| 478 | ISY | Prunus mume |
| 479 | ISY | Ziziphus jujuba |
| 480 | ISY | Prunus persica |
| 481 | ISY | Sesamum indicum |
| 482 | ISY | Panicum hallii |
| 483 | ISY | Fragaria vesca subsp. vesca |
| 484 | ISY | Setaria italica |
| 485 | ISY | Populus trichocarpa |
| 486 | ISY | Juglans regia |
| 487 | ISY | Jatropha curcas |
| 488 | ISY | Hevea brasiliensis |
| 489 | ISY | Camptotheca acuminata |
| 490 | ISY | Malus domestica |
| 491 | ISY | Panicum hallii |
| 492 | ISY | Arachis duranensis |
| 493 | ISY | Catharanthus roseus |
| 494 | ISY | Spinacia oleracea |
| 495 | ISY | Trifolium subterraneum |
| 496 | ISY | Ziziphus jujuba |
| 497 | ISY | Medicago truncatula |
| 498 | ISY | Medicago truncatula |
| 499 | ISY | Medicago truncatula |
| 500 | ISY | Spinacia oleracea |
| 501 | ISY | Juglans regia |
| 502 | ISY | Populus tremuloides |
| 503 | ISY | Vitis vinifera |
| 504 | ISY | Vitis vinifera |
| 505 | ISY | Daucus carota subsp. sativus |
| 506 | ISY | Dendrobium catenatum |
| 507 | ISY | Passiflora incarnata |
| 508 | ISY | Prunus avium |
| 509 | ISY | Daucus carota subsp. sativus |
| 510 | ISY | Solanum tuberosum |
| 511 | ISY | Setaria italica |
| 512 | ISY | Antirrhinum majus |
| 513 | ISY | Coffea canephora |
| 514 | ISY | Panicum hallii |
| 515 | ISY | Oryza sativa Japonica Group |
| 516 | ISY | Setaria italica |
| 517 | ISY | Sesamum indicum |
| 518 | ISY | Digitalis purpurea |
| 519 | ISY | Digitalis lanata |
| 783 | CYB5R | Catharanthus roseus |
| 784 | CYB5R | Nepeta cataria |
| 785 | CYB5R | Arabidopsis thaliana |
| 786 | CYB5R | Catharanthus roseus |
| 787 | CYB5R | Nepeta cataria |
| 788 | CYB5R | Arabidopsis thaliana |
| 1695 | ISY | Phialophora attae |
| 1696 | ISY | Tarenaya spinosa |
| 1697 | ISY | Trifolium pratense |
| 1698 | ISY | Oryza glumipatula |
| 1699 | ISY | Triticum aestivum |
| 1700 | ISY | Oryza glumipatula |
| 1701 | ISY | Madurella mycetomatis |
| 1702 | ISY | Phaedon cochleariae |
| 1703 | ISY | Glycine max |
| 1704 | ISY | Triticum aestivum |
| 1705 | ISY | Olea europaea |
| 1706 | ISY | Camptotheca acuminata |
| 1707 | ISY | Musa acuminata subsp. malaccensis |
| 1708 | ISY | Arabidopsis thaliana |
| 1709 | ISY | Digitalis lanata |
| 1710 | ISY | Musa acuminata subsp. malaccensis |
| 1711 | ISY | Musa acuminata subsp. malaccensis |
| 1712 | ISY | Anthurium amnicola |
| 1713 | ISY | Cinchona_{—}Ledgeriana |
| 1714 | ISY | Triticum aestivum |
| 1715 | ISY | Aegilops tauschii |
| 1716 | ISY | Vinca minor |
| 1717 | ISY | Cinchona pubescens |
| 1718 | ISY | Ophiorrhiza pumila |
| 1719 | ISY | Swertia japonica |
| 1720 | ISY | Lonicera_{—}japonica |
| 1721 | ISY | Rauwolfia serpentina |
| 1722 | ISY | Lonicera japonica |
| 1723 | ISY | Oryza sativa subsp. japonica |
| 1724 | ISY | Phaedon cochleariae |

In some embodiments, the recombinant microbial cell is engineered to express a fusion protein comprising one or more enzymes of the nepetalactol synthesis pathway. The fusion protein may comprise one or more of any one of the enzymes of the nepetalactol synthesis pathway disclosed herein. Without being bound by theory, it is thought that fusion proteins comprising one or more enzymes of the nepetalactol synthesis pathway may increase the flux through the nepetalactol synthesis pathway by enhancing the catalytic efficiency of the fused enzymes. For example, if enzyme 1 (E1) and enzyme 2 (E2) are enzymes of the nepetalactol synthesis pathway, wherein product of E1 is the substrate of E2, then it is thought that an engineered fusion of E1 and E2 may improve the access of E2 to its substrate, due to E2's proximity to E1.

In some embodiments, the recombinant microbial cell is engineered to express a fusion protein comprising GPPS and GES of the nepetalactol synthesis pathway. In some embodiments, the fusion protein comprising GPPS and GES comprises an amino acid sequence comprising at least 80% identity to an amino acid sequence selected from SEQ ID Nos. 608, 609, and 1645-1694. In some embodiments, the fusion protein comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 608, 609, and 1645-1694, including all ranges and subranges therebetween. In some embodiments, the recombinant microbial cell comprises a polynucleotide encoding the fusion protein, wherein the polynucleotide comprises a nucleic acid sequence having at least about 80% identity to a nucleic acid sequence selected from SEQ ID Nos. 1396, 1397, and 1728-1777. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1396, 1397, and 1728-1777, including all ranges and subranges therebetween.

In some embodiments, the recombinant microbial cell is engineered to express a fusion protein comprising G8H and CPR of the nepetalactol synthesis pathway. In some embodiments, the fusion protein comprising G8H and CPR comprises an amino acid sequence comprising at least 80% identity to an amino acid sequence selected from SEQ ID Nos. 610-674. In some embodiments, the fusion protein comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 610-674, including all ranges and subranges therebetween. In some embodiments, the recombinant microbial cell comprises a polynucleotide encoding the fusion protein, wherein the polynucleotide comprises a nucleic acid sequence having at least about 80% identity to a nucleic acid sequence selected from SEQ ID Nos. 1398-1462. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1398-1462, including all ranges and subranges therebetween.

In some embodiments, the recombinant microbial cell is engineered to express a fusion protein comprising G8H, CPR and CYB5 of the nepetalactol synthesis pathway. In some embodiments, the fusion protein comprising G8H, CPR and CYB5 comprises an amino acid sequence comprising at least 80% identity to an amino acid sequence selected from SEQ ID Nos. 675-693. In some embodiments, the fusion protein comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 675-693, including all ranges and subranges therebetween. In some embodiments, the recombinant microbial cell comprises a polynucleotide encoding the fusion protein, wherein the polynucleotide comprises a nucleic acid sequence having at least about 80% identity to a nucleic acid sequence selected from SEQ ID Nos. 1463-1481. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1463-1481, including all ranges and subranges therebetween.

In some embodiments, the recombinant microbial cell is engineered to express a fusion protein comprising 8HGO and ISY of the nepetalactol synthesis pathway. In some embodiments, the fusion protein comprising 8HGO and ISY comprises an amino acid sequence comprising at least 80% identity to an amino acid sequence selected from SEQ ID Nos. 694-705. In some embodiments, the fusion protein comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 694-705, including all ranges and subranges therebetween. In some embodiments, the recombinant microbial cell comprises a polynucleotide encoding the fusion protein, wherein the polynucleotide comprises a nucleic acid sequence having at least about 80% identity to a nucleic acid sequence selected from SEQ ID Nos. 1482-1493. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1482-1493, including all ranges and subranges therebetween.

In some embodiments, the recombinant microbial cell is engineered to express a fusion protein comprising ISY and NEPS of the nepetalactol synthesis pathway. In some embodiments, the fusion protein comprising ISY and NEPS comprises an amino acid sequence comprising at least 80% identity to an amino acid sequence selected from SEQ ID Nos. 706-717. In some embodiments, the fusion protein comprises an amino acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to an amino acid sequence selected from SEQ ID Nos. 706-717, including all ranges and subranges therebetween. In some embodiments, the recombinant microbial cell comprises a polynucleotide encoding the fusion protein, wherein the polynucleotide comprises a nucleic acid sequence having at least about 80% identity to a nucleic acid sequence selected from SEQ ID Nos. 1494-1505. In some embodiments, the recombinant microbial cell comprises a polynucleotide comprising a nucleic acid sequence having about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to a nucleic acid selected from SEQ ID Nos. 1494-1505, including all ranges and subranges therebetween.

### Additional Genetic Engineering Approaches

In some embodiments, the recombinant microbial cells disclosed herein express altered levels of one or more genes, which affect the production and/or levels of nepetalactol, nepetalactone, dihydronepetalactone, and/or one or more side products, such as geranic acid. In some embodiments, the alteration is an upregulation, while in other embodiments, the alteration is a downregulation. In some embodiments, the recombinant microbial cells are engineered to express the one or more genes from a heterologous promoter. The heterologous promoter may be have a different strength than the native promoter (that is, it may be stronger or weaker than the native promoter), and it may be inducible or constitutive. In some embodiments, the one or more genes may be native to the recombinant microbial cells, while in other embodiments, the one or more genes may be heterologous genes.

In some embodiments, the recombinant microbial cells of this disclosure comprise a deletion or disruption of the one or more genes which affect the production and/or levels of nepetalactol, nepetalactone, dihydronepetalactone, and/or one or more side products. In some embodiments, the recombinant microbial cells of this disclosure may be genetically engineered to downregulate one or more genes using any method known in the art for this purpose, such as replacement of their native promoter with a weaker promoter; insertion of a weaker promoter between the native promoter of the gene and the start codon of the gene; and/or mutagenesis of the coding and/or non-coding regions of the gene.

In some embodiments, the present disclosure teaches reducing the activities of genes which affect the production and/or levels of nepetalactol, nepetalactone, dihydronepetalactone, and/or one or more side products. In some embodiments the activities of these genes are reduced by (i) inhibition or reduction of the expression of the coding genes of the gene; (ii) partial or complete deletion of the coding genes the gene; (iii) expression of non-functional variants of the genes; and/or (iv) inhibition or reduction of the activity of the expressed genes.

In some embodiments, the recombinant microbial cells of this disclosure may be genetically engineered to upregulate one or more genes which affect the production and/or levels of nepetalactol, nepetalactone, dihydronepetalactone, and/or one or more side products using any method known in the art for this purpose, such as replacement of their native promoter with a stronger or constitutive promoter; insertion of a stronger promoter between the native promoter of the gene and the start codon of the gene; and/or mutagenesis of the coding and/or non-coding regions of the gene. In some embodiments, the recombinant microbial cells of this disclosure may be genetically engineered to comprise an expression cassette comprising the gene and a heterologous promoter.

In some embodiments, the one or more genes encode enzymes that contribute to side product formation that impairs the production of nepetalactol, nepetalactone and/or dihydronepetalactone (e.g., genes listed in Table 7). In some embodiments, the one or more genes are annotated as encoding oxidoreductases. In some embodiments, the one or more genes are predicted to encode a protein that contains an oxidoreductase motif/domain using a program known in the art for prediction of protein domains, such as, for example, Pfam and HMM.

In some embodiments, the one or more genes encodes an enzyme that either reduces at least one double bond present in any of the monoterpene intermediates, or reduces or oxidizes at least one alcohol, aldehyde or acid functional groups of any of the monoterpene intermediates, wherein the monoterpene intermediates are intermediates in an enzyme catalyzed pathway contributing to the synthesis of nepetalactol, nepetalactone and/or dihydronepetalactone.

In some embodiments, the one or more genes that are involved in side product formation are selected from the genes listed in Table 7.

TABLE 7
Target genes encoding potential oxidoreductases

| Gene ID | Gene Name | Gene ID | Gene Name | Gene ID | Gene Name |
|---|---|---|---|---|---|
| YHR179W | OYE2 | YML054C | CYB2 | YGL191W | COX13 |
| YPL171C | OYE3 | YML080W | DUS1 | YGL187C | COX4 |
| YMR083W | ADH3 | YLR401C | DUS3 | YNL052W | COX5A |
| YOR374W | ALD4 | YOR246C | ENV9 | YHR051W | COX6 |
| YAL061W | BDH2 | YIL005W | EPS1 | YMR256C | COX7 |
| YHR037W | PUT2 | YFL041W | FET5 | YLR395C | COX8 |
| YDL246C | SOR2 | YMR020W | FMS1 | YDL067C | COX9 |
| YMR169C | ALD3 | YLR214W | FRE1 | YDR019C | GCV1 |
| YER073W | ALD5 | YKL220C | FRE2 | YMR189W | GCV2 |
| YMR110C | HFD1 | YOR381W | FRE3 | YAL044C | GCV3 |
| YBR006W | UGA2 | YOL152W | FRE7 | YOR375C | GDH1 |
| YBR145W | ADH5 | YLR047C | FRE8 | YAL062W | GDH3 |
| YPL061W | ALD6 | YDL215C | GDH2 | YDL171C | GLT1 |
| YDL168W | SFA1 | YDR096W | GIS1 | YMR145C | NDE1 |
| YHR039C | MSC7 | YKL026C | GPX1 | YDL085W | NDE2 |
| YIL124W | AYR1 | YCL035C | GRX1 | YER178W | PDA1 |
| YNL202W | SPS19 | YPL059W | GRX5 | YPR191W | QCR2 |
| YMR170C | ALD2 | YER014W | HEM14 | YFR033C | QCR6 |
| YOR323C | PRO2 | YIR037W | HYR1 | YDR529C | QCR7 |
| YNL134C | | YER051W | JHD1 | YJL166W | QCR8 |
| YJR159W | SOR1 | YJR119C | JHD2 | YER070W | RNR1 |
| YMR303C | ADH2 | YIL125W | KGD1 | YDR178W | SDH4 |
| YOL086C | ADH1 | YIR034C | LYS1 | YGR209C | TRX2 |
| YCL030C | HIS4 | YNR050C | LYS9 | YBR166C | TYR1 |
| YBR046C | ZTA1 | YBR213W | MET8 | YMR318C | ADH6 |
| YBR026C | ETR1 | YBR084W | MIS1 | YAL060W | BDH1 |
| YML131W | | YKR080W | MTD1 | YLR070C | XYL2 |
| YBL069W | AST1 | YML120C | NDI1 | YOR125C | CAT5 |
| YMR152W | YIM1 | YBR035C | PDX3 | YLR056W | ERG3 |
| YCR102C | | YGL205W | POX1 | YGL012W | ERG4 |
| YLR460C | | YBL064C | PRX1 | YMR015C | ERG5 |
| YER101C | AST2 | YGR180C | RNR4 | YMR272C | SCS7 |
| YLL041C | SDH2 | YER169W | RPH1 | YOL059W | GPD2 |
| YOR356W | CIR2 | YBR037C | SCO1 | YOL151W | GRE2 |
| YER069W | ARG5, 6 | YLR164W | SHH4 | YOR136W | IDH2 |
| YDR158W | HOM2 | YJR104C | SOD1 | YKL085W | MDH1 |
| YJL052W | TDH1 | YHR008C | SOD2 | YDL022W | GPD1 |
| YJR009C | TDH2 | YCR083W | TRX3 | YML075C | HMG1 |
| YGR192C | TDH3 | YDR453C | TSA2 | YLR450W | HMG2 |
| YDL124W | | YKL216W | URA1 | YER081W | SER3 |
| YJR096W | | YFR049W | YMR31 | YDL174C | DLD1 |
| YOL165C | AAD15 | YKL069W | | YEL070W | DSF1 |
| YHR104W | GRE3 | YMR009W | ADI1 | YKR009C | FOX2 |
| YKL029C | MAE1 | YPR200C | ARR2 | YBR159W | IFA38 |
| YPL088W | | YJR025C | BNA1 | YKL055C | OAR1 |
| YJR155W | AAD10 | YJR078W | BNA2 | YHR063C | PAN5 |
| YNL331C | AAD14 | YBL098W | BNA4 | YMR226C | |
| YDL243C | AAD4 | YGR255C | COQ6 | YDR541C | |
| YBR149W | ARA1 | YER141W | COX15 | YGL157W | ARI1 |
| YMR041C | ARA2 | YGR088W | CTT1 | YIR036C | IRC24 |
| YIL155C | GUT2 | YHR055C | CUP1-2 | YNL241C | ZWF1 |
| YDR368W | YPR1 | YIL049W | DFG10 | YML056C | IMD4 |
| YGL256W | ADH4 | YDR402C | DIT2 | YDR127W | ARO1 |
| YOR120W | GCY1 | YDL178W | DLD2 | YHR183W | GND1 |
| YPR127W | | YEL071W | DLD3 | YGR256W | GND2 |
| YJL045W | | YIL010W | DOT5 | YJR139C | HOM6 |
| YML086C | ALO1 | YLR405W | DUS4 | YLR432W | IMD3 |
| YOR037W | CYC2 | YNL280C | ERG24 | YBR115C | LYS2 |
| YPL091W | GLR1 | YPR037C | ERV2 | YKL071W | |
| YPL023C | MET12 | YDR518W | EUG1 | YDR197W | CBS2 |
| YLR142W | PUT1 | YMR058W | FET3 | YLR109W | AHP1 |
| YKL148C | SDH1 | YHR176W | FMO1 | YGL160W | AIM14 |
| YMR315W | | YNR060W | FRE4 | YKR066C | CCP1 |
| YEL047C | FRD1 | YOR384W | FRE5 | YDR256C | CTA1 |
| YJR137C | MET5 | YLL051C | FRE6 | YHR053C | CUP1-1 |
| YJR051W | OSM1 | YCL026C-A | FRM2 | YNR015W | SMM1 |
| YHR179W | OYE2 | YBR244W | GPX2 | YKL086W | SRX1 |
| YPL171C | OYE3 | YDR513W | GRX2 | YDR297W | SUR2 |
| YHR106W | TRR2 | YDR098C | GRX3 | YER049W | TPA1 |
| YGR234W | YHB1 | YER174C | GRX4 | YLR043C | TRX1 |
| YKL150W | MCR1 | YDL010W | GRX6 | YML028W | TSA1 |
| YIL043C | CBR1 | YBR014C | GRX7 | YNL229C | URE2 |
| YFL018C | LPD1 | YLR364W | GRX8 | YIL111W | COX5B |
| YFR030W | MET10 | YIR038C | GTT1 | YPR167C | MET16 |
| YGL125W | MET13 | YCL026C-B | HBN1 | YHR001W-A | QCR10 |
| YBR221C | PDB1 | YER205C | HMX1 | YGR183C | QCR9 |
| YPL107W | | YLL057C | JLP1 | YGR204W | ADE3 |
| YML051W | GAL80 | YJR070C | LIA1 | YGL148W | ARO2 |
| YGL094C | PAN2 | YLR011W | LOT6 | YBL045C | COR1 |
| YLR084C | RAX2 | YOR288C | MPD1 | YLR038C | COX12 |
| YNL187W | SWT21 | YOL088C | MPD2 | YNL009W | IDP3 |
| YHR009C | TDA3 | YER042W | MXR1 | YIL094C | LYS12 |
| YML087C | AIM33 | YCL033C | MXR2 | YOL126C | MDH2 |
| YPL017C | IRC15 | YIL066C | RNR3 | YDL078C | MDH3 |
| YPR074C | TKL1 | YBR024W | SCO2 | YIL074C | SER33 |
| YHR079C | IRE1 | YNL037C | IDH1 | YGL185C | |
| YBR117C | TKL2 | YDL066W | IDP1 | YOR388C | FDH1 |
| YPL113C | | YLR174W | IDP2 | YNL274C | GOR1 |
| YGL039W | | | | | |

In some embodiments, the oxidoreductase is encoded by a gene selected from FMS1, SUR2, SWT1, QCR9, NCP1 and GDP1. In some embodiments, the recombinant microbial cells disclosed herein comprise a deletion of a gene encoding FMS1 oxidoreductase. In some embodiments, the recombinant microbial cells disclosed herein comprise a deletion of a gene encoding SUR2 oxidoreductase. In some embodiments, the recombinant microbial cells disclosed herein comprise a heterologous promoter operably linked to a gene encoding the oxidoreductase. In some embodiments, the heterologous promoter is a weaker promoter, as compared to the native promoter of the gene encoding the oxidoreductase. In some embodiments, the heterologous promoter is TDH3 or YEF3. In some embodiments, the recombinant microbial cells disclosed herein comprise TDH3 promoter operably linked to a gene encoding SWT1 oxidoreductase. In some embodiments, the recombinant microbial cells disclosed herein comprise YEF3 promoter operably linked to a gene encoding QCR9 oxidoreductase. In some embodiments, the recombinant microbial cells disclosed herein comprise an expression cassette comprising a gene encoding the oxidoreductase operatively linked to a promoter. In some embodiments, the recombinant microbial cells disclosed herein comprise an expression cassette comprising a gene encoding NCP1 oxidoreductase or GPD1 oxidoreductase operatively linked to GAL7 promoter.

In some embodiments, the recombinant microbial cells disclosed herein produce higher levels of nepetalactol, higher levels of nepetalactone, higher levels of dihydronepetolactone, and/or lower levels of geranic acid, as compared to a control recombinant cell, wherein the control recombinant cell has wild type levels of the oxidoreductase.

In some embodiments, the one or more genes comprises genes that encode enzymes catalyzing the transfer of at least one acetyl group to one or more alcohol ends of monoterpene intermediates that would result in unwanted side products, thus impairing the production of nepetalactol, nepetalactone and/or dihydronepetalactone. In some embodiments, the one or more genes is ATF1 (gene ID—YOR377W).

### Genetic Engineering of the DXP Pathway

In some embodiments, the recombinant microbial cells of this disclosure are engineered to upregulate one or more enzymes of the 1-deoxy-D-xylulose-5-phosphate pathway (DXP pathway) or the alcohol-dependent hemiterpene pathway. Without being bound by theory, it is thought that the overexpression of one or more enzymes of the DXP pathway may increase the flux through the DXP pathway to increase the amounts of IPP or DMAPP produced in the recombinant microbial cells of this disclosure, and thereby contribute to the increase in flux through the nepetalactol synthesis pathway, resulting in an increased amount of nepetalactol/nepetalactone/dihydronepetalactone in the recombinant microbial cells of this disclosure.

The DXP pathway is initiated with a thiamin diphosphate-dependent condensation between D-glyceraldehyde 3-phosphate and pyruvate to produce DXP, which is then reductively isomerized to 2-C-methyl-D-erythritol 4-phosphate (MEP) by DXP reducto-isomerase (DXR/IspC). Subsequent coupling between MEP and cytidine 5′-triphosphate (CTP) is catalyzed by CDP-ME synthetase (IspD) and produces methylerythritol cytidyl diphosphate (CDP-ME). An ATP-dependent enzyme (IspE) phosphorylates the C₂ hydroxyl group of CDP-ME, and the resulting 4-diphosphocytidyl-2-C-methyl-D-erythritol-2-phosphate (CDP-MEP) is cyclized by IspF to 2-C-methyl-D-erythritol-2,4-cyclodiphosphate (MEcPP). IspG catalyzes the ring-opening of the cyclic pyrophosphate and the C₃-reductive dehydration of MEcPP to 4-hydroxy-3-methyl-butenyl 1-diphosphate (HMBPP). The final step of the MEP pathway is catalyzed by IspH and converts HMBPP to both IPP and DMAPP (see FIG. 11).

In some embodiments, the recombinant microbial cells of this disclosure may comprise one or more polynucleotide(s) encoding one or more of the following enzymes of the DXP pathway: 1-Deoxy-D-xylulose 5-phosphate synthase (DXS), 1-Deoxy-D-xylulose 5-phosphate reductoisomerase (DXR), CDP-ME synthetase (IspD), IspE, IspF, and IspH. In some embodiments, the recombinant microbial cells of this disclosure may comprise one or more polynucleotide(s) encoding each of the following enzymes of the DXP pathway: 1-Deoxy-D-xylulose 5-phosphate synthase (DXS), 1-Deoxy-D-xylulose 5-phosphate reductoisomerase (DXR), CDP-ME synthetase (IspD), IspE, IspF, and IspH. Further details of the pathway are provided in Lund et al., ACS Synth. Biol. 2019, 8, 2, 232-238; and Zhao et al., Annu Rev Biochem. 2013; 82:497-530, the contents of each of which is incorporated herein by reference in their entireties for all purposes.

In some embodiments, the recombinant microbial cell is engineered to overexpress one or more of the enzymes of the following enzymes of the DXP pathway: 1-Deoxy-D-xylulose 5-phosphate synthase (DXS), 1-Deoxy-D-xylulose 5-phosphate reductoisomerase (DXR), CDP-ME synthetase (IspD), IspE, IspF, and IspH. In some embodiments, the recombinant microbial cell is engineered to overexpress all of the following enzymes of the DXP pathway: 1-Deoxy-D-xylulose 5-phosphate synthase (DXS), 1-Deoxy-D-xylulose 5-phosphate reductoisomerase (DXR), CDP-ME synthetase (IspD), IspE, IspF, and IspH. The amount of the enzyme expressed by the recombinant microbial cell may be higher than the amount of that corresponding enzyme in a wild type microbial cell by about 1.25 fold to about 20 fold, for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 75 fold, about 80 fold, about 85 fold, about 90 fold, about 95 fold, or about 100 fold, including all the subranges and values that lie therebetween.

In some embodiments the recombinant microbial cell has been modified to contain a heterologous promoter operably linked to one or more endogenous gene encoding an enzyme of the DXP pathway. In some embodiments, the heterologous promoter is a stronger promoter, as compared to the native promoter. In some embodiments, the recombinant microbial cell is engineered to express an enzyme of the DXP pathway constitutively. For instance, in some embodiments, the recombinant microbial cell may express an enzyme of the DXP pathway at a time when the enzyme is not expressed by the wild type microbial cell.

In other embodiments, the present disclosure envisions overexpressing one or more genes encoding one or more enzymes of the DXP pathway by increasing the copy number of said gene. Thus, in some embodiments, the recombinant microbial cell comprises at least one additional copy of a DNA sequence encoding an enzyme of the DXP pathway, as compared to a wild type microbial cell. In some embodiments, the recombinant microbial cell comprises 1 to 40 additional copies of a DNA sequence encoding an enzyme of the DXP pathway, as compared to a wild type microbial cell. For instance, the recombinant microbial cell may comprise 1, 2, 3, 4, 5, 6, 7, 8, 9, 10, 15, 20, 25, 30, 35, or 40 additional copies of the DNA sequence, compared to a wild type microbial cell, including all ranges and subranges therebetween.

In some embodiments, the present disclosure teaches methods of increasing nepetalactol biosynthesis by expressing one or more mutant genes encoding one or more enzymes of the DXP pathway. Thus, in some embodiments, the recombinant microbial cell comprises a DNA sequence encoding for one or more mutant DXP pathway enzymes. In some embodiments, the one or more mutant DXP pathway enzymes are more catalytically active than the corresponding wild type enzyme. In some embodiments, the one or more mutant DXP pathway enzymes have a higher k_{Cat}, as compared to the wild type enzyme. In some embodiments, the one or more mutant DXP pathway enzymes that are more catalytically active than the wild type enzyme, are insensitive to negative regulation, such as, for example, allosteric inhibition.

### Methods of Producing Nepetalactol, Nepetalactone and Dihydronepetalactone

The disclosure provides methods of producing nepetalactol, nepetalactone and/or dihydronepetalactone using any one of the recombinant microbial cells of this disclosure.

The disclosure provides methods of producing nepetalactol from a carbon source, comprising (a) providing any one of the recombinant microbial cells disclosed herein which is capable of producing nepetalactol from glucose; and (b) cultivating the recombinant microbial cell in a suitable cultivation medium comprising glucose or any comparable carbon source, thereby producing nepetalactol. In some embodiments, the carbon source is glucose, galactose, glycerol, and/or ethanol. In some embodiments, the carbon source is glucose.

The disclosure also provides methods producing nepetalactol comprising (a) providing any one of the recombinant microbial cells disclosed herein comprising one or more polynucleotides encoding a heterologous nepetalactol synthase (NEPS); and (b) cultivating the recombinant microbial cell in a suitable cultivation medium comprising a substrate feed. In some embodiments, the substrate feed is glucose or any comparable carbon source. In some embodiments, the substrate feed is any one or more of the substrates listed in Table 1 or Table 2, thereby producing nepetalactol.

The disclosure provides methods of producing a specific ratio of nepetalactol stereoisomers comprising (a) providing any one of the recombinant microbial cells disclosed herein comprising one or more polynucleotides encoding a heterologous nepetalactol synthase (NEPS); and (b) cultivating the recombinant microbial cell in a suitable cultivation medium comprising glucose or any comparable carbon source; or any one or more of the substrates listed in Table 1 or Table 2, thereby producing the specific ratio of nepetalactol stereoisomers. In some embodiments, the method produces cis, trans-nepetalactol in an amount that is more than 50% (for example, more that 55%, more than 60%, more than 65%, more than 70%, more than 75%, more than 80%, more that 85%, more than 90%, more than 95%, more than 99%, or an amount of 100%, including all values and subranges that lie therebetween) of the total amount of nepetalactol stereoisomers produced. In some embodiments, the method produces trans, cis-nepetalactol in an amount that is more than 50% (for example, more that 55%, more than 60%, more than 65%, more than 70%, more than 75%, more than 80%, more that 85%, more than 90%, more than 95%, more than 99%, or an amount of 100%, including all values and subranges that lie therebetween) of the total amount of nepetalactol stereoisomers produced. In some embodiments, the method produces trans, trans-nepetalactol in an amount that is more than 50% (for example, more that 55%, more than 60%, more than 65%, more than 70%, more than 75%, more than 80%, more that 85%, more than 90%, more than 95%, more than 99%, or an amount of 100%, including all values and subranges that lie therebetween) of the total amount of nepetalactol stereoisomers produced. In some embodiments, the method produces cis, cis-nepetalactol in an amount that is more than 50% (for example, more that 55%, more than 60%/c, more than 65%, more than 70%, more than 75%, more than 80%, more that 85%, more than 90%, more than 95%, more than 99%, or an amount of 100%, including all values and subranges that lie therebetween) of the total amount of nepetalactol stereoisomers produced.

The disclosure also provides methods producing nepetalactone comprising (a) providing any one of the recombinant microbial cells disclosed herein comprising one or more polynucleotides encoding a heterologous nepetalactone oxidoreductase (NOR) that catalyzes the reduction of nepetalactol to nepetalactone; (b) cultivating the recombinant microbial cell in a suitable cultivation medium; and (c) contacting the recombinant microbial cell with nepetalactol to form nepetalactone. In some embodiments, the recombinant microbial cell is cultivated in a suitable cultivation medium comprising nepetalactol. In some embodiments, the recombinant microbial cell is cultivated in a suitable cultivation medium comprising glucose or any comparable carbon source, such that nepetalactol is produced in the recombinant microbial cell. In some embodiments, the recombinant microbial cell is cultivated in a suitable cultivation medium comprising any one or more of the substrates listed in Table 1 or Table 2, such that nepetalactol is produced in the recombinant microbial cell.

The disclosure provides methods of producing a specific ratio of nepetalactone stereoisomers comprising (a) providing any one of the recombinant microbial cells disclosed herein comprising one or more polynucleotides encoding a heterologous nepetalactone oxidoreductase (NOR) that catalyzes the reduction of nepetalactol to nepetalactone; and (b) cultivating the recombinant microbial cell in a suitable cultivation medium comprising glucose or any comparable carbon source; or any one or more of the substrates listed in Table 1 or Table 2, thereby producing the specific ratio of nepetalactone stereoisomers. In some embodiments, the method produces cis, trans-nepetalactone in an amount that is more than 50% (for example, more that 55%, more than 60%, more than 65%, more than 70%, more than 75%, more than 80%, more that 85%, more than 90%, more than 95%, more than 99%, or an amount of 100%, including all values and subranges that lie therebetween) of the total amount of nepetalactone stereoisomers produced. In some embodiments, the method produces trans, cis-nepetalactone in an amount that is more than 50% (for example, more that 55%, more than 60%, more than 65%, more than 70%, more than 75%, more than 80%, more that 85%, more than 90%, more than 95%, more than 99%, or an amount of 100%, including all values and subranges that lie therebetween) of the total amount of nepetalactone stereoisomers produced. In some embodiments, the method produces trans, trans-nepetalactone in an amount that is more than 50% (for example, more that 55%, more than 60%, more than 65%, more than 70%, more than 75%, more than 80%, more that 85%, more than 90%, more than 95%, more than 99%, or an amount of 100%, including all values and subranges that lie therebetween) of the total amount of nepetalactone stereoisomers produced. In some embodiments, the method produces cis, cis-nepetalactone in an amount that is more than 50% (for example, more that 55%, more than 60%, more than 65%, more than 70%, more than 75%, more than 80%, more that 85%, more than 90%, more than 95%, more than 99%, or an amount of 100%, including all values and subranges that lie therebetween) of the total amount of nepetalactone stereoisomers produced.

The disclosure also provides methods producing dihydronepetalactone comprising (a) providing any one of the recombinant microbial cells disclosed herein comprising one or more polynucleotides encoding a heterologous dihydronepetalactone dehydrogenase (DND) that catalyzes the reduction of nepetalactone to dihydronepetalactone; (b) cultivating the recombinant microbial cell in a suitable cultivation medium; and (c) contacting the recombinant microbial cell with nepetalactone to form dihydronepetalactone. In some embodiments, the recombinant microbial cell is cultivated in a suitable cultivation medium comprising nepetalactone. In some embodiments, the recombinant microbial cell is cultivated in a suitable cultivation medium comprising glucose or any comparable carbon source, such that nepetalactone is produced in the recombinant microbial cell. In some embodiments, the recombinant microbial cell is cultivated in a suitable cultivation medium comprising any one or more of the substrates listed in Table 1 or Table 2, such that nepetalactone is produced in the recombinant microbial cell.

In some embodiments, the heterologous NEPS, NOR, or DND is derived from another microbial species, a plant cell or a mammalian cell. In some embodiments, the polynucleotide is derived from any one of the source organisms listed in the Sequence Listing, Table 3, Table 4, Table 5, or Table 6. In some embodiments, the polynucleotide is derived from Camptotheca acuminate, Catharanthus roseus, Rauvolfia serpentina, or Vinca minor.

In some embodiments, the polynucleotide encodes a protein derived from a plant of the genus Nepeta. In some embodiments, the polynucleotide is derived from a plant of any one of the following species: Nepeta mussinii, Nepeta cataria, Nepeta adenophyta, Nepeta agrestis, Nepeta alaghezi, Nepeta alatavica, Nepeta algeriensis, Nepeta amicorum, Nepeta amoena, Nepeta anamurensis, Nepeta annua, Nepeta apudeji, Nepeta argolica, Nepeta assadii, Nepeta assurgens, Nepeta astorensis, Nepeta atlantica, Nepeta autraniana, Nepeta azurea, Nepeta badachschanica, Nepeta bakhtiarica, Nepeta ballotifolia, Nepeta balouchestanica, Nepeta barfakensis, Nepeta baytopii, Nepeta bazoftica Jamza, Nepeta bellevii, Nepeta betonicifolia, Nepeta binaloudensis, Nepeta bodeana, Nepeta boissieri, Nepeta bokhonica, Nepeta bombaiensis, Nepeta bornmuelleri, Nepeta botschantzevii, Nepeta brachyantha, Nepeta bracteata, Nepeta brevifolia, Nepeta bucharica, Nepeta caerulea, Nepeta caesarea, Nepeta campestris, Nepeta camphorate, Nepeta campylantha, Nepeta cephalotes, Nepeta chionophila, Nepeta ciliaris, Nepeta cilicica, Nepeta clarkei, Nepeta coerulescens, Nepeta concolor, Nepeta conlerta, Nepeta congesta, Nepeta connate, Nepeta consanguinea, Nepeta crinite, Nepeta crispa, Nepeta curviflora, Nepeta cyunea, Nepeta cyrenaica, Nepeta czegemensis, Nepeta daenensis, Nepeta deflersiana, Nepeta densiflora, Nepeta dentate, Nepeta denudate, Nepeta dirmencii, Nepeta discolor, Nepeta distans, Nepeta duthiei, Nepeta elliptica, Nepeta elymaitica, Nepeta erecta, Nepeta eremokosmos, Nepeta eremophila, Nepeta eriosphaera, Nepeta eriostachya, Nepeta ernesti-mayeri, Nepeta everardii, Nepeta faassenii, Nepeta flavida, Nepeta floccose, Nepeta foliosa, Nepeta fordii, Nepeta formosa, Nepeta freitagii, Nepeta glechomifolia, Nepeta gloeocephala, Nepeta glomerata, Nepeta glomerulosa, Nepeta glutinosa, Nepeta gontscharovii, Nepeta govaniana, Nepeta gracililora, Nepeta granatensis, Nepeta grandiflora, Nepeta grata, Nepeta griffithii, Nepeta heliotropfiolia, Nepeta hemsleyana, Nepeta henanensis, Nepeta hindostana, Nepeta hispanica, Nepeta hormozganica, Nepeta humilis, Nepeta hymenodonta, Nepeta isaurica, Nepeta ispahanica, Nepeta italic, Nepeta jakupicensis, Nepeta jomdaensis, Nepeta juncea, Nepeta knorringiana, Nepeta koeieana, Nepeta kokamirica, Nepeta kokanica, Nepeta komarovii, Nepeta kotschvi, Nepeta kurdica, Nepeta kurramensis, Nepeta ladanolens, Nepeta laevigata, Nepeta lagopsis, Nepeta lamiifolia, Nepeta lamiopsis, Nepeta lasiocephala, Nepeta latifolia, Nepeta leucolaena, Nepeta linearis, Nepeta lipskyi, Nepeta longibracteata, Nepeta longijlora, Nepeta longituba, Nepeta ludlow-hewittii, Nepeta macrosiphon, Nepeta mahanensis, Nepeta manchuriensis, Nepeta mariae, Nepeta maussarifi, Nepeta melissifolia, Nepeta membranmfolia, Nepeta menthoides Nepeta meyeri, Nepeta micrantha, Nepeta minuticephala, Nepeta mirzayanii, Nepeta mollis, Nepeta monocephala, Nepeta monticola, Nepeta multibracteata, Nepeta multicaulis, Nepeta multifidi, Nepeta natanzensis, Nepeta nawarica, Nepeta nepalensis, Nepeta nepetella, Nepeta nepetellae, Nepeta nepetoides, Nepeta nervosa, Nepeta nuda, Nepeta obtusicrena, Nepeta odorifera, Nepeta olgae, Nepeta orphanidea, Nepeta pabotii, Nepeta paktiana, Nepeta pamirensis, Nepeta parnassica, Nepeta paucifolia, Nepeta persica, Nepeta petraea, Nepeta phyllochlamys, Nepeta pilinux, Nepeta podlechin, Nepeta podostachys, Nepeta pogonosperma, Nepeta polyodonta, Nepeta praetervisa, Nepeta prattii, Nepeta prostrata, Nepeta pseudokokanica, Nepeta pubescens, Nepeta pungens, Nepeta racemose, Nepeta raphanorhiza, Nepeta rechingern, Nepeta rivularis, Nepeta roopiana, Nepeta rtanjensis, Nepeta rubella, Nepeta rugose, Nepeta saccharata, Nepeta santoana, Nepeta saturejoides, Nepeta schiraziana, Nepeta schmidi, Nepeta schugnanica, Nepeta scordotis, Nepeta septemcrenata, Nepeta sessilis, Nepeta shahmirzadensis, Nepeta sheilae, Nepeta sibirica, Nepeta sorgerae, Nepeta sosnovskyi, Nepeta souliei, Nepeta spathuhfera, Nepeta sphaciotica, Nepeta spruneri, Nepeta stachyoides, Nepeta staintonii, Nepeta stenantha, Nepeta stewartiana, Nepeta straussii, Nepeta stricta, Nepeta suavis, Nepeta subcaespitosa, Nepeta subhastata, Nepeta subincisa, Nepeta subintegra, Nepeta subsessilis, Nepeta sudanica, Nepeta sulfiriflora, Nepeta sulphurea, Nepeta sungpanensis, Nepeta supine, Nepeta taxkorganica, Nepeta tenuiflora, Nepeta tenuifolia, Nepeta teucriifolia, Nepeta teydea, Nepeta tibestica, Nepeta tmolea, Nepeta trachonitica, Nepeta transiliensis, Nepeta trautvetteri, Nepeta trichocalyx, Nepeta tuberosa, Nepeta tytthantha, Nepeta uberrima, Nepeta ucranica, Nepeta veitchii, Nepeta velutina, Nepeta tiscida, Nepeta viviani, Nepeta wettsteinii, Nepeta wilsonii, Nepeta woodiana, Nepeta yanthina, Nepeta yesoensis, Nepeta zandaensis, or Nepeta zangezura.

In some embodiments of the methods and recombinant microbial cells disclosed herein, the one or more polynucleotides are codon optimized for expression in the recombinant microbial host cell. In some embodiments, the polynucleotides disclosed herein are inserted into a suitable region of the recombinant microbial cell genome; or into a centromeric or episomal plasmid under any promoter that is known and commonly used in the art.

The disclosure also provides methods of producing nepetalactol, nepetalactone or dihydronepetalactone ex vivo or in vitro, comprising bringing a substrate in contact with one or more enzymes and cofactors required for the enzymatic conversion of the substrate to nepetalactol, nepetalactone or dihydronepetalactone, thereby forming nepetalactol, nepetalactone or dihydronepetalactone. In some embodiments, the substrate is glucose or a comparable carbon source, such as galactose, glycerol and ethanol. In some embodiments, the substrate may be selected from those listed in Table 1 or Table 2, such as, for example 8-hydroxygeraniol. In some embodiments, the one or more enzymes are expressed ex vivo or in vitro (through cell-free expression). In some embodiments, the one or more enzymes are expressed in recombinant microbial cells of this disclosure, followed by the isolation and purification of the enzymes through cell lysis and protein purification steps for use in the ex vivo or in vitro production of nepetalactol, nepetalactone or dihydronepetalactone.

(a) Host Cells: As used herein, the term “microbial cell” includes, but is not limited to, the two prokaryotic domains, Bacteria and Archaea, as well as eukaryotic fungi and protists. However, in certain aspects, “higher” eukaryotic organisms such as insects, plants, and animals may be utilized in the methods taught herein.

Suitable host cells include, but are not limited to: bacterial cells, algal cells, plant cells, fungal cells, insect cells, and mammalian cells. In one illustrative embodiment, suitable host cells include E. coli (e.g., SHuffle® competent E. coli available from New England BioLabs in Ipswich, Mass.).

Other suitable host organisms of the present disclosure include microorganisms of the genus Corynebacterium. In some embodiments, Corynebacterium strains/species include: C. efficiens, with the deposited type strain being DSM44549, C. glutamicum, with the deposited type strain being ATCC13032, and C. ammoniagenes, with the deposited type strain being ATCC6871. In some embodiments, the host cell of the present disclosure is C. glutamicum.

Suitable host strains of the genus Corynebacterium, in particular of the species Corynebacterium glutamicum, are in particular the known wild-type strains: Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacteriur flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

The term “Micrococcus glutamicus” has also been in use for C. glutamicum. Some representatives of the species C. efficiens have also been referred to as C. thermoaminogenes in the prior art, such as the strain FERM BP-1539, for example.

In some embodiments, the host cell of the present disclosure is a eukaryotic cell. Suitable eukaryotic host cells include, but are not limited to: fungal cells, algal cells, insect cells, animal cells, and plant cells. Suitable fungal host cells include, but are not limited to: Ascorycota, Basidiomycota, Deuteromycota, Zygomycota, Fungi imperfecti. The fungal host cells include yeast cells and filamentous fungal cells. Suitable filamentous fungi host cells include, for example, any filamentous forms of the subdivision Eumycotina and Oomycota. (see, e.g., Hawksworth et al., In Ainsworth and Bisby's Dictionary of The Fungi, 8^{th} edition, 1995, CAB International, University Press, Cambridge, UK, which is incorporated herein by reference). Filamentous fungi are characterized by a vegetative mycelium with a cell wall composed of chitin, cellulose and other complex polysaccharides. The filamentous fungi host cells are morphologically distinct from yeast.

In certain illustrative, but non-limiting embodiments, the filamentous fungal host cell may be a cell of a species of: Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella, or teleomorphs, or anamorphs, and synonyms or taxonomic equivalents thereof. In one embodiment, the filamentous fungus is selected from the group consisting of A. nidulans, A. oryzae, A. sojae, and Aspergilli of the A. niger Group. In an embodiment, the filamentous fungus is Aspergillus niger.

In some embodiments, the host cells may comprise specific mutants of a fungal species. Examples of such mutants can be strains that protoplast very well; strains that produce mainly or, more preferably, only protoplasts with a single nucleus; strains that regenerate efficiently in microtiter plates, strains that regenerate faster and/or strains that take up polynucleotide (e.g., DNA) molecules efficiently, strains that produce cultures of low viscosity such as, for example, cells that produce hyphae in culture that are not so entangled as to prevent isolation of single clones and/or raise the viscosity of the culture, strains that have reduced random integration (e.g., disabled non-homologous end joining pathway) or combinations thereof.

In some embodiments, the host cell comprises a specific mutant strain, which lacks a selectable marker gene such as, for example, uridine-requiring mutant strains. These mutant strains can be either deficient in orotidine 5 phosphate decarboxylase (OMPD) or orotate p-ribosyl transferase (OPRT) encoded by the pyrG or pyrE gene, respectively (T. Goosen et al., Curr Genet. 1987, 11:499 503; J. Begueret et al., Gene. 1984 32:487 92.

In some embodiments, the host cell comprises specific mutant strains that possess a compact cellular morphology characterized by shorter hyphae and a more yeast-like appearance.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In certain embodiments, the host cell is an algal cell such as, Chlamydomonas (e.g., C. reinhardrii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Biiidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas. In some embodiments, the host cell is Corynebacterium glutamicum.

In some embodiments, the bacterial host strain is an industrial strain. Numerous bacterial industrial strains are known and suitable in the methods and compositions described herein.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

In some embodiments, the host cell may be any animal cell type, including mammalian cells, for example, human (including 293, WI38, PER.C6 and Bowes melanoma cells), mouse (including 3T3, NS0, NS1, Sp2/0), hamster (CHO, BHK), monkey (COS, FRhL, Vero), and hybridoma cell lines.

In various embodiments, strains that may be used in the practice of the disclosure including both prokaryotic and eukaryotic strains, are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

In some embodiments, the methods of the present disclosure are also applicable to multi-cellular organisms. The organisms can comprise a plurality of plants such as Grarineae, Fetucoideae, Poacoideae, Agrostis, Phleum, Dactylis, Sorgum, Setaria, Zea, Oryza, Triticum, Secale, Avena, Hordeum, Saccharum, Poa, Festuca, Stenotaphrum, Cynodon, Coix, Olyreae, Phareae, Compositae, Nicotiana, or Leguminosae. For example, the plants can be corn, rice, soybean, cotton, wheat, rye, oats, barley, pea, beans, lentil, peanut, yam bean, cowpeas, velvet beans, clover, alfalfa, lupine, vetch, lotus, sweet clover, wisteria, sweet pea, sorghum, millet, sunflower, canola or the like. Similarly, the organisms can include a plurality of animals such as non-human mammals, fish, insects, or the like.

(b) Genetic engineering methods: The host cells described herein may comprise one or more vectors comprising one or more nucleic acid sequences encoding the enzymes disclosed herein. Vectors useful in the methods described herein can be linear or circular. Vectors may integrate into a target genome of a host cell or replicate independently in a host cell. Vectors may include, for example, an origin of replication, a multiple cloning site (MCS), and/or a selectable marker. An expression vector typically includes an expression cassette containing regulatory elements, such as a promoter, a ribosome binding sequence (RBS) and/or a downstream terminator sequence that facilitate expression of a polynucleotide sequence (often a coding sequence) in a particular host cell. Non-limiting examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif. (1990), the contents of which are incorporated herein by reference in its entirety for all purposes.

The host cells of this disclosure may be prepared using conventional techniques of molecular biology (including recombinant techniques), microbiology, cell biology, and biochemistry, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR The Polymerase Chain Reaction,” (Mullis et al., eds., 1994); Singleton et al., Dictionary of Microbiology and Molecular Biology 2nd ed., J. Wiley & Sons (New York, N.Y. 1994), the contents of each of which are incorporated herein by reference in their entireties for all purposes.

Vectors or other polynucleotides may be introduced into host cells by any of a variety of standard methods, such as transformation, conjugation, electroporation, nuclear microinjection, transduction, transfection (e.g., lipofection mediated or DEAEDextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Patent Pub. Nos. 2009/0203102, 2010/0048964, and 2010/0003716, and International Publication Nos. WO 2009/076676, WO 2010/003007, and WO 2009/132220, the contents of each of which are incorporated herein by reference in their entireties for all purposes.

In some embodiments, the method of introducing one or more vectors into the host cell comprises methods of looping out selected regions of DNA from the host organisms. The looping out method can be as described in Nakashima et al 2014 “Bacterial Cellular Engineering by Genome Editing and Gene Silencing.” Int. J. Mol. Sci. 15(2), 2773-2793. In some embodiments, the present disclosure teaches looping out selection markers from positive transformants. Looping out deletion techniques are known in the art, and are described in (Tear et al. 2014 “Excision of Unstable Artificial Gene-Specific inverted Repeats Mediates Scar-Free Gene Deletions in Escherichia coli.” Appl. Biochem. Biotech. 175: 1858-1867). The looping out methods can be performed using single-crossover homologous recombination or double-crossover homologous recombination. In one embodiment, looping out of selected regions as described herein can entail using single-crossover homologous recombination as described herein.

First, loop out vectors are inserted into selected target regions within the genome of the host organism (e.g., via homologous recombination, CRISPR, or other gene editing technique). In one embodiment, single-crossover homologous recombination is used between a circular plasmid or vector and the host cell genome in order to loop-in the circular plasmid or vector. The inserted vector can be designed with a sequence which is a direct repeat of an existing or introduced nearby host sequence, such that the direct repeats flank the region of DNA slated for looping and deletion. Once inserted, cells containing the loop out plasmid or vector can be counter selected for deletion of the selection region (e.g., lack of resistance to the selection gene).

Persons having skill in the art will recognize that the description of the loopout procedure represents but one illustrative method for deleting unwanted regions from a genome. Indeed the methods of the present disclosure are compatible with any method for genome deletions, including but not limited to gene editing via CRISPR, TALENS, FOK, or other endonucleases. Persons skilled in the art will also recognize the ability to replace unwanted regions of the genome via homologous recombination techniques.

In some embodiments, the host cell cultures are grown to an optical density at 600 nm of 1-500, such as an optical density of 50-150. Microbial (as well as other) cells can be cultured in any suitable medium including, but not limited to, a minimal medium, i.e., one containing the minimum nutrients possible for cell growth. Minimal medium typically contains: (1) a carbon source for microbial growth; (2) salts, which may depend on the particular microbial cell and growing conditions; and (3) water. Suitable media can also include any combination of the following: a nitrogen source for growth, a sulfur source for growth, a phosphate source for growth, metal salts for growth, vitamins for growth, and other cofactors for growth.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (levulose), and galactose; illustrative oligosaccharides include dextran or glucan, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids. Minimal medium can be supplemented with one or more selective agents, such as antibiotics.

To produce nepetalactol, nepetalactone, and/or dihydronepetalactone, the culture medium can include, and/or is supplemented during culture with, glucose and/or a nitrogen source such as urea, an ammonium salt, ammonia, or any combination thereof. In some embodiments, the culture medium includes and/or is supplemented to include any carbon source of the nepetalactone biosynthetic pathway, for example, as shown in FIG. 1. In some embodiments, the culture medium includes and/or is supplemented to include geraniol and/or 8-hydroxygeraniol. In some embodiments, the culture medium includes and/or is supplemented to include any carbon source of the nepetalactone biosynthetic pathway in the range of about 0.1-100 g/L.

Materials and methods suitable for the maintenance and growth of microbial (and other) cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass. In general, cells are grown and maintained at an appropriate temperature, gas mixture, and pH (such as about 20° C. to about 37° C., about 0% to about 84% CO2, and a pH between about 3 to about 9). In some aspects, cells are grown at 35° C. In certain embodiments, such as where thermophilic bacteria are used as the host cells, higher temperatures (e.g., 50° C.-75° C.) may be used. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fedbatch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that can be consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at a rate that can be supported by the amount of sugar in the cell medium. In some embodiments, sugar does not accumulate during the time the cells are cultured. In various embodiments, the cells are cultured under limited sugar conditions for times greater than or about 1, 2, 3, 5, 10, 15, 20, 25, 30, 35, 40, 50, 60, or 70 hours or even up to about 5-10 days. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some aspects, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.60% (w/v), 0.5% (w/v), 0.4% (w/v), 0.3% (w/v), 0.2% (w/v), or 0.1% (w/v) glucose. In some cultures, significantly higher levels of sugar (e.g., glucose) are used, e.g., at least 10% (w/v), 20% (w/v), 30% (w/v), 40% (w/V), 50% (w/v), 60% (w/v), 70% (w/v), or up to the solubility limit for the sugar in the medium, including any ranges and subranges therebetween. In some embodiments, the sugar levels fall within a range of any two of the above values, e.g.: 0.1-10% (w/v), 1.0-20% (w/v), 10-70% (w/v), 20-60% (w/v), or 30-50% (w/v). Furthermore, different sugar levels can be used for different phases of culturing. For fed-batch culture (e.g., of E. coli, S. cerevisiae or C. glutamicum), the sugar level can be about 10-200 g/L (1-20% (w/v)) in the batch phase and then up to about 500-700 g/L (50-70% in the feed).

Additionally, the minimal medium can be supplemented with 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract, including any ranges and subranges therebetween. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), or 0.02% (w/v) yeast extract, including any ranges and subranges therebetween. In some cultures, significantly higher levels of yeast extract can be used, e.g., at least 1.5% (w/v), 2.0% (w/v), 2.5% (w/v), or 3% (w/v). In some cultures (e.g., of E. coli, S. cerevisiae or C. glutamicum), the yeast extract level falls within a range of any two of the above values, e.g.: 0.5-3.0% (w/v), 1.0-2.5% (w/v), or 1.5-2.0% (w/v).

Illustrative materials and methods suitable for the maintenance and growth of host cells are further described in Examples 1 and 2.

### Two-Phased Fermentation Process

In some embodiments, the disclosure provides a bi-phasic fermentation process capable of generating sufficient cell biomass and maintaining key factors for production. The bi-phasic fed-batch fermentation process disclosed herein allows for optimization of growth and production of the product of interest and an in-situ product extraction. The advantages of using such a fermentation process is that the product is continuously extracted from the aqueous phase and into the organic phase during the course of fermentation. The typical fermentation process consists of a seed train and a fed batch main fermentation.

In some embodiments, the seed train starts with a glycerol stock banked in media suitable for the strain as per standard methods. In some embodiments, the seed train process has a two-step shake flask seed train that allows for growing the cell-line to high enough densities, and also creates an environment (e.g. media and pH) similar to the fermentation process. In some embodiments, a fermentation seed tank can be used to further increase the amount of biomass prior to inoculation in the main fermentation vessel and further synchronize the cells prior to inoculation in the main tank. In some embodiments, the seed tank matches similar parameters to the batch phase of the main fermentation and is typically run without a feeding strategy in place, however this can be adjusted depending on the scale of the process. In some embodiments, media components can be altered depending on process conditions.

In some embodiments, the main fermentation process consists of a batch phase followed by a fed batch portion. The batch phase of the fermentation contains nutrients needed to harbor growth of the microorganism and where needed, a chemical repressor, pending expression control as illustrated in Example 12. In some embodiments, an organic solvent is added to the batch portion of the fermentation. In some embodiments the organic solvent can be fed in at a later stage. In some embodiments, the organic solvent is added upon induction of the microbial strain to produce the product. In some embodiments the organic solvent is added before the induction of the microbial strain to produce the product.

In some embodiments, the main fermentation process is temperature regulated (e.g. 30° C.), pH controlled typically one sided but could be two sided (e.g. pH 5.0 set point controlled with ammonium hydroxide or similar), and dissolved oxygen maintained at a predetermined setpoint (e.g. DO: 30% or similar). In some embodiments, the present disclosure teaches that during the course of the batch phase of fermentation a typical DO trend is observed after which a DO and pH signal are used to trigger the addition of an inducer (when required) and then the feeding regime. In some embodiments, fermentation tanks are aerated by sparging air. In some embodiments, the fermentation tanks comprise cascade control on agitation to maintain DO set point. In some embodiments, the fermentation tanks are supplemented with oxygen when necessary.

In some embodiments, the present disclosure teaches that during the fed-batch portion of fermentation carbon substrate (e.g. glucose) and media are fed into the fermentation vessel. In some embodiments, the media contains inducer and/or lacking repressor as illustrated in Example 12 (depending on the expression system used). Thus, in some embodiments, the present disclosure teaches a feeding profile that is fixed feed, DO-Stat, pH-stat, dynamic feed, or similar depending on the process parameters.

In some embodiments, the present disclosure teaches that the fermentation tank are run till final volume is reached after which typical shutdown procedures are initiated. In some embodiments, antifoams are used to mitigate foaming events. In addition, media components for fermentation can be defined or undefined depending on the overall impact to process dynamics and economic considerations. The process outlined here discusses a fed batch fermentation however the production of nepetalactol and/or its derivatives is not be limited to a single fermentation process.

In some embodiments, the post fermentation tank liquid is drained and centrifugation is performed to separate out the respective fractions. Then further downstream processing is carried out to separate and purify product.

In some embodiments, the present disclosure teaches that key factors that ensure increased production of target products include feed profile, temperature, O2, induction, dissolved oxygen levels (DO), pH, agitation, aeration, second phase and media composition.

In some embodiments, the fermentation process utilizes a polymer to aid in product isolation. In some embodiments, the polymer is silicone- or non-silicone-based. In some embodiments, the polymers can be homopolymers, copolymers, with varying archetypes such as block, random cross-linked (or not). The polymers may be used in a liquid or solid state, and they may have varying molecular weight distributions. The polymers can comprise polyester, polyamide, polyether, and/or polyglycol. In some embodiments, a commercial polymer may be used, for example PolyTHF, Hytrel, PT-series, or Pebax.

In some embodiments, the fermentation process utilizes solvent extraction to aid in product isolation. In some embodiments, the organic solvent that can be used for bi-phasic fermentation is dodecane.

Without being bound by theory, it is thought that the bi-phasic fermentation process disclosed herein enables precise control of growth of the recombinant microbial cells, generating sufficient biomass, and reducing product and byproduct toxicity, thereby enabling high level transcription of the requisite genes for maximum productivity of the target products. In some embodiments, the byproduct may be a metabolic by product such as citrate or ethanol, or a main pathway byproduct.

### Dynamic Control Systems

In some embodiments, the disclosure provides dynamic control systems comprising one or more genetic switches, which are regulated by a small molecule. In some embodiments, the genetic switches control the transcription of the one or more polynucleotides disclosed herein in the recombinant microbial cells of this disclosure. In some embodiments, the small molecule is an amino acid, a phosphate source, or a nitrogen source. In some embodiments, the small molecule is capable of activating transcription, while in other embodiments, the small molecule is capable of repressing transcription.

Without being bound by theory, it is thought that the genetic switches disclosed herein allow for more control of transcription and subsequent expression of the one or more polynucleotides disclosed herein, in order to mitigate the metabolic burden of expression and the toxicity of intermediate compounds formed during the synthesis of nepetalactol/nepetalactone/dihydronepetalactone. In some embodiments, the dynamic control systems facilitate control of product synthesis, thus avoiding toxicity during early stages of the fermentation process. In some embodiments, the present disclosure teaches that dynamic modulation of gene expression levels result in increased function of the nepetalactol/nepetalactone/dihydronepetalactone biosynthetic pathways.

A summary of the sequences of the present disclosure, included in the sequence listing, is provided in Table 8, below.

TABLE 8
List of SEQ ID Nos of protein sequences and the corresponding DNA sequences encoding each.

| Protein | | | DNA |
|---|---|---|---|
| SEQ ID | Gene | | SEQ ID |
| NO. | name | Source organism | NO. |
| 1 | GPPS | Saccharomyces cerevisiae | 789 |
| 2 | GPPS | Saccharomyces cerevisiae | 790 |
| 3 | GPPS | Abies grandis | 791 |
| 4 | GPPS | Catharanthus roseus | 792 |
| 5 | GPPS | Picea abies | 793 |
| 6 | GPPS | Geobacillussp.WSUCF1 | 794 |
| 7 | GPPS | Saccharomycescerevisiae(strainATCC204508/S288c)(Baker's yeast) | 795 |
| 8 | GPPS | Saccharomycescerevisiae(strainATCC204508/S288c)(Baker's yeast) | 796 |
| 9 | GPPS | Saccharomycescerevisiae(strainATCC204508/S288c)(Baker's yeast) | 797 |
| 10 | GPPS | Neosartorya fumigata (strain ATCC MYA-4609/Af293/CBS 101355/FGSC A1100) | 798 |
| | | (Aspergillus fumigatus) | |
| 11 | GPPS | Catharanthus roseus (Madagascar periwinkle) (Vinca rosea) | 799 |
| 12 | GPPS | Rhizobium acidisoli | 800 |
| 13 | GPPS | Escherichiacoli(strainK12) | 801 |
| 14 | GPPS | Escherichiacoli(strainK12) | 802 |
| 15 | GPPS | Brucella suis (strain ATCC 23445/NCTC 10510) | 803 |
| 16 | GPPS | Arabidopsisthaliana(Mouse-earcress) | 804 |
| 17 | GPPS | Buchneraaphidicolasubsp.Acyrthosiphonpisum(strainAPS)(Acyrthosiphonpisumsymbioticbacterium) | 805 |
| 18 | GPPS | Dendroctonus ponderosae (Mountain pine beetle) | 806 |
| 19 | GPPS | Picea abies (Norway spruce) (Picea excelsa) | 807 |
| 20 | GPPS | Abies grandis (Grand fir) (Pinus grandis) | 808 |
| 21 | GPPS | Corynebacterium glutamicum (strain ATCC 13032/DSM 20300/JCM 1318/LMG 3730/NC1MB 10025) | 809 |
| 22 | GPPS | Vitisvinifera(Grape) | 810 |
| 23 | GPPS | Picea abies (Norway spruce) (Picea excelsa) | 811 |
| 24 | GPPS | Picea abies (Norway spruce) (Picea excelsa) | 812 |
| 25 | GPPS | Sus scrofa (Pig) | 813 |
| 26 | GPPS | Acyrthosiphon pisum (Pea aphid) | 814 |
| 27 | GPPS | Mycobacterium tuberculosis | 815 |
| 28 | GPPS | Staphylococcus aureus (strain NCTC 8325) | 816 |
| 29 | GPPS | Geobacillussp.WSUCF1 | 817 |
| 30 | GPPS | Saccharomycescerevisiae(strainATCC204508/S288c)(Baker's yeast) | 818 |
| 31 | GPPS | Neosartorya fumigata (strain ATCC MYA-4609/Af293/CBS 101355/FGSC A1100) | 819 |
| | | (Aspergillus fumigatus) | |
| 32 | GPPS | Neosartorya fumigata (strain ATCC MYA-4609/Af293/CBS 101355/FGSC A1100) | 820 |
| | | (Aspergillus fumigatus) | |
| 33 | GPPS | Catharanthus roseus (Madagascar periwinkle) (Vinca rosea) | 821 |
| 34 | GPPS | Rhizobium acidisoli | 822 |
| 35 | GPPS | Escherichiacoli(strainK12) | 823 |
| 36 | GPPS | Brucella suis (strain ATCC 23445/NCTC 10510) | 824 |
| 37 | GPPS | Arabidopsisthaliana(Mouse-earcress) | 825 |
| 38 | GPPS | Buchneraaphidicolasubsp.Acyrthosiphonpisum(strainAPS)(Acyrthosiphonpisumsymbioticbacterium) | 826 |
| 39 | GPPS | Dendroctonus ponderosae (Mountain pine beetle) | 827 |
| 40 | GPPS | Picea abies (Norway spruce) (Picea excelsa) | 828 |
| 41 | GPPS | Abies grandis (Grand fir) (Pinus grandis) | 829 |
| 42 | GPPS | Corynebacterium glutamicum (strain ATCC 13032/DSM 20300/JCM 1318/LMG 3730/NC1MB 10025) | 830 |
| 43 | GPPS | Vitisvinifera(Grape) | 831 |
| 44 | GPPS | Picea abies (Norway spruce) (Picea excelsa) | 832 |
| 45 | GPPS | Picea abies (Norway spruce) (Picea excelsa) | 833 |
| 46 | GPPS | Picea abies (Norway spruce) (Picea excelsa) | 834 |
| 47 | GPPS | Picea abies (Norway spruce) (Picea excelsa) | 835 |
| 48 | GPPS | Picea abies (Norway spruce) (Picea excelsa) | 836 |
| 49 | GPPS | Sus scrofa (Pig) | 837 |
| 50 | GPPS | Acyrthosiphon pisum (Pea aphid) | 838 |
| 51 | GPPS | Mycobacteriumtuberculosis | 839 |
| 52 | GPPS | Staphylococcus aureus (strain NCTC 8325) | 840 |
| 53 | GPPS | Geobacillussp.WSUCF1 | 841 |
| 54 | GPPS | Geobacillussp.WSUCF1 | 842 |
| 55 | GPPS | Geobacillussp.WSUCF1 | 843 |
| 56 | GPPS | Geobacillussp.WSUCF1 | 844 |
| 57 | GPPS | Rhizobium acidisoli | 845 |
| 58 | GPPS | Rhizobium acidisoli | 846 |
| 59 | GPPS | Rhizobium acidisoli | 847 |
| 60 | GPPS | Escherichiacoli(strainK12) | 848 |
| 61 | GPPS | Escherichiacoli(strainK12) | 849 |
| 62 | GPPS | Escherichiacoli(strainK12) | 850 |
| 63 | GPPS | Brucella suis (strain ATCC 23445/NCTC 10510) | 851 |
| 64 | GPPS | Brucella suis (strain ATCC 23445/NCTC 10510) | 852 |
| 65 | GPPS | Buchneraaphidicolasubsp.Acyrthosiphonpisum(strainAPS)(Acyrthosiphonpisumsymbioticbacterium) | 853 |
| 66 | GPPS | Buchneraaphidicolasubsp.Acyrthosiphonpisum(strainAPS)(Acyrthosiphonpisumsymbioticbacterium) | 854 |
| 67 | GPPS | Buchneraaphidicolasubsp.Acyrthosiphonpisum(strainAPS)(Acyrthosiphonpisumsymbioticbacterium) | 855 |
| 68 | GPPS | Dendroctonus ponderosae (Mountain pine beetle) | 856 |
| 69 | GPPS | Picea abies (Norway spruce) (Picea excelsa) | 857 |
| 70 | GPPS | Picea abies (Norway spruce) (Picea excelsa) | 858 |
| 71 | GPPS | Picea abies (Norway spruce) (Picea excelsa) | 859 |
| 72 | GPPS | Abies grandis (Grand fir) (Pinus grandis) | 860 |
| 73 | GPPS | Abies grandis (Grand fir) (Pinus grandis) | 861 |
| 74 | GPPS | Abies grandis (Grand fir) (Pinus grandis) | 862 |
| 75 | GPPS | Picea abies (Norway spruce) (Picea excelsa) | 863 |
| 76 | GPPS | Picea abies (Norway spruce) (Picea excelsa) | 864 |
| 77 | GPPS | Picea abies (Norway spruce) (Picea excelsa) | 865 |
| 78 | GPPS | Sus scrofa (Pig) | 866 |
| 79 | GPPS | Staphylococcus aureus (strain NCTC 8325) | 867 |
| 80 | GPPS | Staphylococcus aureus (strain NCTC 8325) | 868 |
| 81 | GPPS | Staphylococcus aureus (strain NCTC 8325) | 869 |
| 82 | GPPS | Geobacillussp.WSUCF1 | 870 |
| 83 | GPPS | Saccharomycescerevisiae(strainATCC204508/S288c)(Baker's yeast) | 871 |
| 84 | GPPS | Neosartorya fumigata (strain ATCC MYA-4609/Af293/CBS 101355/FGSC A1100) (Aspergillus fumigatus) | 872 |
| 85 | GPPS | Catharanthus roseus (Madagascar periwinkle) (Vinca rosea) | 873 |
| 86 | GPPS | Catharanthus roseus (Madagascar periwinkle) (Vinca rosea) | 874 |
| 87 | GPPS | Catharanthus roseus (Madagascar periwinkle) (Vinca rosea) | 875 |
| 88 | GPPS | Rhizobium acidisoli | 876 |
| 89 | GPPS | Escherichiacoli(strainK12) | 877 |
| 90 | GPPS | Brucella suis (strain ATCC 23445/NCTC 10510) | 878 |
| 91 | GPPS | Arabidopsisthaliana(Mouse-earcress) | 879 |
| 92 | GPPS | Arabidopsisthaliana(Mouse-earcress) | 880 |
| 93 | GPPS | Arabidopsisthaliana(Mouse-earcress) | 881 |
| 94 | GPPS | Buchneraaphidicolasubsp.Acyrthosiphonpisum(strainAPS)(Acyrthosiphonpisumsymbioticbacterium) | 882 |
| 95 | GPPS | Dendroctonus ponderosae (Mountain pine beetle) | 883 |
| 96 | GPPS | Picea abies (Norway spruce) (Picea excelsa) | 884 |
| 97 | GPPS | Abies grandis (Grand fir) (Pinus grandis) | 885 |
| 98 | GPPS | Corynebacterium glutamicum (strain ATCC 13032/DSM 20300/JCM 1318/LMG 3730/NCIMB 10025) | 886 |
| 99 | GPPS | Vitisvinifera(Grape) | 887 |
| 100 | GPPS | Vitisvinifera(Grape) | 888 |
| 101 | GPPS | Vitisvinifera(Grape) | 889 |
| 102 | GPPS | Picea abies (Norway spruce) (Picea excelsa) | 890 |
| 103 | GPPS | Sus scrofa (Pig) | 891 |
| 104 | GPPS | Acyrthosiphon pisum (Pea aphid) | 892 |
| 105 | GPPS | Mycobacteriumtuberculosis | 893 |
| 106 | GPPS | Mycobacteriumtuberculosis | 894 |
| 107 | GPPS | Mycobacteriumtuberculosis | 895 |
| 108 | GPPS | Staphylococcus aureus (strain NCTC 8325) | 896 |
| 109 | GPPS | Picea abies | 897 |
| no | GPPS | Abies grandis | 898 |
| 111 | GPPS | Catharanthus roseus | 899 |
| 112 | GPPS | Picea abies | 900 |
| 113 | GPPS | Abies grandis | 901 |
| 114 | GPPS | Catharanthus roseus | 902 |
| 115 | GPPS | Abies grandis | 903 |
| 116 | GPPS | Catharanthus roseus and S. cerevisiae | 904 |
| 117 | GPPS | Picea abies | 905 |
| 118 | GPPS | Humulus lupulus | 906 |
| 119 | GPPS | Humulus lupulus | 907 |
| 120 | GPPS | Mentha × piperita | 908 |
| 121 | GPPS | Mentha × piperita | 909 |
| 122 | GPPS | Catharanthus roseus | 910 |
| 123 | GPPS | Catharanthus roseus | 911 |
| 124 | GPPS | Nepeta cataria | 912 |
| 125 | GPPS | Nepeta cataria | 913 |
| 126 | GPPS | Streptomyces aculeolatus | 914 |
| 127 | GPPS | Streptomyces sp. KO-3988 | 915 |
| 128 | GPPS | Streptomyces cinnamonensis | 916 |
| 129 | GPPS | Streptomyces longwoodensis | 917 |
| 130 | GPPS | Streptomyces sp. GKU 895 | 918 |
| 131 | GPPS | Streptomyces sp. NRRL S-37 | 919 |
| 132 | GPPS | Streptomyces aculeolatus | 920 |
| 133 | GPPS | Streptomyces sp. KO-3988 | 921 |
| 134 | GPPS | Streptomyces cinnamonensis | 922 |
| 135 | GPPS | Streptomyces longwoodensis | 923 |
| 136 | GPPS | Streptomyces sp. GKU 895 | 924 |
| 137 | GPPS | Streptomyces sp. NRRL S-37 | 925 |
| 138 | GPPS | Penicillium aethiopicum | 926 |
| 139 | GPPS | Penicillium aethiopicum | 927 |
| 140 | GES | Ocimum basilicum (Sweet basil) | 928 |
| 141 | GES | Catharanthus roseus | 929 |
| 142 | GES | Ocimum basilicum | 930 |
| 143 | GES | Valeriana officinalis | 931 |
| 144 | GES | Catharanthus roseus | 932 |
| 145 | GES | Ocimum basilicum | 933 |
| 146 | GES | Valeriana officinalis | 934 |
| 147 | GES | Catharanthus roseus | 935 |
| 148 | GES | Ocimum basilicum | 936 |
| 149 | GES | Perilla citriodora | 937 |
| 150 | GES | Valeriana officinalis | 938 |
| 151 | GES | Rosa hybrid cultivar | 939 |
| 152 | GES | Arabidopsis thaliana | 940 |
| 153 | GES | Catharanthus roseus | 941 |
| 154 | GES | Ocimum basilicum | 942 |
| 155 | GES | Perilla citriodora | 943 |
| 156 | GES | Valeriana officinalis | 944 |
| 157 | GES | Vinca minor | 945 |
| 158 | GES | Cinchona pubescens | 946 |
| 159 | GES | Rauvolfia serpentina | 947 |
| 160 | GES | Swertia japonica | 948 |
| 161 | GES | Coffea canephora | 949 |
| 162 | GES | Citrus unshiu | 950 |
| 163 | GES | Citrus unshiu | 951 |
| 164 | GES | Glycine soja | 952 |
| 165 | GES | Cynara cardunculus var. scolymus | 953 |
| 166 | GES | Dorcoceras hygrometricum | 954 |
| 167 | GES | Dorcoceras hygrometricum | 955 |
| 168 | GES | Helianthus annuus | 956 |
| 169 | GES | Actinidia chinensis var. chinensis | 957 |
| 170 | GES | Cinchona ledgeriana | 958 |
| 171 | GES | Lonicera japonica | 959 |
| 172 | GES | Cinchona pubescens | 960 |
| 173 | GES | Nepeta mussinii | 961 |
| 174 | GES | Nepeta cataria | 962 |
| 175 | GES | Nepeta cataria | 963 |
| 176 | GES | Phyla dulcis | 964 |
| 177 | GES | Vitis vinifera | 965 |
| 178 | GES | Catharanthus roseus | 966 |
| 179 | GES | Olea europaea | 967 |
| 180 | GES | Valeriana officinalis | 968 |
| 181 | GES | Valeriana officinalis | 969 |
| 182 | GES | Valeriana officinalis | 970 |
| 183 | GES | Pogostemon cablin | 971 |
| 184 | GES | Picrorhiza kurrooa | 972 |
| 185 | GES | Gentiana rigescens | 973 |
| 186 | GES | Camptotheca acuminata | 974 |
| 187 | GES | Osmanthus fragrans | 975 |
| 188 | GES | synthetic construct | 976 |
| 189 | GES | Phaseolus lunatus | 977 |
| 190 | GES | unknown | 978 |
| 191 | GES | Vigna angularis var. angularis | 979 |
| 192 | GES | Vitis vinifera | 980 |
| 193 | GES | Coffea arabica | 981 |
| 194 | GES | Coffea canephora | 982 |
| 195 | GES | Glycine soja | 983 |
| 196 | GES | Glycine soja | 984 |
| 197 | GES | Vigna angularis | 985 |
| 198 | GES | Glycine max | 986 |
| 199 | GES | Cajanus cajan | 987 |
| 200 | GES | Cajanus cajan | 988 |
| 201 | GES | Vitis vinifera | 989 |
| 202 | GES | Vitis vinifera | 990 |
| 203 | GES | Glycine max | 991 |
| 204 | GES | Lupinus angustifolius | 992 |
| 205 | GES | Handroanthus impetiginosus | 993 |
| 206 | GES | Handroanthus impetiginosus | 994 |
| 207 | GES | Lactuca sativa | 995 |
| 208 | GES | Parasponia andersonii | 996 |
| 209 | GES | Trema orientalis | 997 |
| 210 | GES | unknown | 998 |
| 211 | GES | unknown | 999 |
| 212 | GES | Ricinus communis | 1000 |
| 213 | GES | Medicago truncatula | 1001 |
| 214 | GES | Cicer arietinum | 1002 |
| 215 | GES | Glycine max | 1003 |
| 216 | GES | Glycine max | 1004 |
| 217 | GES | Phaseolus vulgaris | 1005 |
| 218 | GES | Phaseolus vulgaris | 1006 |
| 219 | GES | Phaseolus vulgaris | 1007 |
| 220 | GES | Morus notabilis | 1008 |
| 221 | GES | Vitis vinifera | 1009 |
| 222 | GES | Sesamum indicum | 1010 |
| 223 | GES | Jatropha curcas | 1011 |
| 224 | GES | Erythranthe guttata | 1012 |
| 225 | GES | Vigna radiata var. radiata | 1013 |
| 226 | GES | Vigna radiata var. radiata | 1014 |
| 227 | GES | Arachis duranensis | 1015 |
| 228 | GES | Vigna angularis | 1016 |
| 229 | GES | Vigna angularis | 1017 |
| 230 | GES | Lupinus angustifolius | 1018 |
| 231 | GES | Cajanus cajan | 1019 |
| 232 | GES | Cajanus cajan | 1020 |
| 233 | GES | Manihot esculenta | 1021 |
| 234 | GES | Hevea brasiliensis | 1022 |
| 235 | GES | Helianthus annuus | 1023 |
| 236 | GES | Olea europaea var. sylvestris | 1024 |
| 237 | GES | Lactuca sativa | 1025 |
| 238 | GES | Citrus clementina | 1026 |
| 239 | GES | Medicago truncatula | 1027 |
| 240 | GES | Cicer arietinum | 1028 |
| 241 | GES | Citrus sinensis | 1029 |
| 242 | GES | Vigna angularis | 1030 |
| 243 | GES | Helianthus annuus | 1031 |
| 244 | GES | Helianthus annuus | 1032 |
| 245 | GES | Helianthus annuus | 1033 |
| 246 | GES | Olea europaea var. sylvestris | 1034 |
| 247 | GES | Olea europaea var. sylvestris | 1035 |
| 248 | GES | Olea europaea var. sylvestris | 1036 |
| 249 | GES | Olea europaea var. sylvestris | 1037 |
| 250 | G6H | Catharanthus roseus | 1038 |
| 251 | G8H | Catharanthus roseus | 1039 |
| 252 | G8H | Catharanthus roseus | 1040 |
| 253 | G8H | Catharanthus roseus | 1041 |
| 254 | G8H | Catharanthus roseus | 1042 |
| 255 | G6H | Catharanthus roseus | 1043 |
| 256 | G8H | Catharanthus roseus | 1044 |
| 257 | G8H | Catharanthus roseus | 1045 |
| 258 | G8H | Catharanthus roseus | 1046 |
| 259 | G8H | Catharanthus roseus | 1047 |
| 260 | G6H | Catharanthus roseus | 1048 |
| 261 | G8H | Catharanthus roseus | 1049 |
| 262 | G8H | Catharanthus roseus | 1050 |
| 263 | G8H | Catharanthus roseus | 1051 |
| 264 | G8H | Nepeta cataria | 1052 |
| 265 | G6H | Nepeta mussinii | 1053 |
| 266 | G8H | Nepeta cataria | 1054 |
| 267 | G6H | Nepeta mussinii | 1055 |
| 268 | G8H | Nepeta cataria | 1056 |
| 269 | G8H | Nepeta mussinii | 1057 |
| 270 | G6H | Nepeta cataria | 1058 |
| 271 | G8H | Nepeta mussinii | 1059 |
| 272 | G6H | Vigna angularis | 1060 |
| 273 | G8H | Bacillus megaterium NBRC 15308 | 1061 |
| 274 | G8H | Bacillus megaterium NBRC 15308 | 1062 |
| 275 | G6H | Camptotheca acuminata | 1063 |
| 276 | G8H | Vinca minor | 1064 |
| 277 | G6H | Ophiorrhiza pumila | 1065 |
| 278 | G8H | Rauvolfia serpentina | 1066 |
| 279 | G8H | Lonicera japonica | 1067 |
| 280 | G8H | Erythranthe guttata | 1068 |
| 281 | G8H | Picrorhiza kurrooa | 1069 |
| 282 | G6H | Olea europaea | 1070 |
| 283 | G8H | Gentiana rigescens | 1071 |
| 284 | G8H | Nepeta cataria | 1072 |
| 285 | CPR | Arabidopsis thaliana | 1073 |
| 286 | CPR | Catharanthus roseus | 1074 |
| 287 | CPR | Catharanthus roseus | 1075 |
| 288 | CPR | Arabidopsis thaliana | 1076 |
| 289 | CPR | Catharanthus roseus | 1077 |
| 290 | CPR | Arabidopsis thaliana | 1078 |
| 291 | CPR | Catharanthus roseus | 1079 |
| 292 | CPR | Nepeta mussinii | 1080 |
| 293 | CPR | Camptotheca acuminata | 1081 |
| 294 | CPR | Arabidopsis thaliana | 1082 |
| 295 | CPR | Arabidopsis thaliana | 1083 |
| 296 | CPR | Nepeta mussinii | 1084 |
| 297 | CPR | Camptotheca acuminata | 1085 |
| 298 | CPR | Nepeta mussinii | 1086 |
| 299 | CPR | Camptotheca acuminata | 1087 |
| 300 | G8H | Swertia mussotii | 1088 |
| 301 | G8H | Camptotheca acuminata | 1089 |
| 302 | G8H | Lonicera japonica | 1090 |
| 303 | G8H | Erythranthe guttata | 1091 |
| 304 | G8H | Erythranthe guttata | 1092 |
| 305 | G8H | Nepeta cataria | 1093 |
| 306 | G8H | Picrorhiza kurrooa | 1094 |
| 307 | G8H | Picrorhiza kurrooa | 1095 |
| 308 | G8H | Nepeta mussinii | 1096 |
| 309 | G8H | Olea europaea | 1097 |
| 310 | G8H | Sesamum indicum | 1098 |
| 311 | G8H | Coffea canephora | 1099 |
| 312 | G8H | Dorcoceras hygrometricum | 1100 |
| 313 | G8H | Gentiana rigescens | 1101 |
| 314 | G8H | Vinca minor | 1102 |
| 315 | G8H | Ophiorrhiza pumila | 1103 |
| 316 | G8H | Rauvolfia serpentina | 1104 |
| 317 | G8H | Cinchona calisaya | 1105 |
| 318 | G8H | Tabernaemontana elegans | 1106 |
| 319 | G8H | Catharanthus roseus | 1107 |
| 320 | G8H | Catharanthus roseus | 1108 |
| 321 | G8H | Catharanthus roseus | 1109 |
| 322 | G8H | Catharanthus roseus | 1110 |
| 323 | CYB5 | Catharanthus roseus | 1111 |
| 324 | CYB5 | Yarrowia lipolytica CLIB122 | 1112 |
| 325 | CYB5 | Nepeta cataria | 1113 |
| 326 | CYB5 | Catharanthus roseus | 1114 |
| 327 | CYB5 | Nepeta cataria | 1115 |
| 328 | CYB5 | Artemesia annua | 1116 |
| 329 | CYB5 | Arabidopsis thaliana | 1117 |
| 330 | 8HGO | Catharanthus roseus (Madagascar periwinkle) (Vinca rosea) | 1118 |
| 331 | 8HGO | Catharanthus roseus | 1119 |
| 332 | 8HGO | Nepeta cataria | 1120 |
| 333 | 8HGO | Sesamum indicum | 1121 |
| 334 | 8HGO | Camptotheca acuminata | 1122 |
| 335 | 8HGO | Sesamum indicum | 1123 |
| 336 | 8HGO | Swertia japonica | 1124 |
| 337 | 8HGO | Ophiorrhiza pumila | 1125 |
| 338 | 8HGO | Cinchona ledgeriana | 1126 |
| 339 | 8HGO | Lonicera japonica | 1127 |
| 340 | 8HGO | Coffea canephora | 1128 |
| 341 | 8HGO | Rauvolfia serpentina | 1129 |
| 342 | 8HGO | Gentiana rigescens | 1130 |
| 343 | 8HGO | Catharanthus roseus | 1131 |
| 344 | 8HGO | Nepeta cataria | 1132 |
| 345 | 8HGO | Ocimum basilicum | 1133 |
| 346 | 8HGO | Sesamum indicum | 1134 |
| 347 | 8HGO | Capsicum annuum | 1135 |
| 348 | 8HGO | Camptotheca acuminata | 1136 |
| 349 | 8HGO | Solanum tuberosum | 1137 |
| 350 | 8HGO | Sesamum indicum | 1138 |
| 351 | 8HGO | Swertia japonica | 1139 |
| 352 | 8HGO | Ophiorrhiza pumila | 1140 |
| 353 | 8HGO | Cinchona ledgeriana | 1141 |
| 354 | 8HGO | Lonicera japonica | 1142 |
| 355 | 8HGO | Coffea canephora | 1143 |
| 356 | 8HGO | Rauvolfia serpentina | 1144 |
| 357 | 8HGO | Gentiana rigescens | 1145 |
| 358 | 8HGO | Catharanthus roseus | 1146 |
| 359 | 8HGO | Olea europaea subsp. europaea | 1147 |
| 360 | 8HGO | Sesamum indicum | 1148 |
| 361 | 8HGO | Olea europaea | 1149 |
| 362 | 8HGO | Erythranthe guttata | 1150 |
| 363 | 8HGO | Catharanthus roseus | 1151 |
| 364 | 8HGO | Ocimum basilicum | 1152 |
| 365 | 8HGO | Camptotheca acuminata | 1153 |
| 366 | 8HGO | Swertia japonica | 1154 |
| 367 | 8HGO | Cinchona ledgeriana | 1155 |
| 368 | 8HGO | Rauvolfia serpentina | 1156 |
| 369 | ISY | Arabidopsis thaliana (Mouse-earcress) | 1157 |
| 370 | ISY | Digitalis lanata (Grecian foxglove) | 1158 |
| 371 | ISY | Nepeta mussinii | 1159 |
| 372 | ISY | Nepeta cataria | 1160 |
| 373 | ISY | Catharanthus roseus (Madagascar periwinkle) (Vinca rosea) | 1161 |
| 374 | ISY | Catharanthus roseus | 1162 |
| 375 | ISY | Nepeta mussinii | 1163 |
| 376 | ISY | Nepeta cataria | 1164 |
| 377 | ISY | Olea europaea | 1165 |
| 378 | ISY | Catharanthus roseus | 1166 |
| 379 | ISY | Nepeta mussinii | 1167 |
| 380 | ISY | Nepeta cataria | 1168 |
| 381 | ISY | Nicotiana tabacum | 1169 |
| 382 | ISY | Elaeis guineensis | 1170 |
| 383 | ISY | Citrus clementina | 1171 |
| 384 | ISY | Sesamum indicum | 1172 |
| 385 | ISY | Camptotheca acuminata | 1173 |
| 386 | ISY | Cinchona pubescens | 1174 |
| 387 | ISY | Ophiorrhiza pumila | 1175 |
| 388 | ISY | Lonicera japonica | 1176 |
| 389 | ISY | Digitalis purpurea | 1177 |
| 390 | ISY | Antirrhinum majus | 1178 |
| 391 | ISY | Trifolium subterraneum | 1179 |
| 392 | ISY | Corchorus capsularis | 1180 |
| 393 | ISY | Nicotiana tabacum | 1181 |
| 394 | ISY | Panicum hallii | 1182 |
| 395 | ISY | Medicago truncatula | 1183 |
| 396 | ISY | Juglans regia | 1184 |
| 397 | ISY | Triticum urartu | 1185 |
| 398 | ISY | Citrus clementina | 1186 |
| 399 | ISY | Panicum hallii | 1187 |
| 400 | ISY | Prunus persica | 1188 |
| 401 | ISY | Tarenaya hassleriana | 1189 |
| 402 | ISY | Capsicum baccatum | 1190 |
| 403 | ISY | Medicago truncatula | 1191 |
| 404 | ISY | Nicotiana sylvestris | 1192 |
| 405 | ISY | Oryza sativa Japonica Group | 1193 |
| 406 | ISY | Oryza sativa Japonica Group | 1194 |
| 407 | ISY | Cynara cardunculus var. scolymus | 1195 |
| 408 | ISY | Ornithogalum longebracteatum | 1196 |
| 409 | ISY | Allium ursinum | 1197 |
| 410 | ISY | Convallaria majalis | 1198 |
| 411 | ISY | Populus trichocarpa | 1199 |
| 412 | ISY | Sorghum bicolor | 1200 |
| 413 | ISY | Zea mays | 1201 |
| 414 | ISY | Daucus carota subsp. sativus | 1202 |
| 415 | ISY | Nepeta cataria | 1203 |
| 416 | ISY | Catharanthus roseus | 1204 |
| 417 | ISY | Dichanthelium oligosanthes | 1205 |
| 418 | ISY | Sorghum bicolor | 1206 |
| 419 | ISY | Tarenaya hassleriana | 1207 |
| 420 | ISY | Citrus sinensis | 1208 |
| 421 | ISY | Picea sitchensis | 1209 |
| 422 | ISY | Cajanus cajan | 1210 |
| 423 | ISY | Citrus clementina | 1211 |
| 424 | ISY | Aquilegia coerulea | 1212 |
| 425 | ISY | Lonicera japonica | 1213 |
| 426 | ISY | Olea europaea subsp. europaea | 1214 |
| 427 | ISY | Thlaspi densiflorum | 1215 |
| 428 | ISY | Stellaria media | 1216 |
| 429 | ISY | Erysimum crepidifolium | 1217 |
| 430 | ISY | Morus notabilis | 1218 |
| 431 | ISY | Helianthus annuus | 1219 |
| 432 | ISY | Capsicum annuum | 1220 |
| 433 | ISY | Macleaya cordata | 1221 |
| 434 | ISY | Citrus clementina | 1222 |
| 435 | ISY | Arachis ipaensis | 1223 |
| 436 | ISY | Vitis vinifera | 1224 |
| 437 | ISY | Hevea brasiliensis | 1225 |
| 438 | ISY | Dorcoceras hygrometricum | 1226 |
| 439 | ISY | Brassica napus | 1227 |
| 440 | ISY | Ziziphus jujuba | 1228 |
| 441 | ISY | Punica granatum | 1229 |
| 442 | ISY | Capsicum baccatum | 1230 |
| 443 | ISY | Carica papaya | 1231 |
| 444 | ISY | Gossypium hirsutum | 1232 |
| 445 | ISY | Cucumis sativus | 1233 |
| 446 | ISY | Citrus clementina | 1234 |
| 447 | ISY | Catharanthus roseus | 1235 |
| 448 | ISY | Fragaria vesca subsp. vesca | 1236 |
| 449 | ISY | Prunus avium | 1237 |
| 450 | ISY | Salvia rosmarinus | 1238 |
| 451 | ISY | Elaeis guineensis | 1239 |
| 452 | ISY | Erythranthe guttata | 1240 |
| 453 | ISY | Helianthus annuus | 1241 |
| 454 | ISY | Genlisea aurea | 1242 |
| 455 | ISY | Arabidopsis thaliana | 1243 |
| 456 | ISY | Lupinus angustifolius | 1244 |
| 457 | ISY | Ananas comosus | 1245 |
| 458 | ISY | Beta vulgaris subsp. vulgaris | 1246 |
| 459 | ISY | Gossypium raimondii | 1247 |
| 460 | ISY | Citrus sinensis | 1248 |
| 461 | ISY | Amborella trichopoda | 1249 |
| 462 | ISY | Musa acuminata subsp. malaccensis | 1250 |
| 463 | ISY | Zostera marina | 1251 |
| 464 | ISY | Cephalotus follicularis | 1252 |
| 465 | ISY | Ipomoea nil | 1253 |
| 466 | ISY | Ricinus communis | 1254 |
| 467 | ISY | Elaeis guineensis | 1255 |
| 468 | ISY | Citrus clementina | 1256 |
| 469 | ISY | Musa acuminata subsp. malaccensis | 1257 |
| 470 | ISY | Theobroma cacao | 1258 |
| 471 | ISY | Gomphocarpus fruticosus | 1259 |
| 472 | ISY | Lupinus angustifolius | 1260 |
| 473 | ISY | Brachypodium distachyon | 1261 |
| 474 | ISY | Oryza brachyantha | 1262 |
| 475 | ISY | Catharanthus roseus | 1263 |
| 476 | ISY | Populus euphratica | 1264 |
| 477 | ISY | Catharanthus roseus | 1265 |
| 478 | ISY | Prunus mume | 1266 |
| 479 | ISY | Ziziphus jujuba | 1267 |
| 480 | ISY | Prunus persica | 1268 |
| 481 | ISY | Sesamum indicum | 1269 |
| 482 | ISY | Panicum hallii | 1270 |
| 483 | ISY | Fragaria vesca subsp. vesca | 1271 |
| 484 | ISY | Setaria italica | 1272 |
| 485 | ISY | Populus trichocarpa | 1273 |
| 486 | ISY | Juglans regia | 1274 |
| 487 | ISY | Jatropha curcas | 1275 |
| 488 | ISY | Hevea brasiliensis | 1276 |
| 489 | ISY | Camptotheca acuminata | 1277 |
| 490 | ISY | Malus domestica | 1278 |
| 491 | ISY | Panicum hallii | 1279 |
| 492 | ISY | Arachis duranensis | 1280 |
| 493 | ISY | Catharanthus roseus | 1281 |
| 494 | ISY | Spinacia oleracea | 1282 |
| 495 | ISY | Trifolium subterraneum | 1283 |
| 496 | ISY | Ziziphus jujuba | 1284 |
| 497 | ISY | Medicago truncatula | 1285 |
| 498 | ISY | Medicago truncatula | 1286 |
| 499 | ISY | Medicago truncatula | 1287 |
| 500 | ISY | Spinacia oleracea | 1288 |
| 501 | ISY | Juglans regia | 1289 |
| 502 | ISY | Populus tremuloides | 1290 |
| 503 | ISY | Vitis vinifera | 1291 |
| 504 | ISY | Vitis vinifera | 1292 |
| 505 | ISY | Daucus carota subsp. sativus | 1293 |
| 506 | ISY | Dendrobium catenatum | 1294 |
| 507 | ISY | Passiflora incarnata | 1295 |
| 508 | ISY | Prunus avium | 1296 |
| 509 | ISY | Daucus carota subsp. sativus | 1297 |
| 510 | ISY | Solanum tuberosum | 1298 |
| 511 | ISY | Setaria italica | 1299 |
| 512 | ISY | Antirrhinum majus | 1300 |
| 513 | ISY | Coffea canephora | 1301 |
| 514 | ISY | Panicum hallii | 1302 |
| 515 | ISY | Oryza sativa Japonica Group | 1303 |
| 516 | ISY | Setaria italica | 1304 |
| 517 | ISY | Sesamum indicum | 1305 |
| 518 | ISY | Digitalis purpurea | 1306 |
| 519 | ISY | Digitalis lanata | 1307 |
| 520 | NOR | Nepeta mussinii | 1308 |
| 521 | NOR | Nepeta mussinii | 1309 |
| 522 | NOR | Nepeta cataria | 1310 |
| 523 | NOR | Nepeta cataria | 1311 |
| 524 | NOR | Nepeta cataria | 1312 |
| 525 | NOR | Nepeta cataria | 1313 |
| 526 | NOR | Nepeta cataria | 1314 |
| 527 | NOR | Nepeta cataria | 1315 |
| 528 | NOR | Nepeta cataria | 1316 |
| 529 | NOR | Nepeta cataria | 1317 |
| 530 | NOR | Nepeta cataria | 1318 |
| 531 | NOR | Nepeta cataria | 1319 |
| 532 | NOR | Nepeta cataria | 1320 |
| 533 | NOR | Nepeta cataria | 1321 |
| 534 | NOR | Nepeta cataria | 1322 |
| 535 | NOR | Nepeta cataria or Nepeta mussinii | 1323 |
| 536 | NOR | Nepeta cataria or Nepeta mussinii | 1324 |
| 537 | NOR | Nepeta cataria or Nepeta mussinii | 1325 |
| 538 | NOR | Nepeta cataria or Nepeta mussinii | 1326 |
| 539 | NOR | Nepeta cataria or Nepeta mussinii | 1327 |
| 540 | NOR | Nepeta cataria or Nepeta mussinii | 1328 |
| 541 | NOR | Nepeta cataria or Nepeta mussinii | 1329 |
| 542 | NOR | Nepeta cataria or Nepeta mussinii | 1330 |
| 543 | NOR | Nepeta cataria or Nepeta mussinii | 1331 |
| 544 | NOR | Nepeta cataria or Nepeta mussinii | 1332 |
| 545 | NOR | Nepeta cataria or Nepeta mussinii | 1333 |
| 546 | NOR | Nepeta cataria or Nepeta mussinii | 1334 |
| 547 | NOR | Nepeta cataria or Nepeta mussinii | 1335 |
| 548 | NOR | Nepeta cataria or Nepeta mussinii | 1336 |
| 549 | NOR | Nepeta cataria or Nepeta mussinii | 1337 |
| 550 | NOR | Nepeta cataria or Nepeta mussinii | 1338 |
| 551 | NOR | Nepeta cataria or Nepeta mussinii | 1339 |
| 552 | NOR | Nepeta cataria | 1340 |
| 553 | NOR | Nepeta cataria | 1341 |
| 554 | NOR | Nepeta cataria | 1342 |
| 555 | NOR | Nepeta cataria | 1343 |
| 556 | NOR | Nepeta cataria | 1344 |
| 557 | NOR | Nepeta cataria | 1345 |
| 558 | NOR | Nepeta cataria | 1346 |
| 559 | NOR | Nepeta cataria | 1347 |
| 560 | NOR | Nepeta cataria | 1348 |
| 561 | NOR | Nepeta cataria | 1349 |
| 562 | NOR | Nepeta cataria | 1350 |
| 563 | NOR | Nepeta cataria | 1351 |
| 564 | NOR | Nepeta cataria | 1352 |
| 565 | NOR | Nepeta cataria | 1353 |
| 566 | NOR | Nepeta cataria | 1354 |
| 567 | NOR | Nepeta cataria | 1355 |
| 568 | NOR | Nepeta cataria | 1356 |
| 569 | NOR | Nepeta cataria | 1357 |
| 570 | NOR | Nepeta cataria | 1358 |
| 571 | NOR | Nepeta cataria | 1359 |
| 572 | NOR | Nepeta cataria | 1360 |
| 573 | NOR | Nepeta cataria | 1361 |
| 574 | NOR | Nepeta cataria | 1362 |
| 575 | NOR | Nepeta cataria | 1363 |
| 576 | NOR | Nepeta cataria | 1364 |
| 577 | NOR | Nepeta cataria | 1365 |
| 578 | NOR | Nepeta cataria | 1366 |
| 579 | NOR | Nepeta cataria | 1367 |
| 580 | NOR | Nepeta cataria | 1368 |
| 581 | NOR | Nepeta cataria | 1369 |
| 582 | NOR | Nepeta cataria | 1370 |
| 583 | NOR | Nepeta cataria | 1371 |
| 584 | NOR | Nepeta cataria | 1372 |
| 585 | NOR | Nepeta cataria | 1373 |
| 586 | NOR | Nepeta cataria | 1374 |
| 587 | NOR | Nepeta cataria | 1375 |
| 588 | NOR | Nepeta cataria | 1376 |
| 589 | NOR | Nepeta cataria | 1377 |
| 590 | NOR | Nepeta cataria | 1378 |
| 591 | NOR | Nepeta cataria/mussinii | 1379 |
| 592 | NOR | Nepeta cataria/mussinii | 1380 |
| 593 | NOR | Nepeta cataria/mussinii | 1381 |
| 594 | NOR | Nepeta cataria/mussinii | 1382 |
| 595 | NOR | Nepeta cataria/mussinii | 1383 |
| 596 | NOR | Nepeta cataria/mussinii | 1384 |
| 597 | NOR | Nepeta cataria/mussinii | 1385 |
| 598 | NOR | Nepeta cataria/mussinii | 1386 |
| 599 | NOR | Nepeta cataria/mussinii | 1387 |
| 600 | NOR | Nepeta cataria/mussinii | 1388 |
| 601 | NOR | Nepeta cataria/mussinii | 1389 |
| 602 | NOR | Nepeta cataria/mussinii | 1390 |
| 603 | NOR | Nepeta cataria/mussinii | 1391 |
| 604 | NOR | Nepeta cataria/mussinii | 1392 |
| 605 | NOR | Nepeta cataria/mussinii | 1393 |
| 606 | NOR | Nepeta cataria/mussinii | 1394 |
| 607 | NOR | Nepeta cataria/mussinii | 1395 |
| 608 | GPPS-GES | Valeriana officinalis/Saccharomyces cerevisiae | 1396 |
| 609 | GPPS-GES | Catharanthus roseus and S. cerevisiae | 1397 |
| 610 | G8H-CPR | engineered fusion | 1398 |
| 611 | G8H-CPR | engineered fusion | 1399 |
| 612 | G8H-CPR | engineered fusion | 1400 |
| 613 | G8H-CPR | engineered fusion | 1401 |
| 614 | G8H-CPR | engineered fusion | 1402 |
| 615 | G8H-CPR | engineered fusion | 1403 |
| 616 | G8H-CPR | engineered fusion | 1404 |
| 617 | G8H-CPR | engineered fusion | 1405 |
| 618 | G8H-CPR | engineered fusion | 1406 |
| 619 | G8H-CPR | engineered fusion | 1407 |
| 620 | G8H-CPR | engineered fusion | 1408 |
| 621 | G8H-CPR | engineered fusion | 1409 |
| 622 | G8H-CPR | engineered fusion | 1410 |
| 623 | G8H-CPR | engineered fusion | 1411 |
| 624 | G8H-CPR | engineered fusion | 1412 |
| 625 | G8H-CPR | engineered fusion | 1413 |
| 626 | G8H-CPR | engineered fusion | 1414 |
| 627 | G8H-CPR | engineered fusion | 1415 |
| 628 | G8H-CPR | engineered fusion | 1416 |
| 629 | G8H-CPR | engineered fusion | 1417 |
| 630 | G8H-CPR | engineered fusion | 1418 |
| 631 | G8H-CPR | engineered fusion | 1419 |
| 632 | G8H-CPR | engineered fusion | 1420 |
| 633 | G8H-CPR | engineered fusion | 1421 |
| 634 | G8H-CPR | engineered fusion | 1422 |
| 635 | G8H-CPR | engineered fusion | 1423 |
| 636 | G8H-CPR | engineered fusion | 1424 |
| 637 | G8H-CPR | engineered fusion | 1425 |
| 638 | G8H-CPR | engineered fusion | 1426 |
| 639 | G8H-CPR | engineered fusion | 1427 |
| 640 | G8H-CPR | engineered fusion | 1428 |
| 641 | G8H-CPR | engineered fusion | 1429 |
| 642 | G8H-CPR | engineered fusion | 1430 |
| 643 | G8H-CPR | engineered fusion | 1431 |
| 644 | G8H-CPR | engineered fusion | 1432 |
| 645 | G8H-CPR | engineered fusion | 1433 |
| 646 | G8H-CPR | engineered fusion | 1434 |
| 647 | G8H-CPR | engineered fusion | 1435 |
| 648 | G8H-CPR | engineered fusion | 1436 |
| 649 | G8H-CPR | engineered fusion | 1437 |
| 650 | G8H-CPR | engineered fusion | 1438 |
| 651 | G8H-CPR | engineered fusion | 1439 |
| 652 | G8H-CPR | engineered fusion | 1440 |
| 653 | G8H-CPR | engineered fusion | 1441 |
| 654 | G8H-CPR | engineered fusion | 1442 |
| 655 | G8H-CPR | engineered fusion | 1443 |
| 656 | G8H-CPR | engineered fusion | 1444 |
| 657 | G8H-CPR | engineered fusion | 1445 |
| 658 | G8H-CPR | engineered fusion | 1446 |
| 659 | G8H-CPR | engineered fusion | 1447 |
| 660 | G8H-CPR | engineered fusion | 1448 |
| 661 | G8H-CPR | engineered fusion | 1449 |
| 662 | G8H-CPR | engineered fusion | 1450 |
| 663 | G8H-CPR | engineered fusion | 1451 |
| 664 | G8H-CPR | engineered fusion | 1452 |
| 665 | G8H-CPR | engineered fusion | 1453 |
| 666 | G8H-CPR | engineered fusion | 1454 |
| 667 | G8H-CPR | engineered fusion | 1455 |
| 668 | G8H-CPR | engineered fusion | 1456 |
| 669 | G8H-CPR | engineered fusion | 1457 |
| 670 | G8H-CPR | engineered fusion | 1458 |
| 671 | G8H-CPR | engineered fusion | 1459 |
| 672 | G8H-CPR | engineered fusion | 1460 |
| 673 | G8H-CPR | engineered fusion | 1461 |
| 674 | G8H-CPR | engineered fusion | 1462 |
| 675 | G8H-CPR-CYB5 | engineered fusion | 1463 |
| 676 | G8H-CPR-CYB5 | engineered fusion | 1464 |
| 677 | G8H-CPR-CYB5 | engineered fusion | 1465 |
| 678 | G8H-CPR-CYB5 | engineered fusion | 1466 |
| 679 | G8H-CPR-CYB5 | engineered fusion | 1467 |
| 680 | G8H-CPR-CYB5 | engineered fusion | 1468 |
| 681 | G8H-CPR-CYB5 | engineered fusion | 1469 |
| 682 | G8H-CPR-CYB5 | engineered fusion | 1470 |
| 683 | G8H-CPR-CYB5 | engineered fusion | 1471 |
| 684 | G8H-CPR-CYB5 | engineered fusion | 1472 |
| 685 | G8H-CPR-CYB5 | engineered fusion | 1473 |
| 686 | G8H-CPR-CYB5 | engineered fusion | 1474 |
| 687 | G8H-CPR-CYB5 | engineered fusion | 1475 |
| 688 | G8H-CPR-CYB5 | engineered fusion | 1476 |
| 689 | G8H-CPR-CYB5 | engineered fusion | 1477 |
| 690 | G8H-CPR-CYB5 | engineered fusion | 1478 |
| 691 | G8H-CPR-CYB5 | engineered fusion | 1479 |
| 692 | G8H-CPR-CYB5 | engineered fusion | 1480 |
| 693 | G8H-CPR-CYB5 | engineered fusion | 1481 |
| 694 | 8HGO-ISY | engineered fusion | 1482 |
| 695 | 8HGO-ISY | engineered fusion | 1483 |
| 696 | 8HGO-ISY | engineered fusion | 1484 |
| 697 | 8HGO-ISY | engineered fusion | 1485 |
| 698 | 8HGO-ISY | engineered fusion | 1486 |
| 699 | 8HGO-ISY | engineered fusion | 1487 |
| 700 | 8HGO-ISY | engineered fusion | 1488 |
| 701 | 8HGO-ISY | engineered fusion | 1489 |
| 702 | 8HGO-ISY | engineered fusion | 1490 |
| 703 | 8HGO-ISY | engineered fusion | 1491 |
| 704 | 8HGO-ISY | engineered fusion | 1492 |
| 705 | 8HGO-ISY | engineered fusion | 1493 |
| 706 | ISY-NEPS | engineered fusion | 1494 |
| 707 | ISY-NEPS | engineered fusion | 1495 |
| 708 | ISY-NEPS | engineered fusion | 1496 |
| 709 | ISY-NEPS | engineered fusion | 1497 |
| 710 | ISY-NEPS | engineered fusion | 1498 |
| 711 | ISY-NEPS | engineered fusion | 1499 |
| 712 | ISY-NEPS | engineered fusion | 1500 |
| 713 | ISY-NEPS | engineered fusion | 1501 |
| 714 | ISY-NEPS | engineered fusion | 1502 |
| 715 | ISY-NEPS | engineered fusion | 1503 |
| 716 | ISY-NEPS | engineered fusion | 1504 |
| 717 | ISY-NEPS | engineered fusion | 1505 |
| 718 | NEPS | Nepeta mussinii | 1506 |
| 719 | NEPS | Nepeta mussinii | 1507 |
| 720 | NEPS | Catharanthus roseus | 1508 |
| 721 | NEPS | Camptotheca acuminata | 1509 |
| 722 | NEPS | Vinca minor | 1510 |
| 723 | NEPS | Rauvolfia serpentina | 1511 |
| 724 | NEPS | Catharanthus roseus | 1512 |
| 725 | NEPS | Camptotheca acuminata | 1513 |
| 726 | NEPS | Vinca minor | 1514 |
| 727 | NEPS | Rauvolfia serpentina | 1515 |
| 728 | NEPS | Nepeta mussinii | 1516 |
| 729 | NEPS | Nepeta mussinii | 1517 |
| 730 | NEPS | Catharanthus roseus | 1518 |
| 731 | NEPS | Camptotheca acuminata | 1519 |
| 732 | NEPS | Vinca minor | 1520 |
| 733 | NEPS | Rauvolfia serpentina | 1521 |
| 734 | NEPS | Andrographis_{—}paniculata | 1522 |
| 735 | NEPS | Gentiana triflora | 1523 |
| 736 | NEPS | Coffea canephora | 1524 |
| 737 | NEPS | Ophiorrhiza_{—}pumila | 1525 |
| 738 | NEPS | Phelline_{—}lucida | 1526 |
| 739 | NEPS | Vitex_{—}agnus_{—}castus | 1527 |
| 740 | NEPS | Valeriana_{—}officianalis | 1528 |
| 741 | NEPS | Stylidium_{—}adnatum | 1529 |
| 742 | NEPS | Verbena_{—}hastata | 1530 |
| 743 | NEPS | Byblis_{—}gigantea | 1531 |
| 744 | NEPS | Pogostemon_sp. | 1532 |
| 745 | NEPS | Strychnos_{—}spinosa | 1533 |
| 746 | NEPS | Corokia_{—}cotoneaster | 1534 |
| 747 | NEPS | Oxera_{—}neriifolia | 1535 |
| 748 | NEPS | Buddleja_sp. | 1536 |
| 749 | NEPS | Gelsemium_{—}sempervirens | 1537 |
| 750 | NEPS | Utricularia_sp. | 1538 |
| 751 | NEPS | Scaevola_sp. | 1539 |
| 752 | NEPS | Menyanthes_{—}trifoliata | 1540 |
| 753 | NEPS | Pinguicula_{—}caudata | 1541 |
| 754 | NEPS | Psychotria_{—}ipecacuanha | 1542 |
| 755 | NEPS | Dipsacus_{—}sativum | 1543 |
| 756 | NEPS | Exacum_{—}affine | 1544 |
| 757 | NEPS | Chionanthus_{—}retusus | 1545 |
| 758 | NEPS | Allamanda_{—}cathartica | 1546 |
| 759 | NEPS | Phyla_{—}dulcis | 1547 |
| 760 | NEPS | Ligustrum_{—}sinense | 1548 |
| 761 | NEPS | Pyrenacantha_{—}malvifolia | 1549 |
| 762 | NEPS | Sambucus_{—}canadensis | 1550 |
| 763 | NEPS | Leonurus_{—}japonicus | 1551 |
| 764 | NEPS | Ajuga_{—}reptans | 1552 |
| 765 | NEPS | Paulownia_{—}fargesii | 1553 |
| 766 | NEPS | Caiophora_{—}chuquitensis | 1554 |
| 767 | NEPS | Plantago_{—}maritima | 1555 |
| 768 | NEPS | Antirrhinum_{—}braun | 1556 |
| 769 | NEPS | Cyrilla_{—}racemiflora | 1557 |
| 770 | NEPS | Hydrangea_{—}quercifolia | 1558 |
| 771 | NEPS | Cinchona pubescens | 1559 |
| 772 | NEPS | Actinidia chinensis var. chinensis | 1560 |
| 773 | NEPS | Swertia japonica | 1561 |
| 774 | NEPS | Sesamum indicum | 1562 |
| 775 | NOR | Isodon_{—}rubescens | 1563 |
| 776 | NOR | Prunella_{—}vulgaris | 1564 |
| 777 | NOR | Agastache_{—}rugosa | 1565 |
| 778 | NOR | Melissa_{—}officinalis | 1566 |
| 779 | NOR | Micromeria_{—}fruticosa | 1567 |
| 780 | NOR | Plectranthus_{—}caninus | 1568 |
| 781 | NOR | Rosmarinus officinalis | 1569 |
| 782 | NOR | Nepeta mussinii | 1570 |
| 783 | CYB5R | Catharanthus roseus | 1571 |
| 784 | CYB5R | Nepeta cataria | 1572 |
| 785 | CYB5R | Arabidopsis thaliana | 1573 |
| 786 | CYB5R | Catharanthus roseus | 1574 |
| 787 | CYB5R | Nepeta cataria | 1575 |
| 788 | CYB5R | Arabidopsis thaliana | 1576 |
| 1642 | NOR | Nepeta cataria | 1725 |
| 1643 | NOR | Nepeta cataria | 1726 |
| 1644 | NOR | Nepeta cataria | 1727 |
| 1645 | GPPS-GES | engineered fusion | 1728 |
| 1646 | GPPS-GES | engineered fusion | 1729 |
| 1647 | GPPS-GES | engineered fusion | 1730 |
| 1648 | GPPS-GES | engineered fusion | 1731 |
| 1649 | GPPS-GES | engineered fusion | 1732 |
| 1650 | GPPS-GES | engineered fusion | 1733 |
| 1651 | GPPS-GES | engineered fusion | 1734 |
| 1652 | GPPS-GES | engineered fusion | 1735 |
| 1653 | GPPS-GES | engineered fusion | 1736 |
| 1654 | GPPS-GES | engineered fusion | 1737 |
| 1655 | GPPS-GES | engineered fusion | 1738 |
| 1656 | GPPS-GES | engineered fusion | 1739 |
| 1657 | GPPS-GES | engineered fusion | 1740 |
| 1658 | GPPS-GES | engineered fusion | 1741 |
| 1659 | GPPS-GES | engineered fusion | 1742 |
| 1660 | GPPS-GES | engineered fusion | 1743 |
| 1661 | GPPS-GES | engineered fusion | 1744 |
| 1662 | GPPS-GES | engineered fusion | 1745 |
| 1663 | GPPS-GES | engineered fusion | 1746 |
| 1664 | GPPS-GES | engineered fusion | 1747 |
| 1665 | GPPS-GES | engineered fusion | 1748 |
| 1666 | GPPS-GES | engineered fusion | 1749 |
| 1667 | GPPS-GES | engineered fusion | 1750 |
| 1668 | GPPS-GES | engineered fusion | 1751 |
| 1669 | GPPS-GES | engineered fusion | 1752 |
| 1670 | GPPS-GES | engineered fusion | 1753 |
| 1671 | GPPS-GES | engineered fusion | 1754 |
| 1672 | GPPS-GES | engineered fusion | 1755 |
| 1673 | GPPS-GES | engineered fusion | 1756 |
| 1674 | GPPS-GES | engineered fusion | 1757 |
| 1675 | GPPS-GES | engineered fusion | 1758 |
| 1676 | GPPS-GES | engineered fusion | 1759 |
| 1677 | GPPS-GES | engineered fusion | 1760 |
| 1678 | GPPS-GES | engineered fusion | 1761 |
| 1679 | GPPS-GES | engineered fusion | 1762 |
| 1680 | GPPS-GES | engineered fusion | 1763 |
| 1681 | GPPS-GES | engineered fusion | 1764 |
| 1682 | GPPS-GES | engineered fusion | 1765 |
| 1683 | GPPS-GES | engineered fusion | 1766 |
| 1684 | GPPS-GES | engineered fusion | 1767 |
| 1685 | GPPS-GES | engineered fusion | 1768 |
| 1686 | GPPS-GES | engineered fusion | 1769 |
| 1687 | GPPS-GES | engineered fusion | 1770 |
| 1688 | GPPS-GES | engineered fusion | 1771 |
| 1689 | GPPS-GES | engineered fusion | 1772 |
| 1690 | GPPS-GES | engineered fusion | 1773 |
| 1691 | GPPS-GES | engineered fusion | 1774 |
| 1692 | GPPS-GES | engineered fusion | 1775 |
| 1693 | GPPS-GES | engineered fusion | 1776 |
| 1694 | GPPS-GES | engineered fusion | 1777 |
| 1695 | ISY | Phialophora attae | 1778 |
| 1696 | ISY | Tarenaya spinosa | 1779 |
| 1697 | ISY | Trifolium pratense | 1780 |
| 1698 | ISY | Oryza glumipatula | 1781 |
| 1699 | ISY | Triticum aestivum | 1782 |
| 1700 | ISY | Oryza glumipatula | 1783 |
| 1701 | ISY | Madurella mycetomatis | 1784 |
| 1702 | ISY | Phaedon cochleariae | 1785 |
| 1703 | ISY | Glycine max | 1786 |
| 1704 | ISY | Triticum aestivum | 1787 |
| 1705 | ISY | Olea europaea | 1788 |
| 1706 | ISY | Camptotheca acuminata | 1789 |
| 1707 | ISY | Musa acuminata subsp. malaccensis | 1790 |
| 1708 | ISY | Arabidopsis thaliana | 1791 |
| 1709 | ISY | Digitalis lanata | 1792 |
| 1710 | ISY | Musa acuminata subsp. malaccensis | 1793 |
| 1711 | ISY | Musa acuminata subsp. malaccensis | 1794 |
| 1712 | ISY | Anthurium amnicola | 1795 |
| 1713 | ISY | Cinchona_{—}Ledgeriana | 1796 |
| 1714 | ISY | Triticum aestivum | 1797 |
| 1715 | ISY | Aegilops tauschii | 1798 |
| 1716 | ISY | Vinca minor | 1799 |
| 1717 | ISY | Cinchona pubescens | 1800 |
| 1718 | ISY | Ophiorrhiza pumila | 1801 |
| 1719 | ISY | Swertia japonica | 1802 |
| 1720 | ISY | Lonicera_{—}japonica | 1803 |
| 1721 | ISY | Rauwolfia serpentina | 1804 |
| 1722 | ISY | Lonicera japonica | 1805 |
| 1723 | ISY | Oryza sativa subsp. japonica | 1806 |
| 1724 | ISY | Phaedon cochleariae | 1807 |

It is to be understood that the description above as well as the examples that follow are intended to illustrate, and not limit, the scope of the invention. Other aspects, advantages and modifications within the scope of the invention will be apparent to those skilled in the art to which the invention pertains.

All patents, patent applications, references, and journal articles cited in this disclosure are expressly incorporated herein by reference in their entireties for all purposes.

### EXAMPLES

### Example 1: Cloning and Expression of Nepetalactone Oxidoreductases in Escherichia coli Capable of Converting Nepetalactol to Nepetalactone

### Identification of NOR Candidates

Publicly available next-generation RNA sequencing data from Nepeta cataria was obtained from NCBI (SRR5150709). The reads were extracted and assembled into a transcriptome. The protein sequence for horse liver alcohol dehydrogenase (HLADH) was used as a BLAST query to identify alcohol dehydrogenases candidates from Nepeta cataria that might catalyze conversion of nepetalactol to nepetalactone.

Thirty-nine candidates were identified and the coding sequences were codon optimized for expression in E. coli. The codon-optimized nucleotide sequences were synthesized with an upstream T7 promoter and a ribosome binding site (RBS) and a downstream T7 terminator sequence by Integrated DNA Technologies (IDT). Synthesized DNA was retrieved as plasmids containing the expression cassettes within a backbone containing the kanamycin resistance marker provided by IDT.

### Heterologous Expression of NOR Candidates

The plasmids were individually transformed into chemically competent BL21 (DE3) cells. pUC19 was also transformed into BL21 (DE3) to produce a strain that could serve as a negative control. Transformants were selected and grown overnight with shaking in LB medium containing kanamycin. Glycerol stocks were prepared by mixing overnight culture with 50% glycerol in a 1:1 ratio. Glycerol stocks were frozen at −80° C.

BL21 (DE3) strains were streaked out on LB plates containing kanamycin from glycerol stock and grown overnight at 37° C. A single colony was inoculated into 4 mL of LB medium containing kanamycin in 15 mL disposable culture tubes and incubated overnight at 30° C. with shaking at 250 rpm. 500 μL of the overnight culture was subcultured into 50 mL of LB medium containing kanamycin in a 250 mL baffled flask. The culture was grown at 37° C. and the optical density at 600 nm (OD600) was monitored. When OD600 reached between 0.6-1, the cultures were cooled on ice for 15 minutes. The cultures were then induced with 100 μM of isopropyl β-D-1-thiogalactopyranoside and incubated at 15° C. with shaking at 250 rpm for roughly 20 hours. Cultures were pelleted by centrifugation in 50 mL centrifuge tubes. The supernatant was decanted and the pellets were frozen at −20° C. for later processing.

### In Vitro Characterization of NOR Candidates

Pellets were thawed on ice and resuspended with 3 mL of cold lysis buffer: 50 mM sodium phosphate, pH=7.4, 100 mM sodium chloride. All remaining steps were performed either on ice or at 4° C. The cell mixture was transferred to a 15 mL centrifuge tube and disrupted with three rounds of sonication using the Branson Sonitier 450 with a double-level microtip at 70% amplitude. A single round of sonication consisted of 6 cycles of 10 seconds with the sonicator on, and 10 seconds off Between each round, the cell mixture was allowed to sit on ice for a minute to cool. The lysed cell mixture was transferred to 1.7 mL centrifuge tubes and centrifuged at maximum speed in a microcentrifuge for 20 minutes. The supernatant (clarified cell lysate) was collected in a separate tube and used for in vitro characterization.

The in vitro reactions were setup as follows: 2 μL of 100 mM NAD+ or NADP+ and 10 μL of 100 uM nepetalactol was added to 188 μL of the clarified cell lysate. The reactions were incubated at 30° C. shaking at 200 rpm for 2 hours. As a positive control, 2 μL of 100 mM NAD+, 2 μL of 100 mM NADP+ and 10 μL of 100 μM nepetalactone was added to 186 μL of clarified lysate from a strain harboring pUC 19 and incubated for 1 hr. The reactions were extracted with one volume of ethyl acetate. The organic layer was withdrawn and analyzed with gas chromatography coupled to mass spectrometry (GC-MS). Authentic standards were run to confirm identities of analytes.

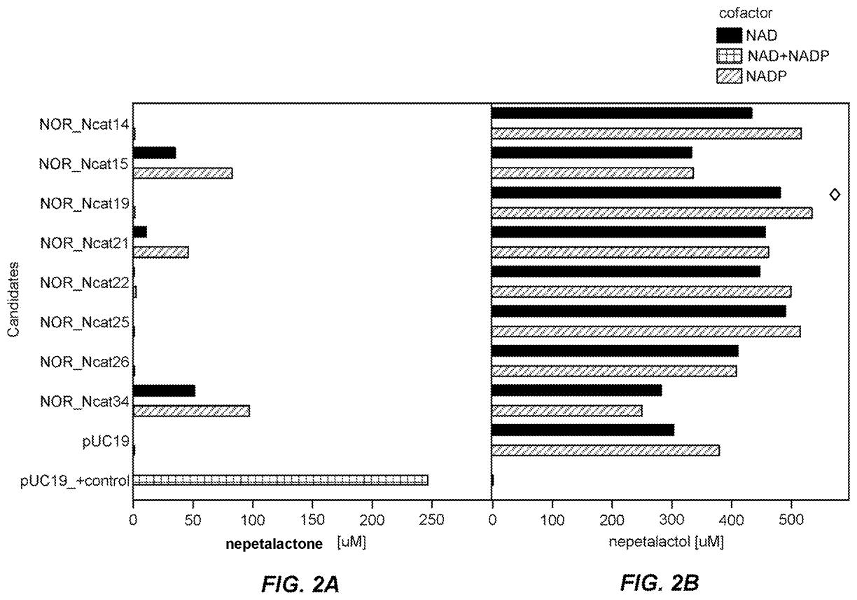

The results are shown in FIG. 2. Three candidate genes NcatNORI5 (protein SEQ ID NO: 561), NcatNOR21 (protein SEQ ID NO: 566), and NcatNOR34 (protein SEQ ID NO: 578) [(DNA SEQ ID NOs: 1725-1727)] were found to encode NORs which can oxidize nepetalactol to nepetalactone, the first such demonstration.

### Example 2—Expression and Activities of Various Iridoid Synthases

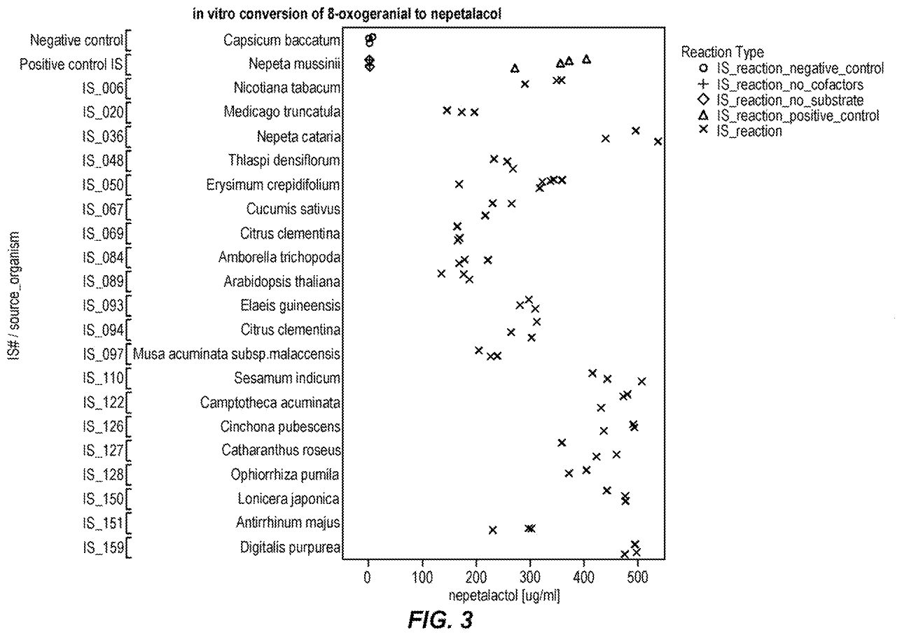

A variety of iridoid synthases (ISYs, SEQ ID NOs: 1181, 1256, 1257, 1306, 30 1191, 1255, 1269, 1203, 1791, 1801, 1215, 1281, 1190, 1217, 1800, 1234, 1277, 1233, 1300, 1249, 1805) were heterologously expressed in E. coli from a plasmid using a T7 expression system. E. coli cultures were grown until OD600—0.6 and induced with 1 mM IPTG and grown for 7.5 h at 28° C. or 20 h at 15° C. Cells were harvested and chemically lysed by Bugbuster HT (EMD Millipore) following manufacturer's instructions. Cell lysates were clarified by centrifugation and were tested for in vitro conversion of 8-oxogeranial to nepetalactol in the presence of NADH and NADPH (see FIG. 3). 2 μL of cell lysate was added to a reaction mixture containing 200 mM HEPES, pH=7.3, 100 μM of 8-oxogeranial, 100 μM NADH and 100 μM of NADPH. The reaction mixture was extracted with 300 μL of ethyl acetate. The organic extract was analyzed by LC-MS for quantification of nepetalactol.

### Example 3: Cloning and Expression of Nepetalactol Synthases Capable of Producing Nepetalactol

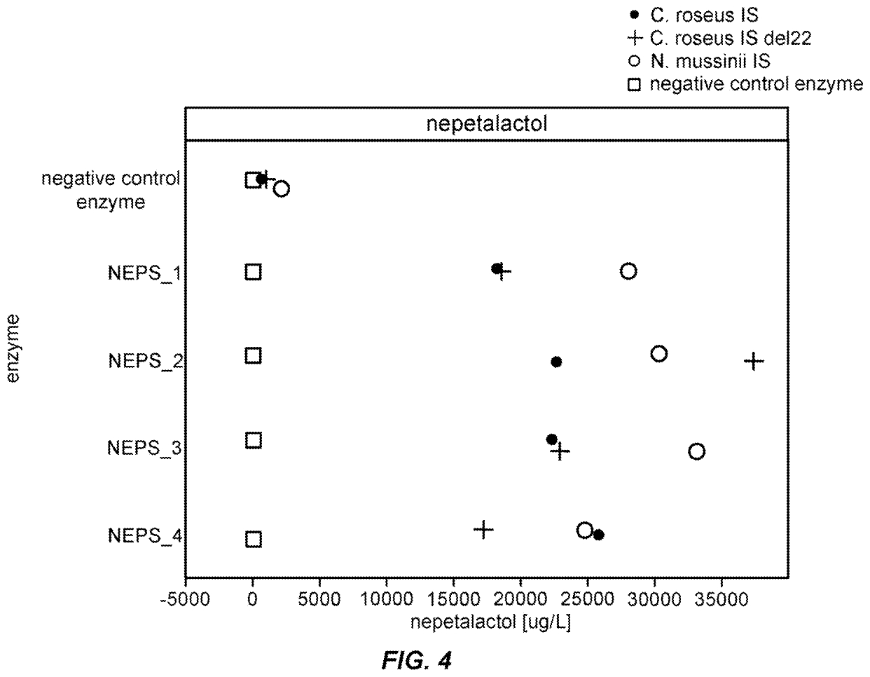

Four putative nepetalactol synthases (NEPS_1 to NEPS_4; DNA SEQ ID NO: 1518-1521; protein SEQ ID NOs: 730-733) were identified by examining publicly available transcriptome data (medicinalplantgenomics.msu.edu) from four plant species that are known to produce monoterpene indole alkaloids (Catharanthus roseus, Camptotheca acuminata, Vinca minor, and Rauvolfia serpentina). Transcripts that encoded these NEPS were highly co-expressed with biosynthetic gene homologs that catalyze the formation of loganic acid from geraniol, which proceeds through the intermediate, nepetalactol. This analysis suggested the involvement of these NEPS candidates in the biosynthesis of loganic acid from geraniol, perhaps in nepetalactol formation. All four NEPSs were heterologously expressed in E. coli from a plasmid using a T7 expression system. E. coli cultures were grown until OD600˜0.6 and induced with 100 μM IPTG and grown for 16 h at 16° C. Cells were harvested and chemically lysed by Bugbuster HT (EMD Millipore) following manufacturer's instructions. Cell lysates were clarified by centrifugation. NEPS activity was tested individually by the addition of 10 μL of cell lysate to a reaction mixture containing 50 mM HEPES, pH=7.3, 500 μM of 8-oxogeranial, 1 mM NADPH and 10 μL of cell lysate that contains one of three iridoid synthases (ISY) in a final volume of 200 μL. The ISY s include Catharanthus roseus iridoid synthase (ISY; SEQ ID NO. 1162), C. roseus ISY “del22” (SEQ ID NO. 1166), which is truncated at the N-terminus by 22 amino acids, and Nepeta mussinii ISY (SEQ ID NO. 1159) (see FIG. 4). The reaction mixture was extracted with 300 μL of ethyl acetate, and the organic layer was analyzed by LC-MS for the quantification of nepetalactol. In every case, the presence of the NEPS enhanced production of nepetalactol (11- to 40-fold increase) compared to in vitro reactions that contained cell lysate from E. coli that did not express NEPS.

### Example 4—Expression and Activities of Various 8-Hydroxygeraniol Oxidoreductases

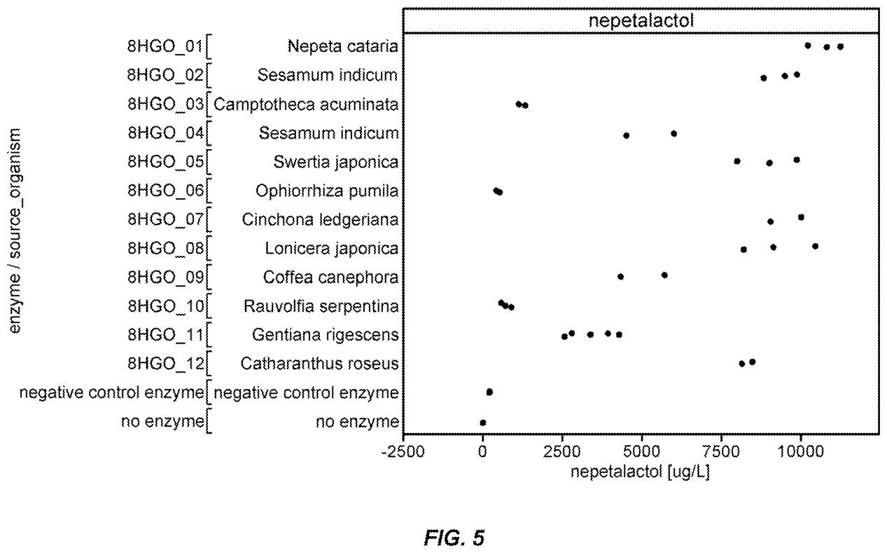

A variety of 8-hydroxygeraniol oxidoreductases (8HGOs; SEQ ID NO: 1132, 1134, 1136, 1138-1146) were heterologously expressed in E. coli from a plasmid using a T7 expression system. E. coli cultures were grown until OD600—0.6 and induced with 100 μM IPTG and grown for 16 h at 16° C. Cells were harvested and chemically lysed by Bugbuster HT (EMD Millipore) following manufacturer's instructions. Cell lysates were clarified by centrifugation. 8HGO activity was tested by the addition of 1 μL of cell lysate to a reaction mixture containing 50 mM of bis-tris propane, pH=9.0, 1 mM NADPH, 1 mM NAD+, 500 μM of 8-hydroxygeraniol, 1 μL of cell lysate containing Nepeta mussinii ISY (SEQ ID NO: 1159) and 1 μL of cell lysate containing NEPS_1 (SEQ ID NO: 1518) in a final reaction volume of 100 μL. The reaction mixture was extracted with 300 μL of ethyl acetate, and the organic layer was analyzed by LC-MS for quantification of nepetalactol. (see FIG. 5).

### Example 5—Cloning and Expression of Nepetalactone Oxidoreductases in Saccharomyces cerevisiae Capable of Converting Nepetalactol to Nepetalactone

### Identification of NOR Candidates

An additional list of seventeen candidates were identified from the de novo transcriptome assembly produced above in EXAMPLE 1. Briefly, hmmscan from the software, HMMER was used to functionally annotate all predicted peptides from the assembly based on their best matching Pfam hidden markov model (HMM) by E-value. All HMMs related to oxidoreductase activity were investigated further by BLAST and filtered to remove sequences with high sequence identity to any sequences from the non-redundant database to further narrow the list of candidates. The sequences of these candidates and the original thirty-nine candidates described in EXAMPLE 1 were codon-optimized for expression in S. cerevisiae (SEQ ID NO: 1340-1395) and were synthesized by a third-party and cloned into the 2p plasmid backbone, pESC-URA.

### Heterologous Expression and Testing of NOR Candidates

The plasmids were individually transformed into chemically competent Saccharomyces cerevisiae cells as described in EXAMPLE 2. Transformants were selected on SD-URA agar plates. Three to four replicates were picked into SD-URA liquid medium and cultured at 30° C. for one to two days with shaking at 1000 rpm. Cultures were glycerol stocked at a final concentration of 16.6% glycerol and stored at −80° C. until later use.

10 μL of the glycerol stocked strains was inoculated into 300 μL of minimal media lacking uracil, and containing 4% glucose in 96-well plates to produce seed cultures. The plates were incubated at 30° C. at 1000 rpm for 1-2 days. 10 μL of the seed cultures was then inoculated into 300 μL of minimal media lacking uracil, and containing 2% galactose and 100 mg/L of nepetalactol. 30 μL of methyl oleate was next added to the wells. The main culture plates were further incubated at 30° C., 1000 rpm for 24 hours before assays were performed to assess cell growth and titer. Cell growth and titer assays were performed as described above in EXAMPLE 2.

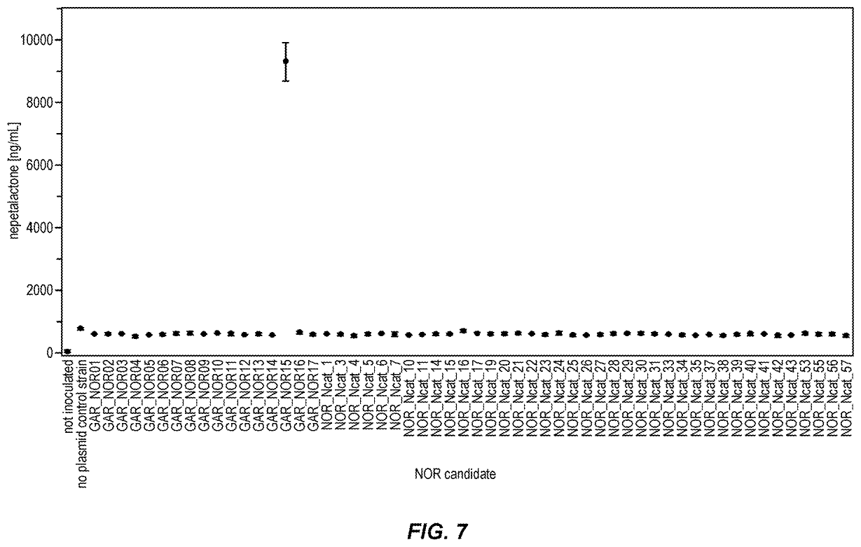

All tested strains produced at least some basal level of nepetalactone (−600 ug/L; see FIG. 7), including a control strain that did not contain a plasmid for expression of a NOR candidate. No nepetalactone was observed in the non-inoculated control wells. Altogether, these results suggest that Saccharomyces cerevisiae has low background levels of NOR activity. One of the tested strains expressing GAR_NOR15 (SEQ ID NO: 1393) produced significantly more nepetalactone (93 mg/L), far exceeding basal levels, and demonstrating that this heterologous protein candidate has activity for converting nepetalactol into nepetalactone.

### Example 6—Characterization of Other NEPS Enzymes

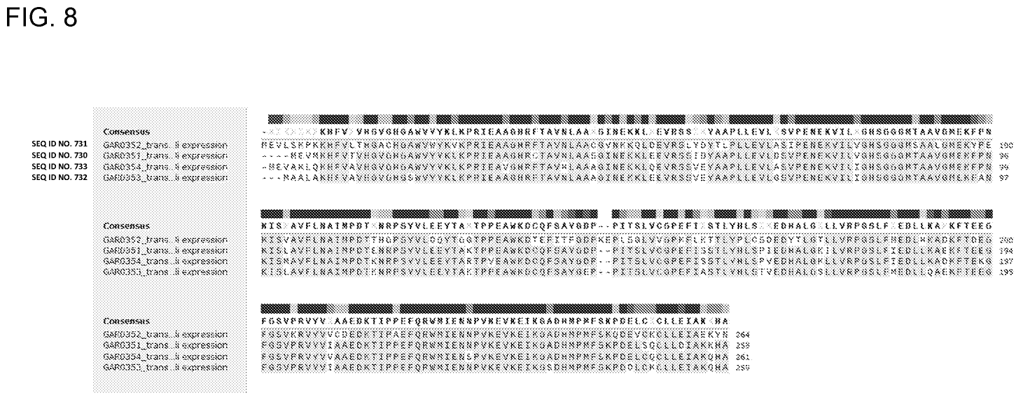

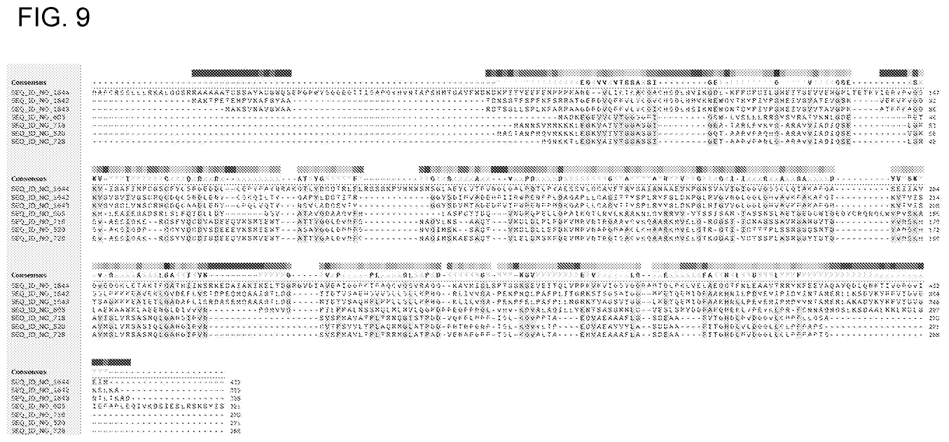

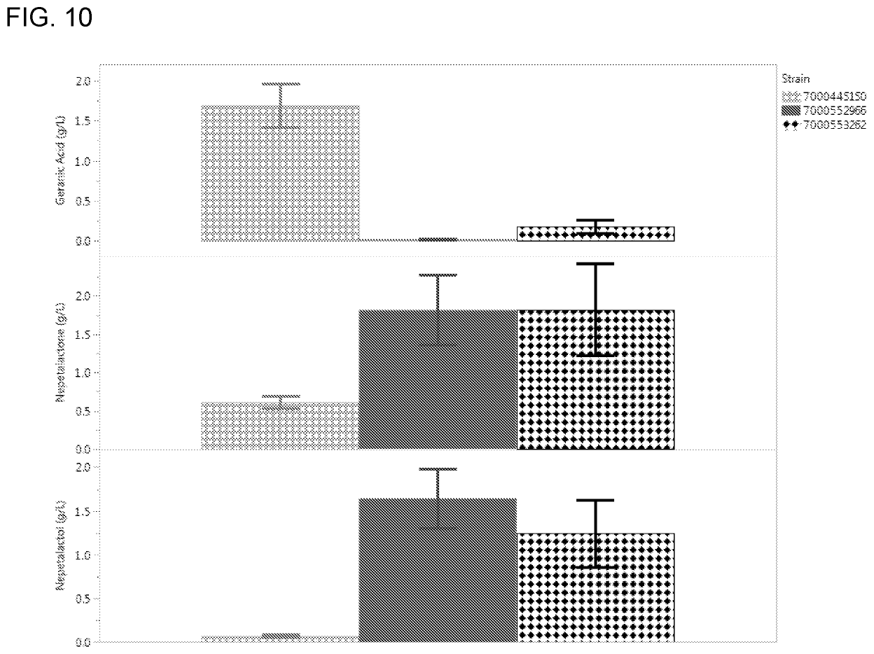

Proteins predicted to be NEPS enzymes were identified as comprising amino acid sequences SEQ ID Nos. 718-774. Four of these proteins (comprising amino acid sequences of SEQ ID Nos. 730-733) were tested and were confirmed to have NEPS enzymatic activity (see Example 3). A sequence alignment of these four sequences is shown in FIG. 8. A Hidden Markov model (HMM) analysis of these four protein sequences showed that they share a Pfam domain pfam12697. The presence of the Pfam domain pfam12697 distinguishes these NEPS enzymes from the NEPS enzymes described thus far (see, for e.g., Lichman et al., Nature Chemical Biology, Vol. 15 Jan. 2019, 71-79), which do not contain this protein domain. This domain essentially spans the entire length of the sequences shown in FIG. 8, which are roughly 260 amino acids long. The domain maps to the following portions of the sequences shown in FIG. 8: SEQ ID NO 730: amino acids 8-246; SEQ ID NO 731: amino acids 11-253; SEQ ID NO 732: amino acids 9-247; SEQ ID NO 733: amino acids 11-249.

Additionally, other proteins predicted to be NEPS enzymes comprising amino acid sequences of SEQ ID Nos. 734-774 will be tested for NEPS enzymatic activity of converting an enol intermediate substrate to nepetalactol and characterized as described above.

A protein BLAST was performed for SEQ ID NO: 720 to identify more proteins with predicted NEPS enzymatic activity. Similar BLAST results are expected for proteins with the amino acid sequences of SEQ ID Nos. 718, 719, and 721-774. The proteins predicted as being NEPS enzymes will be tested for NEPS enzymatic activity of converting an enol intermediate substrate to nepetalactol. Additionally, the ratio of nepetalactol stereoisomers produced by each of the NEPS enzymes will also be measured, thereby identifying NEPS enzymes, and variants thereof, which can produce defined ratios of nepetalactol stereoisomers.

### Example 7—Characterization of Other NOR Enzymes

Proteins predicted to be NOR enzymes were identified as comprising amino acid sequences SEQ ID Nos. 520-607, 775-782 and 1642-1644. A MUSCLE protein alignment was performed of NOR enzymes comprising the amino acid sequences of SEQ ID NO 605, 718, 728, 1642, 1643, and 1644; and the NOR comprising SEQ ID NO: 520 described in the art previously (see Lichman et al. Nature Chemical Biology, Vol. 15 Jan. 2019, 71-79). The results showed that there is less than 20% identity between the NORs of this disclosure and the NOR described previously in the art, as shown in FIG. 11, demonstrating that the genus of NORs described in this disclosure is novel over the existing knowledge in the art.

A protein BLAST search was performed for each individual sequence to identify more proteins with predicted NOR enzymatic activity. Further an InterProScan was performed for SEQ ID NO 520 (NEPS1 of Lichman et al.) and NOR sequences comprising amino acid sequences SEQ ID NOs 605, 1642-1644 disclosed herein, and the results are shown in Table 9.

TABLE 9

| | | | Amino acids |
|---|---|---|---|
| SEQ ID | | | spanning the |
| NO. | Domains | ID | domain |
| 520 | Short-chain dehydrogenase/reductase | IPR002347 | 19-36; 91-102; 167- |
| | SDR | | 186; 188-205; 226-246 |
| 520 | NAD(P)-binding domain superfamily | IPR036291 | 16-263 |
| 605 | NAD-dependent epimerase/dehydratase | IPR001509 | 9-241 |
| 605 | NAD(P)-binding domain superfamily | IPR036291 | 3-315 |
| 1642 | GroES-like superfamily | IPR011032 | 19-184 |
| 1642 | NAD(P)-binding domain superfamily | IPR036291 | 157-321 |
| 1642 | Polyketide synthase, enoylreductase | IPR020843 | 23-351 |
| | domain | | |
| 1642 | Alcohol dehydrogenase, N-terminal | IPR013154 | 38-151 |
| 1642 | Alcohol dehydrogenase, C-terminal | IPR013149 | 194-317 |
| 1642 | Alcohol dehydrogenase, zinc-type, | IPR002328 | 71-85 |
| | conserved site | | |
| 1643 | GroES-like superfamily | IPR011032 | 16-178 |
| 1643 | NAD(P)-binding domain superfamily | IPR036291 | 151-315 |
| 1643 | Polyketide synthase, enoylreductase | IPR020843 | 17-345 |
| | domain | | |
| 1643 | Alcohol dehydrogenase, N-terminal | IPR013154 | 32-144 |
| 1643 | Alcohol dehydrogenase, C-terminal | IPR013149 | 188-311 |
| 1643 | Alcohol dehydrogenase, zinc-type, | IPR002328 | 75-79 |
| | conserved site | | |
| 1644 | GroES-like superfamily | IPR011032 | 61-260 |
| 1644 | NAD(P)-binding domain superfamily | IPR036291 | 266-399 |
| 1644 | Polyketide synthase, enoylreductase | IPR020843 | 72-432 |
| | domain | | |
| 1644 | Alcohol dehydrogenase, N-terminal | IPR013154 | 89-195 |
| 1644 | Alcohol dehydrogenase, C-terminal | IPR013149 | 264-394 |

These results show that the NOR sequences of this disclosure contain different domains as compared to the NOR described in Lichman et al., which contains the short-chain dehydrogenase/reductase SDR, and the NAD(P)-binding domain superfamily.

Additionally, other proteins disclosed herein which are predicted to be NOR enzymes will be tested for NOR enzymatic activity of converting a nepetalactol substrate to nepetalactone and further characterized as described above.

### Example 8—Introduction of a Partial Biosynthetic Pathway for Nepetalactone into Yeast Plasmid/DNA Design

Genes were synthesized by a third-party and plasmids were assembled by standard DNA assembly methods either in-house or by a third-party. The plasmid DNA was then used to chromosomally integrate the metabolic pathway inserts into Saccharomyces cerevisiae. Plasmids were designed for ‘two plasmid, split-marker’ integrations. Briefly, two plasmids were constructed for each targeted genomic integration. The first plasmid contains an insert made up of the following DNA parts listed from 5′ to 3′: 1) a 5′ homology arm to direct genomic integration; 2) a payload consisting of cassettes for heterologous gene expression; 3) the 5′ half of a URA3 selection marker cassette. The second plasmid contains an insert made up of the following DNA parts listed from 5′ to 3′: 1) the 3′ half of a URA3 selection marker cassette with 100 bp or more DNA overlap to the 3′ end of the 5′ half of the URA selection marker cassette used in the first plasmid; 2) an optional payload consisting of cassettes for heterologous gene expression: 3) a 3′ homology arm to direct genomic integration. The inserts of both plasmids are flanked by meganuclease sites. Upon digestion of the plasmids using the appropriate meganucleases, 20 inserts are released and transformed into cells as linear fragments. A triple-crossover event allows integration of the desired heterologous genes and reconstitution of the full URA3 marker allowing selection for uracil prototrophy. For recycling of the URA3 marker, the URA3 cassette is flanked by 100-200 bp direct repeats, allowing for loop-out and counterselection with 5-Fluoroorotic Acid (5-FOA).

Cassettes for heterologous expression contain the gene coding sequence under the transcriptional control of a promoter and terminator. Promoters and terminators may be selected from any elements native to S. cerevisiae. Promoters may be constitutive or inducible. Inducible promoters include the bi-directional pGAL1/pGAL1O (pGAL1-10) promoter and pGAL 7 promoter, which are induced by galactose.

### Strain Construction

Cells were grown in yeast extract peptone dextrose (YPD) overnight at 30° C., shaking at 250 rpm. The cells were diluted to an optical density at 600 nm (OD600)=0.2 in 50 mL of YPD and grown to an OD600=0.6-0.8. Cells were harvested by centrifugation, washed with water, washed with 100 mM lithium acetate, and resuspended in 100 mM lithium acetate to a final OD600=100. 15 μL of the cell resuspension was directly added to the DNA. A PEG mixture containing 100 μL of 50% w/v PEG3350, 4 μL of 10 mg/mL salmon sperm DNA, 15 μL of 1 M lithium acetate was added to the DNA and 5 cell mixture, and well-mixed. The transformation mix was incubated at 30° C. for 30 min and 42° C. for 45 min.

Following heat-shock, the transformation mix was plated on agar plates containing synthetic defined minimal yeast media lacking uracil (SD-URA). Plates were incubated at 30° C. for 2-3 days. Up to eight transformants were picked for each targeted 10 strain into 1 mL of SD-URA liquid media of a 96-well plate and grown at 30° C. with shaking at 1000 rpm and 90% relative humidity (RH). Cultures were lysed using Zymolyase, and a PCR was performed using the resulting lysate to verify successful integration using primers that targeted the 5′ integration junction. Glycerol stocks were prepared from the cultures at a final concentration of 16.6% glycerol and were stored at −80° C. for later use.

To recycle the URA3 selection marker, selected strains were inoculated into SD-URA and grown overnight at 30° C., 1000 rpm and 90% RH. Strains were then plated onto 0.1% 5-FOA plates (Teknova) and incubated at 30° C. for 2-3 days. Single colonies were re-streaked onto 0.1% 5-FOA plates. Single colonies were selected from the re-streak and colony PCR was performed in order to verify loop-out of the URA3 marker. Colonies were also tested for lack of growth in liquid SD-URA medium. Further integrations were performed as described above.

### Strain Cultivation and Target Compound Production

From the frozen glycerol stocks, successful integrants were inoculated into a seed plate containing 300 μL of SD-URA. The 96-well plate was incubated at 30° C., 1000 rpm, 90% RH for 48 hours. For each successfully built strain, three biological replicates were tested. If fewer than three successful transformants were obtained for each targeted strain genotype, the existing biological replicates were duplicated. Strains were randomized across a 96-well plate. After the 48 hours of growth, 8 μL of the cultures from the seed plates were used to inoculate a main cultivation plate containing 250 μL of minimal medium with 2°/o glucose and grown for 16 hour at 30° C., 1000 rpm, 90% RH. 50 μL of minimal medium with 12% galactose was added to the cultures to induce expression of heterologous genes under the control of galactose promoters, followed by the addition of 30 μL of methyl oleate. After 9 hours of additional growth, 3 μL of a 50 mg/mL substrate feed (geraniol or 8-hydroxygeraniol) prepared in DMSO was dispensed into the cultures. Cells were grown for an additional 15 hours before assays were performed to assess cell growth and titer.

Cell density was determined using a spectrophotometer by measuring the absorbance of each well at 600 nm. 20 μL of culture was diluted into 180 μL of 175 mM sodium phosphate buffer, pH 7.0 in a clear-bottom plate. The plates were shaken for 25 sat 750 rpm immediately before being measured on a Tecan M1 000 spectrophotometer. A non-inoculated control well was included as a blank. 300 μL of ethyl acetate was added to the cultures. The plates were sealed with a PlateLoc Thermal Microplate Sealer and the plates were shaken for one min at 750 rpm. The plates were centrifuged and the ethyl acetate layer was collected and analyzed by liquid chromatography coupled to mass spectrometry (LC-MS). Target analytes were quantified against authentic standards.

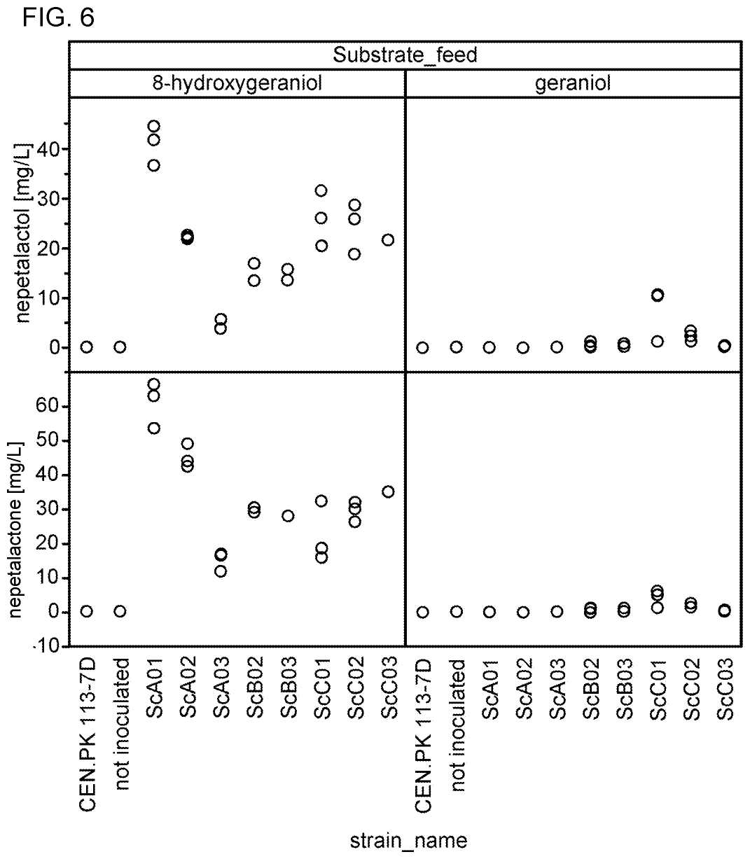

FIG. 6 displays the nepetalactone and nepetalactol titers of several engineered strains compared to non-inoculated control wells and the wild-type strain, CEN.PK113-7D. Table 10 shows the strain genotypes of engineered strains. Gene deletions are indicated by Δ. “iholl” indicates that the cassette has been integrated at a neutral locus, specifically, an intergenic region between HOL1 and a proximal gene.

TABLE 10

| strain | |
|---|---|
| name | genotype |
| ScA01 | Δadh6: prGAL1-10:RsNEPS, Nc8HGO; pGAL7:NmISY; pGAL1-10:Cc8HGO, NcNOR; URA3 |
| ScA02 | Δoye2: pGAL1-10:RsNEPS, Nc8HGO; pGAL7:NmISY; pGAL1-10:Cc8HGO, NcNOR; URA3 |
| ScA03 | ihol1: pGAL1-10:RsNEPS, Nc8HGO; pGAL7:NmISY; pGAL1-10:Cc8HGO, NcNOR; URA3 |
| ScB02 | ihol1: pGAL1-10:RsNEPS, Nc8HGO; pGAL7:NmISY; pGAL1-10:Cc8HGO, NcNOR |
| | Δprb1: ADE1; pGAL7:NmG8H; pGAL1-10:CrCYB5, CrCPR; URA3 |
| ScB03 | ihol1: pGAL1-10:RsNEPS, Nc8HGO: pGAL7:NmISY; pGAL1-10:Cc8HGO, NcNOR |
| | Δpep4: ADE1; pGAL7:NmG8H; pGAL1-10:CrCYB5, CrCPR; URA3 |
| ScC01 | ihol1: pGAL1-10:RsNEPS, Nc8HGO; pGAL7:NmISY: pGAL1-10:Cc8HGO, NcNOR |
| | Δprb1: ADE1; pGAL7:NmG8H; pGAL1-10:CrCYB5, CrCPR |
| | Δho: pGAL1-10:ObGES, ScERG20(WW); URA3 |
| ScC02 | ihol1: pGAL1-10:RsNEPS, Nc8HGO; pGAL7:NmISY; pGAL1-10:Cc8HGO, NcNOR |
| | Δprb1: ADE1; pGAL7:NmG8H; pGAL1-10:CrCYB5, CrCPR |
| | Δho: pGAL1-10:ObGES, ScERG20(WW); pGAL1:ScERG20(WW); URA3 |
| ScC03 | ihol1: pGAL1-10:RsNEPS, Nc8HGO; pGAL7:NmISY; pGAL1-10:Cc8HGO, NcNOR |
| | Δprb1: ADE1; pGAL7:NmG8H; pGAL1-10:CrCYB5, CrCPR |
| | Δho: pGAL1-10:ObGES, ScERG20(WW); pGAL1-10:ScERG20(WW), ObGES; URA3 |

Table 11 shows the gene names and their corresponding source organisms that were introduced into the engineered strains.

TABLE 11

| | | SEQ ID |
|---|---|---|
| gene name | source organism | NO. |
| ScERG20(WW) | Saccharomyces cerevisiae | 789 |
| ObGES | Ocimurn basilicum | 930 |
| NmG8H | Nepeta mussinn | 1054 |
| CrCPR | Catharanthus roseus | 1075 |
| CrCYB5 | Catharanthus roseus | 1114 |
| Nc8HGO | Nepeta cataria | 1120 |
| Cc8HGO | Coffea canephora | 1128 |
| NmISY | Nepeta mussinii | 1163 |
| RsNEPS | Rauvolfia serpentina | 1511 |
| NcNOR | Nepeta cataria | 1393 |

All engineered strains in FIG. 6 produced nepetalactone and nepetalactol with an 8-hydroxygeraniol feed with maximum titers of 66.7 mg/L nepetalactone and 44.4 mg/L nepetalactol. Under identical conditions, no nepetalactone and nepetalactol was observed in the non-inoculated control wells and the wild-type strain. Only some of the engineered strains produced the same products with a geraniol substrate feed; generally, the titers were lower with a geraniol substrate feed with maximum titers of 6.1 mg/L nepetalactone and 10.6 mg/L nepetalactol. With the geraniol substrate feed, no nepetalactone and nepetalactol was observed in wells that were noninoculated or that contained the wild-type strain. Only the cis, trans-nepetalactone isomer was produced.

### Example 9—Construction of a Complete Nepetalactone Biosynthetic Pathway in Yeast to Enable Production from Glucose

Strains were designed with the intent of producing nepetalactone from glucose as the primary carbon source. This was achieved by the overexpression of the native mevalonate pathway in addition to the biosynthetic genes required to convert IPP and DMAPP into nepetalactone.

The below strains were generated using the methods described above in Example 8. Briefly, DNA was designed as multiple pieces with overlaps for homologous recombination. Homology arms of length 250-500 bp were designed to target the DNA for insertion into the genome by double crossover homologous recombination. In some cases, integration results in deletion of a locus, and in other cases, integration occurs in an intergenic region. Transformations were plated on selection media depending on the marker that was used. Colonies were cultured in selection media and were screened by diagnostic PCR to verify successful integration.

For construction of Strain X1, DNA that was designed for the heterologous expression of ERG10, ERG13, tHMGR, ERG12, ERG8 and ERG19 at the TRP1 locus with KlURA3 as the selection marker was integrated into wild-type CEN.PK113-7D with the native URA3 cassette deleted. The KIURA3 cassette was flanked by direct repeats to enable counter-selection in the presence of 5-FOA. The integration deletes TRP1, enabling its use as a marker for the subsequent transformation.

For construction of Strain X2, DNA that was designed for the heterologous expression of ObGES, AgGPPS, tHMGR, ERG20(WW) and IDI1 at the LEU2 locus with CgTRP1 as the selection marker was integrated into Strain X1. The integration deletes LEU2, enabling its use as a marker for the subsequent transformation. ObGES and AgGPPS were fused to an N-terminal GB1 tag.

For construction of Strain X3, DNA that was designed for the heterologous expression of CrCPR, VaG8H, NmISY, CrG8H, AtCPR, and Cr8HGO at the OYE2 locus with CgLEU2 as the selection marker was transformed into Strain X2. NmISY and Cr8HGO were fused to a GB1 tag.

For construction of Strain X4, DNA that was designed for the heterologous expression of Ncat_NOR_34 at the OYE3 locus with KanMX as the selection marker was transformed into Strain X3. Ncat_NOR_34 was fused to a GB1 tag. The KlURA3 cassette integrated at the TRP1 locus was removed by counter-selection on 5-FOA to generate Strain X4 Δura3.

For construction of Strain X5, DNA that was designed for knockout of GAL1 with KIURA3 as the selection marker was transformed into Strain X4 Δura3. The KIURA3 cassette flanked by direct repeats and was removed by counter-selection on 5-FOA to generate Strain X5 Δura3.

For construction of Strain X6 (7000445150), DNA that was designed for the integration of NcNOR, Cl8HGO, OpISY, RsNEPS, and RsNEPS with KlURA3 as the selection marker was transformed into Strain X5 Δura3.

### Final Genotype of Strain X6 (7000445150):

Δtrp1: pGAL7-ERG10-tERG10, pGAL10-ERG13-tGAL10, pGAL1-tHMGR-tHMG1, scar, pGAL1-ERG12-tERG12, pGAL10-ERG8-tGAL10, pGAL7-ERG19-tERG19

Δleu2: pGAL10-GB1_ObGES-tLEU2, pGAL1-GB1_AgGPPS-tCYC1, CgTRP1, pGAL1-tHMGR-tHMG1, pGAL1-ERG20(WW)-tGAL10, pGAL7-IDI1-tiDI1

Δoye2: pGAL7-CrCPR-tSPO1, pGAL10-VaG8H-tGAL10, pGAL1-GB1_NmISY-tAIP, CgLEU2, pGAL1-CrG8H1-tTIP1, pGAL10-AtCPR-tGAL10, pGAL7-GB1_Cr8HGO-tTPS1

Δoye3: pGAL1-NOR_Ncat_34-tGRE3, KanMX

Δgal1: scar

Δadh6: pGAL10-NcNOR-tSPO1, pGAL1-Cl8HGO-tPHO5, KlURA3, pGAL7-OpISY-tPGK1, pGAL1-RsNEPS1-tCYC1, pGAL10-RsNEPS2-tADH1

TABLE 12

| | SEQ ID |
|---|---|
| gene name | NO. |
| ERG10 | 1826 |
| ERG13 | 1827 |
| tHMGR | 1828 |
| ERG12 | 1829 |
| ERG8 | 1830 |
| ERG19 | 1831 |
| GB1_ObGES | 1832 |
| GB1_AgGPPS | 1833 |
| ERG20(WW) | 1834 |
| IDI1 | 1835 |
| CrCPR | 1836 |
| VaG8H | 1837 |
| GB1_NmISY | 1838 |
| CrG8H1 | 1839 |
| AtCPR | 1840 |
| GB1_Cr8HGO | 1841 |
| GB1_NOR_Ncat_34 | 1842 |
| NcNOR | 1393 |
| Cl8HGO | 1126 |
| OpISY | 1175 |
| RsNEPS1 | 1515 |
| RsNEPS2 | 1511 |

### Example 10—Construction of an Improved Nepetalactone-Producing Strain by Targeted Engineering of the P450 Step

Improved nepetalactone-producing strains were generated by focused engineering of the cytochrome P450 complex. This engineering was intended to shift the distribution of geraniol-derived products, specifically from geranic acid to nepetalactol and nepetalactone.

For construction of Strain X7, DNA that was designed for the knockout of the KanMX marker by insertion of the KIURA3 cassette was transformed into Strain X5. The KIURA3 cassette was flanked by direct repeats, and was removed by counter-selection in the presence of 5-FOA to generate Strain X7 Δura3.

For construction of Strain X8, DNA that was designed for the heterologous expression of NcNOR, Cc8HGO, NmISY, Nc8HGO, RsNEPS2 with KlURA3 as the selection marker was transformed into Strain X7 Δura3.

For construction of Strain X9, DNA that was designed for the knock-out of KIURA3 with the KanMX marker as the selection marker was transformed into Strain X8.

For construction of Strain X10A (7000552966), DNA that was designed for the heterologous expression of NcG8H-CrCPR fusion, NcG8H, AtCPR, and AtCYBR with KlURA3 as the selection marker was transformed into Strain X9. For construction of Strain X10B (7000553262), DNA that was designed for the heterologous expression of CrG8H, NcG8H, CaCPR, CrCYB5, and NcCYBR with KIURA3 as the selection marker was transformed into Strain X9.

### Final Genotype of Strain X10A:

Δtrp1: pGAL7-ERG10-tERG10, pGAL10-ERG13-tGAL10, pGAL1-tHMGR-tHMG1, scar, pGAL1-ERG12-tERG12, pGAL10-ERG8-tGAL10, pGAL7-ERG19-tERG19

Δleu2: pGAL10-GB1_ObGES-tLEU2, pGAL1-GB1_AgGPPS-tCYC1, CgTRP1, pGAL1-tHMGR-tHMG1, pGAL1-ERG20(WW)-tGAL10, pGAL7-IDI1-tIDI1,

Δoye2: pGAL7-CrCPR-tSPO1, pGAL10-VaG8H-tGAL10, pGAL1-GB1_NmISY-tAIP, CgLEU2, pGAL1-CrG8H1-tTIP1, pGAL10-AtCPR-tGAL10, pGAL7-GB1_Cr8HGO-tTPS1

Δoye3: pGAL1-NOR_Ncat_34-tGRE3, scar

Δgal1: scar

Δadh6: pGAL10-NcNOR-tSPO1, pGAL1-Cc8HGO-tPHO5, KanMX, pGAL7-NmISY-tPGK1, pGAL1-Nc8HGO-tCYC1, pGAL10-RsNEPS2-tADH1

iMGA1: pGAL1-NcG8H_CrCPR-tADH1, pGAL10-NcG8H-tCYC1, pGAL3-AtCPR-tPGK1, KlURA3, pYEF3-AtCYBR-tSPO1

Final genotype of Strain X10B (7000553262) is identical to Strain X10A (7000552966) except for the following integration at iMGA1:

iMGA1: pGAL1-CrG8H2-tADH1, pGAL10-NcG8H-tCYC1, pGAL3-CaCPR-tPGK1, KlURA3, pPGK1-CrCYB5-tPHO5, pYEF3-NcCYBR-tSPO1

TABLE 13
Additional genes:

| | Nucleic | Amino |
|---|---|---|
| | acid SEQ | acid SEQ |
| gene name | ID NO. | ID NO. |
| Cc8HGO | 1128 | 340 |
| NmISY | 1163 | 375 |
| Nc8HGO | 1120 | 332 |
| RsNEPS2 | 1511 | 723 |
| NcG8H_CrCPR | 1421 | 633 |
| NcG8H | 1056 | 268 |
| AtCPR | 1078 | 290 |
| AtCYBR | 1573 | 785 |
| CrG8H2 | 1843 | 1825 |
| CaCPR | 1087 | 299 |
| CrCYB5 | 1114 | 326 |
| NcCYBR | 1572 | 784 |

### Example 11—Cloning and Expression of Dihydronepetalactone Dehydrogenases Capable of Converting Nepetalactone to Dihydronepetalactone (Prophetic)

Knockout libraries and overexpression libraries will be used to test whether there is a native enzyme that has the activity to convert nepetalactone to dihydronepetalactone in microbes, such as S. cereivisae. Another approach to identify dihydronepetalactone dehydrogenases involves identifying proteins predicted to be DND enzymes using BLAST. A MUSCLE protein alignment is performed with all the relevant DND sequences. HMMER was used to functionally annotate all predicted peptides based on their best matching Pfam hidden markov model (HMM) by E-value. All HMMs related to oxidoreductase activity were investigated further by BLAST and filtered to remove sequences with high sequence identity to any sequences from the non-redundant database to further narrow the list of candidates. The sequences of these candidates were codon-optimized for expression in S. cerevisiae and/or E. coli and were synthesized by a third party and cloned into an expression vector for characterization. The proteins predicted as being DND enzymes are tested for DND enzymatic activity of converting a nepetalactone substrate to dihydronepetalactone.

### Example 12—Control of Biosynthetic Pathway Expression by Various Repressors/Inducers in Saccharomyces cerevisiae (Prophetic)

To control expression of pathway genes, native and non-native promoters regulated by a repressor and/or inducer are used on a gene(s) within the pathway. In some cases regulated promoters are modified to use less or different repressors and/or inducers that are economical at scale. S. cerevisiae was engineered to contain the promoter and required regulatory genes to ensure tight controllable expression and therefore production of nepetalactol and/or its derivatives.

We find that due to the toxicity of intermediates, byproducts, and products of the downstream pathway, expression of a gene or multiple genes, controlled expression of a selected gene(s) by various repressors and/or inducers allows us to build up cell mass prior to production of toxic material and then express the required genes producing our desired toxic product at higher titers.

### Example 13—Gene Up- or Down-Regulation to Increase Production of Geraniol-Derived Terpenoids

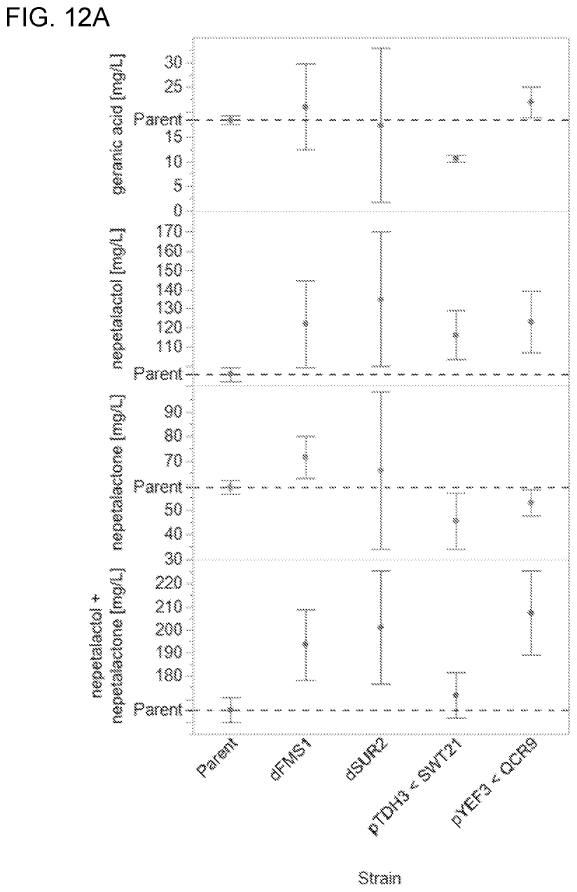

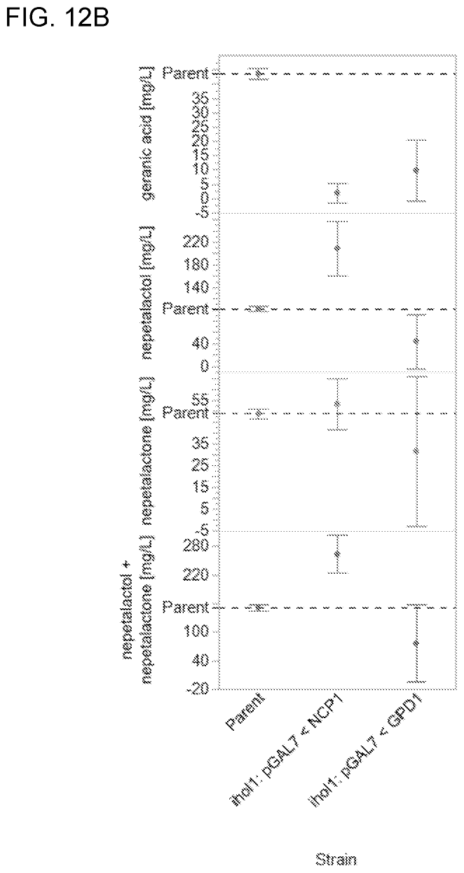

We found that upregulation, downregulation, or knock-out of specific genes, such as genes encoding oxidoreductases, within the host organism reduced byproduct accumulation (for example, geranic acid) or increased production of nepetalactol or nepetalactone. FIG. 12A shows the titers of geranic acid, nepetalactol and nepetalactone, and the combined titer of nepetalactol and nepetalactone in exemplary engineered strains compared to their parent strain, labeled as Parent. A complete gene deletion of FMS1 and SUR2 independently improved titers of nepetalactol over the parent strain. Deletion of FMS1 also improved nepetalactone titers over the parent strain. An insertion of the TDH3 promoter sequence between SWT21 and its native promoter reduced the levels of the by-product, geranic acid and increased nepetalactol titer compared to the parent strain, but decreased nepetalactone titer compared to the parent strain. An insertion of the YEF3 promoter sequence between QCR9 and its native promoter noticeably improved nepetalactol levels compared to the parent strain.

FIG. 12B shows the titers of geranic acid, nepetalactol and nepetalactone, and the combined titer of nepetalactol and nepetalactone in exemplary engineered strains compared to their parent strain, labeled as Parent. Note that the parent strain here is different from that shown in FIG. 12A. The insertion of a gene cassette containing the GAL7 promoter driving the expression of NCP1 at a neutral locus such as in intergenic region between HOL1 and a proximal gene, resulted in reduced geranic acid levels, and increased nepetalactol levels compared to the parent strain. The insertion of a gene cassette containing the GAL7 promoter driving the expression of GPD1 at the same neutral locus resulted in reduced geranic acid levels, but also had a negative effect on nepetalactol titers compared to the control.

The nucleic acid sequences of the genes, constructs and promoters used in these experiments are listed below in Table 14.

TABLE 14

| | SEQ ID |
|---|---|
| Sequence name | NO: |
| FMS1 | 1844 |
| SUR2 | 1845 |
| pTDH3 | 1846 |
| SWT21 | 1847 |
| pYEF3 | 1848 |
| QCR9 | 1849 |
| pGAL7 | 1850 |
| NCP1 | 1851 |
| GPD1 | 1852 |
| construct 1/2 for ihol1: pGAL7 < NCP1; | 1853 |
| plasmid 1/2 for ihol1: pGAL7 < GPD1 | |
| construct for pYEF3 < QCR9 | 1854 |
| construct for dFMS1 | 1855 |
| construct for pTHD3 < QCR9 | 1856 |
| construct for dSUR2 | 1857 |
| construct 2/2 for ihol1: pGAL7 < NCP1 | 1858 |
| construct 2/2 for ihol1: pGAL7 < GPD1 | 1859 |

These results show that alteration of the levels of certain gene products, such as oxidoreductases, can affect the levels of metabolites, such as nepetalactol and nepetalactone, produced. Therefore, modulation of oxidoreductases can result in the generation of microbial cells disclosed herein, which are capable of producing high yields of nepetalactol, nepetalactone and dihydronepetalactone.

Other genes in the host organism will similarly be upregulated or downregulated to test the effect on the production of geraniol, nepetalactol or nepetalactone. Potential target genes include, but are not limited to, the genes listed in Table 7. Upregulation or downregulation will be done by replacing the native promoter of the gene with one that is stronger or weaker, respectively. Modulation of gene expression will also be achieved by insertion of a terminator sequence followed by a stronger or weaker promoter in between the target gene and native promoter. For down-regulation, activity will be completely abolished by knocking-out the gene either partially or entirely. These manipulations will be performed by standard molecular biology methods where DNA is designed for double-crossover homologous recombination with the added insertion of a KIURA3 cassette or other marker for selection.

### Example 14—Production and Extraction of Geraniol-Derived Terpenoids Using Bi-Phasic Fermentation

Strains 7000445150 (see Example 9) and strains 7000552966 & 7000553262 (see Example 10) were grown using the biphasic fermentation process disclosed herein. Briefly, the fermentation conditions comprised of a temperature of 30 degrees C., pH of 5.0, dissolved oxygen of 30-50%, with a 10% methyl oleate as overlay and a glucose-limited fed-batch phase.

The first strain, 7000445150, accumulates >1.5 g/L of geranic acid, >0.5 g/L nepetalactone, and <0.1 g/L nepetalactol. After a subsequent round of engineering, the two additional strains, 7000552966 & 7000553262, show <0.25 g/L of geranic acid, and >1 g/L of both nepetalactol and nepetalactone. FIG. 12 shows a distribution of three geraniol-derived terpenoids, geranic acid, nepetalactol, and nepetalactone produced by these strains.

### Further Embodiments

Further embodiments contemplated by the disclosure are listed below:

Embodiment 1: A recombinant microbial cell capable of producing nepetalactol from a sugar substrate without additional precursor supplementation.

Embodiment 1.1: The recombinant microbial cell of embodiment 1, wherein the sugar substrate is selected from the group consisting of glucose, sucrose, maltose, and lactose.

Embodiment 1.2: The recombinant microbial cell of embodiment 1.1, wherein the sugar substrate is glucose.

Embodiment 2: The recombinant microbial cell of any one of the embodiments 1-1.2, wherein the recombinant microbial cell is capable of producing industrially relevant quantities of nepetalactol of greater than 1 gram per liter.

Embodiment 3: The recombinant microbial cell of any one of the embodiments 1-2, wherein the recombinant microbial cell comprises one or more polynucleotide(s) encoding each of the following heterologous enzymes: a geraniol diphosphate synthase (GPPS), a geranyl diphosphate diphosphatase (geraniol synthase, GES), a geraniol 8-hydroxylase (G8H), a cytochrome P450 reductase (CPR) capable of promoting regeneration of the redox state of the G8H, a cytochrome B5 (CYTB5) capable of promoting regeneration of the redox state of the G8H, an 8-hydroxygeraniol dehydrogenase (8HGO), an iridoid synthase (ISY), and a nepetalactol synthase (NEPS).

Embodiment 4: The recombinant microbial cell of embodiment 3, wherein the recombinant microbial cell is engineered to overexpress one or more enzymes from the mevalonate pathway selected from the group consisting of; acetyl-coA acetyltransferase (ERG10), hydroxymethylglutaryl-coA synthase (ERG13), HMG-CoA reductase (tHMG), mevalonate kinase (ERG12), phosphomevalonate kinase (ERG8), mevalonate decarboxylase (ERG19), and IPP isomerase (IDI).

Embodiment 4.1: The recombinant microbial cell of embodiment 4, wherein the tHMG is truncated to lack the membrane-binding region.

Embodiment 5: The recombinant microbial cell of embodiments 3-4.1, wherein the recombinant microbial cell is capable of producing industrially relevant quantities of nepetalactone of greater than 1 gram per liter, and wherein the recombinant microbial cell comprises a polynucleotide encoding for a nepetalactol oxidoreductase (NOR) heterologous enzyme.

Embodiment 6: The recombinant microbial cell of embodiments 3 or 4.1, wherein the recombinant microbial cell is capable of producing industrially relevant quantities of dihydronepetalactone of greater than 1 gram per liter, and wherein the recombinant microbial cell comprises one or more polynucleotides encoding each of the following heterologous enzymes: a nepetalactol oxidoreductase (NOR), and a dihydronepetalactone dehydrogenase (DND) capable of converting nepetalactone to dihydronepetalactone.

Embodiment 7: The recombinant microbial cell of any one of embodiments 3-6, wherein the polynucleotides encoding for heterologous enzymes are codon optimized for expression in the recombinant microbial cell.

Embodiment 8: The recombinant microbial cell of any one of embodiments 3-7, wherein the recombinant microbial cell is from a genus selected from the group consisting of: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomyces, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

Embodiment 9: The recombinant microbial cell of any one of embodiments 1-7, wherein the recombinant microbial cell is Saccharomyces cerevisiae.

Embodiment 10: The recombinant microbial cell of any one of embodiments 1-7, wherein the recombinant microbial cell is Escherichia coli.

Embodiment 11: A method for the production of nepetalactol from a sugar substrate, said method comprising: (a) providing a recombinant microbial cell according to any one of embodiments 1-10; and (b) cultivating the recombinant microbial cell in a suitable cultivation medium comprising the sugar substrate, thereby producing nepetalactol.

Embodiment 11.1: The method of embodiment 11, wherein the sugar substrate is selected from the group consisting of glucose, sucrose, maltose, and lactose.

Embodiment 11.2: The method of embodiment 11.1, wherein the sugar substrate is glucose.

Embodiment 12: A method for the production of nepetalactone from a sugar substrate, said method comprising: (a) providing a recombinant microbial cell according to any one of embodiments 5-10; and (b) cultivating the recombinant microbial cell in a suitable cultivation medium comprising the sugar substrate, thereby producing nepetalactone.

Embodiment 12.1: The method of embodiment 12, wherein the sugar substrate is selected from the group consisting of glucose, sucrose, maltose, and lactose.

Embodiment 12.2: The method of embodiment 12.1, wherein the sugar substrate is glucose.

Embodiment 13: A method for the production of dihydronepetalactone from a sugar substrate, said method comprising: (a) providing a recombinant microbial cell according to any one of embodiments 6-10; and (b) cultivating the recombinant microbial cell in a suitable cultivation medium comprising the sugar substrate, thereby producing dihydronepetalactone.

Embodiment 13.1: The method of claim 13, wherein the sugar substrate is selected from the group consisting of glucose, sucrose, maltose, and lactose.

Embodiment 13.2: The method of claim 13.1, wherein the sugar substrate is glucose.

Embodiment 14: A recombinant microbial cell capable of producing nepetalactone, wherein said recombinant microbial cell comprises a nucleic acid encoding for a heterologous nepetalactol oxidoreductase (NOR) enzyme that catalyzes the reduction of nepetalactol to nepetalactone.

Embodiment 14.1: The recombinant microbial cell of embodiment 14, wherein the NOR enzyme is also capable of catalyzing the cyclization of an enol intermediate to nepetalactol.

Embodiment 15: The recombinant microbial cell of embodiment 14 or 14.1, wherein the recombinant microbial cell is capable of producing industrially relevant quantities of nepetalactone of greater than 1 gram per liter.

Embodiment 16: The recombinant microbial cell of any one of embodiments 14-15, wherein the recombinant microbial cell comprises one or more polynucleotide(s) encoding one or more heterologous enzymes selected from the group consisting of: a geraniol diphosphate synthase (GPPS), a geranyl diphosphate diphosphatase (geraniol synthase, GES), a geraniol 8-hydroxylase (G8H), a cytochrome P450 reductase (CPR) capable of promoting regeneration of the redox state of the G8H, a cytochrome B5 (CYTB5) capable of promoting regeneration of the redox state of the G8H, an 8-hydroxygeraniol dehydrogenase (8HGO), an iridoid synthase (ISY), and a nepetalactol synthase (NEPS).

Embodiment 16.1: The recombinant microbial cell of any one of embodiments 14-15, wherein the recombinant microbial cell comprises a polynucleotide encoding a heterologous geraniol diphosphate synthase (GPPS).

Embodiment 16.2: The recombinant microbial cell of any one of embodiments 14-15, wherein the recombinant microbial cell comprises a polynucleotide encoding a heterologous geranyl diphosphate diphosphatase (geraniol synthase, GES).

Embodiment 16.3: The recombinant microbial cell of any one of embodiments 14-15, wherein the recombinant microbial cell comprises a polynucleotide encoding a heterologous geraniol 8-hydroxylase (G8H).

Embodiment 16.4: The recombinant microbial cell of any one of embodiments 14-15, wherein the recombinant microbial cell comprises a polynucleotide encoding a heterologous cytochrome P450 reductase (CPR) capable of promoting regeneration of the redox state of geraniol 8-hydroxylase.

Embodiment 16.5: The recombinant microbial cell of any one of embodiments 14-15, wherein the recombinant microbial cell comprises a polynucleotide encoding a heterologous cytochrome B5 (CYTB5) capable of promoting regeneration of the redox state of geraniol 8-hydroxylase.

Embodiment 16.6: The recombinant microbial cell of any one of embodiments 14-15, wherein the recombinant microbial cell comprises a polynucleotide encoding a heterologous 8-hydroxygeraniol dehydrogenase (8HGO).

Embodiment 16.7: The recombinant microbial cell of any one of embodiments 14-15, wherein the recombinant microbial cell comprises a polynucleotide encoding a heterologous iridoid synthase (ISY).

Embodiment 16.8: The recombinant microbial cell of any one of embodiments 14-15, wherein the recombinant microbial cell comprises a polynucleotide encoding a heterologous nepetalactol synthase (NEPS).

Embodiment 17: The recombinant microbial cell of any one of embodiments 14-16.8, wherein the recombinant microbial cell is engineered to overexpress one or more enzymes from the mevalonate pathway selected from the group consisting of: acetyl-coA acetyltransferase (ERG10), hydroxymethylglutaryl-coA synthase (ERG13), HMG-CoA reductase (tHMG), mevalonate kinase (ERG12), phosphomevalonate kinase (ERG8), mevalonate decarboxylase (ERG19), and IPP isomerase (IDI).

Embodiment 17.1: The recombinant microbial cell of any one of embodiments 14-16.8, wherein the recombinant microbial cell is engineered to overexpress acetyl-coA acetyltransferase (ERG10).

Embodiment 17.2: The recombinant microbial cell of any one of embodiments 14-16.8, wherein the recombinant microbial cell is engineered to overexpress hydroxymethylglutaryl-coA synthase (ERG13).

Embodiment 17.3: The recombinant microbial cell of any one of embodiments 14-16.8, wherein the recombinant microbial cell is engineered to overexpress HMG-CoA reductase (tHMG).

Embodiment 17.4: The recombinant microbial cell of embodiment 17.3, wherein the tHMG is truncated to lack the membrane-binding region.

Embodiment 17.5: The recombinant microbial cell of any one of embodiments 14-16.8, wherein the recombinant microbial cell is engineered to overexpress mevalonate kinase (ERG12).

Embodiment 17.6: The recombinant microbial cell of any one of embodiments 14-16.8, wherein the recombinant microbial cell is engineered to overexpress phosphomevalonate kinase (ERG8)

Embodiment 17.7: The recombinant microbial cell of any one of embodiments 14-16.8, wherein the recombinant microbial cell is engineered to overexpress mevalonate decarboxylase (ERG19).

Embodiment 17.8: The recombinant microbial cell of any one of embodiments 14-16.8, wherein the recombinant microbial cell is engineered to overexpress IPP isomerase (IDI).

Embodiment 18: A method for the production of nepetalactone, said method comprising: (a) providing a recombinant microbial cell according to any one of embodiments 14-17.8: (b) cultivating the recombinant microbial cell in a suitable cultivation medium; and (c) contacting the recombinant microbial cell with nepetalactol substrate to form nepetalactone.

Embodiment 19: A recombinant microbial cell capable of producing dihydronepetalactone, wherein said recombinant microbial cell comprises a nucleic acid encoding for a heterologous dihydronepetalactone dehydrogenase (DND) enzyme capable of converting nepetalactone to dihydronepetalactone.

Embodiment 20: The recombinant microbial cell of embodiment 19, wherein the recombinant microbial cell is capable of producing industrially relevant quantities of dihydronepetalactone of greater than 1 gram per liter.

Embodiment 21: The recombinant microbial cell of embodiment 19 or 20, wherein the recombinant microbial cell comprises one or more polynucleotide(s) encoding one or more heterologous enzymes selected from the group consisting of: a geraniol diphosphate synthase (GPPS), a geranyl diphosphate diphosphatase (geraniol synthase, GES), a geraniol 8-hydroxylase (G8H), a cytochrome P450 reductase (CPR) capable of promoting regeneration of the redox state of the G8H, a cytochrome B5 (CYTB5) capable of promoting regeneration of the redox state of the G8H, an 8-hydroxygeraniol dehydrogenase (8HGO), an iridoid synthase (ISY), a nepetalactol synthase (NEPS), and nepetalactol oxidoreductase (NOR).

Embodiment 21.1: The recombinant microbial cell of any one of embodiments 19-21, wherein the recombinant microbial cell comprises a polynucleotide encoding a heterologous geraniol diphosphate synthase (GPPS).

Embodiment 21.2: The recombinant microbial cell of any one of embodiments 19-21, wherein the recombinant microbial cell comprises a polynucleotide encoding a heterologous geranyl diphosphate diphosphatase (geraniol synthase, GES).

Embodiment 21.3: The recombinant microbial cell of any one of embodiments 19-21, wherein the recombinant microbial cell comprises a polynucleotide encoding a heterologous geraniol 8-hydroxylase (G8H).

Embodiment 21.4: The recombinant microbial cell of any one of embodiments 19-21, wherein the recombinant microbial cell comprises a polynucleotide encoding a heterologous cytochrome P450 reductase (CPR) capable of promoting regeneration of the redox state of geraniol 8-hydroxylase.

Embodiment 21.5: The recombinant microbial cell of any one of embodiments 19-21, wherein the recombinant microbial cell comprises a polynucleotide encoding a heterologous cytochrome B5 (CYTB5) capable of promoting regeneration of the redox state of geraniol 8-hydroxylase.

Embodiment 21.6: The recombinant microbial cell of any one of embodiments 19-21, wherein the recombinant microbial cell comprises a polynucleotide encoding a heterologous 8-hydroxygeraniol dehydrogenase (8HGO).

Embodiment 21.7: The recombinant microbial cell of any one of embodiments 19-21, wherein the recombinant microbial cell comprises a polynucleotide encoding a heterologous iridoid synthase (ISY).

Embodiment 21.8: The recombinant microbial cell of any one of embodiments 19-21, wherein the recombinant microbial cell comprises a polynucleotide encoding a heterologous nepetalactol synthase (NEPS).

Embodiment 21.9: The recombinant microbial cell of any one of embodiments 19-21, wherein the recombinant microbial cell comprises a polynucleotide encoding a heterologous nepetalactol oxidoreductase (NOR).

Embodiment 22: The recombinant microbial cell of any one of embodiments 19-21.9, wherein the recombinant microbial cell is engineered to overexpress one or more enzymes from the mevalonate pathway selected from the group consisting of; acetyl-coA acetyltransferase (ERG10), hydroxymethylglutaryl-coA synthase (ERG13), HMG-CoA reductase (tHMG), mevalonate kinase (ERG12), phosphomevalonate kinase (ERG8), mevalonate decarboxylase (ERG19), and IPP isomerase (IDI).

Embodiment 22.1: The recombinant microbial cell of any one of embodiments 19-22, wherein the recombinant microbial cell is engineered to overexpress acetyl-coA acetyltransferase (ERG10).

Embodiment 22.2: The recombinant microbial cell of any one of embodiments 19-22, wherein the recombinant microbial cell is engineered to overexpress hydroxymethylglutaryl-coA synthase (ERG13).

Embodiment 22.3: The recombinant microbial cell of any one of embodiments 19-22, wherein the recombinant microbial cell is engineered to overexpress HMG-CoA reductase (tHMG).

Embodiment 22.4: The recombinant microbial cell of embodiment 22.3, wherein the tHMG is truncated to lack the membrane-binding region.

Embodiment 22.5: The recombinant microbial cell of any one of embodiments 19-22, wherein the recombinant microbial cell is engineered to overexpress mevalonate kinase (ERG12).

Embodiment 22.6: The recombinant microbial cell of any one of embodiments 19-22, wherein the recombinant microbial cell is engineered to overexpress phosphomevalonate kinase (ERG8).

Embodiment 22.7: The recombinant microbial cell of any one of embodiments 19-22, wherein the recombinant microbial cell is engineered to overexpress mevalonate decarboxylase (ERG19).

Embodiment 22.8: The recombinant microbial cell of any one of embodiments 19-22, wherein the recombinant microbial cell is engineered to overexpress IPP isomerase (IDI).

Embodiment 23: A method for the production of dihydronepetalactone, said method comprising: (a) providing a recombinant microbial cell according to any one of embodiments 19-22.8; (b) cultivating the recombinant microbial cell in a suitable cultivation medium; and (c) contacting the recombinant microbial cell with nepetalactone substrate to form dihydronepetalactone.

Embodiment 24: A bioreactor for producing a desired product selected from the group consisting of nepetalactol, nepetalactone, and dihydronepetalactone, said bioreactor containing a composition comprising a first phase and a second phase, wherein the first phase is an aqueous phase comprising a microbial cell capable of synthesizing the product, and wherein the second phase comprises an organic solvent and at least a portion of the desired product synthesized by the microbial cell.

Embodiment 25: The bioreactor of embodiment 24, wherein the microbial cell is the recombinant microbial cell of any one of embodiments 1-10, 14-17.8, or 19-22.8.

Embodiment 26: The bioreactor of embodiment 24 or 25, wherein the organic solvent is selected from the group consisting of: corn oil, dodecane, hexadecane, oleyl alcohol, butyl oleate, dibutyl phthalate, dodecanol, dioctyl phthalate, farnesene, methyl oleate and isopropyl myristate.

Embodiment 27: The bioreactor of embodiment 24 or 25, wherein the organic solvent comprises one or more of olive oil, sesame oil, castor oil, cotton-seed oil, soybean oil, butane, pentane, heptane, octane, isooctane, nonane, decane, methyl oleate and terpene.

Embodiment 27.1 The bioreactor of embodiment 24 or 25, wherein the organic solvent is a polymer.

Embodiment 27.2 The bioreactor of embodiment 27.1, wherein the polymer is selected from the group consisting of PolyTHF, Hytrel, PT-series, and Pebax.

Embodiment 27.3: The bioreactor of embodiment 24 or 25, wherein the organic solvent comprises a polymer.

Embodiment 28: The bioreactor of any one of embodiments 25-27, wherein said bioreactor comprises a control mechanism configured to control at least one or more of pH, solvent, temperature, and dissolved oxygen.

Embodiment 29: A method for producing a desired product selected from the group consisting of nepetalactol, nepetalactone, and dihydronepetalactone, said method comprising the steps of: a) growing an aqueous culture of microbial cells configured to produce the desired product in response to a chemical inducer, in the absence of the chemical inducer; b) contacting the microbial cells with the chemical inducer; and c) adding an organic solvent to the induced aqueous culture, said organic solvent having low solubility with the aqueous culture, wherein product secreted by the microbial cells accumulates in the organic solvent, thereby reducing contact of the product with the microbial cells.

Embodiment 30: The method of embodiment 29, wherein the microbial cells comprise the recombinant microbial cell of any one of embodiments 1-10, 14-17.8, or 19-22.8.

Embodiment 31: The method of embodiment 29 or 30, wherein the organic solvent is selected from the group consisting of: corn oil, dodecane, hexadecane, oleyl alcohol, butyl oleate, dibutyl phthalate, dodecanol, dioctyl phthalate, farnesene, and isopropyl myristate.

Embodiment 32: The method of any one of embodiments 29-31, wherein the organic solvent comprises one or more of olive oil, sesame oil, castor oil, cotton-seed oil, soybean oil, butane, pentane, heptane, octane, isooctane, nonane, decane, and terpene.

Embodiment 32.1 The method of embodiment 29 or 30, wherein the organic solvent is a polymer.

Embodiment 32.2 The method of embodiment 32.1, wherein the polymer is selected from the group consisting of PolyTHF, Hytrel, PT-series, and Pebax.

Embodiment 32.3: The bioreactor of embodiment 29 or 30, wherein the organic solvent comprises a polymer.

Embodiment 33: The method of any one of embodiments 29-32, wherein the culture is a fed-batch culture.

Embodiment 34: The method of embodiment 33, wherein the organic solvent is added as part of a fed batch portion.

Embodiment 35: The method of any one of embodiments 29-34, comprising the step of: d) removing at least a portion of the organic solvent from the culture, thereby harvesting the desired product.

### Additional Embodiments

1. A recombinant microbial cell capable of producing nepetalactol from a microbial feedstock without additional nepetalactol precursor supplementation.
2. The recombinant microbial cell of embodiment 1, wherein the microbial feedstock comprises an carbon source selected from the group consisting of glucose, sucrose, maltose, lactose, glycerol, and ethanol.
3. The recombinant microbial cell of embodiment 2, wherein the carbon source is glucose.
4. The recombinant microbial cell of any one of embodiments 1-3, wherein the recombinant microbial cell comprises a polynucleotide encoding for a heterologous nepetalactol synthase (NEPS) enzyme.
5. The recombinant microbial cell of any one of embodiments 1-4, wherein the recombinant microbial cell comprises one or more polynucleotide(s) encoding each of the following heterologous enzymes: a geraniol diphosphate synthase (GPPS), a geranyl diphosphate diphosphatase (geraniol synthase, GES), a geraniol 8-hydroxylase (G8H), a cytochrome P450 reductase (CPR) capable of promoting regeneration of the redox state of the G8H, a cytochrome B5 (CYTB5) capable of promoting regeneration of the redox state of the G8H, an 8-hydroxygeraniol dehydrogenase (8HGO), an iridoid synthase (ISY), and a nepetalactol synthase (NEPS).
6. The recombinant microbial cell of any one of embodiments 4-5, wherein the heterologous NEPS enzyme exhibits at least 90%, 95%, 97%, or 100% sequence identity with an amino acid sequence selected from the group consisting of SEQ ID Nos 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, and 774.
7. The recombinant microbial cell of any one of embodiments 4-6, wherein the heterologous NEPS enzyme exhibits at least 90%, 95%, 97%, or 100% sequence identity with an amino acid sequence selected from the group consisting of SEQ ID Nos SEQ ID Nos 730, 731, 732, and 733.
8. The recombinant microbial cell of any one of embodiments 1-7, wherein the recombinant microbial cell is engineered to overexpress one or more enzymes from the mevalonate pathway selected from the group consisting of: acetyl-coA acetyltransferase (ERG10), hydroxymethylglutaryl-coA synthase (ERG13). HMG-CoA reductase (tHMG), mevalonate kinase (ERG12), phosphomevalonate kinase (ERG8), mevalonate decarboxylase (ERG19), and IPP isomerase (IDI).
9. The recombinant microbial cell of embodiment 8, wherein the tHMG is truncated to lack the membrane-binding region.
9.1 The recombinant microbial cell of any one of embodiments 1-9, wherein the recombinant microbial cell is capable of producing industrially relevant quantities of nepetalactol of greater than 400, 450, 500, 550, 600, 650, 700, 750, 800, 850, 900, 950, 1000, 1050, 1100, 1150, 1200, 1250, 1300, 1350, 1400, 1450, or 1500 micrograms of nepetalactol per liter of culture.
10. The recombinant microbial cell of any one of embodiments 1-9.1, wherein the recombinant microbial cell comprises a polynucleotide encoding for a nepetalactol oxidoreductase (NOR) heterologous enzyme.
11. The recombinant microbial cell of embodiment 10, wherein the recombinant microbial cell is capable of producing industrially relevant quantities of nepetalactone of greater than 400, 450, 500, 550, 600, 650, 700, 750, 800, 850, 900, 950, 1000, 1050, 1100, 1150, 1200, 1250, 1300, 1350, 1400, 1450, or 1500 micrograms of nepetalactone per liter of culture.
12. The recombinant microbial cell of any one of embodiments 10-11, wherein the NOR enzyme exhibits at least 90%, 95%, 97%, or 100% sequence identity with an amino acid sequence selected from the group consisting of SEQ ID Nos 520-607, 775-782 and 1642-1644.
13. The recombinant microbial cell of any one of embodiments 10-12, wherein the NOR enzyme exhibits at least 90%, 95%, 97%, or 100% sequence identity with of SEQ ID No 605.
14. The recombinant microbial cell of any one of embodiments 1-13 wherein the recombinant microbial cell comprises one or more polynucleotides encoding each of the following heterologous enzymes: a nepetalactol oxidoreductase (NOR), and a dihydronepetalactone dehydrogenase (DND) capable of converting nepetalactone to dihydronepetalactone.
15. The recombinant microbial cell of any one of embodiments 4-14, wherein the polynucleotides encoding for heterologous enzymes are codon optimized for expression in the recombinant microbial cell.
16. The recombinant microbial cell of any one of embodiments 1-15, wherein the recombinant microbial cell is from a genus selected from the group consisting of: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomyces, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.
17. The recombinant microbial cell of any one of embodiments 1-16, wherein the recombinant microbial cell is Saccharomyces cerevisiae.
18. The recombinant microbial cell of any one of embodiments 1-17, wherein the recombinant microbial cell is Escherichia coli.
19. The recombinant microbial cell of any one of embodiments 1-18, wherein the recombinant microbial cell expresses altered levels of an oxidoreductase, as compared to a wild type microbial cell.
20. The recombinant microbial cell of embodiment 19, wherein the oxidoreductase is encoded by a gene selected from OYE2, OYE3, ADH3, ALD4, BDH2, PUT2, SOR2, ALD3, ALD5, HFD1, UGA2, ADH5, ALD6, SFA1, MSC7, AYR1, SPS19, ALD2, PRO2, SOR1, ADH2, ADH1, HIS4, ZTA1, ETR1, AST1, YIM1, AST2, SDH2, CIR2, ARG5,6, HOM2, TDH1, TDH2, TDH3, AAD15, CYB2, DUS1, DUS3, ENV9, EPS1, FET5, FMS1, FRE1, FRE2, FRE3, FRE7, FRE8, GDH2, GIS1, GPX1, GRX1, GRX5, HEM14, HYR1, JHD1, JHD2, KGD1, LYS1, LYS9, MET8, MIS1, MTD1, NDI1, PDX3, POX1, PRX1, RNR4, RPH1, SCO1, SHH4, SOD1, SOD2, TRX3, TSA2, URA1, YMR31, COX13, COX4, COX5A, COX6, COX7, COX8, COX9, GCV1, GCV2, GCV3, GDH1, GDH3, GLT1, NDE1, NDE2, PDA1, QCR2, QCR6, QCR7, QCR8, RNR1, SDH4, TRX2, TYR1, ADH6, BDH1, XYL2, CAT5, ERG3, ERG4, ERG5, SCS7, GPD2, GRE2, IDH2, MDH1, GPD1, HMG1, HMG2, SER3, DLD1, DSF1, GRE3, MAE1, AAD10, AAD14, AAD4, ARA1, ARA2, GUT2, YPR1, ADH4, GCY1, ALO1, CYC2, GLR1, MET12, PUT1, SDH1, FRD1, MET5, OSM1, OYE2, OYE3, TRR2, YHB1, MCR1, CBR1, LPD1, MET10, MET13, PDB1, GAL80, PAN2, RAX2, SWT21, TDA3, AIM33, IRC15, TKL1, ADI1, ARR2, BNA1, BNA2, BNA4, COQ6, COX15, CTT1, CUP1-2, DFG10, DIT2, DLD2, DLD3, DOT5, DUS4, ERG24, ERV2, EUG1, FET3, FMO1, FRE4, FRE5, FRE6, FRM2, GPX2, GRX2, GRX3, GRX4, GRX6, GRX7, GRX8, GTT1, HBN1, HMX1, JLP1, LIA1, LOT6, MPD1, MPD2, MXR1, MXR2, RNR3, SCO2, FOX2, IFA38, OAR1, PAN5, ARI1, IRC24, ZWF1, IMD4, ARO1, GND1, GND2, HOM6, IMD3, LYS2, CBS2, AHP1, AIM14, CCP1, CTA1, CUP1-1, SMM1, SRX1, SUR2, TPA1, TRX1, TSA1, URE2, COX5B, MET16, QCR10, QCR9, ADE3, ARO2, COR1, COX12, IDP3, LYS12, MDH2, MDH3, SER33, IRE1, TKL2, IDH1, IDP1, IDP2, FDH1, GORI and NCP1.
21. The recombinant microbial cell of embodiment 19 or embodiment 20, wherein the oxidoreductase is encoded by a gene selected from FMS1, SUR2, SWT1, QCR9, NCP1 and GDP1.
22. The recombinant microbial cell of any one of embodiments 19-21, wherein the recombinant microbial cell comprises a deletion of a gene encoding the oxidoreductase.
23. The recombinant microbial cell of any one of embodiments 20-22, wherein the recombinant microbial cell comprises a mutation in a gene encoding the oxidoreductase.
24. The recombinant microbial cell of embodiment 23, wherein the mutation is an insertion, a deletion, a substitution of one or more amino acids in the coding and/or non-coding regions of the gene.
25. The recombinant microbial cell of any one of embodiments 19-24, wherein the recombinant microbial cell comprises a deletion of the gene encoding FMS1 oxidoreductase.
26. The recombinant microbial cell of any one of embodiments 19-25, wherein the recombinant microbial cell comprises a deletion of a gene encoding SUR2 oxidoreductase.
27. The recombinant microbial cell of any one of embodiments 19-26, wherein the recombinant microbial cell comprises a heterologous promoter operably linked to a gene encoding the oxidoreductase.
28. The recombinant microbial cell of embodiment 27, wherein the heterologous promoter is a weaker promoter, as compared to the native promoter of the gene encoding the oxidoreductase.
29. The recombinant microbial cell of embodiment 27 or 28, wherein the heterologous promoter is TDH3 or YEF3.
30. The recombinant microbial cell of any one of embodiments 19-29, wherein the recombinant microbial cell comprises TDH3 promoter operably linked to a gene encoding SWT1 oxidoreductase.
31. The recombinant microbial cell of any one of embodiments 19-30, wherein the recombinant microbial cell comprises YEF3 promoter operably linked to a gene encoding QCR9 oxidoreductase.
32. The recombinant microbial cell of any one of embodiments 19-31, wherein the recombinant microbial cell comprises an expression cassette comprising a gene encoding the oxidoreductase operatively linked to a promoter.
33. The recombinant microbial cell of any one of embodiments 19-32, wherein the recombinant microbial cell comprises an expression cassette comprising a gene encoding NCP1 oxidoreductase or GPD1 oxidoreductase operatively linked to GAL7 promoter.
34. A method for the production of nepetalactol from a sugar substrate, said method comprising: (a) providing a recombinant microbial cell according to any one of embodiments 1-33; and (b) cultivating the recombinant microbial cell in a suitable cultivation medium comprising the microbial feedstock, thereby producing nepetalactol.
35. The method of embodiment 34, wherein the sugar substrate is selected from the group consisting of glucose, sucrose, maltose, lactose, glycerol, and ethanol.
36. The method of embodiment 35, wherein the sugar substrate is glucose.
37. A method for the production of nepetalactone from a sugar substrate, said method comprising:
   (a) providing a recombinant microbial cell according to any one of embodiments 12-33; and
   (b) cultivating the recombinant microbial cell in a suitable cultivation medium comprising the microbial feedstock, thereby producing nepetalactone.
38. The method of embodiment 37, wherein the sugar substrate is selected from the group consisting of glucose, sucrose, maltose, lactose, glycerol, and ethanol.
39. The method of embodiment 38, wherein the sugar substrate is glucose.
40. A method for the production of dihydronepetalactone from a sugar substrate, said method comprising: (a) providing a recombinant microbial cell according to any one of embodiments 14-33; and (b) cultivating the recombinant microbial cell in a suitable cultivation medium comprising the microbial feedstock, thereby producing dihydronepetalactone.
41. The method of embodiment 40, wherein the sugar substrate is selected from the group consisting of glucose, sucrose, maltose, lactose, glycerol, and ethanol.
42. The method of embodiment 41, wherein the sugar substrate is glucose.
43. A recombinant microbial cell capable of producing nepetalactone, wherein said recombinant microbial cell comprises a nucleic acid encoding for a heterologous nepetalactol oxidoreductase (NOR) enzyme that catalyzes the reduction of nepetalactol to nepetalactone.
44. The recombinant microbial cell of embodiment 43, wherein the NOR enzyme is also capable of catalyzing the cyclization of an enol intermediate to nepetalactol.
45. The recombinant microbial cell of embodiment 43 or 44, wherein the recombinant microbial cell is capable of producing industrially relevant quantities of nepetalactone of greater than 400, 450, 500, 550, 600, 650, 700, 750, 800, 850, 900, 950, 1000, 1050, 1100, 1150, 1200, 1250, 1300, 1350, 1400, 1450, or 1500 micrograms of nepetalactone per liter of culture.
46. The recombinant microbial cell of any one of embodiments 43-45, wherein the recombinant microbial cell comprises one or more polynucleotide(s) encoding one or more heterologous enzymes selected from the group consisting of: a geraniol diphosphate synthase (GPPS), a geranyl diphosphate diphosphatase (geraniol synthase, GES), a geraniol 8-hydroxylase (G8H), a cytochrome P450 reductase (CPR) capable of promoting regeneration of the redox state of the G8H, a cytochrome B5 (CYTB5) capable of promoting regeneration of the redox state of the G8H, an 8-hydroxygeraniol dehydrogenase (8HGO), an iridoid synthase (ISY), and a nepetalactol synthase (NEPS).
47. The recombinant microbial cell of any one of embodiments 43-46, wherein the recombinant microbial cell comprises a polynucleotide encoding a heterologous geraniol diphosphate synthase (GPPS).
48. The recombinant microbial cell of any one of embodiments 43-47, wherein the recombinant microbial cell comprises a polynucleotide encoding a heterologous geranyl diphosphate diphosphatase (geraniol synthase, GES).
49. The recombinant microbial cell of any one of embodiments 43-48, wherein the recombinant microbial cell comprises a polynucleotide encoding a heterologous geraniol 8-hydroxylase (G8H).
50. The recombinant microbial cell of any one of embodiments 43-49, wherein the recombinant microbial cell comprises a polynucleotide encoding a heterologous cytochrome P450 reductase (CPR) capable of promoting regeneration of the redox state of geraniol 8-hydroxylase.
51. The recombinant microbial cell of any one of embodiments 43-50, wherein the recombinant microbial cell comprises a polynucleotide encoding a heterologous cytochrome B5 (CYTB5) capable of promoting regeneration of the redox state of geraniol 8-hydroxylase.
52. The recombinant microbial cell of any one of embodiments 43-51, wherein the recombinant microbial cell comprises a polynucleotide encoding a heterologous 8-hydroxygeraniol dehydrogenase (8HGO).
53. The recombinant microbial cell of any one of embodiments 43-52, wherein the recombinant microbial cell comprises a polynucleotide encoding a heterologous iridoid synthase (ISY).
54. The recombinant microbial cell of any one of embodiments 43-53, wherein the recombinant microbial cell comprises a polynucleotide encoding a heterologous nepetalactol synthase (NEPS).
55. The recombinant microbial cell of any one of embodiments 43-54, wherein the recombinant microbial cell is engineered to overexpress one or more enzymes from the mevalonate pathway selected from the group consisting of; acetyl-coA acetyltransferase (ERG10), hydroxymethylglutaryl-coA synthase (ERG13), HMG-CoA reductase (tHMG), mevalonate kinase (ERG12), phosphomevalonate kinase (ERG8), mevalonate decarboxylase (ERG19), and IPP isomerase (IDI).
56. The recombinant microbial cell of any one of embodiments 43-55, wherein the recombinant microbial cell is engineered to overexpress acetyl-coA acetyltransferase (ERG10).
57. The recombinant microbial cell of any one of embodiments 43-56, wherein the recombinant microbial cell is engineered to overexpress hydroxymethylglutaryl-coA synthase (ERG13).
58. The recombinant microbial cell of any one of embodiments 43-57, wherein the recombinant microbial cell is engineered to overexpress HMG-CoA reductase (tHMG).
59. The recombinant microbial cell of any one of embodiments 43-58, wherein the tHMG is truncated to lack the membrane-binding region.
60. The recombinant microbial cell of any one of embodiments 43-59, wherein the recombinant microbial cell is engineered to overexpress mevalonate kinase (ERG12).
61. The recombinant microbial cell of any one of embodiments 43-60, wherein the recombinant microbial cell is engineered to overexpress phosphomevalonate kinase (ERG8)
62. The recombinant microbial cell of any one of embodiments 43-61, wherein the recombinant microbial cell is engineered to overexpress mevalonate decarboxylase (ERG19).
63. The recombinant microbial cell of any one of embodiments 43-62, wherein the recombinant microbial cell is engineered to overexpress IPP isomerase (IDI).
64. A method for the production of nepetalactone, said method comprising: (a) providing a recombinant microbial cell according to any one of embodiments 43-63; (b) cultivating the recombinant microbial cell in a suitable cultivation medium; and (c) contacting the recombinant microbial cell with nepetalactol substrate to form nepetalactone.
65. A recombinant microbial cell capable of producing dihydronepetalactone, wherein said recombinant microbial cell comprises a nucleic acid encoding for a heterologous dihydronepetalactone dehydrogenase (DND) enzyme capable of converting nepetalactone to dihydronepetalactone.
66. The recombinant microbial cell of embodiment 65, wherein the recombinant microbial cell is capable of producing industrially relevant quantities of dihydronepetalactone of greater than 1 gram per liter.
67. The recombinant microbial cell of embodiment 65 or 66, wherein the recombinant microbial cell comprises one or more polynucleotide(s) encoding one or more heterologous enzymes selected from the group consisting of; a geraniol diphosphate synthase (GPPS), a geranyl diphosphate diphosphatase (geraniol synthase, GES), a geraniol 8-hydroxylase (G8H), a cytochrome P450 reductase (CPR) capable of promoting regeneration of the redox state of the G8H, a cytochrome B5 (CYTB5) capable of promoting regeneration of the redox state of the G8H, an 8-hydroxygeraniol dehydrogenase (8HGO), an iridoid synthase (ISY), a nepetalactol synthase (NEPS), and nepetalactol oxidoreductase (NOR).
68. The recombinant microbial cell of any one of embodiments 65-67, wherein the recombinant microbial cell comprises a polynucleotide encoding a heterologous geraniol diphosphate synthase (GPPS).
69. The recombinant microbial cell of any one of embodiments 65-68, wherein the recombinant microbial cell comprises a polynucleotide encoding a heterologous geranyl diphosphate diphosphatase (geraniol synthase, GES).
70. The recombinant microbial cell of any one of embodiments 65-69, wherein the recombinant microbial cell comprises a polynucleotide encoding a heterologous geraniol 8-hydroxylase (G8H).
71. The recombinant microbial cell of any one of embodiments 65-70, wherein the recombinant microbial cell comprises a polynucleotide encoding a heterologous cytochrome P450 reductase (CPR) capable of promoting regeneration of the redox state of geraniol 8-hydroxylase.
72. The recombinant microbial cell of any one of embodiments 65-71, wherein the recombinant microbial cell comprises a polynucleotide encoding a heterologous cytochrome B5 (CYTB5) capable of promoting regeneration of the redox state of geraniol 8-hydroxylase.
73. The recombinant microbial cell of any one of embodiments 65-72, wherein the recombinant microbial cell comprises a polynucleotide encoding a heterologous 8-hydroxygeraniol dehydrogenase (8HGO).
74. The recombinant microbial cell of any one of embodiments 65-73, wherein the recombinant microbial cell comprises a polynucleotide encoding a heterologous iridoid synthase (ISY).
75. The recombinant microbial cell of any one of embodiments 65-74, wherein the recombinant microbial cell comprises a polynucleotide encoding a heterologous nepetalactol synthase (NEPS).
76. The recombinant microbial cell of any one of embodiments 65-75, wherein the recombinant microbial cell comprises a polynucleotide encoding a heterologous nepetalactol oxidoreductase (NOR).
77. The recombinant microbial cell of any one of embodiments 65-76, wherein the recombinant microbial cell is engineered to overexpress one or more enzymes from the mevalonate pathway selected from the group consisting of: acetyl-coA acetyltransferase (ERG10), hydroxymethylglutaryl-coA synthase (ERG13), HMG-CoA reductase (tHMG), mevalonate kinase (ERG12), phosphomevalonate kinase (ERG8), mevalonate decarboxylase (ERG19), and IPP isomerase (IDI).
78. The recombinant microbial cell of any one of embodiments 65-77, wherein the recombinant microbial cell is engineered to overexpress acetyl-coA acetyltransferase (ERG10).
79. The recombinant microbial cell of any one of embodiments 65-78, wherein the recombinant microbial cell is engineered to overexpress hydroxymethylglutaryl-coA synthase (ERG13).
80. The recombinant microbial cell of any one of embodiments 65-79, wherein the recombinant microbial cell is engineered to overexpress HMG-CoA reductase (tHMG).
81. The recombinant microbial cell of embodiment 80, wherein the tHMG is truncated to lack the membrane-binding region.
82. The recombinant microbial cell of any one of embodiments 65-81, wherein the recombinant microbial cell is engineered to overexpress mevalonate kinase (ERG12).
83. The recombinant microbial cell of any one of embodiments 65-82, wherein the recombinant microbial cell is engineered to overexpress phosphomevalonate kinase (ERG8).
84. The recombinant microbial cell of any one of embodiments 65-83, wherein the recombinant microbial cell is engineered to overexpress mevalonate decarboxylase (ERG19).
85. The recombinant microbial cell of any one of embodiments 65-84, wherein the recombinant microbial cell is engineered to overexpress IPP isomerase (IDI).
86. A method for the production of dihydronepetalactone, said method comprising: (a) providing a recombinant microbial cell according to any one of embodiments 65-85; (b) cultivating the recombinant microbial cell in a suitable cultivation medium; and (c) contacting the recombinant microbial cell with nepetalactone substrate to form dihydronepetalactone.
87. A for producing a desired product selected from the group consisting of nepetalactol, nepetalactone, and dihydronepetalactone, said bioreactor containing a composition comprising a first phase and a second phase, wherein the first phase is an aqueous phase comprising a microbial cell capable of synthesizing the product, and wherein the second phase comprises an organic solvent and at least a portion of the desired product synthesized by the microbial cell.
88. The bioreactor of embodiment 87, wherein the microbial cell is the recombinant microbial cell of any one of embodiments 1-33, 43-63 and 65-85.
89. The bioreactor of embodiment 87 or 88, wherein the organic solvent is selected from the group consisting of: corn oil, dodecane, hexadecane, oleyl alcohol, butyl oleate, dibutyl phthalate, dodecanol, dioctyl phthalate, farnesene, methyl oleate, and isopropyl myristate.
90. The bioreactor of embodiment 87 or 88, wherein the organic solvent comprises one or more of olive oil, sesame oil, castor oil, cotton-seed oil, soybean oil, butane, pentane, heptane, octane, isooctane, nonane, decane, methyl oleate, and terpene.
91. The bioreactor of embodiment 87 or 88, wherein the organic solvent is a polymer.
92. The bioreactor of embodiment 91, wherein the polymer is selected from the group consisting of PolyTHF, Hytrel, PT-series, and Pebax.
93. The bioreactor of embodiment 87 or 88, wherein the organic solvent comprises a polymer.
94. The bioreactor of any one of embodiments 87-93, wherein said bioreactor comprises a control mechanism configured to control at least one or more of pH, solvent, temperature, and dissolved oxygen.
95. A method for producing a desired product selected from the group consisting of nepetalactol, nepetalactone, and dihydronepetalactone, said method comprising the steps of;
   a) growing an aqueous culture of microbial cells configured to produce the desired product in response to a chemical inducer/repressor, in the absence of the chemical inducer or presence of the chemical repressor;
   b) contacting the microbial cells with the chemical inducer and/or depletion of the repressor; and
   c) adding an organic solvent to the producing aqueous culture, said organic solvent having low solubility with the aqueous culture, wherein product secreted by the microbial cells accumulates in the organic solvent, thereby reducing contact of the product with the microbial cells.
96. The method of embodiment 95, wherein the organic solvent is added at the time the aqueous culture is grown.
97. The method of embodiment 95 or 96, wherein the microbial cells comprise the recombinant microbial cell of any one of embodiments 1-33, 43-63 and 65-85.
98. The method of any one of embodiments 95-97, wherein the organic solvent is selected from the group consisting of: corn oil, dodecane, hexadecane, oleyl alcohol, butyl oleate, dibutyl phthalate, dodecanol, dioctyl phthalate, farnesene, and isopropyl myristate.
99. The method of any one of embodiments 95-97, wherein the organic solvent comprises one or more of olive oil, sesame oil, castor oil, cotton-seed oil, soybean oil, butane, pentane, heptane, octane, isooctane, nonane, decane, and terpene.
100. The method of any one of embodiments 95-97, wherein the organic solvent is a polymer.
101. The method of embodiment 100, wherein the polymer is selected from the group consisting of PolyTHF, Hytrel, PT-series, and Pebax.
102. The method of any one of embodiments 95-97, wherein the organic solvent comprises a polymer.
103. The method of any one of embodiments 95-102, wherein the culture is a fed-batch culture.
104. The method of embodiment 95-103, wherein the organic solvent is added as part of a fed batch portion.
105. The method of any one of embodiments 95-104, comprising the step of: d) removing at least a portion of the organic solvent from the culture, thereby harvesting the desired product.
106. A recombinant microbial cell comprising a polynucleotide encoding for a heterologous nepetalactol synthase (NEPS) enzyme.
107. The recombinant microbial cell of any one of embodiment 106, wherein the heterologous NEPS enzyme exhibits at least 90%, 95%, 97%, or 100% sequence identity with an amino acid sequence selected from the group consisting of SEQ ID Nos 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, and 774.
108. The recombinant microbial cell of any one of embodiments 106-107, wherein the heterologous NEPS enzyme exhibits at least 90%, 95%, 97%, or 100% sequence identity with an amino acid sequence selected from the group consisting of SEQ ID Nos SEQ ID Nos 730, 731, 732, and 733.
109. The recombinant microbial cell of any one of embodiments 106-108, wherein the recombinant microbial cell comprises one or more polynucleotide(s) encoding each of the following heterologous enzymes: a geraniol diphosphate synthase (GPPS), a geranyl diphosphate diphosphatase (geraniol synthase, GES), a geraniol 8-hydroxylase (G8H), a cytochrome P450 reductase (CPR) capable of promoting regeneration of the redox state of the G8H, a cytochrome B5 (CYTB5) capable of promoting regeneration of the redox state of the G8H, an 8-hydroxygeraniol dehydrogenase (8HGO), an iridoid synthase (ISY).
110. A recombinant microbial cell comprising a polynucleotide encoding for a nepetalactol oxidoreductase (NOR) heterologous enzyme.
111. The recombinant microbial cell of embodiment 110, wherein the recombinant microbial cell is capable of producing industrially relevant quantities of nepetalactone of greater than 400, 450, 500, 550, 600, 650, 700, 750, 800, 850, 900, 950, 1000, 1050, 1100, 1150, 1200, 1250, 1300, 1350, 1400, 1450, or 1500 micrograms of nepetalactone per liter of culture.
112. The recombinant microbial cell of any one of embodiments 110-111, wherein the NOR enzyme exhibits at least 90%, 95%, 97%, or 100% sequence identity with an amino acid sequence selected from the group consisting of SEQ ID Nos 520-607, 775-782 and 1642-1644.
113. The recombinant microbial cell of any one of embodiments 110-112, wherein the NOR enzyme exhibits at least 90%, 95%, 97%, or 100% sequence identity with SEQ ID No 605.
114. A recombinant microbial cell capable of producing nepetalactol, wherein the recombinant microbial cell expresses altered levels of an oxidoreductase, as compared to a wild type microbial cell.
115. The recombinant microbial cell of embodiment 114, wherein the oxidoreductase is encoded by a gene selected from OYE2, OYE3, ADH3, ALD4, BDH2, PUT2, SOR2, ALD3, ALD5, HFD1, UGA2, ADH5, ALD6, SFA1, MSC7, AYR1, SPS19, ALD2, PRO2, SOR1, ADH2, ADH1, HIS4, ZTA1, ETR1, AST1, YIM1, AST2, SDH2, CIR2, ARG5,6, HOM2, TDH1, TDH2, TDH3, AAD15, CYB2, DUS1, DUS3, ENV9, EPS1, FET5, FMS1, FRE1, FRE2, FRE3, FRE7, FRE8, GDH2, GIS1, GPX1, GRX1, GRX5, HEM14, HYR1, JHD1, JHD2, KGD1, LYS1, LYS9, MET8, MIS1, MTD1, NDI1, PDX3, POX1, PRX1, RNR4, RPH1, SCO1, SHH4, SOD1, SOD2, TRX3, TSA2, URA1, YMR31, COX13, COX4, COX5A, COX6, COX7, COX8, COX9, GCV1, GCV2, GCV3, GDH1, GDH3, GLT1, NDE1, NDE2, PDA1, QCR2, QCR6, QCR7, QCR8, RNR1, SDH4, TRX2, TYR1, ADH6, BDH1, XYL2, CAT5, ERG3, ERG4, ERG5, SCS7, GPD2, GRE2, IDH2, MDH1, GPD1, HMG1, HMG2, SER3, DLD1, DSF1, GRE3, MAE1, AAD10, AAD14, AAD4, ARA1, ARA2, GUT2, YPR1, ADH4, GCY1, ALO1, CYC2, GLR1, MET12, PUT1, SDH1, FRD1, MET5, OSM1, OYE2, OYE3, TRR2, YHB1, MCR1, CBR1, LPD1, MET10, MET13, PDB1, GAL80, PAN2, RAX2, SWT21, TDA3, AIM33, IRC15, TKL1, ADI1, ARR2, BNA1, BNA2, BNA4, COQ6, COX15, CTT1, CUP1-2, DFG10, DIT2, DLD2, DLD3, DOT5, DUS4, ERG24, ERV2, EUG1, FET3, FMO1, FRE4, FRE5, FRE6, FRM2, GPX2, GRX2, GRX3, GRX4, GRX6, GRX7, GRX8, GTT1, HBN1, HMX1, JLP1, LIA1, LOT6, MPD1, MPD2, MXR1, MXR2, RNR3, SCO2, FOX2, IFA38, OAR1, PAN5, ARI1, IRC24, ZWF1, IMD4, ARO1, GND1, GND2, HOM6, IMD3, LYS2, CBS2, AHP1, AIM14, CCP1, CTA1, CUP1-1, SMM1, SRX1, SUR2, TPA1, TRX1, TSA1, URE2, COX5B, MET16, QCR10, QCR9, ADE3, ARO2, COR1, COX12, IDP3, LYS12, MDH2, MDH3, SER33, IRE1, TKL2, IDH1, IDP1, IDP2, FDH1, GORI and NCP1.
116. The recombinant microbial cell of embodiment 114 or embodiment 115, wherein the oxidoreductase is encoded by a gene selected from FMS1, SUR2, SWT1, QCR9, NCP1 and GDP1.
117. The recombinant microbial cell of any one of embodiments 114-116, wherein the recombinant microbial cell comprises a deletion of a gene encoding the oxidoreductase.
118. The recombinant microbial cell of any one of embodiments 114-117, wherein the recombinant microbial cell comprises a mutation in a gene encoding the oxidoreductase.
119. The recombinant microbial cell of embodiment 118, wherein the mutation is an insertion, a deletion, a substitution of one or more amino acids in the coding and/or non-coding regions of the gene.
120. The recombinant microbial cell of any one of embodiments 114-119, wherein the recombinant microbial cell comprises a deletion of a gene encoding FMS1 oxidoreductase.
121. The recombinant microbial cell of any one of embodiments 114-120, wherein the recombinant microbial cell comprises a deletion of a gene encoding SUR2 oxidoreductase.
122. The recombinant microbial cell of any one of embodiments 114-121, wherein the recombinant microbial cell comprises a heterologous promoter operably linked to a gene encoding the oxidoreductase.
123. The recombinant microbial cell of embodiment 122, wherein the heterologous promoter is a weaker promoter, as compared to the native promoter of the gene encoding the oxidoreductase.
124. The recombinant microbial cell of embodiment 122 or 123, wherein the heterologous promoter is TDH3 or YEF3.
125. The recombinant microbial cell of any one of embodiments 114-124, wherein the recombinant microbial cell comprises TDH3 promoter operably linked to a gene encoding SWT1 oxidoreductase.
126. The recombinant microbial cell of any one of embodiments 114-125, wherein the recombinant microbial cell comprises YEF3 promoter operably linked to a gene encoding QCR9 oxidoreductase.
127. The recombinant microbial cell of any one of embodiments 114-126, wherein the recombinant microbial cell comprises an expression cassette comprising a gene encoding the oxidoreductase operatively linked to a promoter.
128. The recombinant microbial cell of any one of embodiments 114-127, wherein the recombinant microbial cell comprises an expression cassette comprising a gene encoding NCP1 oxidoreductase or GPD1 oxidoreductase operatively linked to GAL7 promoter.
129. The recombinant microbial cell of any one of embodiments 114-128, wherein the recombinant microbial cell produces higher levels of nepetalactol and/or lower levels of geranic acid, as compared to a control recombinant cell, wherein the control recombinant cell has wild type levels of the oxidoreductase.
130. The recombinant microbial cell of any one of embodiments 114-129, wherein the recombinant microbial cell comprises a polynucleotide encoding a nepetalactol oxidoreductase (NOR) enzyme.
131. The recombinant microbial cell of embodiment 130, wherein the recombinant microbial cell produces one or more of the following: higher levels of nepetalactol, higher levels of nepetalactone, and lower levels of geranic acid, as compared to a control recombinant cell, wherein the control recombinant cell has wild type levels of the oxidoreductase.
132. The recombinant microbial cell of any one of embodiments 114-131, wherein the recombinant microbial cell comprises one or more polynucleotides encoding each of the following heterologous enzymes: a nepetalactol oxidoreductase (NOR), and a dihydronepatalactone dehydrogenase (DND) capable of converting nepetalactone to dihydronepetalactone.
133. The recombinant microbial cell of embodiment 132, wherein the recombinant microbial cell produces one or more of the following: higher levels of nepetalactol, higher levels of nepetalactone, higher levels of dihydronepetalactone, and lower levels of geranic acid, as compared to a control recombinant cell, wherein the control recombinant cell has wild type levels of the oxidoreductase.
134. A method of producing nepetalactol, said method comprising: (a) providing a recombinant microbial cell of any one of embodiments 114-133; and (b) cultivating the recombinant microbial cell in a suitable cultivation medium; (c) contacting the recombinant microbial cell with a nepetalactol precursor to form nepetalactol.
135. A method of producing nepetalactone, said method comprising: (a) providing a recombinant microbial cell of any one of embodiments 130-133; and (b) cultivating the recombinant microbial cell in a suitable cultivation medium; (c) contacting the recombinant microbial cell with a nepetalactone precursor to form nepetalactone.
136. A method of producing dihydronepetalactone, said method comprising: (a) providing a recombinant microbial cell of embodiment 132 or 133; and (b) cultivating the recombinant microbial cell in a suitable cultivation medium; (c) contacting the recombinant microbial cell with a dihydronepetalactone precursor to form dihydronepetalactone.
137. A method for the production of nepetalactol or nepetalactone, said method comprising: (a) providing a recombinant microbial cell according to any one of embodiments 1-136; (b) cultivating the recombinant microbial cell in a suitable cultivation medium; and (c) contacting the recombinant microbial cell with nepetalactol substrate to form nepetalactone.
138. A recombinant microbial cell comprising a nucleic acid encoding for an iridiod synthase (ISY) enzyme exhibiting at least 85%, 90%, 95%, 97%, or 100% sequence identity with any one of the ISY enzymes listed in FIG. 3 or 4 or Tables 6 or 8.
139. A recombinant microbial cell comprising a nucleic acid encoding for an 8-hydroxygeraniol (8HGO) enzyme exhibiting at least 85%, 90%, 95%, 97%, or 100% sequence identity with any one of the 8HGO enzymes listed in FIG. 5 or table 8.

### INCORPORATION BY REFERENCE

All references, articles, publications, patents, patent publications, and patent applications cited herein are incorporated by reference in their entireties for all purposes. International PCT application No. PCT/US2018/067333, filed on Dec. 21, 2018 is hereby incorporated by reference in its entirety for all purposes. U.S. provisional Application No. 62/609,272, filed on Dec. 21, 2017, U.S. Provisional Application 62/609,279, filed on Dec. 21, 2017, and U.S. Provisional Application 62/669,919, filed on May 10, 2018, are each hereby incorporated by reference in their entireties for all purposes. However, mention of any reference, article, publication, patent, patent publication, and patent application cited herein is not, and should not, be taken as an acknowledgment or any form of suggestion that they constitute valid prior art or form part of the common general knowledge in any country in the world.

1.-137. (canceled)

138. A recombinant microbial cell capable of producing nepetalactol, wherein the recombinant microbial cell expresses an altered level of an oxidoreductase, as compared to a wild type microbial cell, wherein the oxidoreductase is selected from FMS1, SUR2, SWT21, QCR9, and NCP1.

139. The recombinant microbial cell of claim 138, wherein the oxidoreductase comprises an amino acid sequence encoded by a nucleic acid sequence selected from the group consisting of SEQ ID No. 1844, 1845, 1847, 1849, and 1851.

140. The recombinant microbial cell of claim 138, wherein the recombinant microbial cell is capable of producing: (a) higher levels of nepetalactol, (b) lower levels of geranic acid, or (c) a combination thereof, as compared to a control microbial cell without the altered oxidoreductase level.

141. The recombinant microbial cell of claim 138, wherein the recombinant microbial cell is capable of producing nepetalactol at a level of at least about 0.10 g/L.

142. The recombinant microbial cell of claim 138, wherein the recombinant microbial cell comprises a heterologous nepetalactol synthase (NEPS) enzyme.

143. The recombinant microbial cell of claim 142, wherein the recombinant microbial cell comprises each of the following heterologous enzymes: a geraniol diphosphate synthase (GPPS), a geranyl diphosphate diphosphatase (geraniol synthase, GES), a geraniol 8-hydroxylase (G8H), a cytochrome P450 reductase (CPR) capable of promoting regeneration of the redox state of the G8H, a cytochrome 5 (CYTB5) capable of promoting regeneration of the redox state of the G8H, an 8-hydroxygeraniol dehydrogenase (8HGO), and an iridoid synthase (ISY).

144. The recombinant microbial cell of claim 142, wherein the heterologous NEPS enzyme has at least 90% sequence identity to an amino acid sequence selected from the group consisting of SEQ ID Nos. 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, and 774.

145. The recombinant microbial cell of claim 138, wherein the recombinant microbial cell is engineered to overexpress one or more enzymes from the mevalonate pathway selected from the group consisting of: acetyl-coA acetyltransferase (ERG10), hydroxymethyglutarylcoA synthase (ERG13), HMG-CoA reductase (tHMG), mevalonate kinase (ERG12), phosphomevalonate kinase (ERG8), mevalonate decarboxylase (ERG19), and IPP isomerase (IDI), as compared to a wild type microbial cell.

146. The recombinant microbial cell of claim 138, wherein the recombinant microbial cell comprises a heterologous nepetalactol oxidoreductase (NOR) enzyme.

147. The recombinant microbial cell of claim 146, wherein the NOR enzyme has at least 90% sequence identity to an amino acid sequence selected from the group consisting of SEQ ID Nos. 520-607, 775-782 and 1642-1644.

148. The recombinant microbial cell of claim 146, wherein the recombinant microbial cell is capable of producing one or more of the following: (a) higher levels of nepetalactone, (b) higher levels of nepetalactol, and (c) lower levels of geranic acid, as compared to a control microbial cell, wherein the control microbial cell has wild type levels of the oxidoreductase.

149. The recombinant microbial cell of claim 138, wherein the recombinant microbial cell expresses a reduced level of the oxidoreductase, as compared to the wild type microbial cell.

150. The recombinant microbial cell of claim 149, wherein the recombinant microbial cell comprises a deletion of the oxidoreductase encoding gene.

151. The recombinant microbial cell of claim 150, wherein oxidoreductase is FMS1 or SUR2.

152. The recombinant microbial cell of claim 149, wherein the recombinant microbial cell comprises a heterologous promoter expressing the oxidoreductase, wherein the heterologous promoter is a weaker promoter, as compared to the native promoter of the gene encoding the oxidoreductase.

153. The recombinant microbial cell of claim 152, wherein the weaker promoter is a TDH3 promoter or a YEF3 promoter.

154. The recombinant microbial cell of claim 153, wherein the recombinant microbial cell comprises: (a) the TDH3 promoter expressing SWT21, or (b) the YEF3 promoter expressing QCR9.

155. The recombinant microbial cell of claim 138, wherein the recombinant microbial cell expresses an increased level of the oxidoreductase, as compared to the wild type microbial cell.

156. The recombinant microbial cell of claim 155, wherein the recombinant microbial cell comprises a heterologous promoter expressing the oxidoreductase, wherein the heterologous promoter is a stronger promoter, as compared to the native promoter of the gene encoding the oxidoreductase.

157. The recombinant microbial cell of claim 156, wherein the stronger promoter is a GAL7 promoter.

158. The recombinant microbial cell of claim 157, wherein the recombinant microbial cell comprises the GAL7 promoter expressing NCP1.

159. The recombinant microbial cell of claim 138, wherein the recombinant microbial cell belongs to a genus selected from the group consisting of: Agrobacterium, Alicyclobaeilius, Anabaena, Anacystis, Acmetobacter, Acidothermus, Arthrobacter, Azobacter, Bacillus, Bifidobacterium, Brevibaeierium, Bulynvibrio, Buchnera, Campestns, Camplyobacter, Clostridium, Corynebacterium, Chromatium, Coprococcus, Escherichia, Enterococcus, Enterobacter, Erwmia, Fusobacterium, Faeealibacterium, Francisella, Flavobacterium, Geobacillus, Haemophilus, Helicobacter, Klebsiella, Lactobacillus, Lactcoccus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomyces, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

160. The recombinant microbial cell of claim 138, wherein the recombinant microbial cell is Saccharomyces cerevisiae.

161. A method of producing nepetalactol, comprising: (a) providing a recombinant microbial cell of claim 138; (b) cultivating the recombinant microbial cell in a cultivation medium capable of supporting growth of the recombinant microbial cell; and (c) contacting the recombinant microbial cell with a nepetalactol precursor to form nepetalactol.

162. A method of producing nepetalactone, comprising: (a) providing a recombinant microbial cell of claim 146; (b) cultivating the recombinant microbial cell in a cultivation medium capable of supporting growth of the recombinant microbial cell; and (c) contacting the recombinant microbial cell with a nepetalactone precursor to form nepetalactone.
